(19) United States
(12) Patent Application Publication
(10) Pub. No.: US 2006/0160794 A1
(43) Pub. Date: Jul. 20, 2006

(54) Tachykinin receptor antagonists

(76) Inventors: Albert Kudzovi Amegadzie, Indianapolis, IN (US); Kevin Matthew Gardinier, Indianapolis, IN (US); Erik James Hembre, Indianapolis, IN (US); Philip Arthur Hipskind, New Palestine, IN (US); Louis Nickolaus Jungheim, Indianapolis, IN (US); Brian Stephen Muehl, Greenwood, IN (US); Kenneth Allen Savin, Indianapolis, IN (US); Keneth Jeff Thrasher, Indianapolis, IN (US); Steven A Boyd, Longmont, CO (US)

Correspondence Address: ELI LILLY & COMPANY, PATENT DIVISION, P.O. BOX 6288, INDIANAPOLIS, IN, 46206-6288, US

(21) Appl. No.: 10/559,852

(22) PCT Filed: Jun. 3, 2004
(86) PCT No.: PCT/US04/15579

§ 371 Date: Dec. 7, 2005

Related U.S. Application Data
(60) Provisional application No. 60/477,885, filed on Jun. 12, 2003.

(51) Int. Cl. A61K 31/541; A61K 31/5377; A61K 31/496; A61K 31/454; A61K 31/4439; A61K 31/4192; A61K 31/4172; C07D 403/02; C07D 413/02; C07D 417/02

(52) U.S. Cl. 514/227.5; 514/235.5; 514/254.05; 514/326; 514/341; 514/359; 514/400; 544/60; 544/139; 544/370; 546/209; 546/268.4; 546/272.7; 548/261; 548/335.5

(57) ABSTRACT

The present invention relates to selective NK-1 receptor antagonists of Formula (I) or a pharmaceutically acceptable salt thereof, for the treatment of disorders associated with an excess of tachykinins.

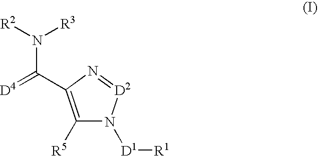

The present invention provides compounds of Formula (I), compositions thereof, and a method of antagonizing the NK-1 subtype of tachykinin receptor that comprises administering to a patient in need thereof an effective amount of a compound of Formula (I). In addition, the present invention relates to processes for preparing the compounds of Formula I and intermediates thereof.

Tachykinins are a family of peptides that are widely distributed in both the central and peripheral nervous systems. These peptides exert a number of biological effects through actions at tachykinin receptors. To date, three such receptors have been characterized, including the NK-1, NK-2, and NK-3 subtypes of tachykinin receptor.

The role of the NK-1 receptor subtype in numerous disorders of the central nervous system and the periphery has been thoroughly demonstrated in the art. For instance, NK-1 receptors are believed to play a role in depression, anxiety, and central regulation of various autonomic, as well as cardiovascular and respiratory functions. NK-1 receptors in the spinal cord are believed to play a role in pain transmission, especially the pain associated with migraine and arthritis. In the periphery, NK-1 receptor activation has been implicated in numerous disorders, including various inflammatory disorders, asthma, and disorders of the gastrointestinal and genitourinary tract.

There is an increasingly wide recognition that selective NK-1 receptor antagonists would prove useful in the treatment of many diseases of the central nervous system and the periphery. While many of these disorders are being treated by new medicines, there are still many shortcomings associated with existing treatments. For example, the newest class of anti-depressants, selective serotonin reuptake inhibitors (SSRIs), are increasingly prescribed for the treatment of depression; however, SSRIs have numerous side effects, including nausea, insomnia, anxiety, and sexual dysfunction. This could significantly affect patient compliance rate. As another example, current treatments for chemotherapy-induced nausea and emesis, such as the 5-HT₃ receptor antagonists, are ineffective in managing delayed emesis. The development of NK-1 receptor antagonists will therefore greatly enhance the ability to treat such disorders more effectively. Thus, the present invention provides a class of potent, non-peptide NK-1 receptor antagonists, compositions comprising these compounds, and methods of using the compounds.

The present invention provides compounds of Formula (I): 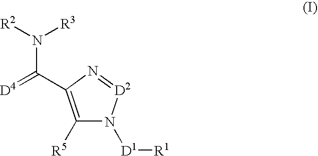
wherein:
D¹ is a C₁-C₃ alkane-diyl;
D² is CH or nitrogen;
D⁴ is oxygen or sulfur;
R¹ is phenyl,
   which phenyl is optionally substituted with one to three substitutents independently selected from the group consisting of halo, C₁-C₄ alkyl, C₁-C₄ alkoxy, cyano, difluoromethyl, trifluoromethyl, and trifluoromethoxy;
R² is selected from the group consisting of hydroxy, C₁-C₄ alkyl, optionally substituted phenyl, naphthyl, C₃-C₁₀ cycloalkyl, pyridyl, optionally substituted pyrrolidinyl, optionally substituted piperidinyl,
   which C₁-C₄ alkyl is optionally substituted with hydroxy, C₁-C₂ alkoxy, optionally substituted phenyl, pyridyl, —NR⁶R⁷, or naphthyl;
      which pyridyl is further optionally substituted with one to two halo, C₁-C₃ alkyl;

R³ is C₁-C₄ alkyl, optionally substituted phenyl, —C(O)—R⁴, or —S(O)₂—R⁴,
which C₁-C₄ alkyl is further optionally substituted with R⁴;
R⁴ is optionally substituted phenyl;
or R² and R³, together with the nitrogen to which they are attached, form a 4-11 membered heterocyclic ring,

which heterocyclic ring is further optionally substituted with one to four substituents independently selected from the group consisting of optionally substituted phenyl, C₃-C₆ cycloalkyl, pyridyl, halo, hydroxy, oxo, and C₁-C₄ alkyl;
wherein the C₁-C₄ alkyl is further optionally substituted with one to two substituents selected from the group consisting of C₁-C₃ alkoxy, optionally substituted phenyl, oxo, phenoxy, pyridyl, and pyrrolidinyl;
R⁶ and R⁷ are each independently hydrogen, C₁-C₄ alkyl, —S(O)₂—CH₃, or C₁-C₄ alkoxycarbonyl, or R⁶ and R⁷, together with the nitrogen to which they are attached, form a 4-7 membered saturated heterocyclic ring;
R⁵ is hydrogen, halo, trifluoromethyl, C₁-C₄ alkyl, C₁-C₄ alkoxy, C₃-C₆ cycloalkyl, furyl, pyrazolyl, imidazolyl, —NR¹³R¹⁴, pyridyloxy, benzyloxy, phenyl, phenoxy, pyrrolyl, thienyl, phenylthio, or anilino,
   which phenyl, phenoxy, pyrrolyl, thienyl, phenylthio, or anilino group may be optionally substituted on the ring with one to two substituents independently selected from the group consisting of halo, C₁-C₄ alkyl, C₁-C₄ alkoxy, trifluoromethyl, and —S(O)_{q}(C₁-C₄ alkyl),
or R⁵ is a radical selected from the group consisting of: 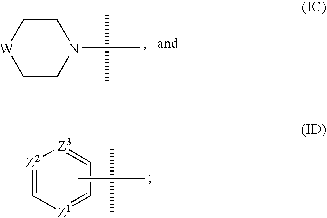
   wherein
W is a bond, —CHR¹⁵—, —C(O)—, —O—, —NR¹⁵—, or —S(O)_{q}—;
   q is 0, 1, or 2;
   R¹⁵ is selected from the group consisting of hydrogen, hydroxy, C₁-C₄ alkyl, acetyl, carbamoyl, phenyl, benzyl, and —S(O)₂CH₃;
Z¹, Z², and Z³ are each independently CH or nitrogen;
R¹³ and R¹⁴ are each independently hydrogen, C₁-C₄ alkyl, —S(O)₂—CH₃ or C₃-C₆ cycloalkyl;
   wherein the C₁-C₄ alkyl is optionally substituted with one C₁-C₂ alkoxy or di(C₁-C₂ alkyl)amino;
or R¹³ and R¹⁴, together with the nitrogen to which they are attached, form a 4-7 membered saturated heterocyclic ring;
   which 4-7 membered saturated heterocyclic ring is further optionally substituted with one to two C₁-C₂ alkyl;
or a pharmaceutically acceptable salt thereof;
with the proviso that the following compounds are not claimed:
[5-methyl-1-(3-pyrrolidin-1-ylpropyl)-1H-1,2,3-triazol-4-yl]piperazin-1-yl-methanone; {1-[2-(4-nitrophenyl)ethyl]-5-methyl-1H-1,2,3-triazol-4-yl }piperazin-1-yl-methanone; [1-(4-methoxybenzyl)-5-methyl-1H-1,2,3-triazol-4-yl]piperazin-1-yl-methanone; [5-methyl-1-(3-imidazol-1-ylpropyl)-1H-1,2,3-triazol-4-yl]piperazin-1-yl-methanone; (5-methyl-1-benzyl-1H- 1,2,3-triazol-4-yl)piperazin-1-yl-methanone; (1-benzyl-5-methyl-1H-1,2,3-triazol-4-yl)-1,4-diazepan-1-yl-methanone;
[1-(3,5-bis-trifluoromethyl-benzyl)-5-morpholin-4-yl-1H-[1,2,3]triazol-4-yl]-morpholin-4-yl-methanone; 1-(3,5-bis-trifluoromethyl-benzyl)-5-pyridin-4-yl-1H-[1,2,3]triazole-4-carboxylic acid (2-amino-ethyl)-(2-chloro-benzyl)-amide dihydrochloride; 1-(3,5-bis-trifluoromethyl-benzyl)-5-morpholin-4-yl-1H-[1,2,3]triazole-4-carboxylic acid (2-amino-ethyl)-(2-chloro-benzyl)-amide hydrochloride; 1-(3,5-bis-trifluoromethyl-benzyl)-5-morpholin-4-yl-1H-[1,2,3]triazole-4-carboxylic acid (2-amino-ethyl)-[1-(2-chlorophenyl)-ethyl)-amide dihydrochloride; 1-(3,5-bis-trifluoromethyl-benzyl)-5-pyridyl-4-yl-1H-[1,2,3]triazole-4-carboxylic acid (2-amino-ethyl)-[1-(2-chloro-phenyl)-ethyl]-amide dihydrochloride;
{2-[[1-(3,5-bis-trifluoromethyl-benzyl)-5-pyridin-4-yl-1H-[1,2,3]triazole-4-carbonyl]-(2-chloro-benzyl)-amino]-ethyl}-carbamic acid tert-butyl ester; {2-[[1-(3,5-bis-trifluoromethyl-benzyl)-5-chloro-1H-[1,2,3]triazole-4-carbonyl]-(2-chloro-benzyl)-amino]-ethyl}-carbamic acid tert-butyl ester; (2-{[1-(3,5-bis-trifluoromethyl-benzyl)-5-chloro-1H-[1,2,3]triazole-4-carbonyl]-[1-(2-chloro-phenyl)-ethyl]-amino}-ethyl)-carbamic acid tert-butyl ester; (2-{[1-(3,5-bis-trifluoromethyl-benzyl)-5-pyridin-4-yl-1H-[1,2,3]triazole-4-carbonyl]-[1-(2-chloro-phenyl)-ethyl]-amino}-ethyl)-carbamic acid tert-butyl ester; {2-[[1-(3,5-bis-trifluoromethyl-benzyl)-5-morpholin-4-yl-1H-[1,2,3]triazole-4-carbonyl]-(2-chloro-benzyl)-amino]-ethyl}-carbamic acid tert-butyl ester; and (2-{[1-(3,5-bis-trifluoromethyl-benzyl)-5-morpholin-4-yl -1H-[1,2,3]triazole-4-carbonyl]-[1-(2-chloro-phenyl)-ethyl]-amino}-ethyl)-carbamic acid tert-butyl ester.

The compounds of Formula I are antagonists of tachykinin receptors. Specifically, the compounds of Formula I are antagonists of the NK-1 subtype of tachykinin receptor. Because these compounds inhibit the physiological effects associated with an excess of tachykinins, the compounds are useful in the treatment of numerous disorders related to tachykinin receptor activation. These disorders include: anxiety, depression, psychosis, and schizophrenia and other psychotic disorders; neurodegenerative disorders such as dementia, including senile dementia of the Alzheimer's type, Alzheimer's disease, AIDS-associated dementia, and Down's syndrome; seizure disorders, such as epilepsy; demyelinating diseases such as multiple sclerosis and amyotrophic lateral sclerosis and other neuropathological disorders, such as peripheral neuropathy, diabetic and chemotherapy-induced neuropathy, and post-herpetic and other neuralgias; acute and chronic obstructive airway diseases such as adult respiratory distress syndrome, bronchopneumonia, bronchospasm, chronic bronchitis, drivercough, and asthma; inflammatory diseases such as inflammatory bowel disease, psoriasis, fibrositis, osteoarthritis, and rheumatoid arthritis; disorders of the musculo-skeletal system, such as osteoporosis; allergies such as eczema and rhinitis; hypersensitivity disorders such as poison ivy; ophthalmic diseases such as conjunctivitis, vernal conjunctivitis, and the like; cutaneous diseases such as contact dermatitis, atopic dermatitis, urticaria, and other eczematoid dermatites; addiction disorders such as alcoholism; stress-related somatic disorders; reflex sympathetic dystrophy such as shoulder/hand syndrome; dysthymic disorders; adverse immunological reactions such as rejection of transplanted tissues and disorders related to immune enhancement or suppression such as systemic lupus erythematosis; gastrointestinal disorders or diseases associated with the neuronal control of viscera such as ulcerative colitis, Crohn's disease and irritable bowel syndrome; disorders of bladder function such as bladder detrusor hyper-reflexia and incontinence; atherosclerosis; fibrosin and collagen diseases such as scleroderma and eosinophilic fascioliasis; irritative symptoms of benign prostatic hypertrophy; disorders associated with blood pressure, such as hypertension; or disorders of blood flow caused by vasodilation and vasospastic diseases, such as angina, migraine, and Reynaud's disease; emesis, including chemotherapy-induced nausea and emesis; and pain or nociception, for example, that attributable to or associated with any of the foregoing conditions.

In one embodiment, this invention provides a pharmaceutical composition comprising, as an active ingredient, a compound of Formula I, or a pharmaceutically acceptable salt thereof, in combination with one or more pharmaceutically acceptable carriers, diluents, or excipients.

In a further embodiment, the present invention relates to a method of making a compound represented by Formula I, and intermediates thereof.

In another embodiment, the present invention provides a method of selectively antagonizing an NK-1 receptor by contacting the receptor with a compound of Formula I, or a pharmaceutically acceptable salt thereof.

In another embodiment, this invention provides methods of treating a condition associated with an excess of tachykinins, comprising: administering to a patient in need thereof an effective amount of a compound of Formula I, or a pharmaceutically acceptable salt thereof. That is, the present invention provides for the use of a compound of Formula I, or a pharmaceutical composition thereof, for the treatment of a disorder associated with an excess of tachykinins.

In another aspect, the present invention provides for the use of a compound of Formula I, or a pharmaceutically acceptable salt thereof, in the manufacture of a medicament for antagonizing the NK-1 receptor. Thus, the present invention provides for the use of a compound of Formula I, or a pharmaceutically acceptable salt thereof, in the manufacture of a medicament for the treatment of a disorder associated with an excess of tachykinins by means of the method described above.

Of the disorders listed above, depression, anxiety, schizophrenia and other psychotic disorders, emesis, pain, asthma, inflammatory bowel disease, irritable bowel syndrome, and dermatitis are of importance. Of these disorders, depression and anxiety are of particular importance.

Thus, in a preferred embodiment, the present invention provides a method for treating major depressive disorder, comprising: administering to a patient in need thereof an effective amount of a compound of Formula I, or a pharmaceutically acceptable salt thereof.

In another preferred embodiment, the present invention provides a method for treating generalized anxiety disorder, comprising: administering to a patient in need thereof an effective amount of a compound of Formula I, or a pharmaceutically acceptable salt thereof.

In another preferred embodiment, the present invention provides a method for treating panic disorder, comprising: administering to a patient in need thereof an effective amount of a compound of Formula I, or a pharmaceutically acceptable salt thereof.

In another preferred embodiment, the present invention provides a method for treating obsessive compulsive disorder, comprising: administering to a patient in need thereof an effective amount of a compound of Formula I, or a pharmaceutically acceptable salt thereof.

In another preferred embodiment, the present invention provides a method for treating social phobia, comprising: administering to a patient in need thereof an effective amount of a compound of Formula I, or a pharmaceutically acceptable salt thereof.

In another preferred embodiment, the present invention provides a method for treating irritable bowel syndrome, comprising: administering to a patient in need thereof an effective amount of a compound of Formula I, or a pharmaceutically acceptable salt thereof.

In another preferred embodiment, the present invention provides a method for treating inflammatory bowel disease, comprising: administering to a patient in need thereof an effective amount of a compound of Formula I, or a pharmaceutically acceptable salt thereof.

In another preferred embodiment, the present invention provides a method for treating emesis (including chemotherapy-induced nausea and acute or delayed emesis), comprising: administering to a patient in need thereof an effective amount of a compound of Formula I, or a pharmaceutically acceptable salt thereof.

The terms and abbreviations used in the preparations and examples have their normal meanings unless otherwise designated. For example “° C.” refers to degrees Celsius; “N” refers to normal or normality; “mol” refers to mole or moles; “mmol” refers to millimole or millimoles; “h” refers to hour(s); “eq” refers to equivalent; “g” refers to gram or grams; “L” refers to liter or liters; “mL” refers to milliliter milliliters; “M” refers to molar or molarity; “brine” refers to a saturated aqueous sodium chloride solution; “J” is an NMR coupling constant, reported in hertz; “ES” refers to electrospray; “MS” refers to mass spectrometry; “NMR” refers to nuclear magnetic resonance spectroscopy; “TLC” refers to thin layer chromatography; “ACN” refers to acetonitrile; “DMF” refers to N,N-dimethylformamide; “DMSO” refers to dimethylsulfoxide; “Et₂O” refers to diethyl ether; “EtOAc” refers to ethyl acetate; “MeOH” refers to methanol; “EtOH” refers to ethanol; “iPrOH” refers to isopropanol; “TEA” refers to triethylamine; “TFA” refers to trifluoroacetic acid; “THF” refers to tetrahydrofuran; “HOAt” refers to 1-hydroxy-7-azabenzotriazole; and “HOBt” refers to 1-hydroxy-benzotriazole; “DAST” refers to (Diethylamino)sulfur trifluoride.

As used herein, the term “C₁-C₄ alkyl” refers to straight or branched, monovalent, saturated aliphatic chains of 1 to 4 carbon atoms and includes, but is not limited to, methyl, ethyl, propyl, isopropyl, butyl, isobutyl, sec-butyl, and tert-butyl. The terms “C₁-C₃ alkyl” and “C₁-C₂ alkyl” are encompassed within the definition of “C₁-C₄ alkyl.”

The term “optionally substituted phenyl” refers to a phenyl that is unsubstituted or substituted with one to three substituents independently selected from the group consisting of halo, hydroxy, C₁-C₄ alkyl, C₁-C₄ alkoxy, trifluoromethyl, triflouromethoxy, and —NR^{x}R^{y}, wherein R^{x} is H or C₁-C₄ alkyl, and R^{y} is H, or C₁-C₄ alkyl; or R^{x} and R^{y}, together with the N to which they are attached, form a 4-7 membered saturated heterocyclic ring.

Examples of “4-7 membered saturated heterocyclic rings” include, but are not limited to, azetidinyl, pyrrolidinyl, piperidinyl (piperidyl or piperidino), hexamethyleneiminyl (homopiperidinyl), piperazinyl, and morpholin-4-yl (morpholino).

The term “optionally substituted pyrrolidinyl” refers to a pyrrolidin-1-yl, pyrrolidin-2-yl, or pyrrolidin-3-yl that is unsubstituted or substituted with one substituent selected from C₁-C₃ alkyl, phenyl, or benzyl.

The term “optionally substituted piperidinyl” refers to a piperidin-1-yl (piperidino), piperidin-2-yl, piperidin-3-yl, or piperidin-4-yl that is unsubstituted or substituted with one substituent selected from C₁-C₃ alkyl, phenyl, or benzyl.

When R² and R³, together with the nitrogen to which they are attached, form a “4-11 membered heterocyclic ring,” such 4-11 membered heterocyclic rings include saturated or unsaturated monocyclic heterocyclic rings containing nitrogen, and optionally containing one additional heteroatom selected from nitrogen, oxygen, or sulfur, and further include a bicyclic ring in which any of the above-defined monocyclic heterocyclic rings is fused to a benzene ring. Examples of such 4-11 membered heterocyclic rings include, but are not limited to, pyrrolidinyl, pyrrolyl, diazolidinyl, oxazolidinyl, pyrazolidinyl, thiazolidinyl, piperidino, piperazinyl, hexahydropyridazinyl, indolinyl, benzazepanyl, tetrahydroisoquinolinyl, and tetrahydroquinolinyl.

“C₁-C₃ alkane-diyl” refers to a straight or branched, divalent, saturated aliphatic chain of 1 to 3 carbon atoms and includes, but is not limited to, methylene, ethylene, ethane-1,1-diyl, propane-1,1-diyl, propane-1,2-diyl, propane-1,3-diyl, and propane-2,2-diyl. The term “C₁-C₂ alkane-diyl” is encompassed within the definition of “C₁-C₃ alkane-diyl.”

“C₁-C₄ alkoxy” represents a C₁-C₄ alkyl group, as defined above, linked to the parent molecule through an oxygen atom. Typical C₁-C₄ alkoxy groups include methoxy, ethoxy, propoxy, isopropoxy, butoxy, sec-butoxy, tert-butoxy, and the like. The term “C₁-C₄ alkoxy” includes within its definition the term “C₁-C₃ alkoxy” and “C₁-C₂ alkoxy.”

“C₃-C₁₀ cycloalkyl” represents a saturated monocyclic hydrocarbon ring structure containing from three to six carbon atoms (C₃-C₆ cycloalkyl), and further represents a bicyclic ring in which the above-defined C₃-C₆ cycloalkyl is fused to a benzene ring. Typical C₃-C₁₀ cycloalkyl groups include cyclopropyl, cyclobutyl, cyclopentyl, cyclohexyl, indanyl, tetrahydronaphthyl, and the like.

“Halo,” “halogen,” and “halide” represent a chloro, fluoro, bromo or iodo atom. Preferred halogens include chloro and fluoro.

“C₁-C₄ alkoxycarbonyl” represents a straight or branched C₁-C₄ alkoxy chain, as defined above, that is attached via the oxygen atom of the alkoxy to a carbonyl moiety. Typical C₁-C₄ alkoxycarbonyl groups include methoxycarbonyl, ethoxycarbonyl, propoxycarbonyl, isopropoxycarbonyl, butoxycarbonyl, t-butoxycarbonyl and the like.

The term “Pg” refers to an alcohol, carboxyl, or amino protecting group. Typical protecting groups include tetrahydropyranyl (THP), silanes such as trimethylsilane (TMS), tert-butyldimethylsilyl (TBDMS), and tert-butyldiphenylsilane (TBDPS), methoxymethyl (MOM), benzyl (Bn), p-methoxybenzyl, formyl, acetyl (Ac), and tert-butoxycarbonyl (t-BOC). Typical carboxyl protecting groups may include methyl, ethyl, and tert-butyl. The selection and use of protecting groups is well known and appreciated in the art. See for example, Protecting Groups in Organic Synthesis, Theodora Greene (Wiley-Interscience); Protecting Groups, Philip J. Kocienski, Thieme Medical Publishers, inc: New York 1994, chapters 2,4,6.

It is understood that when any substituent is a pyridyl radical, the radical may be a pyridin-2-yl, pyridin-3-yl, or pyridin-4-yl. When a substituent is furyl or thienyl, the radical may be attached at the 2-, or 3-position of the radical. When a substituent is pyrrolyl or imidazolyl, the radical may be attached at the 1-, 2-, or 3 position of the pyrrolyl, or the 1, 2, or 4 position of the imidazolyl.

The compounds of the present invention may exist as stereoisomers. The Cahn-Prelog-Ingold designations of (R)- and (S)- and the designations of L- and D- for stereochemistry relative to the isomers of glyceraldehyde are used herein to refer to specific isomers. The specific stereoisomers can be prepared by stereospecific synthesis or can be resolved and recovered by techniques known in the art, such as chromatography on chiral stationary phases, and fractional recrystallization of addition salts formed by reagents used for that purpose. Useful methods of resolving and recovering specific stereoisomers are known in the art and described in E. L. Eliel and S. H. Wilen, Stereochemistry of Organic Compounds, (Wiley-Interscience 1994), and J. Jacques, A. Collet, and S. H. Wilen, Enantiomers, Racemates, and Resolutions, Wiley-Interscience 1981). It is understood that the present invention contemplates all enantiomers and mixtures of enantiomers, including racemates.

The skilled artisan will recognize that compounds of the present invention may exist as tautomers. It is understood that tautomeric forms of the compounds of Formula (I) are also encompassed in the present invention.

This invention includes the pharmaceutically acceptable salts of the compounds of Formula I. A compound of this invention can possess a sufficiently basic functional group, which can react with any of a number of inorganic and organic acids, to form a pharmaceutically acceptable salt.

The term “pharmaceutically-acceptable salt” as used herein, refers to a salt of a compound of the above Formula I. It should be recognized that the particular counterion forming a part of any salt of this invention is usually not of a critical nature, so long as the salt as a whole is pharmacologically acceptable and as long as the counterion does not contribute undesired qualities to the salt as a whole.

The compounds of Formula I and the intermediates described herein form pharmaceutically-acceptable acid addition salts with a wide variety of organic and inorganic acids and include the physiologically-acceptable salts which are often used in pharmaceutical chemistry. Such salts are also part of this invention. A pharmaceutically-acceptable acid addition salt is formed from a pharmaceutically-acceptable acid, as is well known in the art. Such salts include the pharmaceutically acceptable salts listed in Journal of Pharmaceutical Science, 66, 2-19 (1977), which are known to the skilled artisan. See also, The Handbook of Pharmaceutical Salts; Properties, Selection, and Use. P. H. Stahl and C. G. Wermuth (ED.s), Verlag, Zurich (Switzerland) 2002.

Typical inorganic acids used to form such salts include hydrochloric, hydrobromic, hydriodic, nitric, sulfuric, phosphoric, hypophosphoric, metaphosphoric, pyrophosphoric, and the like. Salts derived from organic acids, such as aliphatic mono and dicarboxylic acids, phenyl substituted alkanoic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, α-hydroxybutyrate, butyne-1,4-dicarboxylate, hexyne-1,4-dicarboxylate, caprate, caprylate, cinnamate, citrate, formate, fumarate, glycollate, heptanoate, hippurate, lactate, malate, maleate, hydroxymaleate, malonate, mandelate, mesylate, nicotinate, isonicotinate, nitrate, oxalate, phthalate, teraphthalate, propiolate, propionate, phenylpropionate, salicylate, sebacate, succinate, suberate, benzenesulfonate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, p-toluenesulfonate, xylenesulfonate, tartarate, and the like.

As used herein, the term “patient” refers to a mammal that is afflicted with one or more disorders associated with excess tachykinins. Guinea pigs, dogs, cats, rats, mice, horses, cattle, sheep, and humans are examples of mammals within the scope of the meaning of the term. It will be understood that the most preferred patient is a human. It is also understood that this invention relates specifically to the inhibition of mammalian NK-1 receptors.

It is also recognized that one skilled in the art may affect the disorders by treating a patient presently afflicted with the disorders or by prophylactically treating a patient afflicted with the disorders with an effective amount of the compound of Formula I. Thus, the terms “treatment” and “treating” are intended to refer to all processes wherein there may be a slowing, interrupting, arresting, controlling, or stopping of the progression of the disorders described herein, and is intended to include prophylactic treatment of such disorders, but does not necessarily indicate a total elimination of all disorder symptoms.

As used herein, the term “effective amount” of a compound of Formula I refers to an amount that is effective in treating the disorders described herein.

As with any group of pharmaceutically active compounds, some groups are preferred in their end use application. Preferred embodiments of the present invention are discussed below.

Preferred embodiments of 4-11 membered heterocyclic rings are illustrated below. As described above, each of the preferred 4-11 membered heterocyclic rings depicted below may be further optionally substituted with one to four substituents independently selected from the group consisting of optionally substituted phenyl, C₃-C₆ cycloalkyl, pyridyl, halo, hydroxy, oxo, and C₁-C₄ alkyl, wherein the C₁-C₄ alkyl is further optionally substituted with one to two substituents selected from the group consisting of C₁-C₃ alkoxy, optionally substituted phenyl, oxo, phenoxy, pyridyl, and pyrrolidinyl.

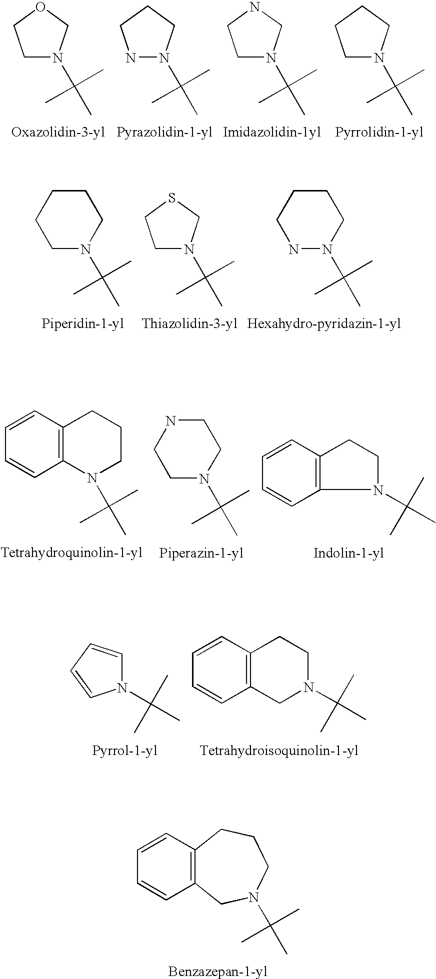

Especially preferred embodiments of the compounds of Formula (I) are given below.:
(a) D¹ is methylene.
(b) D² is nitrogen.
(c) D⁴ is oxygen.
(d) R¹ is phenyl, which phenyl is optionally substituted with one to three substitutents independently selected from the group consisting of halo, C₁-C₄ alkyl, C₁-C₄ alkoxy, cyano, difluoromethyl, trifluoromethyl, and trifluoromethoxy.
(e) R¹is 3,5-bis-trifluoromethyl-phenyl.
(f) R⁵ is a radical of Formula (ID).
(g) R⁵ is phenyl.
(h) R⁵ is pyridin-4-yl.
(i) R⁵ is pyridin-3-yl.
(j) R⁵ is a radical of Formula (IC).
(k) R⁵ is morpholino.
(l) R² is C₁-C₄ alkyl, which C₁-C₄ alkyl is optionally substituted with hydroxy, C₁-C₂ alkoxy, optionally substituted phenyl, pyridyl, —NR⁶R⁷, or naphthyl.
(m) R³ is C₁-C₄ alkyl, which C₁-C₄ alkyl is optionally substituted with R⁴.
(n) R² is 2-chloro-benzyl.
(o) R³ is methyl.
(p) R² and R³, together with the nitrogen to which they are attached, form a 4-11 membered saturated heterocyclic ring, which heterocyclic ring is further optionally substituted with one to four substituents independently selected from the group consisting of optionally substituted phenyl, C₃-C₆ cycloalkyl, pyridyl, halo, hydroxy, oxo, and C₁-C₄ alkyl, wherein the C₁-C₄ alkyl is further optionally substituted with one to two substituents selected from the group consisting of C₁-C₃ alkoxy, optionally substituted phenyl, oxo, phenoxy, pyridyl, and pyrrolidinyl.
(q) R² and R³, together with the nitrogen to which they are attached, form pyrrolidine, which pyrrolidine is further optionally substituted with one to four substituents independently selected from the group consisting of optionally substituted phenyl, C₃-C₆ cycloalkyl, pyridyl, halo, hydroxy, oxo, and C₁-C₄ alkyl, wherein the C₁-C₄ alkyl is further optionally substituted with one to two substituents selected from the group consisting of C₁-C₃ alkoxy, optionally substituted phenyl, oxo, phenoxy, pyridyl, and pyrrolidinyl.
(r) R² and R³, together with the nitrogen to which they are attached, form 2-(2-chloro-phenyl)-pyrrolidine.

### Schemes

The compounds disclosed herein can be made according to the following schemes. The schemes, preparations, and examples should in no way be understood to be limiting in any way as to how the compounds may be made.

The skilled artisan will appreciate that the introduction of certain substituents will create asymmetry in the compounds of Formula (I). The present invention contemplates all stereoisomers, enantiomers, and mixtures of enantiomers, including racemates and diastereomers. It is preferred that the compounds of the invention containing chiral centers are single enantiomers.

As the following schemes, preparations, and examples demonstrate, many of the compounds of the present invention are not only selective NK-1 receptor antagonists, but are also useful intermediates for the preparation of additional compounds of Formula (I). It will be recognized by one of skill in the art that the individual steps in the following schemes may be varied to provide the compounds of Formula (I). The particular order of steps required to produce the compounds of Formula (I) is dependent upon the particular compound being synthesized, the starting compound, and the relative lability of the substituted moieties. Some substituents have been eliminated in the following schemes for the sake of clarity and are not intended to limit the teaching of the schemes in any way. In the schemes below, it will be clear that compounds of Formula (8), (9), and (1 8) are encompassed within the scope of the compounds of Formula (I).

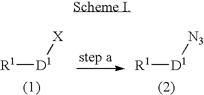

In Scheme I, step a, alkyl azides of Formula (2) can be prepared using standard synthetic methods. For example, see Scriven and Turnbull, Chem. Rev. (1988) 88(2): 351-368.

In the compounds of Formula (1), X may be either a hydroxyl or a leaving group. Suitable leaving groups include halogen, tosylate, mesylate, nosylate, or triflate. Compounds of Formula (1) are readily available or can be readily prepared.

When X of Formula (1) is a hydroxyl group, the alcohol of Formula (1) is mixed with an organic base, typically at approximately 8-12 molar equivalents of organic base per molar equivalent of the alcohol. Suitable organic bases may include triethylamine, diisopropylethylamine, pyridine, collidine, lutadine, or 1,8-diazabicyclo[5,4.0]undec-7-ene, with pyridine being the preferred base. A suitable sulfonylating agent, such as p-toluenesulfonyl chloride, methanesufonyl chloride, p-nitrobenzenesulfonyl chloride, or trifluoromethanesulfonic anhydride, preferably p-toluenesulfonyl chloride, is added in the reaction of step a for the conversion of the hydroxy group of Formula (1) into a suitable leaving group. Typically, the sulfonylating agent is used in slight molar excess to the alcohol of Formula (1).

Azide sources such as NaN₃, LiN₃, or tetrabutylammonium azide (Bu₄NN₃) are acceptable, with NaN₃ being preferred. Typically, about 1-3 molar equivalents of the azide source are used. The reaction of step a is typically carried out in a solvent, such as DMSO/H₂O, N,N-dimethylformamide, tetrahydrofuran, ethanol, methanol, and dioxane, preferably DMSO/H₂O, at temperatures ranging from room temperature to about 80° C. In most cases, the resulting crude azide of Formula (2) can be used without further purification.

When D¹ is methylene, compounds of Formula (1) in which X is a hydroxyl group can be directly converted to the azide. Such reactions are well known and appreciated in the art. For example, see Thompson et al., J. Org. Chem. (1993) 58: 5886-5888. In such reactions, the alcohol of Formula (1) is dissolved in a suitable solvent, such as toluene, benzene, tetrahydrofuran, or dioxane, with the preferred solvent being toluene, and the reaction of step a is carried out using a diphenylphosphoryl azide, followed by a suitable organic base, as described above, with the preferred base being 1,8-diazabicyclo[5,4.0]undec-7-ene. Typically about 1-3 molar equivalents of the azide source are used. The product of Formula (2) can be isolated and purified by techniques well known in the art, such as precipitation, filtration, extraction, evaporation trituration, chromatography, and recrystallization.

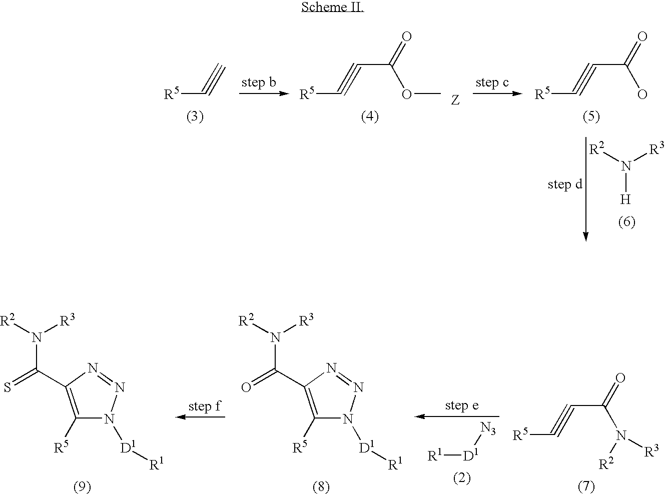

In the reaction of step b, shown in Scheme II, an alkyne of Formula (3) is dissolved in a suitable solvent, typically dichloromethane, chloroform, tetrahydrofuran, dioxane, or diethyl ether, and further reacted with a suitable base, such as lithium diisopropylamide, potassium bis(trimethylsilyl)amide, lithium bis(trimethylsilyl)amide, sodium bis(trimethylsilyl)amide, C₁-C₆ alkylmagnesium bromide, phenylmagnesium bromide, or n-butyllithium, with n-butyllithium being the preferred base. The reaction is carried out with an appropriate chloroformate agent, such as a C₁-C₆ alkyl (e.g., methyl, ethyl, propyl, butyl), aryl (e.g., phenyl), or benzyl chloroformate. Thus, Z is defined in compounds of Formula (4) as C₁-C₆ alkyl, aryl, or benzyl. Generally, the reaction proceeds at temperatures from about −78° C. to ambient temperature. The product of Formula (4) can be isolated and purified by techniques well known in the art, as described above.

In step c, hydrolysis of an alkynyl ester of Formula (4) to give a compound of Formula (5) is well known and appreciated in the art (Larock, R. C., Comprehensive Organic Transformations, 2^{nd} Ed., copyright 1999, John Wiley & Sons, pp 1959-1968). For example, an appropriate ester of Formula (4) is dissolved in a suitable solvent, such as methanol, and is further treated with a suitable base, such as sodium hydroxide, to give a compound of Formula (5).

The reaction of step d, in which a carboxylic acid, such as that of Formula (5), is coupled with an appropriate amine, such as that of Formula (6), under standard peptide coupling conditions, is well known to the skilled artisan. Specifically, the amine and the carboxylic acid are coupled in the presence of a peptide coupling reagent, optionally in the presence of a catalyst. Suitable peptide coupling reagents include N,N′-carbonyldiimidazole (CDI), N,N′-dicyclohexylcarbodiimide (DCC), 1-(3-dimethylaminopropyl)-3-ethylcarbodiimide hydrochloride (EDC), and 1-(3-(1-pyrrolidinyl)propyl)-3-ethylcarbodiimide (PEPC). Suitable catalysts for the coupling reaction include N,N-[dimethyl]-4-aminopyridine (DMAP). All of the reagents are combined in a suitable solvent, typically dichloromethane, chloroform, tetrahydrofuran, dioxane, or diethyl ether, and are stirred for 1 to 72 hours at temperatures ranging from ambient temperature to approximately the reflux temperature of the solvent. The desired product may be isolated and purified by techniques described above. Such coupling reactions are well known and appreciated in the art (Larock, R. C., Comprehensive Organic Transformations, 2^{nd} Ed., copyright 1999, John Wiley & Sons, pp 1941-1949).

Alternatively, a compound of Formula (5) may be converted to an acid chloride, preferably by reaction with oxalyl chloride, and used to acylate the appropriate amine of Formula (6) to give a compound of Formula (7). Such acylation reactions are well known and appreciated in the art (Larock, R. C., Comprehensive Organic Transformations, 2^{nd} Ed., copyright 1999, John Wiley & Sons, pp 1929-1930). The product can be isolated and purified by techniques described above.

In reaction step e, a compound of Formula (2) is reacted with a compound of Formula (7) to give a compound of Formula (8). The reaction is generally carried out in a suitable solvent, such as toluene, benzene, xylene, ethanol, N,N-dimethylformamide, dimethylsufoxide, or tetrahydrofuran, preferably toluene, typically at temperatures ranging from 60-120° C. The product can be isolated and purified by techniques described above.

In the optional reaction of step f, a compound of Formula (8) can be transformed to a thiocarbonyl compound of Formula (9) by [2,4-bis(4-methoxyphenyl)-1,3-dithia-2,4-diphosphetane-2,4-disulfide](Lawesson's Reagent) or phosphorus pentasulfide, typically in a suitable solvent, for example, toluene, ethylene glycol dimethyl ether, benzene, pyridine, xylene, or tetrahydrofuran, preferably toluene. The reaction is generally carried out at temperatures of about room temperature to 100° C. The product can be isolated and purified by techniques described above.

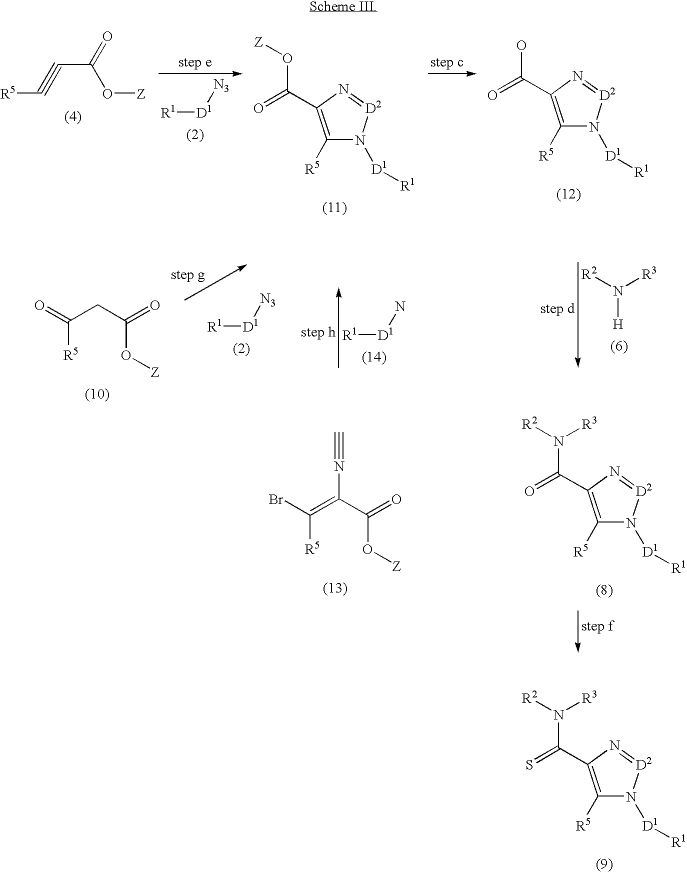

As one of the variations mentioned above, shown in Scheme III, a compound of Formula (4) is cyclized with an azide of Formula (2), as described in step e, to give the ester corresponding to the compound of Formula (11), wherein D² is nitrogen. Subsequent hydrolysis, as taught in step c, followed by amide formation, as taught in step d, gives the desired compound of Formula (8). In the compounds depicted in Scheme III, Z is C₁-C₆ alkyl, aryl, or benzyl.

Another variation for making compounds of Formula (I) is depicted in step g. In step g, the triazole ring of Formula (11), in which D² is nitrogen, is made by reacting a beta keto ester compound of Formula (10), such as a beta keto C₁-C₆ alkyl or benzyl ester, with an azide of Formula (2). Such ring formations are well known and appreciated in the art. See Savini et al., Farmaco (1994) 49(5): 363-370; Martini et al., J. Pharm. Sci. (1988) 77(11): 977-980; Sun et al., Magn. Reson. Chem. (1998) 36(6): 459-460; Settimo et al., Farmaco Ed. Sci. (1983) 38(10): 725-737; Olesen et al., J. Heterocycl. Chem. (1984) 21: 1603-1608; L'abbe et al., Bull. Soc. Chim. Belg. (1987) 96(10): 823-824; Julino et al., J. Chem. Soc. Perkin Trans. I (1998) 10: 1677-1684; Mamedov et al., Chem. Heterocycl. Compd. (Engl. Transl.) (1993) 29(5): 607-611; Wender et al., Tetrahedron Lett. (1987) 28(49): 6125-6128; Freitas et al., J. Heterocycl. Chem. (1995) 32(2): 457-462; Cottrell et al., J. Heterocycl. Chem. (1991) 28(2): 301-304.

The reaction of step g is typically carried out in the presence of a suitable base, such as sodium carbonate, lithium carbonate, sodium alkoxide (such as sodium methanolate or ethanolate), or potassium alkoxide, (such as potassium methanolate or potassium ethanolate), or sodium hydride, with potassium carbonate being a preferred base. Generally, the reaction is carried out using 2-4 molar equivalents of the base in a suitable solvent, such as DMSO, methanol, ethanol, or DMF, with DMSO being a preferred solvent. The azide of Formula (2) and the beta keto ester of Formula (4) are used at roughly molar equivalence. The reaction is carried out at temperatures of about 20-80° C., with reaction times ranging from approximately 4-24 hours. In general, basic conditions are favored for the condensation of the above compounds of Formula (2). The product can be isolated and purified by techniques described above.

Compounds of Formula (11) in which D² is —CH may be made by the reaction of step h. A compound of Formula (13), in which Z can be C₁-C₆ alkyl, aryl, or benzyl, is prepared by methods described herein and by methods described in the art, for example, J. Org. Chem. (1994) 59: 7635. An appropriate compound of Formula (13) can be condensed with an appropriate amine of Formula (14) to give the compound of Formula (11). Appropriate amines of Formula (14) are readily available. The reaction is typically carried out in the presence of a suitable organic base, such as triethylamine, diisopropylethylamine, pyridine, collidine, lutidine, or 1,8-diazabicyclo[5,4.0]undec-7-ene, preferably triethylamine. The reaction is carried out in a suitable solvent, such as 1-methyl-2-pyrrolidinone, DMF, toluene, tetrahydrofuran or chloroform, preferably DMF, at temperatures ranging from about 0 to 80° C. The product can be isolated and purified by standard techniques, as described above.

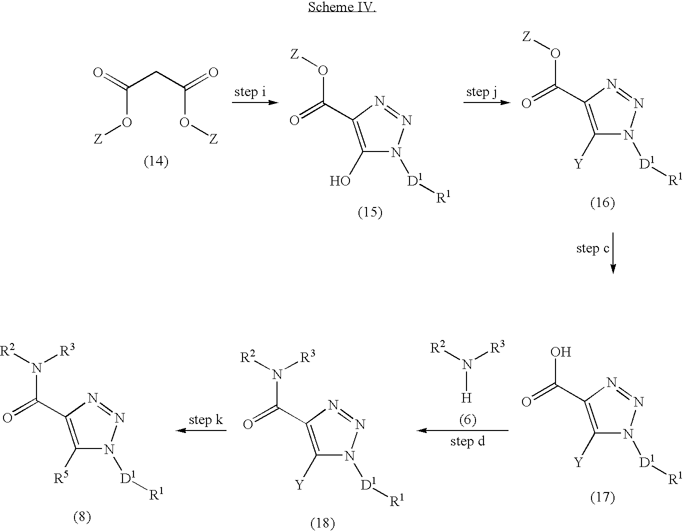

Another variation for making compounds of Formula (I) is depicted in Scheme IV, step i. In step i, the triazole ring of Formula (15), in which D² is nitrogen, is made by reacting a dialkylmalonate of Formula (14) with an azide of Formula (2). The hydroxyl group of the compound of Formula (15) maybe readily converted to the corresponding halide, as shown in step j, to give a compound of Formula (16) wherein Y is a halide. Examples of reagents for this reaction include PCl₅, POCl₃, PBr₃, POBr₃, and thionyl chloride, with PCl₅ as the preferred reagent either neat or in a suitable solvent such as dichloromethane, benzene, or toluene at a temperature between 0 and 100° C. The preferred method is reacting a compound of Formula (15) with PCl₅ in toluene at 40-60° C. This type of transformation is well known and appreciated in the art. See Buckle, D. R.; Rockell, C. J. M. J. Chem. Soc., Perkin I, 1982, 627-630. Subsequent ester hydrolysis, as taught in step c, followed by amide formation, as taught in step d, gives compounds of Formula (18). As shown in step k, the halide of the compound of Formula (18) may be substituted by reaction with an appropriate nucleophile such as, but not limited to, primary amines, secondary amines, alcohols or thiols to further encompass compounds of the present invention to give the desired compounds of Formula (8). Such reactions are well known and appreciated in the art. See March, J., Advanced Organic Chemistry, 1985, John Wiley and Sons, Inc., pp 255-446. In such reactions, the compound of Formula (18) is dissolved in a suitable solvent, such as DMF, THF, DMSO, and reacted with the appropriate nucleophile in the presence of a suitable base. Such bases include triethylamine, potassium carbonate, cesium carbonate or sodium hydride. The reaction is generally carried out at temperatures ranging from room temperature to 100° C. In some cases, the reaction may be carried out neat, using the nucleophile as solvent. The product of Formula (8) can be isolated and purified by techniques described above.

As depicted in Scheme II, a compound of Formula (8) can be transformed to a thiocarbonyl compound of Formula (9) by [2,4-bis(4-methoxyphenyl)-1,3-dithia-2,4-diphosphetane-2,4-disulfide] (Lawesson's Reagent) or phosphorus pentasulfide, typically in a suitable solvent, for example, toluene, ethylene glycol dimethyl ether, benzene, pyridine, xylene, or tetrahydrofuran, preferably toluene. The reaction is generally carried out at temperatures of about room temperature to 100° C. The product can be isolated and purified by techniques described above.

The skilled artisan will appreciate that the compounds of Formula (8), (9), and (18) in Schemes II, III, and IV may be formed into acid addition salts using pharmaceutically acceptable acids. The formation of acid-addition salts is well known and appreciated in the art.

### Preparation 1

### 2-Amino-2-(2-chloro-phenyl)-acetamide hydrochloride

Stir a slurry of 2-chlorobenzaldehyde (43 mL, 380 mmol) and sodium bisulfite (39.5 g) in water (150 mL) and MeOH (150 mL) for 15 min., then add ammonium hydroxide (26 mL, 380 mmol). Stir the mixture for 30 min. at RT, then cool to 0° C. Add MeOH (75 mL) to the mixture, then add a solution of sodium cyanide (18.6 g, 380 mmol) in water (75 mL) dropwise over 15 min. Remove the ice bath and stir overnight. Evaporate off the organics, then extract the aqueous layer with ether three times. Wash the combined ether extracts with water, and brine, dry over Na₂SO₄, filter, and concentrate to approximately 200 mL. Acidify the solution to pH 4.5 with 2 N HCl. Cool the resulting slurry at 4° C. for 30 min., then filter the precipitate and dry under vacuum to afford the title compound (2.1 g, 2.5%) as a white solid. MS(FD) 186.63 (M+). ¹H NMR (400 MHz, DMSO-d₆) δ12.7 (br s, 1H), 7.33 (s, 1H), 7.22 (s, 2H), 5.07 (s, 2H).

### Preparation 2

### [2-(2-Chloro-benzylamino)-ethyl]-carbamic acid tert-butyl ester

Dissolve 2-chlorobenzaldehyde (1.31 g, 9.3 mmol) and t-butyl-N-(2-aminoethyl) carbamate (1 g, 6.2 mmol) in dry MeOH (0.2M) and stir for one hour. Cool the solution to 0° C., and add NaBH₄ (2.81 g, 74.4 mmol). After 15 min., warm the mixture to RT, and stir another hour. Quench with 1N NaOH (400 mL), extract with CH₂Cl₂ (2×250 mL), dry over Na₂SO₄, filter, and concentrate. Use without further purification. ¹H NMR (CDCl₃, 250 MHz) δ7.40-7.22 (m, 4H), 3.90 (s, 2H), 3.25 (q, 2H, J=5.72 Hz), 2.79-2.74 (m, 2H), 1.47 (s, 9H); MS(ES) 285.1 (M+1)⁺.

### Preparation 3

### N¹-(2-Chloro-benzyl)-ethane-1,2-diamine

To a solution of [2-(2-chloro-benzylamino)-ethyl]-carbamic acid tert-butyl ester (450 mg, 1.76 mmol) in CH₂Cl₂ (0.2M), add anisole (571 mg, 5.28 mmol) and trifluoroacetic acid (1.48 mL) and stir at RT. After 12 h, dilute the solution with CH₂Cl₂ (15 mL) and extract with 1N HCl (15 mL). Make the aqueous layer basic with 5 N NaOH (10 mL) and extract with CH₂Cl₂ (25mL), dry over Na₂SO₄, filter, and concentrate. Use crude material without further purification. ¹H NMR (CDCl₃, 250 MHz) δ7.19-7.40 (m, 4H), 3.89 (s, 2H), 2.83-2.85 (m, 2H), 2.68-2.71 (m, 2H); MS(ES) 185.1 (M+1)⁺.

### Preparation 4

### 3-(2-Methyl-benzylamino)-propan-1-ol

Mix 1-bromomethyl-2-methyl-benzene (100 g, 0.5 mol) and 3-amino-1-propanol (340 mL) and stir at RT. After 4 h, dilute the mixture with H₂O (1 L), add 5N NaOH until the solution is basic, and extract with ether (3×1 L). Wash the organic layer with H₂O, and brine, dry over K₂CO₃, filter, and concentrate. Purify by distillation under reduced pressure (120° C., 0.4 mm Hg). Anal. calc'd for C: 73.70%, H: 9.56%, N: 7.81%; Found C: 73.44%, H: 9.36%, N: 7.75%.

### Preparation 5

### (3-Bromo-propyl)-(2-methyl-benzyl)-amine

In a three neck round bottom flask fitted with a thermometer and distillation head, add a solution of 48% aqueous HBr (130 mL) to cooled (5° C.) 3-(2-methyl-benzylamino)-propan-1-ol (46.3 g, 0.26 mol). Heat the resulting solution, distilling off H₂O (91 mL, 110° C. to 124° C.). Cool the solution, filter off the resulting solid, and rinse with H₂O. Recrystallize from iPrOH (500 mL). mp 167-169° C.

### Preparation 6

### 9-Methyl-2,3,4,5-tetrahydro-1H-benzo[c]azepine hydrochloride

Add AlCl₃ (39.9 g, 0.3 mol) to a solution of (3-bromo-propyl)-(2-methyl-benzyl)-amine (3.23 g, 0.10 mol) in decalin (400 mL). Heat the solution to 130° C. for 1 h, then cool in an ice bath and acidify with conc. HCl (100 mL). Wash the resulting solution with ether, make the aqueous layer basic with 5 N NaOH, and extract with ether (three times). Wash the organic layer with brine, dry over K₂CO₃, filter, and concentrate. Purify the liquid by distillation under reduced pressure (b.p. 116-120° C. at 8 mm Hg). Form the HCl salt and recrystallize from EtOAc/MeOH, filter and recrystallize again from iPrOH. m.p. 244-247° C. R_{f}=0.61 (20:1 CHCl₃/MeOH).

### Preparation 7

### 1-(3,5-Bis-trifluoromethyl-benzyl)-5-(2-chloro-phenyl)-1H-[1,2,3]triazole-4-carboxylic acid

Dissolve 1-(3,5 -bis-trifluoromethyl-benzyl)-5-(2-chloro-phenyl)-1H-[1,2,3]triazole-4-carboxylic acid ethyl ester (800 mg, 1.67 mmol) in EtOH (7 mL) and add 1N NaOH (3 mL, 3 mmol). Warm the mixture to 40° C. and stir overnight. Cool the mixture to RT and acidify with IN HCl (5-10 mL). Collect the precipitate by filtration and rinse with H₂O. Dry in a vacuum oven (40° C.) overnight to provide the title compound (680 mg, 90%) as a white solid. R_{f}=0.50 (2:1 CHCl₃/MeOH); MS(ES) 450.1 (M+1)⁺.

By the method of Preparation 7, using the appropriate carboxylic ester, the following compounds are prepared and isolated.

| Prep. # | Product | Data |
|---|---|---|
| 8 | 1-(3,5-Bis-trifluoromethyl-benzyl)-5-(2-fluoro- | Rf=0.47(2:1 CHCl₃/MeOH); |
| | phenyl)-1H-[1,2,3]triazole-4-carboxylic acid | MS(ES) 434.1(M+1)⁺. |
| 9 | 1-(3,5-Bis-trifluoromethyl-benzyl)-5-(3- | Rf=0.50(2:1 CHCl₃/MeOH); |
| | trifluoromethyl-phenyl)-1H-[1,2,3]triazole-4- | MS(ES):484.1(M+1)⁺. |
| | carboxylic acid | |
| 10 | 1-(3,5-Bis-trifluoromethyl-benzyl)-5-(3-methoxy- | Rf=0.60(2:1 CHCl₃/MeOH); |
| | phenyl)-1H-[1,2,3]triazole-4-carboxylic acid | MS(ES):446.1(M+1)⁺. |
| 11 | 1-(3,5-Bis-trifluoromethyl-benzyl)-5-(4-chloro- | Rf=0.57(2:1 CHCl₃/MeOH); |
| | phenyl)-1H-[1,2,3]triazole-4-carboxylic acid | MS(ES):450.1(M+1)⁺. |
| 12 | 1-(3,5-Bis-trifluoromethyl-benzyl)-5-(4-fluoro- | Rf=0.57(2:1 CHCl₃/MeOH); |
| | phenyl)-1H-[1,2,3]triazole-4-carboxylic acid | MS(ES):434.1(M+1)⁺. |
| 13 | 1-(3,5-Bis-trifluoromethyl-benzyl)-5-p-tolyl-1H- | Rf=0.70(2:1 CHCl₃/MeOH); |
| | [1,2,3]triazole-4-carboxylic acid | MS(ES):430.1(M+1)⁺. |
| 14 | 1-(3,5-Bis-trifluoromethyl-benzyl)-5-phenyl-1H- | Rf=0.40(2:1 CHCl₃/MeOH); |
| | [1,2,3]triazole-4-carboxylic acid | MS(ES):416.1(M+1)⁺. |
| 15 | 1-(3,5-bis-trifluoromethyl-benzyl)-5-(4-methoxy- | MS(ES) 446.1(M+1)⁺; |
| | phenyl)-1H-[1,2,3]triazole-4-carboxylic acid | m.p. 172.4-174.0° C. |
| 16 | 1-(3,5-bis-trifluoromethyl-benzyl)-5-m-tolyl-1H- | MS(ES) 430.1(M+1)⁺; |
| | [1,2,3]triazole-4-carboxylic acid | m.p. 153.2-156.0° C. |
| 17 | 1-(3,5-Bis-trifluoromethyl-benzyl)-5-phenyl-1H- | MS(ES) 415.2(M+1)⁺. |
| | imidazole-4-carboxylic acid | |
| 18 | 1-Phenethyl-5-phenyl-1H-imidazole-4-carboxylic | ¹H NMR(DMSO-d₆, 300mHz) δ |
| | acid | 8.75(s, 1H), 7.25-7.55(m, 5H), |
| | | 7.05-6.95(m, 2H), 4.20(m, 2H), |
| | | 2.80(m, 2H). |

### Preparation 19

### (2-Chloro-phenyl)-propynoic acid ethyl ester

Dissolve 1-chloro-2-ethynyl-benzene (0.56 g, 4.1 mmol) in THF (16 mL) and cool to −78° C. Add BuLi (3.0 mL of a 1.6 M solution in hexanes, 4.9 mmol) dropwise, and stir at −78° C. After 30 min., add ethylchloroformate (0.51 mL, 0.58 g, 5.3 mmol) and allow the resulting solution to warm slowly to RT. After 1 hr, quench with H₂O and extract with Et₂O. Wash the organic layer with brine, dry (MgSO₄), filter and concentrate. Use the resulting crude alkynyl ester without further purification. R_{f}=0.49 (10:1 hexanes/EtOAc); ¹H NMR (CDCl₃, 250 MHz) δ7.52 (dd, J=1.5, 7.5 Hz, 1H), 7.30 (m, 2H), 7.18 (td, J=1.5, 7.3 Hz, 1H), 4.23 (q, J=7.2 Hz, 2H), 1.28 (t, J=7.2 Hz, 3H).

By the method of Preparation 19, using the appropriate alkyne starting material, the following compounds are prepared and isolated: (10:1 hexanes/EtOAc)

| Prep. # | Product | Data |
|---|---|---|
| 20 | (2-Fluoro-phenyl)- | R_{f}=0.38(10:1 hexanes/EtOAc); ¹H NMR(CDCl₃, 250MHz) |
| | propynoic acid ethyl ester | δ 7.59(m, 1H), 7.46(m, 1H), 7.21(m, 2H), 4.34(q, |
| | | J=7.2Hz, 2H), 1.42(t, J=7.2Hz, 3H). |
| 21 | (3-Trifluoromethyl- | R_{f}=0.42(10:1 hexanes/EtOAc); ¹H NMR(CDCl₃, 250MHz) |
| | phenyl)-propynoic acid | δ 7.88(s, 1H), 7.79(d, J=7.7Hz, 1H), 7.73(d, J=8.0Hz, |
| | ethyl ester | 1H), 7.55(t, J=7.8, 1H), 4.31(q, J=7.2Hz, |
| | | 2H), 1.39(t, J=7.2Hz, 3H). |
| 22 | (3-Methoxy-phenyl)- | R_{f}=0.32(10:1 hexanes/EtOAc); ¹H NMR(CDCl₃, 250MHz) |
| | propynoic acid ethyl ester | δ 7.19(d, J=7.7Hz, 1H), 7.15(d, J=3.8Hz, |
| | | 1H), 7.08(dt, J=1.2, 6.4Hz, 1H), 7.00(dd, J=1.4, 2.4Hz, |
| | | 1H), 6.89(ddd, J=1.2, 2.6, 8.2Hz, 1H), 4.20(q, J=7.1Hz, |
| | | 2H), 3.71(s, 3H), 1.26(t, J=7.1Hz, 3H). |
| 23 | (4-Chloro-phenyl)- | R_{f}=0.48(10:1 hexanes/EtOAc); ¹H NMR(CDCl₃, 250MHz) |
| | propynoic acid ethyl ester | δ 7.45(d, J=8.5Hz, 2H), 7.29(d, J=8.5Hz, |
| | | 2H), 4.23(q, J=7.2Hz, 2H), 1.29(t, J=7.2Hz, 3H). |
| 24 | (4-Fluoro-phenyl)- | R_{f}=0.42(10:1 hexanes/EtOAc); ¹H NMR(CDCl₃, 250MHz) |
| | propynoic acid ethyl ester | δ 7.52(dd, J=5.3, 8.8Hz, 2H), 7.00(t, J=8.6Hz, |
| | | 2H), 4.23(q, J=7.1Hz, 2H), 1.29(t, J=7.1Hz, 3H). |
| 25 | p-Tolyl-propynoic acid | R_{f}=0.45(10:1 hexanes/EtOAc); ¹H NMR(CDCl₃, 250MHz) |
| | ethyl ester | δ 7.53(d, J=8.2Hz, 2H), 7.22(d, J=8.0Hz, |
| | | 2H), 4.34(q, J=7.1Hz, 2H), 2.42(s, 3H), 1.40(t, J=7.1Hz, |
| | | 3H). |
| 26 | (4-methoxy-phenyl)- | MS(ES) 205.0(M+1)⁺; IR: 2207cm−1 |
| | propynioc acid ethyl ester | |
| 27 | m-tolyl-propynoic acid | MS(ES) 189.1(M+1)⁺; IR: 2218cm−1 |
| | ethyl ester | |
| 28 | pyridin-2-yl-propynoic | MS(ES) 176.0(M+1)⁺. ¹H NMR(400MHz, CDCl₃):δ |
| | acid ethyl ester | 8.62(m, 1H), 7.69(dt, 1H, J=2.0, 7.8Hz), 7.56(dt, 1H, |
| | | J=1.0, 7.8Hz), 7.32(ddd, 1H, J=1.0, 4.9, 7.8Hz), |
| | | 4.28(q, 2H, J=7.3Hz), 1.31(t, 3H, J=7.3Hz). |

### Preparation 29

### N-methyl-N-[3,5-bis-(trifluoromethyl)benzyl]amine

Add methylamine (3.1 mL of a 2M soln in MeOH, 6.2 mmol) to a solution of 3,5-bis-trifluoromethyl-benzaldehyde (1.0 g, 4.1 mmol) in MeOH (3 mL). Stir at RT for 12 h, then cool to 0° C. and add NaBH₄ (310 mg, 8.25 mmol) in batches (caution: gas evolution). Warm the mixture to RT, and stir overnight. Quench with excess 1N NaOH solution and stir for 30 min., then extract with CH₂Cl₂ (2 times). Wash the combined organic layers with brine, dry over Na₂SO₄, filter, and concentrate. Use the crude amine without further purification. MS(ES) 258.2 (M+1)⁺; R_{f}=0.45 (10:1 CHCl3/MeOH).

By the method of Preparation 29, using the appropriate amine and aldehyde, the following compounds are prepared and isolated: (10:1 CHCl₃/MeOH).

| Prep. # | Product | Data |
|---|---|---|
| 30 | N-methyl-N-(2-fluorobenzyl)amine | MS(ES) 140.0(M+1)⁺; |
| | | R_{f}=0.23(10:1 CHCl₃/MeOH); |
| 31 | N-methyl-N-(4-fluorobenzyl)amine | MS(ES) 140.0(M+1)⁺; |
| | | Rf: 0.11(10:1 CHCl3/MeOH) |
| 32 | N-methyl-N-(3-methylbenzyl)amine | MS(ES) 105.1(M+1)⁺; |
| | | Rf: 0.11(10:1 CHCl₃/MeOH); |
| 33 | N-methyl-N-(2-methoxybenzyl)amine | MS(ES) 152.0(M+1)⁺; |
| | | Rf: 0.14(10:1 CHCl₃/MeOH); |
| 34 | N-methyl-N-(3-methoxybenzyl)amine | MS(ES) 152.0(M+1)⁺; |
| | | Rf: 0.12(10:1 CHCl₃/MeOH); |
| 35 | N-methyl-N-(4-methoxybenzyl)amine | MS(ES) 152.1(M+1)⁺; |
| | | Rf: 0.09(10:1 CHCl₃/MeOH); |
| 36 | N-methyl-N-(4-chlorobenzyl)amine | MS(ES) 156.0(M+1)⁺; |
| | | Rf: 0.11(10:1 CHCl₃/MeOH); |
| 37 | N-methyl-N-(3-chlorobenzyl)amine | MS(ES) 156.0(M+1)⁺; |
| | | Rf: 0.17(10:1 CHCl₃/MeOH); |
| 38 | N-methyl-N-(4-trifluoromethylbenzyl)amine | MS(ES) 190.1(M+1)⁺; |
| | | Rf: 0.17(10:1 CHCl₃/MeOH); |
| 39 | N-methyl-N-[4-(1- | MS(ES) 191.1(M+1)⁺; |
| | pyrrolidino)benzyl]amine | Rf: 0.05(10:1 CHCl₃/MeOH); |
| 40 | N-methyl-N-[4-(N,N- | MS(ES) 165.1(M+1)⁺; |
| | dimethylamino)benzyl]amine | Rf: 0.05(10:1 CHCl₃/MeOH); |
| 41 | N-methyl-N-(2-methylbenzyl)amine | MS(ES) 136.1(M+1)⁺; |
| | | Rf: 0.17(10:1 CHCl₃/MeOH); |
| 42 | N-methyl-N-(4-methylbenzyl)amine | MS(ES) 136.1(M+1)⁺; |
| | | Rf: 0.14(10:1 CHCl₃/MeOH) |
| 43 | N-methyl-N-(3-fluorobenzyl)amine | MS(ES) 140.1(M+1)⁺; |
| | | Rf: 0.23(10:1 CHCl₃/MeOH) |
| 44 | N-methyl-N-(2- | MS(ES) 190.0(M+1)⁺; |
| | trifluoromethyl)benzylamine | Rf: 0.37(10:1 CHCl₃/MeOH) |
| 45 | N-methyl-N-(3- | MS(ES) 190.0(M+1)⁺; |
| | trifluoromethylbenzyl)amine | Rf: 0.23(10:1 CHCl₃/MeOH) |
| 46 | methylpyridin-2-ylmethylamine | MS(ES) 123.1(M+1)⁺; |
| | | Rf: 0.05(10:1 CHCl₃/MeOH) |
| 47 | methylpyridin-4-ylmethylamine | MS(ES) 123.0(M+1)⁺; |
| | | Rf: 0.05(10:1 CHCl₃/MeOH) |
| 48 | (±)-N-methyl-N-alpha-methylbenzylamine | MS(ES) 136.1(M+1)⁺; |
| | | Rf: 0.11(10:1 CHCl₃/MeOH) |
| 49 | (±)-N-methyl-N-alpha-methyl-(3- | MS(ES) 170.0(M+1)⁺; |
| | chlorobenzyl)amine | Rf: 0.20(10:1 CHCl₃/MeOH) |
| 50 | N-methyl-N-(2-chloro-6- | MS(ES) 174.0(M+1)⁺; |
| | fluorobenzyl)amine | Rf: 0.37(10:1 CHCl₃/MeOH) |
| 51 | N-methyl-N-(2,6-dichlorobenzyl)amine | MS(ES) 189.9(M+1)⁺; |
| | | Rf: 0.43(10:1 CHCl₃/MeOH) |
| 52 | N-methyl-N-(2,3-dichlorobenzyl)amine | MS(ES) 189.9(M+1)⁺; |
| | | Rf: 0.34(10:1 CHCl₃/MeOH) |
| 53 | N-methyl-N-(2-chloro-4- | MS(ES) 174.0(M+1)⁺; |
| | fluorobenzyl)amine | Rf: 0.25(10:1 CHCl₃/MeOH) |
| 54 | N-methyl-N-(2,4-difluorobenzyl)amine | MS(ES) 158.0(M+1)⁺; |
| | | Rf: 0.26(10:1 CHCl₃/MeOH) |
| 55 | N-methyl-N-(2,6-difluorobenzyl)amine | MS(ES) 158.0(M+1)⁺; |
| | | Rf: 0.37(10:1 CHCl₃/MeOH) |
| 56 | N-methyl-N-(2-bromobenzyl)amine | MS(ES) 140.0(M+1)⁺; |
| | | Rf: 0.31(10:1 CHCl₃/MeOH) |
| 57 | N-methyl-N-(2- | MS(ES) 199.9(M+)⁺; |
| | trifluoromethoxybenzyl)amine | Rf: 0.29(10:1 CHCl₃/MeOH) |
| 58 | N,N-di-(2-chlorobenzyl)amine | MS(ES) 266.1(M+1)⁺; |
| | | Rf: 0.65(10:1 CHCl₃/MeOH) |
| 59 | N,N-di-(2-fluorobenzyl)amine | MS(ES) 234.1(M+1)⁺; |
| | | Rf: 0.59(10:1 CHCl₃/MeOH) |
| 60 | (R)-N-(2-chlorobenzyl)-N-(alpha- | MS(ES) 246.1(M+1)⁺; |
| | methylbenzyl)amine | Rf: 0.64(10:1 CHCl₃/MeOH) |
| 61 | (S)-N-(2-chlorobenzyl)-N-(alpha- | MS(ES) 246.1(M+1)⁺; |
| | methylbenzyl)amine | Rf: 0.64(10:1 CHCl₃/MeOH); |
| 62 | (±)-N-methyl-N-[alpha-methyl-(2- | MS(ES) 170.0(M+1)⁺; |
| | methylbenzyl)]amine | Rf: 0.11(10:1 CHCl₃/MeOH); |
| 63 | (±)-N-methyl-N-[alpha-methyl-(3- | MS(ES) 154.1(M+1)⁺; |
| | fluorobenzyl)]amine | Rf: 0.14(10:1 CHCl₃/MeOH); |
| 64 | (±)-N-methyl-N-[alpha-methyl-(4- | MS(ES) 154.1(M+1)⁺; |
| | fluorobenzyl)]amine | Rf: 0.11(10:1 CHCl₃/MeOH); |
| 65 | N-ethyl-N-benzylamine | MS(ES) 136.1(M+1)⁺; |
| | | Rf: 0.20(10:1 CHCl₃/MeOH); |
| 66 | N-ethyl-N-(2-chlorobenzyl)amine | MS(ES) 170.0(M+1)⁺; |
| | | Rf: 0.37(10:1 CHCl₃/MeOH); |
| 67 | N-methyl-N-(5-chloro-2- | MS(ES) 186.1(M+1)⁺; |
| | methoxybenzyl)amine | Rf: 0.14(10:1 CHCl₃/MeOH); |
| 68 | N-methyl-N-(2-methoxy-5- | MS(ES) 236.1(M+1)⁺; |
| | trifluoromethoxybenzyl)amine | Rf: 0.17(10:1 CHCl₃/MeOH); |
| 69 | N-methyl-N-(5-fluoro-2- | MS(ES) 170.1(M+1)⁺; |
| | methoxybenzyl)amine | Rf: 0.17(10:1 CHCl₃/MeOH); |
| 70 | N-methyl-N-(3-fluoro-5- | MS(ES) 208.1(M+1)⁺; |
| | trifluoromethylbenzyl)amine | Rf: 0.29(10:1 CHCl₃/MeOH); |
| 71 | N-methyl-N-(3,5-dimethylbenzyl)amine | MS(ES) 150.1(M+1)⁺; |
| | | Rf: 0.14(10:1 CHCl₃/MeOH); |
| 72 | N-methyl-N-(3,5-dichlorobenzyl)amine | MS(ES) 190.0(M+1)⁺; |
| | | Rf: 0.26(10:1 CHCl₃/MeOH); |
| 73 | N′-(2-Chlorobenzyl)-N,N-dimethyl-ethane- | MS(ES) 213.2(M+1)⁺; |
| | 1,2-diamine | Rf: 0.16(10:1 CHCl₃/MeOH); |
| 74 | (2-Chloro-benzyl)-(2-pyrrolidin-1-yl- | MS(ES) 239.2(M+1)⁺; |
| | ethyl)-amine | Rf: 0.21(10:1 CHCl₃/MeOH); |
| 75 | (2-Chloro-benzyl)-(2-morpholin-4-yl- | MS(ES) 255.2(M+1)⁺; |
| | ethyl)-amine | Rf: 0.19(10:1 CHCl₃/MeOH); |
| 76 | (3,5-Bis-trifluoromethyl-benzyl)- | MS(ES) 286.1(M+1)⁺; R_{f}=0.39 |
| | isopropyl-amine | (6.7% MeOH/CH₂Cl₂). |
| 77 | (3,5-Bis-trifluoromethyl-benzyl)- | MS(ES) 284.1(M+1)⁺; R_{f}=0.76 |
| | cyclopropyl-amine | (6.7% MeOH/CH₂Cl₂). |

### Preparation 78

### (±)-N-methyl-N-alpha-methyl-[bis-(3,5-trifluoromethyl)benzyl]amine

Dissolve 3,5-bis(trifluoromethyl)acetophenone (4.97 g, 19.4 mmol) in 1,2-dichloroethane (100 mL). Add methylamine (12.5 mL of a 2 M soln. in THF, 25 mmol) followed by sodium triacetoxyborohydride (8.56 g, 40 mmol). Stir the mixture at RT for 3 h., then quench with excess saturated NaHCO₃ solution. Extract with EtOAc twice and wash the combined organic layers with brine. Dry over Na₂SO₄, filter, and concentrate. Use the crude amine without further purification. MS(ES) 272.1 (M+1)⁺; R_{f}=0.54 (10:1 CHCl₃/MeOH).

By the method of Preparation 78, using the appropriate amine and ketone or aldehyde, the following compounds are prepared and isolated:

| Prep. # | Product | Data |
|---|---|---|
| 79 | (±)-1-methylamino-indane | MS(ES) 148.1(M+1)⁺; |
| | | Rf: 0.11(10:1 CHCl₃/MeOH); |
| 80 | (±)-1-methylamino-1,2,3,4- | MS(ES) 162.1(M+1)⁺; |
| | tetrahydronaphthylene | Rf: 0.14(10:1 CHCl₃/MeOH); |
| 81 | (±)-2-methylamino-1,2,3,4- | MS(ES) 162.1(M+1)⁺; |
| | tetrahydronaphthylene | Rf: 0.14(10:1 CHCl₃/MeOH); |
| 82 | (±)-2-(N-methyl- | MS(ES) 186.1(M+1)⁺; |
| | aminomethyl)naphthylene | Rf: 0.17(10:1 CHCl₃/MeOH); |
| 83 | N-benzyl-N-propylamine | MS(ES) 150.1(M+1)⁺; |
| | | Rf: 0.23(10:1 CHCl₃/MeOH); |
| 84 | N-benzyl-N-isopropylamine | MS(ES) 150.1(M+1)⁺; |
| | | Rf: 0.26(10:1 CHCl₃/MeOH); |
| 85 | N-benzyl-N-cyclopropylamine | MS(ES) 148.1(M+1)⁺; |
| | | Rf: 0.49(10:1 CHCl₃/MeOH); |
| 86 | N-(2-chlorobenzyl)-N- | MS(ES) 184.1(M+1)⁺; |
| | propylamine | Rf: 0.40(10:1 CHCl₃/MeOH); |
| 87 | N-(2-chlorobenzyl)-N- | MS(ES) 184.1(M+1)⁺; |
| | isopropylamine | Rf: 0.46(10:1 CHCl₃/MeOH); |
| 88 | N-(2-chlorobenzyl)-N- | MS(ES) 182.1(M+1)⁺; |
| | cyclopropylamine | Rf: 0.63(10:1 CHCl₃/MeOH); |
| 89 | N-isopropyl-N-(2- | MS(ES) 234.1(M+1)⁺. |
| | trifluoromethoxybenzyl)-amine | |

### Preparation 90

### Indan-2-yl -methyl-amine

Add triethylamine (4.7 g, 46.8 mmol) and ethyl chloroformate (2.46 mL, 25.7 mmol) to a solution of 2-aminoindan (3.12 g, 23.4 mmol) in THF (0.1M). After 1 hr, dilute with EtOAc (200 mL), wash with 1 N HCl (200 mL), and brine (200 mL), dry over Na₂SO₄, filter, and concentrate. Dissolve the residue in THF (50 mL) and slowly add LiAlH₄ (94 mL of a 1M soln in THF, 94 mmol). Warm the resulting mixture to reflux. After 3 h., cool to RT and add H₂O (3.6 mL). Stir for 2 min., then add 1N NaOH (3.6 mL) and stir for 5 min. Add more H₂O (10.8 mL) and stir another 5 min. Finally, add Celite and Na₂SO₄, stir 5 min, then filter and concentrate the filtrate to give the title compound. Use without further purification. MS(ES) 148.2 (M+1)⁺; R_{f}=0.18 (10:1 CHCl₃/MeOH).

By the method of Preparation 90, using the appropriate amine, the following compounds are prepared and isolated:

| Prep. # | Product | Data |
|---|---|---|
| 91 | (1-benzyl-piperidin-4-yl)- | MS(ES) 205.3(M+1)⁺; |
| | methyl-amine | Rf: 0.10(10:1 CHCl₃/MeOH); |
| 92 | [2-(2-chlorophenyl)-ethyl]- | MS(ES) 170.1(M+1)⁺; |
| | methyl-amine | Rf: 0.22(10:1 CHCl₃/MeOH); |

### Preparation 93

### 3-Phenyl-propynoic acid benzyl-methyl-amide

Suspend phenylpropiolic acid (4.2 g, 28.7 mmol) and 1-hydroxybenzotriazole hydrate (4.3 g, 32 mmol) in dry CH₂Cl₂ (250 mTL). Add N-benzyl-N-methylamine (3.5 g, 29 mmol) and triethylamine (20 mL, 145 mmol) followed by 1-ethyl-3-(3-dimethylaminopropyl)carbodiimide hydrochloride (6.1 g, 32 mmol). Stir at RT overnight, then dilute with CH₂Cl₂, wash with 1N HCl solution, saturated NaHCO₃ solution, and brine. Dry the organic layer over MgSO₄, filter, and concentrate to give the title compound (3.36 g, 47%) as a yellow oil that solidifies upon standing. Use without further purification. R_{f}=0.38 (2:1 hexanes/EtOAc); MS(ES) 250.1 (M+1)⁺.

By the method of Preparation 93, using the appropriate amine, the following compounds are prepared and isolated.

| Prep. # | Product | Data |
|---|---|---|
| 94 | 3-Phenyl-propynoic acid (3,5-bis- | Rf=0.42(2:1 hexanes/EtOAc); |
| | trifluoromethyl-benzyl)-methyl-amide | MS/ES:386.1(M+1)⁺. |
| 95 | 3-Phenyl-propynoic acid (3,5-dimethyl-benzyl)- | Rf=0.41(2:1 hexanes/EtOAc); |
| | methyl-amide | MS/ES:278.1(M+1)⁺. |
| 96 | 3-Phenyl-propynoic acid (3,5-dichloro-benzyl)- | Rf=0.42(2:1 hexanes/EtOAc); |
| | methyl-amide | MS(ES) 318.1(M+1)⁺. |
| 97 | 3-Phenyl-propynoic acid (5-chloro-2-methoxy- | Rf=0.32(2:1 hexanes/EtOAc); |
| | benzyl)-methyl-amide | MS(ES) 314.1(M+1)⁺. |
| 98 | 3-Phenyl-propynoic acid (5-fluoro-2-methoxy- | Rf=0.31(2:1 hexanes/EtOAc); |
| | benzyl)-methyl-amide | MS(ES) 298.1(M+1)⁺. |
| 99 | 3-Phenyl-propynoic acid (2-methoxy-5- | Rf=0.32(2:1 hexanes/EtOAc); |
| | trifluoromethoxy-benzyl)-methyl-amide | MS(ES) 364.1(M+1)⁺. |
| 100 | 3-Phenyl-propynoic acid (3-fluoro-5- | Rf=0.45(2:1 hexanes/EtOAc); |
| | trifluoromethyl-benzyl)-methyl-amide | MS(ES) 336.1(M+1)⁺. |
| 101 | 3-Phenyl-propynoic acid (2-chloro-benzyl)- | Rf=0.42(2:1 hexanes/EtOAc); |
| | methyl-amide | MS(ES) 284.1(M+1)⁺. |
| 102 | 3-Phenyl-propynoic acid dibenzyl-amide | Rf=0.62(2:1 hexanes/EtOAc); |
| | | MS(ES) 326.2(M+1)⁺. |
| 103 | 3-Phenyl-propynoic acid methyl-phenethyl- | Rf=0.32(2:1 hexanes/EtOAc); |
| | amide | MS(ES) 264.2(M+1)⁺. |

### Preparation 104

### 1-(2-azido-ethyl)-4-fluoro-benzene

Dissolve the 1-(2-chloroethyl)-4-fluorobenzene (1 eq) in DMSO/H₂O (10:1). Add NaN₃ (2 eq) and stir at RT overnight. Dilute with ether, wash with H₂O, and brine. Dry (MgSO₄), and concentrate to give the title compound. Use crude compound without urification. R_{f}=0.48(20:1 hexanes/EtOAc); IR: 2104 cm-1.

By the method of Preparation 104, using the appropriate starting materials, the following compounds are prepared and isolated.

| Prep. # | Product | Data |
|---|---|---|
| 105 | 1-azidomethyl-3,5-bis- | Rf=0.42(20:1 hexanes/EtOAc); IR:2105cm−1 |
| | trifluoromethyl-benzene | |
| 106 | 3,5-dimethylbenzyl azide | Rf=0.68(20:1 hexanes/EtOAc); ¹H NMR(CDCl₃, 250MHz) |
| | | δ 7.03(s, 1H), 6.96(s, 2H), 4.30(s, 2H), 2.37(s, |
| | | 6H). |
| 107 | 3,5-dichlorobenzyl azide | Rf=0.57(20:1 hexanes/EtOAc); ¹H NMR(CDCl₃, 250MHz) |
| | | δ 7.36(m, 1H), 7.25(s, 2H), 4.36(s, 2H). |
| 108 | 3-phenylpropyl azide | Rf=0.57(20:1 hexanes/EtOAc); ¹H NMR(CDCl₃, 250MHz) |
| | | δ 7.05-7.25(m, 5H), 3.19(t, 2H), 2.62(t, 2H), |
| | | 1.83(quint, 2H). |
| 109 | (4-methoxyphenyl)propyl | Rf=0.40(20:1 hexanes/EtOAc); ¹H NMR(CDCl₃, 250MHz) |
| | azide | δ 7.14(d, 2H), 6.88(d, 2H), 3.83(s, 3H), 3.31(t, |
| | | 2H), 2.69(t, 2H), 1.92(quint, 2H). |
| 110 | 1-[4-(2- | Rf=0.11(20:1 hexanes/EtOAc); ¹H NMR(CDCl₃, 250MHz) |
| | azidoethyl)phenyl]-1- | δ 7.91(d, 2H), 7.32(d, 2H), 3.54(t, 2H), 2.93(t, |
| | ethanone | 2H), 2.67(s, 3H). |
| 111 | 4-azidomethylbiphenyl | Rf=0.49(20:1 hexanes/EtOAc); ¹H NMR(CDCl₃, 250MHz) |
| | | δ 7.52(m, 4H), 7.25-7.4(m, 5H), 4.29(s, 2H). |
| 112 | 4-(azidomethyl)-2,6- | Rf=0.24(20:1 hexanes/EtOAc); ¹H NMR(CDCl₃, 250MHz) |
| | dichloropyridine | δ 7.22(s, 2H), 4.37(s, 2H). |
| 113 | 2-chlorobenzyl azide | Rf=0.60(20:1 hexanes/EtOAc); ¹H NMR(CDCl₃, 250MHz) |
| | | δ 7.45(m, 2H), 7.34(m, 2H), 4.54(s, 2H). |
| 114 | 1-phenethyl azide | Rf=0.61(20:1 hexanes/EtOAc); ¹H NMR(CDCl₃, 300MHz) |
| | | δ 7.3-7.4(m, 5H), 4.62(q, J=6.8Hz, 1H), |
| | | 1.54(d, J=6.8Hz, 3H). |
| 115 | 3-fluorobenzyl azide | Rf=0.51(20:1 hexanes/EtOAc); ¹H NMR(CDCl₃, |
| | | 250MHz) δ 7.38(m, 1H), 7.10(m, 3H), 4.39(s, 2H). |
| 116 | 3-(trifluoromethyl)benzyl | Rf=0.46(20:1 hexanes/EtOAc); ¹H NMR(CDCl₃, 250MHz) |
| | azide | δ 7.5-7.7(m, 4H), 4.47(s, 2H). |
| 117 | 2-(trifluoromethyl)benzyl | Rf=0.60(20:1 hexanes/EtOAc); ¹H NMR(CDCl₃, 250MHz) |
| | azide | δ 7.69(d, 1H), 7.62(m, 2H), 7.49(m, 1H), |
| | | 4.61(s, 2H). |
| 118 | 1-(azidomethyl)- | Rf=0.51(20:1 hexanes/EtOAc); ¹H NMR(CDCl₃, 250MHz) |
| | napthylene | δ 8.07(d, 1H), 7.92(m, 2H), 7.45-7.65(m, 4H), |
| | | 4.81(s, 2H). |
| 119 | 3-chlorobenzyl azide | Rf=0.54(20:1 hexanes/EtOAc); ¹H NMR(CDCl₃, 300MHz) |
| | | δ 7.32(m, 3H), 7.21(m, 1H), 4.33(s, 2H). |
| 120 | 2-phenethyl azide | Rf=0.60(20:1 hexanes/EtOAc); ¹H NMR(CDCl₃, 300MHz) |
| | | δ 7.2-7.35(m, 5H), 3.48(t, 2H), 2.87(t, 2H). |
| 121 | benzyl azide | Rf=0.58(20:1 hexanes/EtOAc); ¹H NMR(CDCl₃, 300MHz) |
| | | δ 7.25-7.42(m, 5H), 4.33(s, 2H). |
| 122 | 4-methoxybenzyl azide | Rf=0.38(20:1 hexanes/EtOAc); ¹H NMR(CDCl₃, 300MHz) |
| | | δ 7.25(d, 2H), 6.91(d, 2H), 4.27(s, 2H), 3.82(s, |
| | | 3H). |
| 123 | 3,5-dibromobenzyl azide | Rf=0.57(20:1 hexanes/EtOAc); ¹H NMR(CDCl₃, 250MHz) |
| | | δ 7.67(s, 1H), 7.44(s, 2H), 4.35(s, 2H). |
| 124 | 2-(4-methoxyphenyl)ethyl | Rf=0.40(20:1 hexanes/EtOAc); ¹H NMR(CDCl₃, 250MHz) |
| | azide | δ 7.17(d, 2H), 6.90(d, 2H), 3.84(s, 3H), 3.51(t, |
| | | 2H, 2.88(t, 2H). |
| 125 | (±)-2-azido-1- | Rf=0.63(20:1 hexanes/EtOAc); ¹H NMR(CDCl₃, 250MHz) |
| | phenylpropane | δ 7.2-7.4(m, 5H), 3.73(m, 1H), 2.88(dd, 1H), |
| | | 2.77(dd, 1H), 1.30(d, 3H). |
| 126 | 2-methylbenzyl azide | Rf=0.60(20:1 hexanes/EtOAc); ¹H NMR(CDCl₃, 250MHz) |
| | | δ 7.15(m, 4H), 4.21(s, 2H), 2.29(s, 3H). |
| 127 | 3-methylbenzyl azide | Rf=0.60(20:1 hexanes/EtOAc); ¹H NMR(CDCl₃, 250MHz) |
| | | δ 7.18(m, 1H), 7.05(m, 3H), 4.22(s, 2H), |
| | | 2.30(s, 3H). |
| 128 | 4-methylbenzyl azide | Rf=0.62(20:1 hexanes/EtOAc); ¹H NMR(CDCl₃, 250MHz) |
| | | δ 7.12(s, 4H), 4.21(s, 2H), 2.28(s, 3H). |
| 129 | 2-bromobenzyl azide | Rf=0.57(20:1 hexanes/EtOAc); ¹H NMR(CDCl₃, 250MHz) |
| | | δ 7.53(d, 1H), 7.30(m, 2H), 7.13(m, 1H), |
| | | 4.41(s, 2H). |
| 130 | 2-methoxybenzyl azide | Rf=0.49(20:1 hexanes/EtOAc); ¹H NMR(CDCl₃, 250MHz) |
| | | δ 7.17(m, 2H), 6.34(m, 2H), 4.24(s, 2H), |
| | | 3.73(s, 3H). |
| 131 | 3-methoxybenzyl azide | Rf=0.40(20:1 hexanes/EtOAc); ¹H NMR(CDCl₃, 250MHz) |
| | | δ 7.21(t, 3H), 6.77(m, 3H), 4.22(s, 2H), 3.72(s, |
| | | 3H). |

### Preparation 132

### 2-(2-methyoxyphenyl)ethyl azide

Add pyridine (3.1 g, 39.4 mmol), p-toluenesulfonyl chloride (1.50 g, 7.9 mmol), and DMAP (50 mg) to a solution of 2-(2-methoxyphenyl)ethyl alcohol (1.0 g, 6.6 mmol) in CH₂Cl₂(0.2M) (25 mL). Allow mixture to stir overnight at RT, then dilute with ether (250 mL) and wash with saturated NaHCO₃ (2×150 mL) and brine. Dry over MgSO₄, filter, and concentrate.

Dissolve the crude residue in DMSO (7 mL), add H₂O (0.7 mL), and NaN₃ (850 mg, 13.2 mmol). Warm the mixture to 50° C. and stir for 48 h, then cool to RT and dilute with ether. Wash twice with H₂O, and then with brine, dry over Na₂SO₄, filter, and concentrate to give the title compound as a pale yellow oil. Use without further on. R_{f}=0.43 (10:1 hexanes/EtOAc); ¹H NMR (CDCl₃, 250 MHz) δ7.11 (m, 2H), 6.80 (m, 2H), 3.75 (s, 3H), 3.38 (t, 2H), 2.85 (t, 2H).

By the method of Preparation 132, using the appropriate alcohol, the following compounds are prepared and isolated.

| Prep. # | Product | Data |
|---|---|---|
| 133 | 2-[3,5-bis(trifluoromethyl)- | Rf=0.37(10:1 hexanes/EtOAc); ¹H NMR(CDCl₃, |
| | phenyl]ethyl azide | 250MHz) δ 7.71(s, 1H), 7.62(s, 2H), 3.53(t, 2H), |
| | | 2.93(t, 2H). |
| 134 | 2,2-diphenylethyl azide | Rf=0.41(10:1 hexanes/EtOAc); ¹H NMR(CDCl₃, |
| | | 250MHz) δ 7.2-7.5(m, 10H), 4.28(t, 1H), 3.93(d, |
| | | 2H). |
| 135 | 2-(3-methylphenyl)ethyl | Rf=0.52(10:1 hexanes/EtOAc); ¹H NMR(CDCl₃, |
| | azide | 250MHz) δ 7.23(m, 1H), 7.04(m, 3H), 3.50(t, 2H), |
| | | 2.87(t, 2H), 2.35(s, 3H). |
| 136 | 2-[(3- | Rf=0.47(10:1 hexanes/EtOAc); ¹H NMR(CDCl₃, |
| | trifluoromethyl)phenyl] | 250MHz) δ 7.4-7.6(m, 4H), 3.55(t, 2H), 2.95(t, |
| | ethyl azide | 2H). |
| 137 | 2-[(4- | Rf=0.28(10:1 hexanes/EtOAc); ¹H NMR(CDCl₃, |
| | dimethylamino)phenyl] | 250MHz) δ 7.00(d, 2H), 6.61(d, 2H), 3.35(t, 2H), |
| | ethyl azide | 2.83(s, 6H), 2.71(t, 2H). |

### Preparation 138

### 1-(3-methylphenyl)-1-azidoethane

Dissolve 1-(3-methylphenyl)-1-ethanol (1.36 g, 10 mmol) in dry toluene. Cool to 0° C. and add DPPA (diphenylphosphoryl azide, 3.3 g, 12 mmol) followed by 1,8-diazabicyclo[5.4.0]undec-7-ene (DBU, 1.8 mL, 12 mmol). Warm the resulting mixture to RT and stir overnight, then dilute with H₂O, and extract with ether. Wash the organic layer with 1 N HCl, saturated NaHCO₃, and brine. Dry over MgSO₄, filter, and concentrate to give the title compound (1.3 g, 81%) as a pale yellow oil. Use without further purification. R_{f}=0.66 (20:1 hexanes/EtOAc); ¹H NMR (CDCl₃, 250 MHz) δ7.1-7.4H), 4.61 (q, 1H), 2.42 (2, 3H), 1.56 (d, 3H).

By the method of Preparation 138, using the appropriate alcohol starting material, the following compounds are prepared and isolated.

| Prep. # | Product | Data |
|---|---|---|
| 139 | 1-(4-fluorophenyl)-1- | Rf=0.63(10:1 hexanes/EtOAc); ¹H |
| | azidoethane | NMR(CDCl₃, 250MHz) δ |
| | | 7.2-7.4(m, 2H), 7.1(t, 2H), |
| | | 4.64(q, 1H), 1.55(d, 3H). |
| 140 | (±)-1-[(3- | Rf=0.60(20:1 hexanes/EtOAc); ¹H |
| | trifluoromethyl)phenyl]- | NMR(CDCl₃, 250MHz) |
| | 1-azidoethane | δ 7.5-7.7(m, 3H), 7.35(m, |
| | | 1H), 4.73(q, 1H), 1.59(d, 3H). |

### Preparation 141

### 1-(2-Chloro-phenyl)-pyrazolidin-3-one

Dissolve sodium metal (1.5 g, 64.4 mmol) in n-butanol (25 mL) then add 2-chlorophenylhydrazine hydrochloride (5.0 g, 28.0 mmol). To this mixture, add methyl acrylate (3.8 mL, 42.0 mmol) in a dropwise fashion, then warm the mixture to reflux. After 5 h., add water (100 mL) while the solution is still hot, then adjust the pH of the solution with to pH=6 with 50% aqueous acetic acid. Wash with water and filter the precipitate. Rinse the precipitate with ether and dry on vacuum pump to afford 3.67 g (67%) of the title compound as a white solid. MS(ES) 197.1 (M+1)⁺; R_{f}=0.4

### Preparation 142

### (2-Chloro-4-methyl-phenyl)-methyl-amine

Stir 2-chloro-4-methylaniline (5.0 g, 35.5 mmol) and methyl iodide (2.2 mL, 35.5 mmol) neat at RT. After 12 h, add water and extract with EtOAc. Wash the organic layer with saturated aqueous NaHCO₃, and brine, dry over sodium sulfate, filter, and concentrate. Purify by chromatography on SiO₂ (EtOAc/hexanes gradient) to afford 3.4 g of a 1:1 mix of the title compound and N,N-dimethyl material. Use the mixture without further purification. IS (MS) 156.1 (M+1)⁺; R_{f}=0.90 (20% EtOAc/hexanes).

### Preparation 143

### N′-(2-Chloro-phenyl)-hydrazinecarboxylic acid tert-butyl ester

Dissolve o-chlorophenylhydrazine hydrochloride (5.0 g, 28.0 mmol), potassium carbonate (138 g, 11.6 mmol) and di-t-butyl-dicarbonate (11.6 g, 84.0 mmol) in THF (50 mL) and water (50 mL) and stir at RT. After 4 days, evaporate off the organics, add 20% iPrOH/CHCl₃ and wash with saturated aqueous NaHCO₃, and brine. Dry the organic layer over sodium sulfate, filter, and concentrate to dryness. Purify the residue by chromatography using an EtOAc/hexanes gradient to afford the title compound (5.65 g, 83%) as a white solid. MS(ES) 241.0 (M−1)⁻; R_{f}−0.13 (10% EtOAc/hexanes).

### Preparation 144

### 2-(2-Chloro-phenyl)-pyrazolidine-1-carboxylic acid tert-butyl ester

Dissolve sodium hydride (1.1 g, 27.2 mmol) and 1,3-dibromopropane (1.4 mL, 13.6 mmol) in DMF (100 mL) at 0° C. Add N′-(2-chloro-phenyl)-hydrazinecarboxylic acid tert-butyl ester (3.3 g, 13.6 mmol) and stir at 0° C. After 1 h, quench with water and concentrate to dryness. Dissolve the residue in 20% iPrOH/CHCl₃ and wash with water. Extract the aqueous layer with CHCl₃ and wash the combined organics with saturated aqueous NaHCO₃, and brine. Dry over sodium sulfate, filter, and concentrate to dryness. Purify the residue by chromatography using an EtOAc/hexanes gradient to afford the title compound (3.83 g, 99%) as a yellow oil. MS(ES) 283.1 (M+1)⁺; R_{f}=0.81 (1:1 EtOAc/hexanes).

### Preparation 145

### 1-(2-Chloro-phenyl)-pyrazolidine hydrochloride

Dissolve 2-(2-chloro-phenyl)-pyrazolidine-1-carboxylic acid tert-butyl ester (3.84 g, 13.6 mmol) in a solution of acetic acid saturated with HCl (30 mL) and stir at RT. After 16 h, concentrate the mixture to dryness. Slurry the residue in 1,2-dichloroethane and concentrate to dryness twice. Triturate with ether, filter the precipiate and dry under vacuum to afford the title compound (2.14 g, 72%). MS(ES) 183.0 (M+1)⁺; Anal. calc'd for C₉H₁₁ClN₂.HCl: C, 49.33; H, 5.52; N, 12.79. Found: C, 49.28; H, 5.57; N, 12.70.

### Preparation 146

### (2-Chloro-4-fluoro-phenyl)-methyl-amine

Using a method similar to Preparation 142, with the exception of using 2-chloro-4-fluoroaniline (5.0 g, 34.5 mmol, Aldrich) and methyl iodide (2.2 mL, 34.5 mmol), affords 3.4 g of an approximate 1:1 mix of the title compound and N,N-dimethyl material. Carried on as is without further purification. MS(ES) 160.0 (M+1)⁺; R_{f}=0.9 (20% EtOAc/hexanes).

### Preparation 147

### 2-Chloropyridine-3-carboxaldebyde

Prepare lithium diisopropylamide by the addition of n-butyl lithium (37.5 mL, 0.06 mol, 1.6 M in hexanes) to a solution of diisopropylamine (8.39 mL, 0.06 mol) in THF (150 mL). Cool the mixture to −70° C. and add 2-chloropyridine (4.96 mL, 0.05 mol) dropwise via syringe while stirring. After 1.5 h., add DMF (7.73 mL, 0.10 mol) dropwise via syringe. After another 1.5 h., remove the cooling bath and quench with water as the mixture warms to −25° C. Extract the mixture with EtOAc, dry over sodium sulfate, filter, and concentrate in vacuo. Purify the residue by chromatography on silica gel using 10% EtOAc/hexanes to provide the title aldehyde (2.58 g, 37%) as an off white solid. MS(EI) 140.99 (M⁺); ¹H NMR (d₆ DMSO, 300 MHz) δ10.28 (s, 1H), 8.67 (dd, 1H, J=2.2, 4.8 Hz), 8.27 (dd, 1H, J=2.2, 7.7 Hz), 7.60-7.70 (m, 1H).

### Preparation 148

### (2-Chloro-pyridin-3-ylmethyl)-methyl-amine

Dissolve 2-chloropyridine-3-carboxaldehyde (2.50 g, 17 mmol) in MeOH (20 mL) and add methylamine (15.0 mL of a 2M in MeOH,30 mmol). Stir the resulting mixture at RT. After 24 h, cool the reaction mixture in an ice bath and add sodium borohydride (5.25 g, 0.139 mol) in small portions. Stir the mixture for 2 h., then concentrate in vacuo. Add water, and extract with CH₂Cl₂. Dry the organic extracts over Na₂SO₄, filter, and concentrate. Purify the residue by chromatography on silica gel eluting with a MeOH/CH₂Cl₂ gradient to obtain the title compound (2.23 g, 85%) as a light oil. MS(EI) 156.0 (M⁺); ¹H NMR (d₆ DMSO, 300 MHz) δ8.25-8.30 (m, 1H), 7.87-7.95 (m, 1H), 7.40-7.45 (m, 1H), 3.70 (s, 2H), 2.30 (s, 3H).

### Preparation 149

### 3-chloropyridine-4-carboxaldehyde

Using a method similar to Preparation 147, with the exception of using 3-chloropyridine (4.75 mL, 0.05 mol), affords the title compound as a light yellowish solid. MS(EI) 141.0 (M⁺); ¹H NMR (d₆ DMSO, 300 MHz) δ10.32 (s, 1H), 8.87 (s, 1H), 8.77 (d, 1H, J=4.8 Hz), 7.75 (d, 1H, J=4.8 Hz).

### Preparation 150

### (3-Chloro-pyridin-4-ylmethyl)-methyl-amine

Using a method similar to Preparation 148, with the exception of using 3-chloropyridine-4-carboxaldehyde (2.00 g, 0.014 mol), affords the title compound as a light oil. MS(EI) 156.0 (M⁺); ¹H NMR (d₆ DMSO, 300 MHz) δ8.55 (s, 1H), 8.48 (d, 1H, J=4.8 Hz), 7.54 (d, 1H, J=4.8 Hz), 3.79 (s, 2H), 2.31 (s, 3H).

### Preparation 151

### 4-Chloropyridine-3-carboxaldehyde

Using a method similar to Preparation 147, with the exception of using 4-chloropyridine hydrochloride (3.75 g, 0.025 mol), affords the title compound as a light orange solid. MS(ES) 142.0 (M+1)⁺; R_{f}=0.37 (6% MeOH/CH₂Cl₂).

### Preparation 152

### (4-Chloro-pyridin-3-ylmethyl)-methyl-amine

Using a method similar to Preparation 148, with the exception of using 4-chloropyridine-3-carboxaldehyde (0.80 g, 0.0056 mol), affords the title compound as a light oil. MS(EI) 156.0 (M⁺); ¹H NMR (d₆ DMSO, 300 MHz) δ8.60 (s, 1H), 8.42 (d, 1H, J=5.1 Hz), 7.50 (d, 1H, J=5.1 Hz), 3.75 (s, 2H), 2.29 (s, 3H).

### Preparation 153

### 1-Phenethyl-5-phenyl-1H-[1,2,3]triazole-4-carboxylic acid ethyl ester

Combine ethyl benzoylacetate (1.49 g, 7.76 mmol), 2-phenethyl azide (0.87 g, 6.44 mmol), and potassium carbonate (3.56 g, 25.8 mmol) in DMSO (16 mL) and heat at 50° C. overnight. Dilute the reaction mixture with water and extract with EtOAc. Wash the combined extracts with brine, dry over Na₂SO₄, filter, and concentrate. Purify the residue by chromatography over silica gel using a hexanes/EtOAc gradient to provide the title compound (0.895 g, 43%) as a pale yellow oil. MS(ES) 322.0 (M+1)⁺; Anal. Calc'd for C₁₉H₁₉N₃O₂: C, 71.00; H, 5.96; N, 13.07. Found: C, 71.30; H, 5.84; N, 13.06.

### Preparation 154

### (3-Chloro-pyridin-4-yl)-isopropyl-amine

Combine 3-chloro-4-aminopyridine (3.00 g, 14.6 mmol) and 2-bromopropane (2.20 mL, 23.4 mmol) in a sealed tube and heat the mixture overnight at 100-110° C. Cool the mixture to RT, add aqueous NaHCO₃, and extract with EtOAc. Dry the combined extracts over Na₂SO₄, filter, and concentrate. Purify the residue by chromatography over silica gel using CH₂Cl₂ to provide the title compound (1.72 g, 69%) as a light oil. MS(ES) 170.2 (M+1)⁺; R_{f}=0.71 (25% EtOAc/hexanes).

### Preparation 155

### 1-(3,5-Bis-trifluoromethyl-benzyl)-5-methyl-1H-[1,2,3]triazole-4-carboxylic acid ethyl ester

Combine ethyl acetoacetate (10.0 g, 77.0 mmol), 3,5-bis-trifluoromethyl-benzyl azide (40.3 g, 150 mmol), and potassium carbonate (43 g, 308 mmol) in DMSO (100 mL). Stir 4 days at 50° C., then add water and extract with EtOAc. Wash with water, and brine, dry over sodium sulfate, filter, and concentrate. Dissolve the residue in warm EtOAc (20 mL) and place in a freezer. After 4 h, add hexanes and collect the crystalline material by filtration. Dry under vacuum to afford 21.7 g (74%) of the title compound as a white solid. MS(ES) 382.0 (M+1); R_{f}=0.55 (1:1 EtOAc/hexanes).

### Preparation 156

### (R)-(+)-2-(2-chlorophenyl)-pyrrolidine

To a dry Schlenk flask under nitrogen is added 0.540 goof (R,R)-(+)-ethylene-1,2-bis(η⁵-4,5,6,7-tetrahydro-1-indenyl)titanium difluoride and 120 mL of dry THF. To this solution are added under nitrogen in the following order: 2-(2-chlorophenyl)-pyrroline (15 g), phenylsilane (15 g), pyrrolidine (0.48 mL), and MeOH (0.24 mL). The solution is stirred at RT for 48 h., then the mixture is diluted with 350 mL of diethylether and carefully added with vigorous stirring to 1200 mL of 1M HCl. The aqueous layer is separated and extracted with three portions of diethyl ether (300 mL each). The aqueous layer is made basic with 3M NaOH and extracted with 5 portions of diethyl ether (200 mL each). The combined ether layers are dried over magnesium sulfate and concentrated in vacuo. The residue is purified by vacuum transfer to give the title compound (15 g, 93%) as a colorless oil. ¹H NMR (400 MHz, CDCl₃) δ7.61-7.58 (m, 1H), 7.32-7.30 (m, 1H), 7.26-7.21 (m, 1H), 7.16-7.11 (m, 1H), 4.53 (t, J=, 1H), 3.21-3.16 (m, 1H), 310-3.03 (m, 1H), 2.37-2.28 (m, 1H), 2.04 (br s, 1H), 1.93-1.70 (m,2H), 1.60-1.51 (m, 1H). ^{13 C NMR (}100 MHz, CDCl₃) 25.7, 33.1, 47.2, 59.0, 127.0, 127.4, 127.8, 129.5, 133.1, 143.2.

MS(ES) 182 (M+1)⁺; [α]_{D}=+70.4 (c=0.06, MeOH).

### Preparation 157

### 1-(3,5-Bis-trifluoromethyl-benzyl)-5-hydroxy-1H-[1,2,3]triazole-4-carboxylic acid ethyl ester

Combine a solution of sodium ethoxide (5.5 mL, 21 wt % in EtOH) and diethyl malonate (2.50 mL, 16.5 mmol) in EtOH (26 mL) with a solution of 1-azidomethyl-3,5-bis-trifluoromethyl-benzene (4.40 g, 16.3 mmol) in EtOH (6 mL) and heat to 80° C. After 7 h, cool to RT and concentrate the mixture under reduced pressure. Dissolve the viscous oil in H₂O (20 mL), and add 1N HCl until the solution reaches pH 2. Collect the precipitate by filtration and dry under reduced pressure to give the title compound (5.42 g, 87%) as a white solid, MS(ES) 384.0 (M+1)⁺; ¹H NMR (400 MHz, CHCl₃) δ8.05 (s, 1H), 7.92 (s, 2H), 5.41 (s, 2H), 4.15 (q, 2H, J=7.3 Hz), 1.22 (t, 3H, J=7.3 Hz).

### Preparation 158

### 1-(3,5-Bis-trifluoromethyl-benzyl)-5-chloro-1H-[1,2,3]tri-azole-4-carboxylic acid ethyl ester

Add PCl₅ (5.73 g, 27.5 mmol) to a solution of 1-(3,5-bis-trifluoromethyl-benzyl)-5-hydroxy-1H-[1,2,3]triazole-4-carboxylic acid ethyl ester (5.30 g, 13.8 mmol) in toluene (150 mL) and heat to 50° C. After 2 h, cool the mixture to RT and concentrate under reduced pressure. Dissolve the residue in ether (100 mL) and wash with saturated NaHCO₃ (2×100 mL) and brine (100 mL), then dry, filter, and concentrate. Purify the crude material by passing through a short plug of silica gel using a linear gradient of 50% to 80% EtOAc/hexanes. Recrystallize from 1:1 diethyl ether:petroleum ether (150 mL) to afford the title compound (3.90 g, 70%) as white plates. MS(ES) 402.0 (M+1)⁺; ¹H NMR (400 MHz, CHCl₃) δ7.88 (s, 1H), 7.76 (s, 2H), 5.67 (s, 2H), 4.43 (q, 2H, J=7.0 Hz), 1.40 (t, 3H, J=7.0 Hz).

### Preparation 159

### 1-(3,5-Bis-trifluoromethyl-benzyl)-1H-[1,2,3]triazole-4-carboxylic acid ethyl ester

Combine 1-azidomethyl-3,5-bis-trifluoromethyl-benzene (340 mg, 1.26 mmol) with a solution of ethyl propiolate (160 mg, 1.63 mmol) in toluene (3.0 mL) and heat to 100° C. for 18 h in a sealed tube. Cool the solution to RT, concentrate in vacuo, and purify the residue by chromatography using a linear gradient of 15% to 50% EtOAc/hexanes to afford the title compound (233 mg, 50%) as a clear, viscous oil that solidified upon standing. MS(ES) 368.2 (M+1)⁺; ¹H NMR (400 MHz, CDCl₃) δ8.08 (s, 1H), 7.78 (s, 1H), 7.73 (s, 2H), 5.70 (s, 2H), 4.41 (q, 2H, J=6.8 Hz), 1.39 (t, 3H, J=7.3 Hz).

Using an analogous method to Preparation 159, with the appropriate starting materials, yields the following compounds.

| Prep. # | Product | Data |
|---|---|---|
| 160 | 1-(3,5-Bis-trifluoromethyl-benzyl)-5- | MS(ES) 368.1(M+1)⁺. |
| | methyl-1H-[1,2,3]triazole-4-carboxylic acid | |
| | methyl ester | |
| 161 | 1-(3,5-Bis-trifluoromethyl-benzyl)-5-ethyl- | MS(ES) 396.1(M+1)⁺. |
| | 1H-[1,2,3]tri-azole-4-carboxylic acid ethyl | |
| | ester | |
| 162 | 1-(3,5-Bis-trifluoromethyl-benzyl)-5- | MS(ES) 396.1(M+1)⁺. |
| | propyl-1H-[1,2,3]triazole-4-carboxylic acid | |
| | methyl ester | |
| 163 | 1-(3,5-Bis-trifluoromethyl-benzyl)-5-butyl- | MS(ES) 410.1(M+1)⁺. |
| | 1H-[1,2,3]triazole-4-carboxylic acid methyl | |
| | ester | |
| 164 | 1-(3,5-Bis-trifluoromethyl-benzyl)-5- | MS(ES−) 434.1(M−1)⁻. |
| | trifluoromethyl-1H-[1,2,3]triazole-4- | |
| | carboxylic acid ethyl ester | |
| 165 | 1-(3,5-bis-trifluoromethyl-benzyl)-5- | MS(ES) 445.2(M+1)⁺; ¹H |
| | pyridin-2-yl-1H-[1,2,3]triazole-4-carboxylic | NMR(400MHz, CDCl₃) δ 8.74(m, 1H), |
| | acid ethyl ester | 7.78(dt, 1H, J=2.0, 7.8Hz), |
| | | 7.73(m, 2H), 7.56(s, 2H), 7.40(ddd, 1H, |
| | | J=1.5, 4.9, 7.3Hz), 5.91(s, 2H), |
| | | 4.37(q, 2H, J=7.3Hz), 1.35(t, 3H, |
| | | J=7.3Hz). |

### Preparation 166

### 1-(3,5-Bis-trifluoromethyl-benzyl)-1H-[1,2,3]triazole-4-carboxylic acid

Combine lithium hydroxide monohydrate (260 mg, 6.20 mmol) with a solution of 1-(3,5-bis-trifluoromethyl-benzyl)-1H-[1,2,3]triazole-4-carboxylic acid ethyl ester (230 mg, 0.626 mmol) in 2:1 dioxane H₂O (6.75 mL) and stir at RT for 3 h. Dilute solution with H₂O (10 mL) and treat with aqueous 1N HCl until pH 3 is obtained. Collect white precipitate by filtration and dry in vacuo to afford the title compound (195 mg, 92%) as a white powder. MS[EI⁻] 338.1 (M−H)⁻. ¹H NMR (400 MHz, DMSO-d₆) δ8.06 (s, 1H), 7.31 (s, 1H), 7.30 (s, 2H), 5.04 (s, 2H).

Using a method analogous to Preparation 166, with the appropriate starting materials, the following compounds may be prepared.

| Prep. # | Product | Data |
|---|---|---|
| 167 | 1-(3,5-Bis-trifluoromethyl-benzyl)-5- | MS[EI⁻] 352.1(M−H)⁻ |
| | methyl-1H-[1,2,3]triazole-4-carboxylic | |
| | acid | |
| 168 | 1-(3,5-Bis-trifluoromethyl-benzyl)-5- | MS[EI−] 366.2(M−H)⁻. |
| | ethyl-1H-[1,2,3]triazole-4-carboxylic acid | |
| 169 | 1-(3,5-Bis-trifluoromethyl-benzyl)-5- | MS[EI−] 380.2(M−H)⁻. |
| | propyl-1H-[1,2,3]triazole-4-carboxylic | |
| | acid | |
| 170 | 1-(3,5-Bis-trifluoromethyl-benzyl)-5- | MS[EI+] 396.1(M+H)⁺, MS[EI−] |
| | butyl-1H-[1,2,3]triazole-4-carboxylic acid | 394.2(M−H)⁻. |
| 171 | 1-(3,5-Bis-trifluoromethyl-benzyl)-5- | MS[EI−] 406.1(M−H)⁻ |
| | trifluoromethyl-1H-[1,2,3]triazole-4- | |
| | carboxylic acid | |
| 172 | 1-Phenethyl-5-phenyl-1H-[1,2,3]triazole- | MS(ES) 294.0(M+1)⁺; Anal. Calc'd |
| | 4-carboxylic acid | for C₁₇H₁₅N₃O₂.0.35H₂O: C, 68.15; H, |
| | | 5.28; N, 14.02. Found: C, 67.87; H, |
| | | 5.08; N, 14.44. |
| 173 | 1-(3,5-Bis-trifluoromethyl-benzyl)-5- | MS(ES) 371.8(M−1)⁻; ¹H NMR(400MHz, |
| | chloro-1H-[1,2,3]triazole-4-carboxylic | DMSO-d6) δ 12.7(br s, 1H), |
| | acid | 7.33(s, 1H), 7.22(s, 2H), 5.07(s, 2H). |
| 174 | 1-(3,5-Bis-trifluoromethyl-benzyl)-5- | MS(ES−) 410.2(M−1)⁻. ¹H NMR(400MHz, |
| | butoxy-1H-[1,2,3]triazole-4-carboxylic | CHCl₃) δ 7.86(s, 1H), 7.77(s, |
| | acid | 2H), 5.43(s, 2H), 4.69(m, 2H), |
| | | 1.63(m, 2H), 1.33(m, 2H), 1.23(m, 2H), |
| | | 0.89(t, 3H, J=6.8Hz). |
| 175 | 5-Benzyloxy-1-(3,5-bis-trifluoromethyl- | MS(ES−) 444.2(M−1)⁻. ¹H NMR(400MHz, |
| | benzyl)-1H-[1,2,3]triazole-4-carboxylic | CHCl₃) δ 7.82(s, 1H), 7.60(s, |
| | acid | 2H), 7.22-7.30(m, 5H), 5.69(s, 2H), |
| | | 5.29(s, 2H). |
| 176 | 1-(3,5-Bis-trifluoromethyl-benzyl)-5- | MS(ES) 384.0(M+1)⁺. |
| | ethoxy-1H-[1,2,3]triazole-4-carboxylic | |
| | acid | |
| 177 | 1-(3,5-Bis-trifluoromethyl-benzyl)-5- | MS(ES) 398.1(M+1)⁺ |
| | propoxy-1H-[1,2,3]triazole-4-carboxylic | |
| | acid | |
| 178 | 5-Chloro-1-(3,5-dichloro-benzyl)-1H- | MS(FAB) 305.9(M+1)⁺. |
| | [1,2,3]triazole-4-carboxylic acid | |
| 179 | 1-(3,5-Bis-trifluoromethyl-benzyl)-5- | MS(ES) 405.2(M+1)⁺; ¹H NMR(400MHz, |
| | pyrrol-1-yl-1H-[1,2,3]triazole-4- | DMSO-d6) δ 13.16(br s, |
| | carboxylic acid | COOH), 8.03(s, 1H), 7.64(s, 2H), |
| | | 6.97(t, 2H, J=2Hz), 6.23(t, 2H, J=2.0Hz), |
| | | 5.69(s, 2H). |
| 180 | 1-(3,5-Bis-trifluoromethyl-benzyl)-5-(1- | MS[ES] 419.3(M+1)⁺. |
| | methyl-1H-pyrrol-2-yl)-1H- | |
| | [1,2,3]triazole-4-carboxylic acid | |
| 181 | 1-(3,5-Bis-trifluoromethyl-benzyl)-5- | ¹H NMR(400MHz, DMSO) δ 9.05(d, |
| | pyrazin-2-yl-1H-[1,2,3]triazole-4- | 1H, J=1.6), 8.67(m, 2H), 8.04(s, 1H), |
| | carboxylic acid | 7.86(s, 2H), 5.86(s, 2H). |
| 182 | 1-(3,5-Bis-trifluoromethyl-benzyl)-5- | MS(ES) 418.1(M+1)⁺ |
| | pyrimidin-5-yl-1H-[1,2,3]triazole-4- | |
| | carboxylic acid | |
| 183 | 1-(3,5-Bis-trifluoromethyl-benzyl)-5-(4- | MS(ES) 462.1(M+1)⁺ |
| | methylsulfanyl-phenyl)-1H- | |
| | [1,2,3]triazole-4-carboxylic acid | |
| 184 | 1-(3,5-bis-trifluoromethyl-benzyl)-5- | MS(ES−) 415.0(M−1)⁻. ¹H NMR(400MHz, |
| | pyridin-2-yl-1H-[1,2,3]triazole-4- | DMSO-d₆) δ 13.2(br s, 1H), |
| | carboxylic acid | 8.70(m, 1H), 8.05(s, 1H), 7.93(dt, 1H, J=2.0, |
| | | 7.8Hz), 7.83(s, 2H), 7.74(m, 1H), |
| | | 7.53(ddd, 1H, J=1.0, 4.9, 7.3Hz), |
| | | 5.88(s, 2H). |
| 185 | 1-(3,5-Bis-trifluoromethyl-benzyl)-5- | MS(ES−) 415.1(M−1)⁻; ¹H NMR(400MHz, |
| | pyridin-3-yl-1H-[1,2,3]triazole-4- | DMSO-d6) δ 13.05(br s, 1H), |
| | carboxylic acid | 8.66(m, 1H), 8.56(d, 1H, J=1.5Hz), |
| | | 8.05(s, 1H), 7.85(dt, 1H, J=2.0, 7.8Hz), |
| | | 7.71(s, 2H), 7.48(dd, 1H, J=4.9, |
| | | 7.8Hz), 5.79(s, 2H). |
| 186 | 1-(3,5-Bis-trifluoromethyl-benzyl)-5- | MS(ES) 417.1(M+1)⁺; ¹H NMR(400MHz, |
| | pyridin-4-yl-1H-[1,2,3]triazole-4- | DMSO-d6) δ 13.17(br s, 1H), |
| | carboxylic acid | 8.67(br s, 2H), 8.04(s, 1H), 7.73(s, |
| | | 2H), 7.45(d, 2H, J=5.4Hz), 5.78(s, |
| | | 2H). |
| 187 | 1-(3,5-Bis-trifluoromethyl-benzyl)-5- | MS(ES−) 416.4(M−1)⁻; ¹H NMR(400MHz, |
| | pyridazin-4-yl-1H-[1,2,3]triazole-4- | DMSO-d6) δ 13.28(br s, 1H), |
| | carboxylic acid | 9.39(dd, 1H, J=0.9, 5.4Hz), 9.30(dd, |
| | | 1H, J=1.0, 2.5Hz), 8.07(s, 1H), |
| | | 7.88(dd, 1H, J=2.4, 5.3Hz), 7.83(s, 2H), |
| | | 5.81(s, 2H). |
| 188 | 1-(3,5-Bis-trifluoromethyl-benzyl)-5- | MS(ES−) 404.3(M−1)⁻. ¹H NMR(400MHz, |
| | furan-2-yl-1H-[1,2,3]triazole-4-carboxylic | DMSO-d6) δ 13.27(br s, 1H), |
| | acid | 8.09(s, 1H), 7.92(d, 1H, J=1.5Hz), |
| | | 7.86(s, 2H), 7.28(d, 1H, J=3.4Hz), |
| | | 6.70(dd, 1H, J=2.0, 3.4Hz), 6.04(s, |
| | | 2H). |
| 189 | 1-(3,5-Bis-trifluoromethyl-benzyl)-5- | MS(ES−) 404.2(M−1)⁻; ¹H NMR(400MHz, |
| | furan-3-yl-1H-[1,2,3]triazole-4-carboxylic | DMSO-d6) δ 13.05(br s, 1H), |
| | acid | 8.08(m, 2H), 7.83(m, 1H), 7.78(s, |
| | | 2H), 6.71(dd, 1H, J=1.0, 2.0Hz), |
| | | 5.87(s, 2H). |
| 190 | 1-(3,5-Bis-trifluoromethyl-benzyl)-5- | MS(ES−) 420.0(M−1)⁻; ¹H NMR(400MHz, |
| | thiophen-2-yl-1H-[1,2,3]triazole-4- | DMSO-d6) δ 13.14(br s, 1H), |
| | carboxylic acid | 8.06(s, 1H), 7.85(dd, 1H, J=1.0, 4.9Hz), |
| | | 7.69(s, 2H), 7.40(dd, 1H, J=1.5, |
| | | 3.4Hz), 7.20(dd, 1H, J=3.4, 4.9Hz), |
| | | 5.84(s, 2H). |
| 191 | 1-(3,5-Bis-trifluoromethyl-benzyl)-5-(5- | MS(ES−) 434.0(M−1)⁻; ¹H NMR(400MHz, |
| | methyl-thiophen-2-yl)-1H-[1,2,3]triazole- | DMSO-d6) δ 13.13(br s, 1H), |
| | 4-carboxylic acid | 8.01(s, 1H), 7.69(s, 2H), 7.18(d, 1H, J=3.4Hz), |
| | | 6.90(dd, 1H, J=1.0, 3.4Hz), |
| | | 5.83(s, 2H), 2.45(d, 3H, J=1.0Hz). |
| 192 | 1-(3,5-bis-trifluoromethyl-benzyl)-5- | MS(ES) 418.1(M+1)⁺; |
| | pyrazin-2-yl-1H-[1,2,3]triazole-4- | |
| | carboxylic acid | |
| 193 | 1-(3,5-bis-trifluoromethyl-benzyl)-5-(4- | MS(ES) 434.0(M+1)⁺; |
| | fluoro-phenyl)-1H-[1,2,3]triazole-4- | |
| | carboxylic acid | |
| 194 | 5-Amino-1-(3,5-bis-trifluoromethyl- | MS(ES) 355.2(M+1)⁺; ¹H NMR(400MHz, |
| | benzyl)-1H-[1,2,3]triazole-4-carboxylic | DMSO-d6) δ 12.51(s, COOH), |
| | acid | 8.09(s, 1H), 7.90(s, 2H), 6.34(s, 2H), |
| | | 5.61(s, 2H). |
| 195 | 1-(3,5-Bis-trifluoromethyl-benzyl)-5- | ¹H NMR(400MHz, DMSO-d6) δ |
| | isopropyl-1H-[1,2,3]triazole-4-carboxylic | 13.08(br s, 1H), 8.14(s, 1H), 7.88(s, 2H), |
| | acid | 5.96(s, 2H), 3.52(quint., 1H, J=7.3), |
| | | 1.19(d, 6H, J=7.0) |

### Preparation 196

### 1-(3,5-Bis-trifluoromethyl-benzyl)-5-butoxy-1H-[1,2,3]triazole-4-carboxylic acid ethyl ester

Combine a solution of 1-(3,5-bis-trifluoromethyl-benzyl)-5-hydroxy-1H-[1,2,3]triazole-4-carboxylic acid ethyl ester (120 mg, 0.31 mmol) in DMF (5.0 mL) with 1-iodobutane (40 μL) and cesium flouride (188 mg, 1.24 mmol) and stir at RT. After 3 h., add cesium carbonate (200 mg). After 16 h., add H₂O (5 mL), stir the solution for 15 min, then extract with ether (3'10 mL). Combine the organic layers and wash with H₂O (10 mL) and brine (10 mL) then dry, filter, and concentrate. Purify the crude material by chromatography on silica gel using 20% EtOAc/bexanes to afford the title compound as a clear, colorless oil. MS(ES) 440.1 (M+1)⁺; ¹H NMR (400 MHz, CHCl₃) δ7.84 (s, 1H), 7.75 (s, 2H), 5.44 (s, 2H), 4.51 (t, 2H, J=6.6 Hz), 4.40 (t, 2H, J=7.0 Hz), 1.63 (m, 2H), 1.40 (t, 3H, J=7.0 Hz), 1.33 (m, 2H), 0.88 (t, 3H, J=7.4 Hz).

Using a method analogous to Preparation 196, with the appropriate starting materials, the following compounds may be prepared.

| Prep.# | Product | Data |
|---|---|---|
| 197 | 5-Benzyloxy-1-(3,5-bis-trifluoromethyl- | MS(ES) 474.1(M+1)⁺; ¹H NMR(400MHz, |
| | benzyl)-1H-[1,2,3]triazole-4-carboxylic | CHCl₃) δ 7.80(s, 1H), 7.56(s, |
| | acid ethyl ester | 2H), 7.17-7.31(m, 5H), 5.54(s, 2H), |
| | | 5.23(s, 2H), 4.45(q, 2H, J=7.0Hz), |
| | | 1.40(t, 3H, J=7.0Hz). |
| 198 | 1-(3,5-Bis-trifluoromethyl-benzyl)-5- | MS(ES) 412.1(M+1)⁺; ¹H NMR(400MHz, |
| | ethoxy-1H-[1,2,3]triazole-4-carboxylic | CHCl₃) δ 7.85(s, 1H), 7.77(s, |
| | acid ethyl ester | 2H), 5.46(s, 2H), 4.59(q, 2H, J=7.5Hz), |
| | | 4.40(q, 2H, J=7.5Hz), 1.41(t, |
| | | 3H, J=7.5Hz), 1.31(t, 3H, J=7.5Hz). |
| 199 | 1-(3,5-Bis-trifluoromethyl-benzyl)-5- | MS(ES) 426.0(M+1)⁺; ¹H NMR(400MHz, |
| | propoxy-1H-[1,2,3]triazole-4-carboxylic | CHCl₃) δ 7.83(s, 1H), 7.75(s, |
| | acid ethyl ester | 2H), 5.46(s, 2H), 4.47(t, 2H, J=6.6Hz), |
| | | 4.38(q, 2H, J=7.1Hz), 1.70(s, |
| | | 2H, J=7.1Hz), 1.39(t, 3H, J=6.6Hz), |
| | | 0.92(t, 3H, J=7.1Hz). |

### Preparation 200

### 1-(3,5-Bis-trifluoromethyl-benzyl)-5-methoxy-1H-[1,2,3]triazole-4-carboxylic acid

Add dimethyl sulphate (0.14 g, 1.15 mmol) to a suspension of 1-(3,5-bis-trifluoromethyl-benzyl)-5-hydroxy-1H-[1,2,3]triazole-4-carboxylic acid ethyl ester (0.21 g, 0.55 mmol) and potassium carbonate (0.40 g, 1.23 mmol) in DMF (2.0 mL) and stir at 60° C. After 18 h., dilute with water and extract with EtOAc. Combine the organic layers and wash with water and brine, then dry, filter, and concentrate to give crude 1-(3,5-bis-trifluoromethyl-benzyl)-5-methoxy-1H-[1,2,3]triazole-4-carboxylic acid ethyl ester (0.22 g, 95%). Dissolve this material in 1:1 dioxane:water (6.0 mL), add lithium hydroxide monohydrate (0.14 g, 3.34 mmol) and stir the mixture at RT. After 3 h, dilute with water and neutralize to pH 7 with 1N aqueous HCl. Collect the white precipitate by filtration and dry under reduced pressure to give the title compound in quantitative yield as a white solid. MS(ES) 370.1 (M+1)⁺; ¹H NMR (400 MHz, d₆-DMSO) δ8.10 (s, 1H), 8.04 (s, 2H), 5.45 (s, 2H), 4.19 (s, 3H).

### Preparation 201

### 3,5-dichlorobenzylazide

Dissolve 3,5-dichlorobenzyl alcohol (10.0 g, 56.0 mmol) in DMF (20 mL) and slowly add thionyl chloride (4.40 mL, 60.0 mmol) to the mixture, while cooling in a water bath. After stirring for 1 h, add K₂CO₃ (15.8 g, 110 mmol) and stir an additional 1 h. Then add DMSO (50 mL) and sodium azide (5.60 g, 86 mmol) and stir the mixture overnight at RT. Dilute the mixture with water and extract with EtOAc. Wash the combined extracts with water and dry over Na₂SO₄. Concentrate to give the title compound (10.11 g, 89%) as an oil. Use without further purification. MS(ES) 201.0 (M+1)⁺.

### Preparation 202

### 1-(3,5-Dichloro-benzyl)-5-hydroxy-1H-[1,2,3]triazole-4-carboxylic acid ethyl ester

Combine diethylmalonate (1.91 g, 11.9 mmol), 3,5-dichlorobenzylazide (2.40 mL, 11.9 mmol), and potassuim carbonate (4.94 g, 35.8 mmol) in DMSO (15 mL) and heat the mixture for 8 h at 50° C. Cool the mixture to RT and dilute with water. Adjust the pH to 5-6 with 1N HCl, and extract with CH₂Cl₂. Wash the combined extracts with water, dry over Na₂SO₄ and concentrate in vacuo. Purify the residue by chromatography over silica gel using a CH₂Cl₂/MeOH gradient to provide 3.28 g of impure product as an oil. Use without further purification. MS(ES) 316.0 (M+1)⁺.

### Preparation 203

### 5-Chloro-1-(3,5-dichloro-benzyl)-1H-[1,2,3]triazole-4-carboxylic acid ethyl ester

Combine 1-(3,5-dichloro-benzyl)-5-hydroxy-1H-[1,2,3]triazole-4-carboxylic acid ethyl ester (3.25 g, 10.3 mmol) with PCl₅ (4.29 g, 20.6 mmol) in toluene (75 mL) and heat at 40-50° C. After 4 h., cool to RT and concentrate the reaction mixture. Add aqueous NaHCO₃ and extract with Et₂O. Dry the combined extracts over Na₂SO₄ and concentrate in vacuo. Purify the residue by chromatography over silica gel using CH₂Cl₂ to provide the title compound (1.83 g) as an impure oil. Use without further purification. MS(ES) 334.0 (M+1)⁺.

### Preparation 204

### 5-chloro-1-(3,5-dichloro-benzyl)-1H-[1,2,3]triazole-4-carboxylic acid (2-chloro-benzyl)-isopropyl-amide

Combine (2-chloro-benzyl)-isopropyl-amine (240 mg, 1.31 mmol) with 5-chloro-1-(3,5-dichloro-benzyl)-1H-[1,2,3]triazole-4-carboxylic acid (400 mg, 1.31 mmol), EDCI (250 mg, 1.30 mmol), HOAt (178 mg, 1.31 mmol), and DIEA (0.20 mL, 1.15 mmol), in DMF (8 mL) and stir the mixture at RT. After 72 h, concentrate the mixture, then dissolve the residue in EtOAc and wash with water. Dry the organic layer over sodium sulfate, filter, and concentrate in vacuo. Purify the residue by chromatography over silica gel using a MeOH/CH₂Cl₂ gradient to provide the title compound (103 mg, 17%) as a white solid. MS(ES) 471.0 (M+)⁺; R_{f}=0.19 (CH₂Cl₂).

### Preparation 205

### 2-Methoxy-5-trifluoromethoxy-benzaldehyde

Combine 4-(trifluoromethoxy)anisole (10.0 g, 52.1 mmol) with hexamethylene tetramine (7.29 g, 52.1 mmol) in trifluoroacetic acid (50 g) and heat the mixture overnight at 80° C. Cool the mixture to RT and concentrate. Dissolve in Et₂O and wash with aqueous NaHCO₃ and brine. Dry over Na₂SO₄, filter and concentrate. Purify the residue by chromatography over silica gel to provide the title compound (3.49 g, 30%) as a light yellow oil. MS(ES) 221.0 (M+1)⁺; R_{f}=0.69 (CH₂Cl₂).

### Preparation 206

### Isopropyl-(2-methoxy-5-trifluoromethoxy-benzyl)-amine

Combine 2-methoxy-5-trifluoromethoxy benzaldehyde (490 mg, 2.23 mmol) and isopropyl amine (197 mg, 3.34 mmol) in 1,2-dichloroethane (15 mL), add sodium triacetoxy-borohydride (945 mg, 4.46 mmol), and stir the mixture overnight at RT. Quench the mixture with water and adjust pH to 8.0 with 1N NaOH. Extract the mixture with dichloromethane, dry the combined extracts over Na₂SO₄, filter and concentrate. Purify the residue over silica gel using a CH₂Cl₂/MeOH gradient to provide the title compound (310 mg, 53%) as a light oil. MS(ES) 264.3 (M+1)⁺.

### Preparation 207

### (2-Methoxy-5-trifluoromethoxy-phenyl)-methanol

Dissolve 2-methoxy-5-trifluoromethoxy benzaldehyde (3.0 g, 13.6 mmol) in MeOH (50 mL) and add sodium borohydride (0.26 g, 6.88 mmol) and stir the mixture at RT until reduction is complete. Concentrate the mixture and dissolve the residue in CH₂Cl₂. Wash with 1N NaOH, water, and brine, dry over sodium sulfate, filter, and concentrate. Purify the residue by chromatography over silica gel using a MeOH/CH₂Cl₂ gradient to provide the title compound (2.88 g, 95%) as a clear oil. MS(EI) 222.1 (M)⁺; R_{f}=0.28 (CH₂Cl₂).

### Preparation 208

### 2-Azidomethyl-1-Methoxy-4-trifluoromethoxy-benzene

Dissolve (2-methoxy-5-trifluoromethoxy-phenyl)-methanol (2.8 g, 12.6 mmol) in DMF (15 mL) and slowly add thionyl chloride (1.00 mL, 13.7 mmol). Stir the mixture for 1 h at RT, then add K₂CO₃ (3.48 g, 25.2 mmol) and stir the resulting mixture an additional 1 h. To this mixture, add sodium azide (1.23 g, 18.9 mmol) and DMSO (15 mL) and stir overnight at RT. Dilute the mixture with water and extract with EtOAc. Wash the combined extracts with water, dry over sodium-sulfate, filter and concentrate to give the title compound 2.14 g (69%) as an oil. MS(EI) 247.1 (M)⁺.

### Preparation 209

### 1-(2-methoxy-5-trifluoromethoxy-benzyl)-5-pyridin-4-yl-1H-[1,2,3]triazole-4-carboxylic acid ethyl ester

Combine ethyl isonicotinoyl acetate (2.13 g, 11.0 mmol), 2-azidomethyl-1-methoxy-4-trifluoromethoxy-benzene (2.10 g, 8.5 mmol), and potassuim carbonate (4.7 g, 34.0 mmol) in DMSO (16 mL) and heat the mixture at 50-60° C. After 72 h, cool the mixture to RT, dilute with water, and extract with EtOAc. Dry the combined extracts over Na₂SO₄, filter, and concentrate. Purify the residue by chromatography over silica gel using a CH₂Cl₂/MeOH gradient to provide the title compound (2.37 g, 38%) as a crystalline solid. MS(ES) 423.2 (M+1)⁺; Analysis for C₁₉H₁₇F₃N₄O₄: Calc'd: C, 54.03; H, 4.06; N, 13.27. Found: C, 54.13; H, 4.16; N, 12.35.

### Preparation 210

### 1-(2-methoxy-5-trifluoromethoxy-benzyl)-5-pyridin-4-yl-1H-[1,2,3]triazole-4-carboxylic acid

Combine 1-(2-methoxy-5-trifluoromethoxy-benzyl)-5-pyridin-4-yl-1H-[[1,2,3]triazole-4-carboxylic acid ethyl ester (1.20 g, 2.84 mmol), 2N aqueous NaOH (8 mL), THF (2 mL), and EtOH (2 mL) and stir at RT until hydrolysis is complete. Remove the organic solvents in vacuo and dilute the mixture with water. Adjust the aqueous mixture to pH 3.0-4.0 with aqueous HCl and extract with CH₂Cl₂. Dry the combined extracts over Na₂SO₄, filter, and concentrate in vacuo to give the title compound (1.08 g, 97%) as an off white solid. MS(ES−) 393.1 (M−1)⁻.

### Preparation 211

### 5-Amino-1-(3,5-bis-trifluoromethyl-benzyl)-1H-[1,2,3]triazole-4-carboxylic acid methyl ester

Combine 1-azidomethyl-3,5-bis-trifluoromethyl-benzene (1.07 g, 3.98 mmol), ethyl cyanoacetate (0.41 g, 3.63 mmol), and sodium methoxide (9.0 mL, 0.5M solution in MeOH) in MeOH (4 mL) and stir at RT. After 48 h, concentrate the reaction mixture, add water and collect the precipitate by filtration and dry under reduced pressure to give the title compound (0.47 g, 34%) as a white solid. MS(ES) 369.2 (M+1)⁺; ¹H NMR (400 MHz, DMSO) δ8.10 (s, 1H), 7.90 (s, 2H), 6.75 (s, NH₂), 5.61 (s, 2H), 3.75 (s, 3H).

### Preparation 212

### 1-(3,5-Bis-trifluoromethyl-benzyl)-5-pyrrol-1-yl-1H-[1,2,3]triazole-4-carboxylic acid methyl ester

Add 2,5-dimethoxyfuran (80 mg, 0.61 mmol) slowly to a solution of 5-amino-1-(3,5-bis-trifluoromethyl-benzyl)-1H-[1,2,3]triazole-4-carboxylic acid methyl ester (210 mg, 0.57 mmol) in glacial acetic acid (3 mL) and heat to reflux. After 2 h, cool to RT, dilute the reaction mixture with water, and extract with EtOAc. Wash the EtOAc extract with water and brine, then dry (Na₂SO4), filter, and concentrate to give the title compound in quantitative yield. Use without further purification. ¹H NMR (400 MHz, CDCl₃) δ7.83 (s, 1H), 7.47 (s, 2H), 6.64 (t, 2H, J=2.0 Hz), 6.45 (t, 2H, J=2.0 Hz), 5.53 (s, 2H), 3.87 (s, 3H).

### Preparation 213

### 3-(1-Methyl-]H-pyrrol-2-yl)-3-oxo-propionic acid ethyl ester

Add 1,1′-carbonyldiimidazole (2.6 g, 16.0 mmol) to a solution of 1-methyl-1H-pyrrole-2-carboxylic acid (2.0 g, 16.0 mmol) in THF (20 mL) and stir at RT. After 12-24 h, add via cannula a preformed solution of ethyl hydrogen malonate (2.5 g, 19.3 mmol) and isopropyl magnesium chloride (19.3 mL of 2M solution in THF) in THF (10 mL) at 0° C. Stir at RT for another 4 h, dilute with water, and extract with EtOAc. Wash the EtOAc extract with water and brine, then dry (Na₂SO₄), filter, and concentrate. Purification by flash chromatography eluting with a linear gradient of 10% to 25% EtOAc in hexanes gives the title compound (1.2 g, 38%). MS(ES−) 194.1 (M−1)⁻. ¹H NMR (400 MHz, CHCl₃) δ6.95 (dd, 1H, J=4.4 Hz, 20), 6.84 (t, 1H, J=2.0 Hz), 6.13 (dd, 1H, J=4.4, 2.0 Hz), 4.19 (q, 2H, J=7.2 Hz), 3.93 (s, 3H), 3.79 (s, 2H), 1.26 (t, 3H, J=7.2 Hz).

The following compound may be prepared using a method similar to the above Preparation.

| Prep. # | Product | Data |
|---|---|---|
| 214 | 3-Oxo-3-pyrazin-2-yl-propionic | MS(ES) 195.0(M+1)⁺ |
| | acid ethyl ester | |

### Preparation 215

### 1-(3,5-Bis-trifluoromethyl-benzyl)-5-(1-methyl-1H-pyrrol-2-yl)-1H-[1,2,3]triazole-4-carboxylic acid ethyl ester

Add 3-(1-methyl-1H-pyrrol-2-yl)-3-oxo-propionic acid ethyl ester (1.0 g, 5.1 mmol) and K₂CO₃ (2.8 g, 20.3 mmol) to a solution of 1-azidomethyl-3,5-bis-trifluoromethyl-benzene (1.4 g, 5.2 mmol) in DMSO. Heat the mixture to 50° C. for 18 h, then cool to RT. Dilute the reaction mixture with water, acidify to pH 4 with 2N HCl, and extract with EtOAc. Wash the EtOAc extract with water and brine, then dry (Na₂SO₄), filter, and concentrate. Purification by flash chromatography eluting with a linear gradient of 15% to 30% EtOAc in hexanes gives the title compound (0.6 g, 40%). MS(ES) 447.0 (M+1)⁺; ¹H NMR (400 MHz, CHCl3) δ7.80 (s, 1H), 7.38 (s, 2H), 6.79 (dd, 1H, J=2.9, 1.9 Hz), 6.31 (dd, 1H, J=3.9, 2.9 Hz), 6.25 (dd, 1H, J=3.9, 1.9 Hz), 5.61 (br s, 2H), 4.35 (q, 2H, J=7.2 Hz), 3.00 (s, 3H), 1.31 (t, 3H, J=7.2 Hz).

Using a method similar to the above Preparation, with the appropriate starting materials, the following compounds may be prepared and isolated.

| Prep. # | Product | Data |
|---|---|---|
| 216 | 1-(3,5-Bis-trifluoromethyl-benzyl)-5-pyrazin- | MS(ES) 446.1(M+1)⁺ |
| | 2-yl-1H-[1,2,3]triazole-4-carboxylic acid | |
| | ethyl ester | |
| 217 | 1-(3,5-Bis-trifluoromethyl-benzyl)-5- | MS(ES) 446.2(M+1)⁺ |
| | pyrimidin-5-yl-1H-[1,2,3]triazole-4- | |
| | carboxylic acid ethyl ester | |
| 218 | 1-(3,5-Bis-trifluoromethyl-benzyl)-5-(4- | MS(ES) 476.1(M+1)⁺ |
| | methylsulfanyl-phenyl)-1H-[1,2,3]triazole-4- | |
| | carboxylic acid methyl ester | |
| 219 | 1-(3,5-Bis-trifluoromethyl-benzyl)-5-pyridin- | MS(ES) 431.1(M+1)⁺; ¹H |
| | 3-yl-1H-[1,2,3]triazole-4-carboxylic acid | NMR(400MHz, CDCl₃) δ 8.76(s, 1H), |
| | methyl ester | 8.49(s, 1H), 7.79(s, 1H), 7.51(m, |
| | | 1H), 7.41(s, 2H), 7.40(m, 1H), |
| | | 5.59(s, 2H), 3.83(s, 3H). |
| 220 | 1-(3,5-Bis-trifluoromethyl-benzyl)-5-pyridin- | MS(ES) 445.2(M+1)⁺; ¹H |
| | 4-yl-1H-[1,2,3]triazole-4-carboxylic acid | NMR(400MHz, CDCl₃) δ 8.74(dd, 2H, J=1.5, |
| | ethyl ester | 4.4Hz), 7.80(s, 1H), 7.45(s, |
| | | 2H), 7.13(dd, 2H, J=2.0, 4.4Hz), |
| | | 5.56(s, 2H), 4.27(q, 2H, J=7.3Hz), |
| | | 1.28(t, 3H, J=7.3Hz). |
| 221 | 1-(3,5-Bis-trifluoromethyl-benzyl)-5- | MS(ES) 446.2(M+1)⁺; ¹H |
| | pyridazin-4-yl-1H-[1,2,3]triazole-4-carboxylic | NMR(400MHz, CDCl₃) δ 9.27(dd, 1H, J=0.9, |
| | acid ethyl ester | 5.4Hz), 9.07(m, 1H), |
| | | 7.81(s, 1H), 7.55(s, 2H), 7.39(dd, 1H, |
| | | J=2.4, 5.4Hz), 5.68(s, 2H), |
| | | 4.25(q, 2H, J=7.3Hz), 1.29(t, 3H, J=7.3Hz). |
| 222 | 1-(3,5-Bis-trifluoromethyl-benzyl)-5-furan-2- | MS(ES) 434.2(M+1)⁺; ¹H |
| | yl-1H-[1,2,3]triazole-4-carboxylic acid ethyl | NMR(400MHz, CDCl₃) δ 7.76(s, 1H), |
| | ester | 7.64(s, 2H), 7.57(m, 1H), 7.44(d, |
| | | 1H, J=3.4Hz), 6.56(dd, 1H, J=2.0, |
| | | 3.4Hz), 5.94(s, 2H), 4.40(q, |
| | | 2H, J=7.3Hz), 1.38(t, 3H, J=7.3Hz). |
| 223 | 1-(3,5-Bis-trifluoromethyl-benzyl)-5-furan-3- | MS(ES) 434.1(M+1)⁺; ¹H |
| | yl-1H-[1,2,3]triazole-4-carboxylic acid ethyl | NMR(400MHz, CDCl₃) δ 7.81(s, 1H), |
| | ester | 7.64(s, 1H), 7.55(m, 3H), 6.41(m |
| | | 1H), 5.65(s, 2H), 4.36(q, 2H, J=7.3Hz), |
| | | 1.34(t, 3H, J=7.3Hz). |
| 224 | 1-(3,5-Bis-trifluoromethyl-benzyl)-5- | MS(es) 450.0(M+1)⁺; ¹H |
| | thiophen-2-yl-1H-[1,2,3]triazole-4-carboxylic | NMR(400MHz, CDCl₃) δ 7.77(s, 1H), |
| | acid ethyl ester | 7.58(dd, 1H, J=1.0, 4.9Hz), |
| | | 7.47(s, 2H), 7.14(dd, 1H, J=3.4, 4.9Hz), |
| | | 7.10(dd, 1H, J=1.0, 3.4Hz), |
| | | 5.63(s, 2H), 4.30(q, 2H, J=7.3Hz), |
| | | 1.26(t, 3H, J=7.3Hz). |
| 225 | 1-(3,5-Bis-trifluoromethyl-benzyl)-5-(5- | MS(ES) 464.0(M+1)⁺; ¹H |
| | methyl-thiophen-2-yl)-1H-[1,2,3]triazole-4- | NMR(400MHz, CDCl₃) δ 7.80(s, 1H), |
| | carboxylic acid ethyl ester | 7.49(s, 2H), 6.90(d, 1H, J=3.9Hz), |
| | | 6.80(m, 1H), 5.64(s, 2H), |
| | | 4.34(q, 2H, J=7.3Hz), 2.51(d, |
| | | 3H, J=1.0Hz), 1.32(t, 3H, J=7.3Hz). |
| 226 | 1-(3,5-bis-trifluoromethyl-benzyl)-5-pyrazin- | MS(ES) 431.3(M+1)⁺; |
| | 2-yl-1H-[1,2,3]triazole-4-carboxylic acid | R_{f}=0.29(1:1 EtOAc/hexanes). |
| | methyl ester | |
| 227 | 1-(3,5-Bis-trifluoromethyl-benzyl)-5- | ¹H NMR(400MHz, CDCl₃) δ |
| | isopropyl-1H-[1,2,3]triazole-4-carboxylic acid | 7.85(s, 1H), 7.57(s, 2H), 5.71(s, 2H), |
| | ethyl ester | 4.43(quart., 2H, J=6.8), |
| | | 3.33(quint., 1H, J=7.1), 1.43(t, 3H, J=6.9), |
| | | 1.25(d, 6H, J=6.6) |

### Preparation 228

### Pyrimidine-5-carboxylic acid methoxy-methyl-amide

Combine EDCI (0.99 g, 5.18 mmol) with a solution of O,N-hydroxylamine hydrochloride (0.51 g, 5.23 mmol), pyrimidine-5-carboxylic (540 mg, 4.35 mmol), triethylamine (1.5 mL, 10.4 mmol), and DMAP (0.64 g, 5.24 mmol) in DMF (10 mL) and stir at RT. After 24 h, treat the reaction mixture with saturated NaHCO₃ and extract with CH₂Cl₂. Wash the organic layer with water, dry over sodium sulfate, filter, and concentrate under reduced pressure. Purification by flash chromatography eluting with a linear gradient of 15% to 30% EtOAc in hexanes gives the title compound (0.15 g, 21%). MS(ES) 168.2 (M+1)⁺; ¹H NMR (400 MHz, CHCl₃) δ9.21 (s, 1H), 9.02 (s, 2H), 3.53 (s, 3H), 3.34 (s, 3H).

Using a method similar to the above Preparation, with the appropriate carboxylic acid starting material, the following compounds may be prepared and isolated.

| Prep. # | Product | Data |
|---|---|---|
| 229 | Pyridazine-4-carboxylic acid | MS(ES) 168.2(M+1)⁺; ¹H NMR(400MHz, |
| | methoxy-methyl-amide | CDCl₃) δ 9.43(m, 1H), 9.32(m, 1H), 7.73(m, |
| | | 1H), 3.55(s, 3H), 3.38(s, 3H). |
| 230 | Thiophene-2-carboxylic acid | MS(ES) 172.0(M+1)⁺. ¹H NMR(400MHz, |
| | methoxy-methyl-amide | CDCl₃) δ 7.94(dd, 1H, J=1.5, 3.4Hz), |
| | | 7.53(dd, 1H, J=1.0, 4.9Hz), 7.08(dd, 1H, J=3.4, |
| | | 4.9Hz), 3.76(s, 3H), 3.35(s, 3H). |
| 231 | 5-Methyl-thiophene-2-carboxylic | MS(ES) 186.0(M+1)⁺. ¹H NMR(400MHz, |
| | acid methoxy-methyl-amide | CDCl₃) δ 7.76(d, 1H, J=3.4Hz), 6.76(m, |
| | | 1H), 3.74(s, 3H), 3.32(s, 3H), 2.49(d, 3H, J=1.0Hz). |

### Preparation 232

### 3-oxo-3-pyrimidin-5-yl-propionic acid ethyl ester

Add n-BuLi (1.12 mL of 1.6M solution in hexane, 1.8 mmol) slowly to a solution of diisopropylamine (0.25 mL, 1.8 mmol) in THF (5 mL) at −78° C. Stir 5 min, then add a solution of EtOAc (0.16 mL, 1.8 mmol) in THF (5 mL). Stir at −78° C. for 25 min, then add pyrimidine-5-carboxylic acid methoxy-methyl-amide (0.14 g, 0.9 mmol). After another 3 h, treat the reaction mixture with 1N HCl solution (25 mL) and extract with EtOAc. Wash the organic extract with water, dry (Na2SO₄), filter, and concentrate under reduced pressure to provide the title compound. Use without further purification. MS(ES) 195.1 (M+1)⁺; ¹H NMR (400 MHz, CDCl₃) δ9.21 (s, 1H), 9.02 (s, 2H), 4.24 (q, 2H, J=7.3 Hz), 3.94 (s, 2H), 1.29 (t, 3H, J=7.3 Hz).

Using a method similar to the above Preparation, with the appropriate amide starting material, the following compounds may be prepared and isolated.

| Prep. # | Product | Data |
|---|---|---|
| 233 | 3-Oxo-3-pyridazin-4- | MS(ES) 195.2(M+1)⁺; ¹H NMR(400MHz, CDCl₃) δ |
| | yl-propionic acid ethyl | 12.43(m, 1H), 9.45(m, 1H), 9.31(d, 1H, J=5.4Hz), |
| | ester | 7.78(m, 1H), 5.85(m, 1H), 4.29(dq, 2H, J=1.5, 7.5Hz), |
| | | 1.34(dt, 3H, J=1.5, 7.4Hz). |
| 234 | 3-Oxo-3-thiophen-2- | MS(ES) 199.0(M+1)⁺; ¹H NMR(400MHz, CDCl₃) δ |
| | yl-propionic acid ethyl | 7.72(m, 1H), 7.68(m, 1H), 7.13(m, 1H), 4.19(q, 2H, J=7.3Hz), |
| | ester | 3.90(s, 2H), 1.24(t, 3H, J=7.3Hz). |
| 235 | 3-(5-Methyl-thiophen- | MS(ES−) 211.2(M−1)⁻; ¹H NMR(400MHz, CDCl₃) δ |
| | 2-yl)-3-oxo-propionic | 7.53(d, 1H, J=3.4Hz), 6.79(dq, 1H, J=1.0, 3.9Hz), 4.18(q, |
| | acid ethyl ester | 2H, J=7.3Hz), 3.83(s, 2H), 2.52(d, 3H, J=1.0Hz), |
| | | 1.24(t, 3H, J=7.3Hz). |

### Preparation 236

### 3-(4-Methylsulfanyl-phenyl)-3-oxo-propionic acid methyl ester

Add 1-(4-methylsulfanyl-phenyl)-ethanone (0.50 g, 3.0 mmol) to a suspension of sodium hydride (0.14 g, 3.1 mmol) in THF (20 mL) and stir the mixture at RT. After 1 h, add dimethyl carbonate (0.64 g, 7.1 mmol) and warm to reflux. After 18 h, dilute the reaction mixture with water, add acetic acid to until the pH=6, then extract with EtOAc. Combine the organic layers and wash with water, and brine, dry over sodium sulfate, filter, and concentrate under reduced pressure. Purification by flash chromatography eluting with a linear gradient of 15% to 35% EtOAc in hexanes gives the title compound (0.60 g, 90%) as a mixture of tautomers. MS(ES) 225.1 (M+1)⁺; ¹H NMR (400 MHz, CHCl₃) δ7.85 (dd, 2H, J=8.9 Hz), 7.28 (dd, 2H, J=8.9 Hz), 3.96 (s, 2H), 3.75 (s, 3H), 2.52 (s, 3H).

### Preparation 237

### 1-(2-chloro-phenyl)-pyrazolidine hydrochloride

Dissolve 2-(2-chloro-phenyl)-pyrazolidine-1-carboxylic acid tert-butyl ester (50 mg, 1 eq) in a solution of acetic acid saturated with HCl (6 mL) and stir at RT. After 6 h, concentrate the mixture to dryness under reduced pressure to give the title compound. MS(IS) 183.0 (M+1)⁺; Analysis calc'd for C₉H₁₁ClN₂.HCl: C, 49.33; H, 5.52; N, 12.79. Found: C, 49.28; H, 5.57; N, 12.70.

Using a method similar to Preparation 237, with the appropriate starting materials, the following compounds may be prepared and isolated.

| Prep. # | Product | Data |
|---|---|---|
| 238 | 2-(2-chloro-4-trifluoromethyl-phenyl)- | MS(ES) 251.0(M+1)⁺; Anal. |
| | pyrazolidine hydrochloride | calc'd for C₁₀H₁₀ClF₃N₂.HCl: C, |
| | | 41.83; H, 3.86; N, 9.75. Found: |
| | | C, 41.45; H, 3.67; N, 9.48. |
| 239 | 2-(2,4-difluoro-phenyl)-pyrazolidine | MS(ES) 185.1(M+1)⁺. |
| | hydrochloride | |
| 240 | 2-(2-chloro-phenyl)-tetrahydro-pyridazine | MS(ES) 197.0(M+1)⁺. |
| | hydrochloride | |

### Preparation 241

### 2-(2-chloro-phenyl)-pyrazolidine-1-carboxylic acid tert-butyl ester

Dissolve NaH (33 mg, 2.0 eq.) and 1, 3-dibromopropane (0.04 mL, 1.0 eq.) in DMF at 0° C. Add N′-(2-chloro-phenyl)-hydrazinecarboxylic acid tert-butyl ester (0.1 g, 1.0 eq.) and stir at 0° C. After 1 h, quench the reaction with water and concentrate the mixture in vacuo. Dissolve the residue in 20% iPrOH/CHCl₃ and wash with water, saturated aqueous NaHCO₃, and brine. Dry the organic layer over Na₂SO₄, filter, and concentrate. Purify the residue by chromatography on silica gel to provide the title compound. MS(ES) 283.1 (M+1)⁺; R_{f}=0.81 (1:1 EtOAc/hexanes).

Using a method similar to Preparation 241, with the appropriate starting materials, owing compounds may be prepared and isolated.

| Prep. # | Product | Data |
|---|---|---|
| 242 | 2-(2-chloro-4-trifluoromethyl-phenyl)- | MS(ES) 351.1(M+1)⁺; |
| | pyrazolidine-1-carboxylic acid tert-butyl | R_{f}=0.50(30% |
| | ester | EtOAc/hexanes) |
| 243 | 2-(2,4-difluoro-phenyl)-pyrazolidine-1- | MS(ES) 285(M+1)⁺; |
| | carboxylic acid tert-butyl ester | R_{f}=0.76(1:1 |
| | | EtOAc/hexanes) |

### Preparation 244

### N′-(2-chloro-phenyl)-hydrazinecarboxylic acid tert-butyl ester

Dissolve 2-chlorophenylhydrazine hydrochloride (5.0 g, 1.0 eq.) in H₂O (50 mL) and THF (50 mL). Add K₂CO₃ (11.6 g, 3.0 eq) and di-t-butyl-dicarbonate (6.1 g) and stir at RT. After 72 h, concentrate the mixture in vacuo. Dissolve the residue in 20% iPrOH/CHCl₃ and wash with water, saturated aqueous NaHCO₃, and brine. Dry the organic layer over Na₂SO₄, filter, and concentrate. Purify the residue by chromatography over silica gel to provide the title compound. MS(ES−) 241.0 (M−1)⁻; R_{f}=0.13 (10% EtOAc/hexanes).

Using a method similar to Preparation 244, with the appropriate starting materials, the following compounds may be prepared and isolated.

| Prep. | | |
|---|---|---|
| # | Product | Data |
| 245 | N′-(2-chloro-4-trifluoromethyl-phenyl)- | MS(ES−) 309.1(M−1)⁻; |
| | hydrazinecarboxylic acid tert-butyl ester | R_{f}=0.38(20% |
| | | EtOAc/hexanes) |
| 246 | N′-(2,4-difluoro-phenyl)- | MS(ES−) 243.1(M−1)⁻; |
| | hydrazinecarboxylic acid tert-butyl ester | R_{f}=0.62(30% |
| | | EtOAc/hexanes) |

### Preparation 247

### 3-Oxo-3-pyrazin-2-yl-propionic acid methyl ester

In a dropwise fashion, add 2-pyrazine methylester (1.0 g, 1.0 eq.) and methyl acetate (1.14 mL, 2.0 eq.) as a solution in toluene (10 mL) to a hot (90° C.) mixture of sodium methoxide (600 mg, 1.5 eq.) in toluene (100 mL). Heat the mixture for 20 h. at 90° C., then cool to RT and concentrate in vacuo. Dissolve the residue in excess methyl acetate, heat at reflux for another 20 h. Cool the mixture to RT, add H₂O, and extract with EtOAc. Dry the organic layer over Na₂SO₄, filter, and concentrate in vacuo to give the title compound that was used without further purification. R_{f}=0.58 (1:1 EtOAc/hexanes).

### Preparation 248

### 2-(2-chloro-phenyl)-tetrahydro-pyridazine-1-carboxylic acid tert-butyl ester

Dissolve NaH (0.17 g, 2.0 eq.) and 1,4-dibromobutane (0.24 mL, 1.0 eq.) in DMF (10 mL) and cool to 0° C. Add N′-(2-chloro-phenyl)-hydrazinecarboxylic acid tert-butyl ester (1.0 g, 1.0 eq.) and stir the mixture for 1 h. at 0° C., then quench with H₂O and concentrate in vacuo. Dissolve the residue in 20% iPrOH/CHCl₃, wash with water, saturated aqueous NaHCO₃, and brine, then dry (Na₂SO₄), filter, and concentrate. Purify the residue by chromatography on silica gel to provide the title compound. MS(ES) 297.1 (M+1)⁺; R_{f}=0.68 (30% EtOAc/hexanes).

### Preparation 249

### 8-chloro-1,2,3,4-tetrahydro-quinoline

Dissolve 8-chloroquinoline (10.0 g, 1.0 eq.) in HOAc (100 mL), add PtO₂ (1.0 g) and shake under hydrogen (45 psi) at RT. After 4 h, remove hydrogen, filter off the catalyst, and concentrate in vacuo. Dissolve the residue in THF, and slurry with polyvinylpyridine, then filter and concentrate in vacuo. Purify the residue by chromatography on silica gel to provide the title compound. MS(ES) 168.0 (M+1)⁺; R_{f}=5% EtOAc/hexanes).

### Preparation 250

### (2,4-dichloro-phenyl)-isopropyl-amine

Combine 2,4-dichloroaniline (800 mg, 5.0 mmol) and 2-bromopropane (0.47 mL, 5.0 mmol) neat in a sealed tube and heat at 100° C. After 16 h, cool to RT, add CHCl₃ and wash with saturated aqueous NaHCO₃, and brine, dry over sodium sulfate, filter, and concentrate. Purify by column chromatography using an EtOAc/hexanes gradient to afford 553 mg (35%) of the title compound as colorless oil. MS(ES) 204.0 (M+1)⁺; R_{f}=0.71 (10% EtOAc/hexanes).

Using a method similar to Preparation 250, with the appropriate starting materials, the following compounds may be prepared and isolated.

| Prep. # | Product | Data |
|---|---|---|
| 251 | (2-chloro-4-fluoro-phenyl)-isopropyl- | MS(ES) 188.0(M+1)⁺; R_{f}=0.75(10% |
| | amine | EtOAc/hexanes). |
| 252 | (2-chloro-4-trifluoromethyl-phenyl)- | R_{f}=0.75(5% EtOAc/hexanes) |
| | isopropyl-amine | |
| 253 | (3,4-difluorophenyl)-isopropyl-amine | MS(ES) 172.1(M+1)⁺; R_{f}=0.36 |
| | | (10% EtOAc/hexanes). |
| 254 | (2,4-dichloro-benzyl)-isopropyl-amine | MS(ES) 218.1(M+1)⁺; R_{f}=0.4(1:1 |
| | | EtOAc/hexanes) |
| 255 | (3,4-difluoro-benzyl)-isopropyl-amine | MS(ES) 196.1(M+1)⁺; R_{f}=0.15(10% |
| | | MeOH/CHCl₃). |
| 256 | (2-chloro-benzyl)-isopropyl-amine | MS(ES) 184.1(M+1)⁺; R_{f}=0.08(1:1 |
| | | EtOAc/hexanes) |
| 257 | (2-chloro-4-fluoro-benzyl)-isopropyl- | MS(ES) 202.0(M+1)⁺; R_{f}=0.23(1:1 |
| | amine | EtOAc/hexanes). |
| 258 | (R)-[1-(2-chloro-phenyl)-ethyl]-isopropyl- | MS(ES) 198(M+1)⁺; R_{f}=0.32(5% |
| | amine | MeOH/CHCl₃). |
| 259 | (2-Chloro-phenyl)-isopropyl-amine | MS(ES) 170.2(M+1)⁺; R_{f}=0.71(25% |
| | | EtOAc/hexanes). |

### Preparation 260

### (2-chloro-phenyl)-(2-pyrrolidin-1-yl-ethyl)-amine

Combine 2-chloroaniline (0.41 mL, 3.9 mmol) and 1-(2-chloroethyl)pyrrolidine hydrochloride (670 mg, 3.9 mmol) in a sealed tube and heat at 100° C. After 16 h, add CHCl₃ and wash with saturated aqueous NaHCO₃ and brine, dry over Na₂SO₄, filter, and concentrate. Purify the residue via radial chromatography using a MeOH/CHCl₃ gradient to afford 384 mg (44%) of the title compound as tan oil. MS(ES) 225.1 (M+1)⁺; R_{f}=0.24 (10% MeOH/CHCl₃).

Using a method similar to Preparation 260, with the appropriate starting materials, owing compounds may be prepared and isolated.

| Prep. # | Product | Data |
|---|---|---|
| 261 | N′-(2-chloro-phenyl)-N,N-dimethyl-ethane- | MS(ES) 199.1(M+1)⁺; |
| | 1,2-diamine | R_{f}=0.25(10% MeOH/CHCl₃). |
| 262 | (2-chloro-phenyl)-(2-piperidin-1-yl-ethyl)- | MS(ES) 239.1(M+1)⁺; |
| | amine | R_{f}=0.42(10% MeOH/CHCl₃). |
| 263 | (2-chloro-phenyl)-(2-morpholin-4-yl-ethyl)- | MS(ES) 241.1(M+1)⁺; |
| | amine | R_{f}=0.50(80% EtOAc/hexanes). |
| 264 | (2-chloro-4-fluoro-phenyl)-(2-pyrrolidin-1-yl- | MS(ES) 243.1(M+1)⁺; |
| | ethyl)-amine | R_{f}=0.23(10% MeOH/CHCl₃). |
| 265 | N′-(2-chloro-4-fluoro-phenyl)-N,N-dimethyl- | MS(ES) 217.1(M+1)⁺; |
| | ethane-1-diamine | R_{f}=0.17(10% MeOH/CHCl₃). |
| 266 | (2-chloro-4-fluoro-phenyl)-(2-morpholin-4-yl- | MS(ES) 259.0(M+1)+; |
| | ethyl)-amine | R_{f}=0.40(80% EtOAc/hexanes). |
| 267 | (2-chloro-4-fluoro-phenyl)-(2-piperidin-1-yl- | MS(ES) 257.1(M+1)+; |
| | ethyl)-amine | R_{f}=0.33(10% MeOH/CHCl₃). |
| 268 | N′-(2,4-dichloro-phenyl)-N,N-dimethyl- | MS(ES) 233.0(M+1)⁺; |
| | ethane-1,2-diamine | R_{f}=0.20(10% MeOH/CHCl₃). |
| 269 | (2,4-dichloro-phenyl)-(2-pyrrolidin-1-yl- | MS(ES) 259.0(M+1)⁺; |
| | ethyl)-amine | R_{f}=0.16(10% MeOH/CHCl₃). |
| 270 | (2-chloro-phenyl)-(2-trimethylsilanyloxy- | MS(ES) 244.1(M+1)⁺; |
| | ethyl)-amine | R_{f}=0.80(20% EtOAc/hexanes). |
| 271 | (R)-[1-(2-chloro-phenyl)-ethyl]-(2-pyrrolidin- | MS(ES) 253.1(M+1)⁺; |
| | 1-yl-ethyl)-amine | R_{f}=0.10(10% MeOH/CHCl₃). |
| 272 | (2-chloro-benzyl)-(2-methoxy-ethyl)-amine | MS(ES) 201.9(M+1)⁺; |
| | | R_{f}=0.36(10% MeOH/CHCl₃). |

### Preparation 273

### (R,S)-{2-[1-(2-chloro-phenyl)-ethylamino]-ethyl}-carbamic acid tert-butyl ester

Add N-(2-aminoethyl)carbamic acid t-butyl ester (10.0 g, 62.0 mmol) to a solution of 2′-chloroacetophenone (11.5 mL, 74.4 mmol) in MeOH (80 mL). Add sodium cyanoborohydride (11.7 g, 186.0 mmol) and acetic acid (5 drops) and stir at RT. After 16 h, quench with H₂O and concentrate the mixture to dryness. Dissolve in 20% iPrOH/CHCl₃ and wash with saturated aqueous NaHCO₃ and brine, dry over Na₂SO₄, filter, and concentrate. Purify the residue by column chromatography using an EtOAc/hexanes gradient to yield 5.5 g (30%) of the title compound as colorless oil, which solidifies upon standing. MS(ES) 299.1 (M+1)⁺; R_{f}=0.34 (1:1 EtOAc/hexanes).

Using a method similar to Preparation 273, with the appropriate starting materials, the following compounds may be prepared and isolated.

| Prep. # | Product | Data |
|---|---|---|
| 274 | [2-(2-Chloro-benzylamino)- | MS(ES) 287.1(M+1)⁺; |
| | ethyl]-carbamic acid | R_{f}=0.28(1:1 EtOAc/hexanes). |
| | tert-butyl ester | |
| 275 | [2-(2-chloro-4-fluoro- | MS(ES) 303.1(M+1)⁺; |
| | benzylamino)-ethyl]-carbamic | R_{f}=0.21(1:1 EtOAc/hexanes). |
| | acid tert-butyl ester | |
| 276 | (2-Chloro-benzyl)-pyridin- | MS(ES) 232.9(M+1)⁺; |
| | 4-yl-methyl-amine | R_{f}=0.20(80% EtOAc/hexanes). |

### Preparation 277

### 2-chloro-N-methyl-benzenesulfonamide

Combine 2-chlorobenzenesulfonyl chloride (5.0 g, 1.0 eq.) and N-methylamine (25 mL of a 2N solution in THF, 2.0 eq.) in a sealed tube with THF (25 mL) and stir at RT. After 16 h, concentrate the mixture in vacuo. Dissolve the residue in 20% iPrOH/CHCl₃, and wash with saturated aqueous NaHCO₃ and brine. Dry the organic layer over Na₂SO₄, filter, and concentrate. Purify the residue by chromatography to give the title compound (94% yield). MS(ES) 205.0 (M+1)⁺; R_{f}=0.70 (1:1 EtOAc/hexanes).

### Preparation 278

### 2-chloro-N-methyl-benzamide

Combine 2-chlorobenzoic acid, (10.0 g, 1 eq), N-methylamine (70 mL of a 2N soln in THF, 1.5 eq.), EDCI (12.2 g, 1.1 eq.), HOAt (8.7 g, 1.1 eq.), TEA (10.0 mL, 1.1 eq.) and DMAP (5 mg) in DMF (50 mL) and stir overnight at RT. Concentrate the mixture to dryness and dissolve in 20% iPrOH/CHCl₃. Wash with saturated aqueous NaHCO₃ and brine. Dry (Na2SO4), filter, and concentrate to dryness. Purify the residue by chromatography to provide the title compound (76% yield). MS(ES) 554.9 (M+1)⁺; R_{f}=0.60 (1:1 EtOAc/hexanes).

### Preparation 279

### 3-methyl-but-2-enoic acid N′-(2-chloro-phenyl)-hydrazide

Dissolve sodium metal (1.5 g, 2.3 eq) in n-butanol (25 mL), then add 2-chlorophenylhydrazine hydrochloride (5.0 g, 1.0 eq.) and stir 15 min. Add methyl,3,3-dimethylacrylate (3.8 mL, 1.5 eq.) dropwise, then heat the mixture to reflux. After 5 h., add H₂O (100 mL) while the solution is still hot, then cool to RT and acidify to pH=6 with 50% aqueous acetic acid. Wash with 1N NaOH, saturated NaHCO₃, and brine. Dry over Na₂SO₄, filter and concentrate. Purify the residue by column chromatography over silica gel to provide the title compound (44% yield). MS(ES) 170.6 (M+1)⁺; R_{f}=0.55 (1:1 EtOAc/hexanes).

### Preparation 280

### (R,S)-2-amino-2-(2-chloro-phenyl)-acetamide hydrochloride

Stir a slurry of 2-chlorobenzaldehyde (43 ml, 1.0 eq) and sodium bisulfite (39.5 g, excess) in H₂O (150 mL) and MeOH (150 mL) for 15 min, then add concentrated ammonium hydroxide (26 mL, 1.0 eq). Stir the mixture for 30 min. at RT, then cool to 0° C. and add MeOH (75 mL) and a solution of sodium cyanide (18.7 g, 1.0 eq) in H₂O (75 mL) dropwise over 15 min. Remove the ice bath and stir overnight. Evaporate off the organics under reduced pressure, then extract the aqueous mixture with ether. Wash the extracts with H₂O and brine, dry over Na₂SO₄, filter, and concentrate down to approximately 200 mL. Acidify the solution to pH 4.5 with 2 N HCl. Cool the slurry in the refrigerator, filter the precipitate, and dry under vacuum to give the title compound (3.3% yield). MS(FD) 186.63 (M+); IR (KBr) 2633.95, 1697.60, 1624.25, 1609.12, 1588.63, 1502.62, 1478.18, 1424.98, 1346.50, 1310.12, 1192.24, 1149.58, 1055.06, 1017.65, 760.25, 668.61, 659.94, 589.72, 478.19 cm⁻¹.

### Preparation 281

### (R/S)-3-amino-3-(2-chloro-phenyl)-propionic acid methyl ester

Add thionyl chloride (18.3 mL, 250 mmol) dropwise to a cooled (0° C.) flask containing MeOH (100 mL) under N₂. After 10 min., add this solution dropwise to a stirred suspension of 3-amino-3-(2-chloro-phenyl)-propionic acid (5.00 g, 25 mmol) in MeOH (50 mL) and allow the mixture to warm to RT. After 48 h., concentrate the mixture, add diethyl ether, and place in a sonicating bath for 10 min. Concentrate in vacuo to get the title compound as a white solid (6.29 g, quantitative yield). MS(ES) 214 (M+1)⁺. ¹H NMR (400 MHz, DMSO) δ3.05 (m, 1H), 3.20 (m, 1H), 3.56 (s, 3H), 4.98 (t, 1H, J=7.3 Hz), 7.51 (m, 2H), 7.54 (m, 1H), 7.81 (m, 1H), 8.84 (br s, 1H).

### Preparation 282

### (R/S)-3-amino-3-(2-chloro-phenyl)-propionic acid

Add 2-chlorobenzaldehyde (5.63 mL, 50 mmol), malonic acid (5.20 g, 50 mmol), ammonium acetate (8.09 g, 105 mmol) and EtOH (20 mL) to a mechanically stirred three-neck flask equipped with a condenser. Heat the mixture to reflux and stir overnight. Cool to RT and filter the precipitate, wash with EtOH and dry under reduced pressure to provide the title compound as a white solid (6.13 g, 61% yield). MS(ES) 200 (M+1)⁺;

¹H NMR (400, MHz, D₂O/DCl) δ2.90 (m, 2H); 4.96 (t, 1H, J=7.8 Hz); 7.15 (m, 2H); 7.26 (m, 2H).

### Preparation 283

### (R/S)-[1-(2-chloro-phenyl)-3-hydroxy-propyl]-carbamic acid tert-butyl ester

Add borane dimethylsulfide complex (12.7 mL of a 2.0M in THF, 25.5 mmol,) dropwise to a 0° C. solution of 3-tert-butoxycarbonylamino-3-(2-chloro-phenyl)-propionic acid methyl ester (2.50 g, 7.97 mmol) in THF (25 mL). Allow the reaction to warm to RT overnight, then quench with MeOH (30 mL), stir 30 min., and concentrate. Dissolve the residue in 20% i-PrOH/CHCl₃, wash with 0.2N HCl, saturated aqueous NaHCO₃, and brine. Dry (MgSO₄) and concentrate in vacuo. Purify the residue by chromatography on silica gel eluting with 0-60% EtOAc/hexanes to provide the title compound as a white solid (2.15 g, 94% yield). MS(ES) 286 (M+1)⁺; R_{f}=0.15 (25% EtOAc/hexanes).

### Preparation 284

### (R/S)-3-tert-butoxycarbonylamino-3-(2-chloro-phenyl)-propionic acid methyl ester

Add di-t-butyl-dicarbonate (6.32 mL, 27.5 mmol), DMAP (0.31 g, 2.5 mmol), and pyridine (4.25 mL, 52.5 mmol) to a stirred suspension of 3-amino-3-(2-chloro-phenyl)-propionic acid methyl ester (6.25 g, 25.0 mmol) and stir at RT. After 16 h, concentrate the mixture and dissolve the residue in 20% i-PrOH/CHCl₃. Wash with 0.1N HCl, saturated NaHCO₃ solution, and brine. Dry (MgSO₄), filter, and concentrate. Purify by chromatography on silica gel, eluting with 0-15% EtOAc/hexanes, to provide the title compound as a white solid (6.2 g, 94% yield). MS(ES) 314(M+1)⁺; R_{f}=0.18 (15% EtOAc/hexanes).

### Preparation 285

### Acetic acid cis-2-(2-chloro-phenyl)-pyrrolidin-3-yl ester

Combine 4-bromo-5-(2-chloro-phenyl)-3,4-dihydro-2H-pyrrole (3.2 g, 12.4 mmol), silver acetate (2.48 g, 14.8 mmol), and potassium acetate (1.82 g, 18.5 mmol) in glacial acetic acid (25 ml). Heat in an oil bath at 100° C. for 1 h. Let cool to RT and remove most of the solvent. Dilute the residue with EtOAc (75 ml) and slowly add saturated aqueous sodium bicarbonate solution (50 ml). Wash the organic phase with brine (50 ml), dry over sodium sulfate, filter and concentrate. Purify the residue by chromatography on silica gel (15% EtOAc/hexanes) to give the desired material as a dark oil (1.34 g, 46%). Dissolve this material in glacial acetic acid and add sodium triacetoxyborohydride (3.58 g, 16.9 mmol). Stir at RT for 48 h, then remove most of solvent. Dilute the residue with EtOAc (75 ml) and slowly add saturated aqueous sodium bicarbonate solution (50 ml). Wash the organic phase with brine (50 ml), dry over sodium sulfate, filter and concentrate. Purify the residue by chromatography on silica gel (0.5% ammonium hydroxide/1% MeOH/dichloromethane) to give title compound as a dark oil (830 mg, 61%). ¹H NMR (CDCl₃, 400 MHz) δ1.95-2.02 (m, 1H),2.07 (s,3H), 2.32-2.41 (m, 1H), 3.03-3.1 (m, 1H), 3.32-3.38 (m, 1H), 4.57 (d, J=4.4 Hz, 1H), 5.65-5.68 (m, 1H), 7.13-7.63 (m, 4H); R_{f}=0.2 (EtOAc, Ninhydrin stain).

### Preparation 286

### [4-(2-Chloro-phenyl)-2-hydroxy-4-oxo-butyl]-carbamic acid tert-butyl ester

Add titanium tetrachloride (1M solution in dichloromethane, 8.4 ml, 8.4 mmol) to a solution of 1-(2-chloro-phenyl)-ethanone (1.24 g, 8.02 mmol) in dichloromethane (20 ml) at −78° C. Stir 10 min then add diisopropylethylamine (965 mg, 7.46 ml) followed by N,N-bis(tert-butoxycarbonyl)glycinal in dichloromethane (20 ml). Continue to stir at −78° C. for 10 min, then warm to 0° C. for 30 min, and then warm to RT. After 2 h, quench the reaction with saturated aqueous NH₄Cl (50 ml, extract with EtOAc (3×40 ml) and wash the combined organic layers with brine (50 ml). Dry over sodium sulfate, filter, and concentrate. Purify the residue by chromatography on silica gel (10% EtOAc/hexanes and 25% EtOAc/hexanes) to give title compound as a viscous oil. ¹H NMR (CDCl₃, 400 MHz) δ1.45 (s, 9H), 3.10 (dd, J=18, 8.4 Hz, 1H), 3.17-3.25 (m, 2H), 3.35-3.42 (m, 1H), 3.50 (br s, 1H), 4.30 (br s, 1H), 5.01 (br s, 1H), 7.32-7.44 (m, 3H), 7.52 (d, J=6.8 Hz, 1H); R_{f}=0.2 (40% EtOAc/hexanes).

### Preparation 287

### [2-(tert-Butyl-dimethyl-silanyloxy)-4-(2-chloro-phenyl)-4-oxo-butyl]-carbamic acid tert-butyl ester

Combine [4-(2-chloro-phenyl)-2-hydroxy-4-oxo-butyl]-carbamic acid tert-butyl ester (570 mg, 1.82 mmol) and imidazole (248 mg, 3.64 mmol) in dichloromethane (5 ml), and chill to 0° C. Add tert-butyldimethylsilyl trifluoromenthanesulfonate (630 μl, 2.74 mmol) and stir for 12 h, allowing to slowly warm to RT. Dilute with EtOAc (40 ml). Wash the organic phase with saturated aqueous NH₄Cl (30 ml) and saturated aqueous NaHCO₃ (30 ml). Dry the organic phase over sodium sulfate, filter, and concentrate. Purify the residue by chromatography on silica gel (5% EtOAc/hexanes) to give the title compound as a colorless, viscous oil (530 mg, 68%). ¹H NMR (CDCl₃, 400 MHz) δ0.04 (s, 3H), 0.11 (s, 3H), 0.85 (s, 9H), 1.43 (s, 9H), 3.07-3.36 (m, 4H), 4.44 (br s, 1H), 4.76 (br s, 1H), 7.29-7.41 (m, 3H), 7.50 (d, J=8 Hz, 1H); R_{f}=0.46 (20% EtOAc/hexanes).

### Preparation 288

### 4-(tert-Butyl-dimethyl-silanyloxy)-2-(2-chloro-phenyl) pyrrolidine

Dissolve [2-(tert-butyl-dimethyl-silanyloxy)-4-(2-chloro-phenyl)-4-oxo-butyl]-carbamic acid tert-butyl ester (530 mg, 1.24 mmol) and pyridine (0.3 ml, 3.72 mmol) in acetonitrile (10 ml) and chill to 0° C. Add iodotrimethylsilane (0.3 ml, 2.11 mmol) and stir 15 min. Allow to warm to RT and stir an additional 30 min. Dilute with EtOAc (40 ml) and wash with saturated aqueous NH₄Cl (2×30 ml). Dry the organic phase over sodium sulfate, filter, and concentrate. Dissolve the residue in glacial acetic acid (10 ml) and quickly add sodium triacetoxyborohydride (526 mg, 2.48 mmol). Stir at RT for 20 min., then remove most of solvent. Dissolve the residue in EtOAc (40 ml) and wash with saturated aqueous sodium bicarbonate solution (40 ml). Dry the organic phase over sodium sulfate, filter and concentrate. Purify the residue by chromatography on neutralized silica gel (10% EtOAc/hexanes) to give title compound as a dark oil. ¹H NMR (CDCl₃, 400 MHz) δ0.00 (s, 3H), 0.03 (s, 3H), 0.83 (s, 9H), 1.60 (ddd, J=12, 7.2, 4 Hz, 1H), 2.0 (br s, 1H), 2.51 (ddd, J=13.8, 8, 6 Hz, 1H), 2.98-3.06 (m, 2H), 4.40-4.44 (m, 1H), 4.55 (t, J=8 Hz, 1H), 7.11 (ddd, J=7.6, 7.6, 2 Hz, 1H), 7.19-7.23 (m, 1H), 7.28 (dd, J=8,1.6 Hz, 1H), 7.66 (dd, J=7.6, 2 Hz, 1H); R_{f}=0.5 (50% EtOAc/hexanes).

### Preparation 289

### [1-(3,5-Bis-trifluoromethyl-benzyl)-5-chloro-1H-[1,2,3]triazol-4-yl]-[3-(2-chloro-phenyl)-piperidin-1-yl)-methanone

To a solution of 1-(3,5-bis-trifluoromethyl-benzyl)-5-chloro-1H-[1,2,3]triazole-4-carboxylic acid (224 mg, 0.60 mmol) in CH₂Cl₂ (0.25 M), add oxalyl chloride (153 mg, 1.2 mmol), followed by a catalytic amount of DMF (1 drop) and stir at RT. After 1 h, concentrate the mixture to dryness. To this residue add a solution of 3-(2-chloro-phenyl)-piperidine (105 mg, 0.54 mmol) in pyridine (0.25 M), add a catalytic amount of DMAP (10 mg) and stir at RT. After 12 h, concentrate the solution. Dilute the residue with CH₂Cl₂ (3 mL) and wash with 1N HCl (3×3 mL), and saturated solution of NaHCO₃ (3 mL). Dry the organic layer, filter and concentrate to provide the title compound that was used without further purification (252 mg, 76%). R_{f}=0.34 2:1 Hex/EtOAc; MS(ES) 551.0 (M+1)⁺.

### Preparation 290

### (2-Chloro-benzyl)-(2,2,2-trifluoro-ethyl)-amine

Combine 2-iodo-1,1,1-trifluoroethane(1.15 g, 5.48 mmol) with 2-chlorobenzyl amine (1.36 g, 9.6 mmol) and heat in a sealed vessel at 100-170° C. After 16 h, cool to RT, quench with aqueous NaHCO₃, and extract with EtOAc. Dry over Na₂SO₄, filter, and concentrate. Purify by the residue by chromatography on silica gel to provide the title compound (33% yield). MS(EI) 223.04 (M⁺); R_{f}=0.81 (CH₂Cl₂).

### Preparation 291

### 2-(2-chloro-phenyl)-pyrrolidine-1-carboxylic acid-tert-butyl ester

Combine 2-(2-chloro-phenyl)-pyrrolidine (2.0 g, 11.0 mmol) with di-t-butyldicarbonate (2.89 g, 13.2 mmol) in a mixture of THF (30 mL) and aqueous NaHCO₃ (30 mL) and stir at RT until the reaction is complete. Dilute the mixture with water and extract with EtOAc. Dry the combined extracts over Na₂SO₄, filter, and concentrate. Purify the residue by chromatography on silica gel to provide the title compound (92% yield). MS(ES) 282.3 (M+1)⁺; R_{f}=0.43 (CH₂Cl₂).

### Preparation 292

### 2-(2-chloro-phenyl)-2-methyl-pyrrolidine-1-carboxylic acid-tert-butyl ester

Combine 2-(2-chloro-phenyl)-pyrrolidine-1-carboxylic acid-tert-butyl ester (2.0 g, 7.12 mmol) and TMEDA (1.16 mL, 14.2 mmol) in THF (100 mL) and cool the mixture to −78° C. Slowly add a solution of s-butyl lithium (1.3 M in cyclohexane, 10.95 mL) and stir for 1-2 h with cooling. Add iodomethane (1.14 mL, 14.2 mmol) in one portion and allow the mixture to stir for 1-2 h while warming to −20° C. Quench the reaction with water and extract with EtOAc. Dry the combined extracts over Na₂SO₄, filter, and concentrate. Purify the residue by chromatography on silica gel to provide the title compound (37% yield). MS(ES) 296.4 (M+1)⁺; R_{f}=0.24 (CH₂Cl₂).

### Preparation 293

### 2-(2-Chloro-phenyl)-2-methyl-pyrrolidine hydrochloride

Dissolve 2-(2-chloro-phenyl)-2-methyl-pyrrolidine-1-carboxylic acid-tert-butyl ester (0.76 g, 2.58 mmol) in acetic acid saturated with HCl (5 mL) and stir at RT. After 4 h, concentrate the mixture under reduced pressure, and then concentrate the residue twice from Et₂O to give the title compound (94% yield) that was used without further purification. MS(ES) 196.0 (M+1)⁺.

### Preparation 294

### 1-(3,5-Bis-trifluoromethyl-benzyl)-5-chloro-1H-[1,2,3]triazole-4-carboxylic acid (2-chloro-phenyl)-isopropyl-amide

Dissolve 1-(3,5-bis-trifluoromethyl-benzyl)-5-chloro-1H-[1,2,3]triazole-4-carboxylic acid (0.25 g, 0.67 mmol) in CH₂Cl₂ (5 mL). Add DMF (1 drop, cat.) and oxalyl chloride (0.18 mL, 2.1 mmol) and stir at RT. After 1 h, concentrate the mixture under reduced pressure, redissolve in Et₂O and concentrate again. Add pyridine (5 mL), (2-chloro-phenyl)-isopropyl-amine (0.113 g, 0.67 mmol), and DMAP (10 mg) and heat to 50° C. until the reaction is complete. Cool to RT, quench the reaction with aqueous NaHCO₃, and extract with EtOAc. Dry the combined extracts over Na₂SO₄, filter, and concentrate. Purify the residue by chromatography on silica gel to provide the title compound. R_{f}=0.60 (6.25% MeOH/CH₂Cl₂).

### Preparation 295

### 1-(3,5-Bis-trifluoromethyl-benzyl)-5-phenyl-1H-imidazole-4-carboxylic acid methyl ester

Add 3,5-bis triflouromethyl benzyl amine (5.66 g, 23.3 mmol) and triethylamine (2.7 mL, 19.4 mmol) to a solution (E/Z-3-bromo-2-methyleneamino-3-phenyl-acrylic acid methyl ester (5.20 g, 19.4 mmol, J. Org. Chem. 1994, 59, 7635) in DMF (60 mL). Stir the reaction mixture at RT for 16 h, then pour into saturated aqueous NaHCO₃ and extract with CH₂Cl₂ (once), and EtOAc (three times). Dry the combined organic layers over magnesium sulfate, filter, and concentrate. Remove excess DMF by azeoptropic distillation at reduced pressure with xylenes. Purify the residue by chromatography on silica gel using a hexanes/EtOAc gradient to yield the title compound (3.0 g, 36%) as a brown-orange solid. ¹H NMR (300 MHz, CDCl₃) 7.79 (s, 1H), 7.75 (s 1H), 7.35-7.5 (m, 3H), 7.25-7.49 (m, 4H), 5.15 (s, 2H), 3.77 (s, 3H); MS(ES) 429.1 (M+1)⁺.

### Preparation 296

### 1-Phenethyl-5-phenyl-1H-imidazole-4-carboxylic acid methyl ester

Using a method similar to the above Preparation, with the appropriate starting materials, the title compound may be prepared and isolated. ¹H NMR 7.55-7.45 (m, 4H), 7.20-7.35 (M, 5H), 6.85-6.75 (m, 2 H), 4.05 (t, 2 H), 3.75 (s, 3H), 2.85 (t, 2H); MS(ES) 307.2 (M+1)⁺.

### Preparation 297

### 1-(3,5-Bis-trifluoromethyl-benzyl)-5-phenyl-1H-[1,2,3]triazole-4-carboxylic acid (2-chloro-4-fluoro-phenyl)-amide

Dissolve 1-(3,5-bis-trifluoromethyl-benzyl)-5-phenyl 1H-[1,2,3]triazole-4-carboxylic acid (398 mg, 0.96 mmol) in 1,2-dichloromethane (2 mL) and DMF (2 drops) and add oxalyl chloride (0.083 mL, 0.96 mmol). After 1 h, concentrate the mixture under reduced pressure and dissolve the residue in pyridine (3 mL). Add 2-chloro-4-fluoroaniline (0.12 mL, 0.96 mmol) and DMAP (5 mg) and heat the mixture for 1 h at 100° C. Then cool the mixture to RT and concentrate under reduced pressure. Dissolve the residue in 20% iPrOH/CHCl₃ and wash with sat. aqueous NaHCO₃ and brine. Dry the organic layer over Na₂SO₄, filter and concentrate. Purify the residue by radial chromatography (MeOH/CHCl₃ gradient) to provide 93 mg (36%) of the title compound as a white foam. MS(ES) 543.0 (M+1)⁺; R_{f}=0.85 (2% MeOH/CHCl₃).

### Preparation 298

### 1-(3,5-Bis-trifluoromethyl-benzyl)-5-hydroxy-1H-[1,2,3]triazole-4-carboxylic acid (2-chloro-benzyl)-methyl-amide

Add 0.5M solution of sodium methoxide in MeOH (4.0 mL, 2.0 mmol) to 1-(3,5-bis-trifluoromethyl-benzyl)-5-chloro-1H-[1,2,3]triazole4-carboxylic acid (2-chloro-benzyl)-methyl-amide (0.2 g, 0.4 mmol) and reflux for 18 h. Acidify the reaction mixture with IN HCl to pH 4, collect precipitate by filtration, and dry to give the product as white powder (0.12 g, 60%). MS(ES) 493.1 (M+1)⁺. ¹H NMR (400 MHz, DMSO, 1:1 mixture of rotamers): δ8.13 (s, 0.5H), 8.12 (s, 0.5H), 8.02 (s, 1H), 7.94 (s, 1H), 7.45 (m, 1H), 7.34 (m, 1H), 7.27 (m, 2H), 5.62 (s, 1H), 5.58 (s, 1H), 5.25 (s, 1H), 4.75 (s, 1H), 3.40 (s, 1.5H), 2.95 (s, 1.5H).

### EXAMPLE 1

### (R)-3-[1-(3,5-Bis-trifluoromethyl-benzyl)-5-phenyl-1H-[1,2,3]triazole-4-carbonyl]-4-phenyl-oxazolidin-2-one.

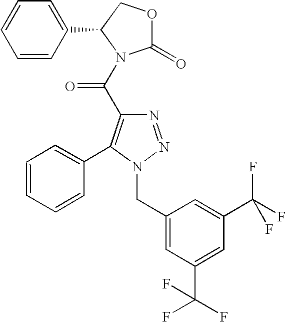

Add triethylamine (0.156 mL, 1.12 mmol) to a slurry of 1-(3,5-Bis-trifluoromethyl-benzyl)-5-phenyl-1H-[1,2,3]triazole-4-carboxylic acid (150 mg, 0.36 mmol) and (R)-(−)-4-phenyl-2-oxazolidinone (46 mg, 0.28 mmol) in toluene (5 mL). Heat the mixture to 90° C., then add pivaloyl chloride (0.044 mL, 0.36 mmol). Reflux overnight, then cool to RT and concentrate under reduced pressure. Dissolve the residue in 20% iPrOH/CHCl₃ and wash with saturated aqueous NaHCO₃, and brine, dry over Na₂SO₄, filter, and concentrate. Purify the residue by radial chromatography (EtOAc/hexanes gradient) to afford the title compound (35 mg, 23%) as a white foam. MS(ES) 561.2 (M+1)⁺; HPLC [40% iPrOH 60% heptane on a chiralpack AD (0.46×25 cm) 1.5 mL/min flow, 0.020 mL Inj. Vol.; 222 nM] R_{f}=10.3 min; 92.9%.

Using the method of Example 1, the following compounds may be prepared and isolated.

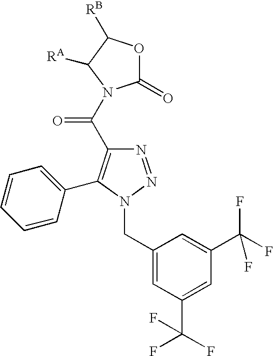

| Ex. # | R^{A} | R^{B} | Data |
|---|---|---|---|
| 2 | (S)- | hydrogen | MS(ES) 561.07 (M + 1)⁺; HPLC (40% |
| | phenyl | | iPrOH 60% heptane on a chiralpack AD |
| | | | (0.46 × 25 cm) 1.5 mL/min flow, |
| | | | 0.020 mL Inj. |
| | | | Vol.; 222 nM) R_{f} = 9.44 min; 94.2%. |
| 3 | (R)- | hydrogen | MS(ES) 575.0 (M + 1)⁺; HPLC (40% iPrOH |
| | benzyl | | 60% heptane on a chiralpack AD (0.46 × 25 |
| | | | cm) 1.5 mL/min flow, 0.020 mL Inj. |
| | | | Vol.; 222 nM) R_{f} = 12.3 min; 95.3%. |
| 4 | (S)- | hydrogen | MS(ES) 575.0 (M + 1)⁺; HPLC (40% iPrOH |
| | benzyl | | 60% heptane on a chiralpack AD (0.46 × 25 |
| | | | cm) 1.5 mL/min flow, 0.020 mL Inj. |
| | | | Vol.; 222 nM) R_{f} = 12.01 min; 92.6%. |
| 5 | (R)- | (S)- | MS(ES) 637.1 (M + 1)⁺. HPLC (40% iPrOH |
| | phenyl | phenyl | 60% heptane on a chiralpack AD (0.46 × 25 |
| | | | cm) 1.5 mL/min flow, 0.020 mL Inj. |
| | | | Vol.; 222 nM) R_{f} = 23.78 min; 99.3%. |
| 6 | (S)- | (R)- | MS(ES) 637.2 (M + 1)⁺. HPLC (40% iPrOH |
| | phenyl | phenyl | 60% heptane on a chiralpack AD (0.46 × 25 |
| | | | cm) 1.5 mL/min flow, 0.020 mL Inj. |
| | | | Vol.; 222 nM) R_{f} = 22.86 min; 96.1%. |
| 7 | (S)- | dimethyl | MS(ES) 589.2 (M + 1)⁺; TLC R_{f} = 0.75 |
| | phenyl | | (50% EtOAc/hexanes). |

### EXAMPLE 8

### 2-[1-(3,5-Bis-trifluoromethyl-benzyl)-5-phenyl-1H-[1,2,3 ]triazole-4-carbonyl]-1-phenyl-pyrazolidin-3-one.

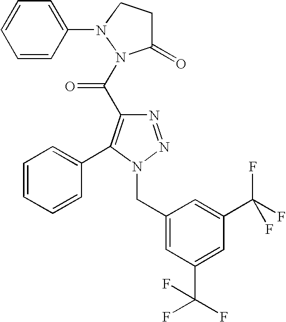

Using a method similar to Example 1, with the exception of using 1-phenyl-3-pyrazolidinone (46 mg, 0.28 mmol, Aldrich), affords the title compound (11.0 mg, 7.5%) as a white foam. MS(ES) 560.0 (M+1)⁺; TLC R_{f}=0.37 (50% EtOAc/hexanes).

### EXAMPLE 9

### 1-(3,5-Bis-trifluoromethyl-benzyl)-5-phenyl-]H-[1,2,3]triazole-4-carboxylic acid (2-chloro-phenyl)-methyl-amide.

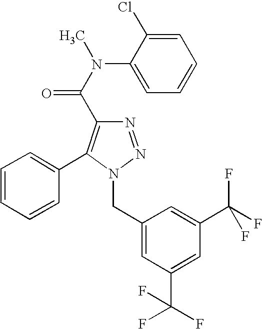

Add oxalyl chloride (0.064 mL, 0.72 mmol) to a solution of 1-(3,5-Bis-trifluoromethyl-benzyl)-5-phenyl-1H-[1,2,3]triazole-4-carboxylic acid (150 mg, 0.36 mmol) and DMF (1 drop) in CH₂Cl₂ (2 mL). Stir the solution for 2.5 h at RT, then concentrate to dryness. Dissolve the residue in 1,2-dichloroethane (DCE) and concentrate to dryness. Dissolve the residue in pyridine (2 mL) and transfer to a sealed tube. Add 2-chloro-N-methylaniline (200 mg, 1.44 mmol) and DMAP (5 mg, cat.) and heat in the sealed tube at 80° C. for 1h. Cool to RT and concentrate to dryness. Dissolve in 20% iPrOH/CHCl₃. Wash with saturated NaHCO₃ and brine, dry over Na₂SO₄, filter and concentrate to dryness. Purify by radial chromatography using an EtOAc/hexanes gradient to afford the title compound (75.4 mg, 39%) as a clear foam/oil. MS(ES) 539.2 (M+1)⁺; HPLC (5-95% 0.1% TFA/water in 3.8 min on YMC ODS (0.46×50mm) .05 mL; 3.0 mL; 25° C.) R_{f}=3.34 min; 99.2%.

Using an analogous procedure to that described above, with the appropriate starting materials, the following compounds may be prepared.

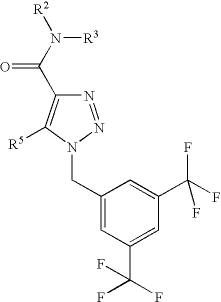

| Ex. # | R² | R³ | R⁵ | Data |
|---|---|---|---|---|
| 10. | hydroxyl | benzyl | phenyl | MS(ES) 521.2 (M + 1)⁺; |
| | | | | ¹H NMR (CDCl₃) δ 7.70 |
| | | | | (m, 1H), 7.10-7.60 (m, 14H), |
| | | | | 5.50-5.60 (m, 3H). |
| 11 | 2,4-dichloro- | methyl | phenyl | MS(ES) 573.0 (M + 1)⁺; |
| | phenyl | | | R_{f} = 0.70 (5% MeOH/CHCl₃). |
| 12 | 2-chloro-4- | methyl | methyl | MS(ES) 491.0 (M + 1)⁺; |
| | methyl-phenyl | | | R_{f} = 0.33 (5% MeOH/CHCl₃). |
| 13 | 2-chloro-4- | methyl | methyl | MS(ES) 495.0 (M + 1)⁺; |
| | fluoro-phenyl | | | R_{f} = 0.60 (5% MeOH/CHCl₃). |
| 14 | 2-chloro- | methyl | methyl | MS(ES) 477.3 (M + 1)⁺; |
| | phenyl | | | R_{f} = 0.31 (5% MeOH/CHCl₃). |

Using a method analogous to Example 9 and the appropriate starting materials, the following compounds may be prepared.

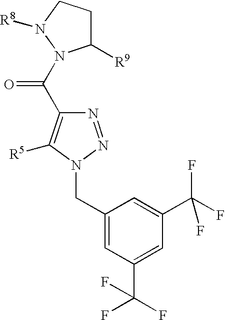

| Ex. # | R⁵ | R⁸ | R⁹ | Data |
|---|---|---|---|---|
| 15 | phenyl | 2-chloro-phenyl | oxo | MS(ES) 594.1 (M + 1)⁺; |
| | | | | R_{f} = 0.6 (50% |
| | | | | EtOAc/hexanes). |
| 16 | methyl | 2-chloro-phenyl | hydrogen | MS(ES) 518.0 (M + 1)⁺; |
| | | | | R_{f} = 0.29 (5% |
| | | | | MeOH/CHCl₃). |

### EXAMPLE 17

### 1-[1-(3,5-Bis-trifluoromethyl-benzyl)-5-phenyl-1H-[1,2,3]triazole-4-carbonyl]-3,(4S)-dimethyl-(5R)-(+)-phenyl-imidazolidin-2-one.

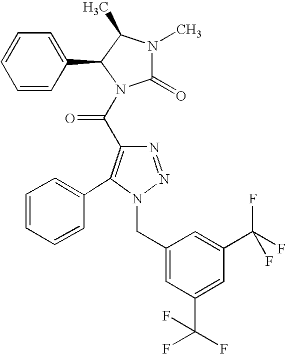

Using a method similar to Example 1, with the exception of using (4S,5R)-(+)-1,5-dimethyl-4-phenyl-2-imidazolidinone (52 mg, 0.28 mmol), affords the title compound (11.7 mg, 7.1%) as a white foam. MS(ES) 588.2 (M+1)⁺; R_{f}=0.54 (80% EtOAc/hexanes).

### EXAMPLE 18

### 1-[1-(3,5-Bis-trifluoromethyl-benzyl)-5-phenyl-1H-[1,2,3]triazole-4-carbonyl]-3 ,(4R)-dimethyl-(5S)-(−)-phenyl-imidazolidin-2-one

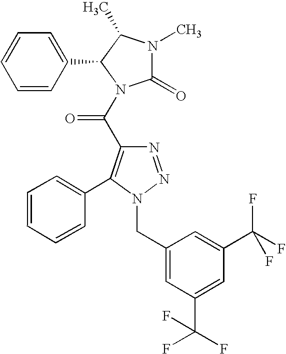

Add oxalyl chloride (0.064 mL, 0.72 mmol) to a solution of 1-(3,5-Bis-trifluoromethyl-benzyl)-5-phenyl-1H-[1,2,3]triazole-4-carboxylic acid (150 mg, 0.36 mmol) in CH₂Cl₂ (2 mL) and DMF (1 drop). Stir the solution for 2 hours at RT, then concentrate to dryness. Dissolve in 1,2-dichloroethane and concentrate to dryness. Dissolve in THF (2 mL) and set aside. This is solution A. Add n-butyllithium (0.15 mL, 0.36 mmol) to a solution of (4R,5S)-(−)-1,5-dimethyl-4-phenyl-2-imidazolidinone (62 mg, 0.32 mmol, Aldrich) in THF (2 mL) at −78° C. Stir for 10 min at −78° C., then add Solution A at −78° C. Stir the mixture for 15 min. at −78° C., then remove cold bath and warm to RT over 1 h. Concentrate to dryness and dissolve in 20% iPrOH/CHCl₃. Wash with saturated aqueous NaHCO₃ and brine, dry over Na₂SO₄, filter, and concentrate. Purify by radial chromatography using an EtOAc/hexanes gradient to afford the title compound (23 mg, 12.5%) as a white foam. MS(ES) 588.3 (M+1)⁺; R_{f}=0.50 (80% EtOAc/hexanes).

### EXAMPLE 19

### 1-(3,5-Bis-trifluoromethyl-benzyl)-5-phenyl-1H-[1,2,3]triazole-4-carboxylic acid (2-chloro-4-fluoro-phenyl)-methyl-amide

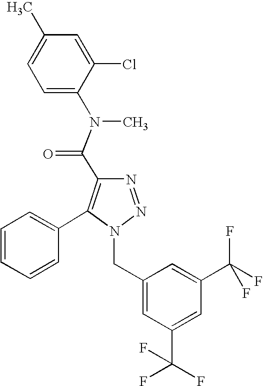

Dissolve 1-(3,5-bis-trifluoromethyl-benzyl)-5-phenyl-1H-[1,2,3]triazole-4-carboxylic acid (2-chloro-4-fluoro-phenyl)-amide (80 mg, 0.15 mmol) in THF (2 mL). Add potassium hexamethyl disilylamide (0.33 mL, 0.17 mmol, 0.5 M in toluene) and methyl iodide (0.011 mL, 0.17 mmol). Stir overnight at RT, then partition between EtOAc and saturated aqueous NaHCO₃. Wash with saturated aqueous NaHCO₃, and brine, dry over sodium sulfate, filter, and concentrate to dryness. Purify the residue by radial chromatography using an EtOAc/hexanes gradient to afford 30 mg (36%) of the title compound as a white foam. MS(ES) 557.0 (M+1)⁺; R_{f}=0.48 (1:1 EtOAc/hexanes).

### EXAMPLE 20

### 1-(3,5-Bis-trifluoromethyl-benzyl)-5-phenyl-1H-[1,2,3]triazole-4-carboxylic acid (2-chloro-4-methyl-phenyl)-methyl-amide

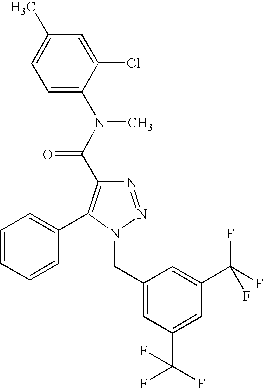

Dissolve 1-(3,5-bis-trifluoromethyl-benzyl)-5-phenyl-1H-[1,2,3]triazole-4-carboxylic acid (100 mg, 0.24 mmol) in CH₂Cl₂ (3 mL) and DMF (1 drop) and add oxalyl chloride (0.042 mL, 0.48 mmol). Stir 1 h at RT, then concentrate. Slurry the residue in 1,2-dichloroethane and concentrate to dryness twice. Dissolve the residue in pyridine (2 mL), add DMAP (5 mg, catalytic) and (2-chloro-4-methyl-phenyl)-methyl-amine (0.74 mg, 0.48 mmol) and heat for 1 h at 100° C. in a sealed tube, then cool to RT and concentrate to dryness. Dissolve in 20% iPrOH/CHCl₃. Wash with saturated aqueous NaHCO₃ and brine, dry over Na₂SO₄, filter, and concentrate. Purify the residue via radial chromatography using a MeOH/CHCl₃ gradient to afford 67 mg (48%) of the title compound as a yellow foam/oil. MS(ES) 553.0 (M+1)⁺; R_{f}=0.42 (5% MeOH/CHCl₃).

### EXAMPLE 21

### 1-(3,5-Bis-trifluoromethyl-benzyl)-5-phenyl-1H-[1,2,3]triazole-4-carboxylic acid (2-chloro-pyridin-3-ylmethyl)-methyl-amide

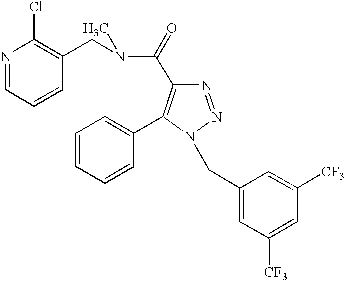

Combine (2-chloro-pyridin-3-ylmethyl)-methyl-amine (0.050 g, 0.32 mmol) with 1-(3,5-bis-trifluoromethyl-benzyl)-5-phenyl-1H-[1,2,3]triazole-4-carboxylic acid (0.10 g, 0.24 mmol), EDCI (0.046 0.24 mmol), 1-hydroxy-7-azabenzotriazole (0.033 g, 0.24 mmol), and N,N-diisopropylethylamine (0.10 mL, 0.56 mmol), in DMF (6 mL) and stir the mixture at RT. After 72 h, concentrate the mixture in vacuo and partition the residue between water and CH₂Cl₂. Separate the layers and dry the CH₂Cl₂ extracts over Na₂SO₄. Filter and concentrate, then purify the residue over silica gel using a MeOH/CH₂Cl₂ gradient to provide the title compound (0.123 g, 92%) as a white solid. MS(ES) 554.1 (M+1)⁺; Anal. Calc'd for C₂₅H₁₈ClF₆N₅O: C, 54.21; H, 3.28; N, 12.64. Found: C, 53.83; H, 3.31; N, 12.33.

Using a method analogous to Example 21, with the appropriate starting materials, the following compounds may be prepared.

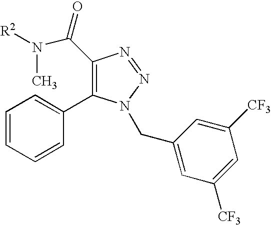

| Ex. # | R² | Data |
|---|---|---|
| 22 | 3-chloro-pyridin- | MS(ES) 554.1 (M + 1)⁺; Anal. Calc'd for |
| | 4-yl-methyl | C₂₅H₁₈ClF₆N₅O: C, 54.21; H, 3.28; N, 12.64. |
| | | Found: C, 53.39; H, 3.49; N, 11.99. |
| 23 | 4-chloro-pyridin- | MS(ES) 554.1 (M + 1)⁺. R_{f} = 0.34 (10:1 |
| | 3-yl-methyl | CHCl₃/MeOH). |

Using a method analogous to Example 21, with the appropriate starting materials, the following compounds may be prepared and isolated.

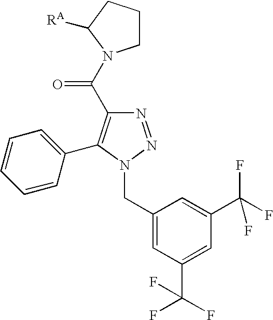

| Ex. # | R^{A} | Data |
|---|---|---|
| 24 | pyridin-2-yl | MS(ES) 546.1 (M + 1)⁺; Anal. Calc'd for |
| | | C₂₇H₂₁F₆N₅O: C, 59.45; H, 3.88; N, 12.84. Found: |
| | | C, 59.29; H, 4.06; N, 13.15. |
| 25 | pyridin-4-yl | MS(ES) 546.1 (M + 1)⁺; Anal. Calc'd for |
| | | C₂₇H₂₁F₆N₅O: C, 59.45; H, 3.88; N, 12.84. Found: |
| | | C, 59.29; H, 3.98; N, 13.12. |
| 26 | benzyl | MS(ES) 559.19 (M + 1)⁺; R_{f} = 0.85 |
| | | (10:1 CHCl₃/MeOH). |
| 27 | phenethyl | MS(ES) 573.2 (M + 1)⁺; R_{f} = 0.76 |
| | | (10:1 CHCl₃/MeOH). |
| 28 | cyclohexyl | MS(ES) 551.2 (M + 1)⁺; R_{f} = 0.62 |
| | | (10:1 CHCl₃/MeOH). |
| 29 | isobutyl | MS(ES) 525.2 (M + l)⁺; R_{f} = 0.53 |
| | | (10:1 CHCl₃/MeOH). |
| 30 | pyridin-3-yl- | MS(ES) 560.1 (M + 1)⁺; R_{f} = 0.28 |
| | methyl | (10:1 CHCl₃/MeOH). |

Using the method similar to Example 21, with the appropriate starting materials, the following compounds may be prepared and isolated.

| Ex. # | Product | Data |
|---|---|---|
| 31 | 1-Phenethyl-5-phenyl-1H- | MS(ES) 559.2(M+1)⁺; |
| | [1,2,3]triazole-4-carboxylic | R_{f}=0.82(10:1 |
| | acid (3,5-bis-trifluoromethyl-benzyl)- | CHCl₃/MeOH) |
| | cyclopropyl-amide | |
| 32 | 1-Phenethyl-5-phenyl-1H- | MS(ES) 561.2(M+1)+; |
| | [1,2,3]triazole-4-carboxylic | R_{f}=0.79(10:1 |
| | acid (3,5-bis-trifluoromethyl-benzyl)- | CHCl₃/MeOH) |
| | isopropyl-amide | |

### EXAMPLE 33

### 1-(3,5-Bis-trifluoromethyl-benzyl)-5-phenyl-1H-[1,2,3]triazole-4-carboxylic acid (2-chloro-phenyl)-isopropyl-amide

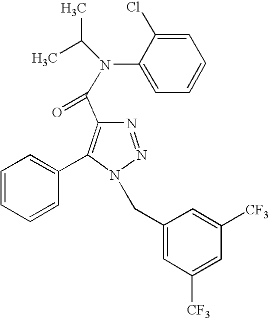

Combine 1-(3,5-Bis-trifluoromethyl-benzyl)-5-phenyl-1H-[1,2,3]triazole-4-carboxylic acid (0.15 g, 0.36 mmol) and DMF (1 drop) in CH₂Cl₂ (5 mL) and slowly add oxalyl chloride (0.10 mL, 1.14 mmol) via syringe and stir until gas evolution ceases. Concentrate the mixture in vacuo and concentrate the residue once from diethyl ether. Dissolve this crude acid chloride in pyridine (5 mL) and add (2-chlorophenyl)-isopropyl-1 mg, 0.36 mmol) and DMAP (3 mg). Heat the mixture at 100° C. for 1 h, then cool to RT and concentrate. Partition the residue between water and EtOAc and dry the combined extracts over Na₂SO₄. Concentrate the extracts and purify the residue by chromatography over silica gel using a CH₂Cl₂/MeOH gradient to provide the title compound (113 mg, 55%) as a thick oil which solidifies. MS(ES) 567.1 (M+1)⁺; R_{f}=0.61 (6.7% MeOH/CH₂Cl₂).

### EXAMPLE 34

### 1-(3,5-Bis-trifluoromethyl-benzyl)-5-phenyl-1H-[1,2,3]triazole-4-carboxylic acid (2-chloro-benzyl)-(2-dimethylamino-ethyl)-amide

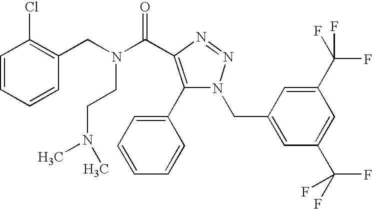

Dissolve 1-(3,5-bis-trifluoromethyl-benzyl)-5-phenyl-1H-[1,2,3]triazole-4-carboxylic acid (160 mg, 0.37 mmol) in dry CH₂Cl₂ (0.2M) add N′-(2-chlorobenzyl)-N,N-dimethyl-ethane-1,2-diamine (78 mg, 0.37 mmol), followed by triethylamine (0.26 mL, 1.85 mmol). After 24 h, dilute with CH₂Cl₂ (2 mL) and wash with 1N NaOH (2×3 mL), dry, filter, and concentrate. Purify the residue by chromatography (50:1 to 20:1 CHCl₃/MeOH gradient) to provide the title compound. MS(ES) 610.1 (M+1)⁺; R_{f}=0.44 (10:1 CHCl₃/MeOH).

By a method analogous to Example 34, the following compounds may be prepared and isolated.

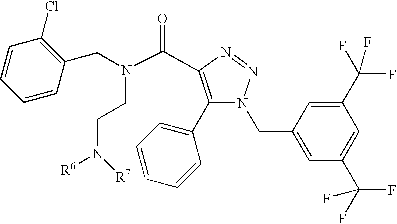

| Ex. # | —NR⁶R⁷ | Data |
|---|---|---|
| 35 | pyrrolidin-1-yl | MS/ES: 636.2 (M + 1); R_{f} = 0.42 |
| | | (10:1 CHCl₃/MeOH). |
| 36 | morpholino | MS/ES: 652.1 (M + 1); R_{f} = 0.15 |
| | | (10:1 CHCl₃/MeOH). |

### EXAMPLE 37

### 5-Phenyl-1-(3-trifluoromethyl-benzyl)-1H-[1,2,3]triazole-4-carboxylic acid benzyl-methyl-amide

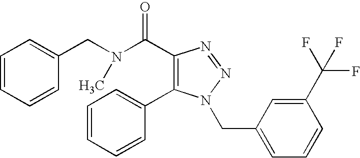

Add 3-(trifluoromethyl)benzyl azide (1.2 eq) to a solution of 3-phenyl-propynoic acid benzyl-methyl-amide (1 eq) in toluene (0.3 M). Heat the resulting solution at 120° C. in a sealed (screw-cap) test tube using a block heater that is placed on an orbital shaker for agitation. After 48 h, cool to RT and apply the reaction mixture directly to the top of a pre-packed silica gel column. Elution with a hexanes/EtOAc gradient provides two regioisomeric triazoles. The desired product is the slower eluting (lower R_{f}) spot. R_{f}=0.18 (2:1 hexanes/EtOAc); MS(ES): 451.2 (M+1)⁺.

Using a method analogous to Example 37, with the appropriate starting materials, the following compounds may be prepared and isolated.

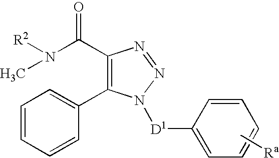

| Ex. # | D¹ | R^{a} | R² | Data |
|---|---|---|---|---|
| 38 | methylene | 2- | benzyl | R_{f} = 0.23 (2:1 hexanes/EtOAc); |
| | | trifluoromethyl | | MS(ES) 451.2 (M + 1)⁺. |
| 39 | methylene | 3-fluoro | benzyl | R_{f} = 0.15 (2:1 hexanes/EtOAc); |
| | | | | MS(ES) 401.2 (M + 1)⁺. |
| 40 | ethylene | hydrogen | benzyl | R_{f} = 0.13 (2:1 hexanes/EtOAc); |
| | | | | MS(ES) 397.2 (M + 1)⁺. |
| 41 | ethylene | 3-methyl | benzyl | R_{f} = 0.15 (2:1 hexanes/EtOAc); |
| | | | | MS(ES) 411.2 (M + 1)⁺. |
| 42 | ethylene | 3-trifluoro- | benzyl | R_{f} = 0.10 (2:1 hexanes/EtOAc); |
| | | methyl | | MS(ES) 465.2 (M + 1)⁺. |
| 43 | propane-2,3-diyl | hydrogen | benzyl | R_{f} = 0.20 (2:1 hexanes/EtOAc); |
| | | | | MS(ES) 411.2 (M + 1)⁺. |
| 44 | methylene | 3,5-bis- | benzyl | R_{f} = 0.15 (2:1 hexanes/EtOAc); |
| | | trifluoromethyl | | MS(ES) 519.2 (M + 1)⁺. |
| 45 | methylene | 3,5-dichloro | benzyl | R_{f} = 0.18 (2:1 hexanes/EtOAc); |
| | | | | MS(ES) 451.1 (M + 1)⁺. |
| 46 | methylene | 3,5-dimethyl | benzyl | R_{f} = 0.23 (2:1 hexanes/EtOAc); |
| | | | | MS(ES) 411.1 (M + 1)⁺. |
| 47 | ethylene | 4-methoxy | 3,5-dimethyl- | R_{f} = 0.13 (2:1 hexanes/EtOAc); |
| | | | benzyl | MS(ES) 455.3 (M + 1)⁺. |
| 48 | ethylene | 4-methoxy | 3,5-dichloro- | R_{f} = 0.13 (2:1 hexanes/EtOAc); |
| | | | benzyl | MS(ES) 495.2 (M + 1)⁺. |
| 49 | ethylene | 4-methoxy | 3-fluoro-5- | R_{f} = 0.15 (2:1 hexanes/EtOAc); |
| | | | trifluoromethyl-benzyl | MS(ES) 513.2 (M + 1)⁺. |
| 50 | ethylene | 3,5-bis- | benzyl | R_{f} = 0.15 (2:1 hexanes/EtOAc); |
| | | trifluoromethyl | | MS(ES) 533.2 (M + 1)⁺. |
| 51 | methylene | 3-chloro | benzyl | R_{f} = 0.15 (2:1 hexanes/EtOAc); |
| | | | | MS(ES) 417.1 (M + 1)⁺. |
| 52 | methylene | 3,5-dibromo | benzyl | R_{f} = 0.20 (2:1 hexanes/EtOAc); |
| | | | | MS(ES) 541.0 (M + 1)⁺. |
| 53 | methylene | 3,5-bis- | phenethyl | R_{f} = 0.20 (2:1 hexanes/EtOAc); |
| | | trifluoromethyl | | MS(ES) 533.2 (M + 1)⁺. |
| 54 | methylene | 3,5-dichloro | phenethyl | R_{f} = 0.18 (2:1 hexanes/EtOAc); |
| | | | | MS(ES) 465.1 (M + 1)⁺. |
| 55 | methylene | hydrogen | 2-chloro-benzyl | R_{f} = 0.23 (2:1 hexanes/EtOAc); |
| | | | | MS(ES) 417.1 (M + 1)⁺. |
| 56 | methylene | 3,5-dimethyl | 2-chloro-benzyl | R_{f} = 0.30 (2:1 hexanes/EtOAc); |
| | | | | MS(ES) 445.2 (M + 1)⁺. |
| 57 | methylene | 3,5-dibromo | 2-chloro-benzyl | R_{f} = 0.26 (2:1 hexanes/EtOAc); |
| | | | | MS(ES) 575.0 (M + 1)⁺. |
| 58 | methylene | 3,5-dichloro | 2-chloro-benzyl | R_{f} = 0.26 (2:1 hexanes/EtOAc); |
| | | | | MS(ES) 485.1 (M + 1)⁺. |
| 59 | methylene | 2-chloro | 2-chloro-benzyl | R_{f} = 0.26 (2:1 hexanes/EtOAc); |
| | | | | MS(ES) 451.1 (M + 1)⁺. |
| 60 | methylene | 3-chloro | 2-chloro-benzyl | R_{f} = 0.20 (2:1 hexanes/EtOAc); |
| | | | | MS(ES) 451.1 (M + 1)⁺. |
| 61 | methylene | 4-methoxy | 2-chloro-benzyl | R_{f} = 0.17 (2:1 hexanes/EtOAc); |
| | | | | MS(ES) 447.1 (M + 1)⁺. |
| 62 | methylene | 3-trifluoro- | 2-chloro-benzyl | R_{f} = 0.26 (2:1 hexanes/EtOAc); |
| | | methyl | | MS(ES) 485.1 (M + 1)⁺. |
| 63 | methylene | 2-methyl | 2-chloro-benzyl | R_{f} = 0.26 (2:1 hexanes/EtOAc); |
| | | | | MS(ES) 431.1 (M + 1)⁺. |
| 64 | methylene | 3-methyl | 2-chloro-benzyl | R_{f} = 0.29 (2:1 hexanes/EtOAc); |
| | | | | MS(ES) 431.1 (M + 1)⁺. |
| 65 | methylene | 4-methyl | 2-chloro-benzyl | R_{f} = 0.29 (2:1 hexanes/EtOAc); |
| | | | | MS(ES) 431.1 (M + 1)⁺. |
| 66 | methylene | hydrogen | 3,5-bis-trifluoro- | R_{f} = 0.32 (2:1 hexanes/EtOAc); |
| | | | methyl-benzyl | MS(ES) 519.1 (M + 1)⁺. |
| 67 | methylene | 2-methyl | 3,5-bis-trifluoro- | R_{f} = 0.34 (2:1 hexanes/EtOAc); |
| | | | methyl-benzyl | MS(ES) 533.1 (M + 1)⁺. |
| 68 | methylene | 3-methyl | 3,5-bis-trifluoro- | R_{f} = 0.34 (2:1 hexanes/EtOAc); |
| | | | methyl-benzyl | MS(ES) 533.1 (M + 1)⁺. |
| 69 | methylene | 4-methyl | 3,5-bis-trifluoro- | R_{f} = 0.29 (2:1 hexanes/EtOAc); |
| | | | methyl-benzyl | MS(ES) 533.1 (M + 1)⁺. |
| 70 | methylene | 2-chloro | 3,5-bis-trifluoro- | R_{f} = 0.29 (2:1 hexanes/EtOAc); |
| | | | methyl-benzyl | MS(ES) 553.0 (M + 1)⁺. |
| 71 | methylene | 3-chloro | 3,5-bis-trifluoro- | R_{f} = 0.26 (2:1 hexanes/EtOAc); |
| | | | methyl-benzyl | MS(ES) 553.1 (M + 1)⁺. |
| 72 | ethylene | 2-methoxy | 3,5-bis-trifluoro- | R_{f} = 0.23 (2:1 hexanes/EtOAc); |
| | | | methyl-benzyl | MS(ES) 563.2 (M + 1)⁺. |
| 73 | ethylene | hydrogen | 3,5-bis-trifluoro- | R_{f} = 0.20 (2:1 hexanes/EtOAc); |
| | | | methyl-benzyl | MS(ES) 533.2 (M + 1)⁺. |
| 74 | ethane-1,1-diyl | 3- | benzyl | R_{f} = 0.23 (2:1 hexanes/EtOAc); |
| | | trifluoromethyl | | MS(ES) 465.2 (M + 1)⁺. |
| 75 | ethane-1,1-diyl | 3- | 2-chloro-benzyl | R_{f} = 0.29 (2:1 hexanes/EtOAc); |
| | | trifluoromethyl | | MS(ES) 499.2 (M + 1)⁺. |
| 76 | methylene | 4-methyl | benzyl | R_{f} = 0.20 (2:1 hexanes/EtOAc); |
| | | | | MS(ES) 397.3 (M + 1)⁺. |
| 77 | methylene | 2-methoxy | benzyl | R_{f} = 0.14 (2:1 hexanes/EtOAc); |
| | | | | MS(ES) 413.2 (M + 1)⁺. |
| 78 | methylene | 3-methoxy | benzyl | R_{f} = 0.14 (2:1 hexanes/EtOAc); |
| | | | | MS(ES) 413.2 (M + 1)⁺. |
| 79 | methylene | 2-bromo | benzyl | R_{f} = 0.20 (2:1 hexanes/EtOAc); |
| | | | | MS(ES) 461.1 (M⁺), 463.1 |
| | | | | (M + 2)⁺. |
| 80 | ethylene | 3- | 3,5-dimethyl- | MS(ES) 493.3 (M + 1)⁺; R_{f} = 0.31 |
| | | trifluoromethyl | benzyl | (2:1 hexanes/EtOAc). |
| 81 | ethylene | 3- | 3,5-dichloro- | MS(ES): 533.1 (M + 1)⁺; R_{f} = |
| | | trifluoromethyl | benzyl | 0.16 (2:1 hexanes/EtOAc). |
| 82 | ethylene | 3- | 3-fluoro-5-trifluoro- | MS(ES) 551.2 (M + 1)⁺; R_{f} = 0.13 |
| | | trifluoromethyl | methyl-benzyl | (2:1 hexanes/EtOAc). |
| 83 | ethylene | 3- | 5-chloro-2- | MS(ES) 529.2 (M + 1)⁺; R_{f} = 0.09 |
| | | trifluoromethyl | methoxy-benzyl | (2:1 hexanes/EtOAc). |
| 84 | ethane-1,1-diyl | 4-fluoro | benzyl | MS(ES) 415.2 (M + 1)⁺; R_{f} = 0.26 |
| | | | | (2:1 hexanes/EtOAc). |
| 85 | ethylene | 3- | 5-fluoro-2- | MS(ES) 513.2 (M + 1)⁺; R_{f} = 0.12 |
| | | trifluoromethyl | methoxy-benzyl | (2:1 hexanes/EtOAc). |
| 86 | ethylene | 3- | 2-methoxy-5-trifluoro- | MS(ES) 579.2 (M + 1)⁺; R_{f} = 0.10 |
| | | trifluoromethyl | methoxy-benzyl | |
| 87 | ethylene | 3- | 2-chloro-benzyl | MS(ES) 499.1 (M + 1)⁺; R_{f} = 0.14 |
| | | trifluoromethyl | | (2:1 hexanes/EtOAc). |
| 88 | ethane-1,1-diyl | 3-methyl | benzyl | MS(ES) 411.2 (M + 1)⁺; R_{f} = 0.30 |
| | | | | (2:1 hexanes/EtOAc). |
| 89 | ethylene | 4-fluoro | benzyl | MS(ES) 415.2 (M + 1)⁺; R_{f} = 0.25 |
| | | | | (2:1 hexanes/EtOAc). |
| 90 | propane- | hydrogen | benzyl | MS(ES) 411.2 (M + 1 )⁺; R_{f} = 0.15 |
| | 1,3-diyl | | | (2:1 hexanes/EtOAc). |
| 91 | propane- | 4-methoxy | benzyl | MS(ES) 441.3 (M + 1)⁺; R_{f} = 0.40 |
| | 1,3-diyl | | | (2:1 hexanes/EtOAc). |
| 92 | ethylene | 4-ethoxy | benzyl | MS(ES) 441.2 (M + 1)⁺; R_{f} = 0.14 |
| | | | | (2:1 hexanes/EtOAc). |
| 93 | methylene | 3,5-bis- | 3-fluoro-5- | MS(ES) 605.2 (M + 1)⁺; R_{f} = 0.28 |
| | | trifluoromethyl | trifluoromethyl-benzyl | (2:1 hexanes/EtOAc). |
| 94 | methylene | 3,5-bis- | 5-fluoro-2- | MS(ES) 567.2 (M + 1)⁺; R_{f} = 0.21 |
| | | trifluoromethyl | methoxy-benzyl | (2:1 hexanes/EtOAc): |
| 95 | methylene | 3,5-bis- | 3,5-dimethyl- | MS(ES) 547.2 (M + 1)⁺; R_{f} = 0.30 |
| | | trifluoromethyl | benzyl | (2:1 hexanes/EtOAc). |
| 96 | methylene | 3,5-bis- | 5-chloro-2- | MS(ES) 583.1 (M + 1)⁺; R_{f} = 0.15 |
| | | trifluoromethyl | methoxy-benzyl | (2:1 hexanes/EtOAc). |
| 97 | methylene | 3,5-bis- | 2-methoxy-5-trifluoro- | MS(ES) 633.2 (M + 1)⁺; R_{f} = 0.30 |
| | | trifluoromethyl | methoxy-benzyl | (2:1 hexanes/EtOAc). |

By a method analogous to Example 37, with the appropriate starting materials, the following compounds may be prepared and isolated.

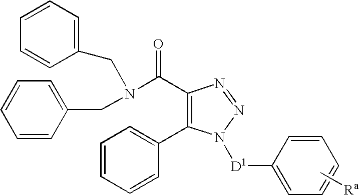

| Ex. # | D¹ | R^{a} | Data |
|---|---|---|---|
| 98 | methylene | 3,5-bis- | R_{f} = 0.38; MS(ES) 595.2 (M + 1) |
| | | trifluoromethyl | |
| 99 | ethylene | 3-trifluoromethyl | R_{f} = 0.36; MS(ES) 541.3 (M + 1); |

### EXAMPLE 100

### 1-Bis-trifluoromethyl-benzyl)-5-phenyl-1H-[1,2,3]triazole-4-carboxylic acid (2-chloro-benzyl)-methyl-amide

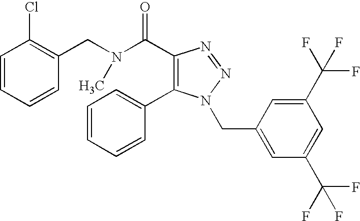

Suspend 1-(3,5-bis-trifluoromethyl-benzyl)-5-phenyl-1H-[1,2,3]triazole-4-carboxylic acid (100 mg, 1 eq) and HOBt (64 mg, 2 eq) in dry CH₂Cl₂ (2.4 mL, 0.1 M solution). Add N-methyl-N-(2-chlorobenzyl) amine (66 mg, 1.5 eq) and triethylamine (0.17 mL, 5 eq) followed by EDCI (92 mg, 2 eq). Stir at RT overnight, then dilute with CH₂Cl₂ (5 mL) and wash with 1N HCl solution, saturated NaHCO₃ solution, and brine. Dry over MgSO₄, filter, and concentrate. Purify the residue by flash chromatography on silica gel using a 4:1 to 1:1 hexanes/EtOAc gradient to provide the title compound (118 mg, 89%) as a pale yellow oil that crystallizes upon standing. R_{f}=0.35 (2:1 hexanes/EtOAc); MS(ES) 553.2 (M+1)⁺.

By a method analogous to Example 100, the following compounds may be prepared and isolated.

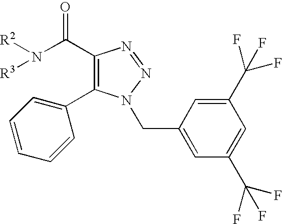

| Ex. # | R² | R³ | Data |
|---|---|---|---|
| 101 | 2-fluoro-benzyl | methyl | R_{f} = 0.25 (2:1 hexanes/EtOAc); |
| | | | MS(ES) 537.2 (M + 1)⁺. |
| 102 | 4-fluoro-benzyl | methyl | R_{f} = 0.15 (2:1 hexanes/EtOAc); |
| | | | MS(ES) 537.2 (M + l)⁺. |
| 103 | 3-methyl-benzyl | methyl | R_{f} = 0.23 (2:1 hexanes/EtOAc); |
| | | | MS(ES) 533.2 (M + 1)⁺. |
| 104 | 2-methoxy-benzyl | methyl | R_{f} = 0.15 (2:1 hexanes/EtOAc); |
| | | | MS(ES) 549.2 (M + 1)⁺. |
| 105 | 3-methoxy-benzyl | methyl | R_{f} = 0.18 (2:1 hexanes/EtOAc); |
| | | | MS(ES) 549.2 (M + 1)⁺. |
| 106 | 4-methoxy-benzyl | methyl | R_{f} = 0.18 (2:1 hexanes/EtOAc); |
| | | | MS(ES) 549.2 (M + 1)⁺. |
| 107 | 4-chloro-benzyl | methyl | R_{f} = 0.23 (2:1 hexanes/EtOAc); |
| | | | MS(ES) 553.2 (M + 1)⁺. |
| 108 | 3-chloro-benzyl | methyl | R_{f} = 0.20 (2:1 hexanes/EtOAc); |
| | | | MS(ES) 553.2 (M + 1)⁺. |
| 109 | 4-trifluoromethyl- | methyl | R_{f} = 0.20 (2:1 hexanes/EtOAc); |
| | benzyl | | MS(ES) 587.2 (M + 1). |
| 110 | 4-pyrrolidin-1-yl- | methyl | R_{f} = 0.18 (2:1 hexanes/EtOAc); |
| | benzyl | | MS(ES) 588.1 (M + 1)⁺ |
| 111 | 4-dimethylamino- | methyl | R_{f} = 0.15 (2:1 hexanes/EtOAc); |
| | benzyl | | MS(ES) 562.1 (M + 1)⁺. |
| 112 | 2-methyl-benzyl | methyl | R_{f} = 0.25 (2:1 hexanes/EtOAc); |
| | | | MS(ES) 533.2 (M + 1)⁺. |
| 113 | 4-methyl-benzyl | methyl | R_{f} = 0.25 (2:1 hexanes/EtOAc); |
| | | | MS(ES) 533.2 (M + 1)⁺. |
| 114 | 3-fluoro-benzyl | methyl | R_{f} = 0.33 (2:1 hexanes/EtOAc); |
| | | | MS(ES) 537.2 (M + 1)⁺. |
| 115 | 2-trifluoromethyl- | methyl | R_{f} = 0.35 (2:1 hexanes/EtOAc); |
| | benzyl | | MS(ES) 587.2 (M + 1)⁺. |
| 116 | 3-trifluoromethyl- | methyl | R_{f} = 0.35 (2:1 hexanes/EtOAc); |
| | benzyl | | MS(ES) 587.2 (M + 1)⁺. |
| 117 | pyridin-2-yl-methyl | methyl | R_{f} = 0.25 (2:1 hexanes/EtOAc); |
| | | | MS(ES) 520.2 (M + 1)⁺. |
| 118 | pyridin-4-yl-methyl | methyl | R_{f} = 0.09 (2:1 hexanes/EtOAc); |
| | | | MS(ES) 520.2 (M + 1)⁺. |
| 119 | 1-phenyl-ethyl | methyl | R_{f} = 0.28 (2:1 hexanes/EtOAc); |
| | | | MS(ES) 533.2 (M + 1)⁺. |
| 120 | 1-(3-chloro-phenyl)- | methyl | R_{f} = 0.35 (2:1 hexanes/EtOAc); |
| | ethyl | | MS(ES) 567.2 (M + 1)⁺. |
| 121 | 2-chloro-6-fluoro- | methyl | R_{f} = 0.30 (2:1 hexanes/EtOAc); |
| | benzyl | | MS(ES) 571.2 (M + 1)⁺. |
| 122 | 2,6-dichloro-benzyl | methyl | R_{f} = 0.35 (2:1 hexanes/EtOAc); |
| | | | MS(ES) 587.1 (M + 1)⁺. |
| 123 | 2,3-dichloro-benzyl | methyl | R_{f} = 0.33 (2:1 hexanes/EtOAc); |
| | | | MS(ES) 587.1 (M + 1)⁺. |
| 124 | 2-chloro-4-fluoro- | methyl | R_{f} = 0.30 (2:1 hexanes/EtOAc); |
| | benzyl | | MS(ES) 571.2 (M + 1)⁺. |
| 125 | 2,4-difluoro-benzyl | methyl | R_{f} = 0.23 (2:1 hexanes/EtOAc); |
| | | | MS(ES) 555.2 (M + 1)⁺. |
| 126 | 2,6-difluoro-benzyl | methyl | R_{f} = 0.28 (2:1 hexanes/EtOAc); |
| | | | MS(ES) 555.2 (M + 1)⁺. |
| 127 | 2-bromo-benzyl | methyl | R_{f} = 0.28 (2:1 hexanes/EtOAc); |
| | | | MS(ES) 597.1 (M+), |
| | | | 599.1 (M + 2)⁺. |
| 128 | 2-trifluoromethoxy- | methyl | R_{f} = 0.30 (2:1 hexanes/EtOAc); |
| | benzyl | | MS(ES) 603.1 (M + 1)⁺. |
| 129 | 2-chloro-benzyl | 2-chloro- | R_{f} = 0.23 (2:1 hexanes/EtOAc); |
| | | benzyl | MS(ES) 663.1 (M + 1)⁺. |
| 130 | 2-fluoro-benzyl | 2-fluoro- | R_{f} = 0.47 (2:1 hexanes/EtOAc); |
| | | benzyl | MS(ES) 631.2 (M + 1)⁺. |
| 131 | 2-chloro-benzyl | 1-phenyl- | R_{f} = 0.53 (2:1 hexanes/EtOAc); |
| | | ethyl | MS(ES) 643.2 (M + 1)⁺. |
| 132 | phenyl | methyl | R_{f} = 0.17 (2:1 hexanes/EtOAc); |
| | | | MS(ES) 505.1 (M + 1)⁺. |
| 133 | 4-methyl-phenyl | methyl | R_{f} = 0.14 (2:1 hexanes/EtOAc); |
| | | | MS(ES) 519.2 (M + 1)⁺. |
| 134 | 3-methyl-phenyl | methyl | R_{f} = 0.17 (2:1 hexanes/EtOAc); |
| | | | MS(ES) 519.2 (M + 1)⁺. |
| 135 | 2-methyl-phenyl | methyl | R_{f} = 0.26 (2:1 hexanes/EtOAc); |
| | | | MS(ES) 519.2 (M + 1)⁺. |
| 136 | 2-chloro-benzyl | 1-phenyl- | R_{f} = 0.26 (2:1 hexanes/EtOAc); |
| | | ethyl | MS(ES) 643.2 (M + l)⁺. |
| 137 | 1-(2-methyl- | methyl | R_{f} = 0.33 (2:1 hexanes/EtOAc); |
| | phenyl)-ethyl | | MS(ES) 547.3 (M + 1)⁺. |
| 138 | 1-(3-fluoro-phenyl)- | methyl | R_{f} = 0.33 (2:1 hexanes/EtOAc); |
| | ethyl | | MS(ES) 551.2 (M + 1)⁺. |
| 139 | 1-(4-fluoro-phenyl)- | methyl | R_{f} = 0.33 (2:1 hexanes/EtOAc); |
| | ethyl | | MS(ES) 551.2 (M + 1)⁺. |
| 140 | 1-(2,3-dichloro- | methyl | R_{f} = 0.17 (2:1 hexanes/EtOAc); |
| | phenyl)-ethyl | | MS(ES) 601.1 (M + 1)⁺. |
| 141 | 1,2,3,4-tetrahydro- | methyl | R_{f} = 0.36 (2:1 hexanes/EtOAc); |
| | naphthalen-1-yl | | MS(ES) 559.2 (M + 1)⁺. |
| 142 | indan-1-yl | methyl | R_{f} = 0.28 (2:1 hexanes/EtOAc); |
| | | | MS(ES) 545.3 (M + l)⁺. |
| 143 | 1,2,3,4-tetrahydro- | methyl | R_{f} = 0.25 (2:1 hexanes/EtOAc); |
| | naphthalen-2-yl | | MS(ES) 559.3 (M + 1)⁺. |
| 144 | 1-naphthalen-2- | methyl | R_{f} = 0.25 (2:1 hexanes/EtOAc); |
| | yl-ethyl | | MS(ES) 583.2 (M + 1)⁺. |
| 145 | 2-chloro-benzyl | ethyl | R_{f} = 0.34 (2:1 hexanes/EtOAc); |
| | | | MS(ES) 567.2 (M + 1)⁺. |
| 146 | cyclo-propyl | 2-chloro- | R_{f} = 0.31 (2:1 hexanes/EtOAc); |
| | | benzyl | MS(ES) 579.2 (M + 1)⁺ |
| 147 | 2-chloro-benzyl | propyl | R_{f} = 0.40 (2:1 hexanes/EtOAc); |
| | | | MS(ES) 581.2 (M + 1)⁺. |
| 148 | 2-chloro-benzyl | isopropyl | R_{f} = 0.40 (2:1 hexanes/EtOAc); |
| | | | MS(ES) 581.2 (M + 1)⁺. |
| 149 | naphthalene-2-yl- | methyl | MS(ES) 569.2 (M + 1)⁺. |
| | methyl | | |
| 150 | isobutyl | methyl | R_{f} = 0.29 (2:1 hexanes/EtOAc); |
| | | | MS(ES) 485.2 (M + 1). |
| 151 | 4-hydroxy-phenyl | methyl | R_{f} = 0.05 (2:1 hexanes/EtOAc); |
| | | | MS(ES) 521.2 (M + 1)⁺. |
| 152 | benzyl | isopropyl | R_{f} = 0.31 (2:1 hexanes/EtOAc); |
| | | | MS(ES) 547.2 (M + 1)⁺. |
| 153 | 2,4-difluoro-phenyl | methyl | MS(ES) 541.1 (M + 1)⁺. |
| 154 | 3-chloro-phenyl | methyl | R_{f} = 0.23 (2:1 hexanes/EtOAc); |
| | | | MS(ES) 539.1 (M + 1)⁺. |
| 155 | cyclohexyl | methyl | MS(ES) 511.2 (M + 1)⁺. |
| 156 | naphthalene-2-yl | methyl | MS(ES) 555.2 (M + 1)⁺. |
| 157 | benzyl | propyl | MS(ES) 547.2 (M + 1)⁺. |
| 158 | 2-(2-chloro-phenyl)- | methyl | MS(ES) 567.2 (M + 1)⁺. |
| | ethyl | | |
| 159 | 4-chloro-phenyl | methyl | R_{f} = 0.17 (2:1 hexanes/EtOAc); |
| | | | MS(ES) 539.1 (M + 1)⁺. |
| 160 | 2-methyl-benzyl | methyl | R_{f} = 0.25 (2:1 hexanes/EtOAc); |
| | | | MS(ES) 533.3 (M + 1)⁺. |
| 161 | 3,4-dichloro-phenyl | methyl | R_{f} = 0.24 (2:1 hexanes/EtOAc); |
| | | | MS(ES) 573.1 (M + 1)⁺. |
| 162 | benzyl | ethyl | R_{f} = 0.30 (2:1 hexanes/EtOAc); |
| | | | MS(ES) 533.2 (M + 1)⁺. |
| 163 | 4-methoxy-phenyl | methyl | R_{f} = 0.12 (2:1 hexanes/EtOAc); |
| | | | MS(ES) 535.2 (M + 1)⁺. |
| 164 | indan-2yl | methyl | R_{f} = 0.26 (2:1 hexanes/EtOAc); |
| | | | MS(ES) 545.3 (M + 1)⁺. |
| 165 | pyridin-2-yl | methyl | R_{f} = 0.08 (2:1 hexanes/EtOAc); |
| | | | MS(ES) 506.2 (M + 1)⁺. |
| 166 | 6-methyl-pyridin-2- | methyl | R_{f} = 0.33 (2:1 hexanes/EtOAc); |
| | yl-methyl | | MS(ES) 534.2 (M + 1)⁺. |
| 167 | cyclopentyl | methyl | R_{f} = 0.24 (2:1 hexanes/EtOAc); |
| | | | MS(ES) 497.2 (M + 1)⁺. |
| 168 | propyl | methyl | R_{f} = 0.22 (2:1 hexanes/EtOAc); |
| | | | MS(ES) 471.1 (M + 1)⁺. |
| 169 | 2-(2-methoxy- | methyl | R_{f} = 0.19 (2:1 hexanes/EtOAc); |
| | phenyl)- | | MS(ES) |
| | 1-methyl-ethyl | | |
| 170 | cyclo-propyl | benzyl | R_{f} = 0.32 (2:1 hexanes/EtOAc); |
| | | | MS(ES) 545.2 (M + 1)⁺ |
| 171 | 4-trifluoromethoxy- | methyl | R_{f} = 0.24 (2:1 hexanes/EtOAc); |
| | phenyl | | MS(ES) 589.1 (M + 1)⁺. |
| 172 | (R)-1-phenyl-ethyl | methyl | MS(ES) 533.2 (M + 1)⁺. |
| 173 | 2-diethylamino- | methyl | R_{f} = 0.07 (2:1 hexanes/EtOAc); |
| | ethyl | | MS(ES) 528.3 (M + 1)⁺. |
| 174 | 2-dimethylamino- | methyl | R_{f} = 0.09 (2:1 hexanes/EtOAc); |
| | ethyl | | MS(ES) 500.1 (M + 1)⁺. |
| 175 | 3-diethylamino- | methyl | R_{f} = 0.03 (2:1 hexanes/EtOAc); |
| | propyl | | MS(ES) 542.3 (M + 1)⁺. |
| 176 | ethyl | ethyl | R_{f} = 0.22 (2:1 hexanes/EtOAc); |
| | | | MS(ES) 471.1 (M + 1)⁺. |
| 177 | (S)-1-phenyl-ethyl | methyl | MS(ES) 533.2 (M + 1)⁺. |
| 178 | ethyl | methyl | R_{f} = 0.16 (2:1 hexanes/EtOAc); |
| | | | MS(ES) 457.1 (M + l)⁺. |
| 179 | 1-benzyl-pyrrolidin- | methyl | R_{f} = 0.25 (2:1 hexanes/EtOAc); |
| | 3-yl | | MS(ES) 588.2 (M + 1)⁺. |
| 180 | 1-methyl-piperidin- | methyl | MS(ES) 526.2 (M + l)⁺. |
| | 4-yl | | |
| 181 | isopropyl | methyl | R_{f} = 0.24 (2:1 hexanes/EtOAc); |
| | | | MS(ES) 471.2 (M + 1)⁺. |
| 182 | 1-benzyl-piperidin- | methyl | R_{f} = 0.32 (2:1 hexanes/EtOAc); |
| | 4-yl | | MS(ES) 602.3 (M + 1)⁺. |

By a method similar to Example 100, using the appropriate starting materials, the following compounds may be prepared and isolated.

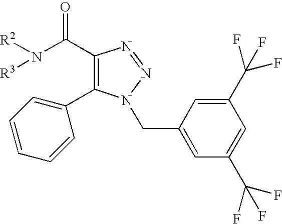

| Ex. # | —NR²R³ | Data |
|---|---|---|
| 183 | 2-phenyl-piperidino | R_{f} = 0.39 (2:1 hexanes/EtOAc); MS(ES) |
| | | 559.3 (M + 1)⁺. |
| 184 | 2-phenyl-pyrrolidin-1-yl | R_{f} = 0.11 (2:1 hexanes/EtOAc); MS(ES) |
| | | 545.3 (M + 1)⁺. |
| 185 | 4,4-dimethyl-2-phenyl- | R_{f} = 0.28 (2:1 hexanes/EtOAc); MS(ES) |
| | pyrrolidin-1-yl | 573.3 (M + 1)⁺. |
| 186 | 3-phenyl-pyrrolidin-1-yl | R_{f} = 0.14 (2:1 hexanes/EtOAc); MS(ES) |
| | | 545.3 (M + 1)⁺. |
| 187 | 3-(2-chloro-phenyl)- | R_{f} = 0.15 (2:1 hexanes/EtOAc); MS(ES) |
| | piperidino | 593.3 (M + 1)⁺. |
| 188 | 3-(3-chloro-phenyl)- | R_{f} = 0.21 (2:1 hexanes/EtOAc); MS(ES) |
| | piperidino | 593.3 (M + 1)⁺. |
| 189 | 2,4-diphenyl-pyrrolidin- | R_{f} = 0.27 (2:1 hexanes/EtOAc); MS(ES) |
| | 1-yl | 621.3 (M + 1)⁺. |
| 190 | 3-(3-trifluoromethyl- | R_{f} = 0.21 (2:1 hexanes/EtOAc); MS(ES) |
| | phenyl)-piperidino | 627.3 (M + 1)⁺. |
| 191 | 2,2-diphenyl-pyrrolidin- | R_{f} = 0.30 (2:1 hexanes/EtOAc); MS(ES) |
| | 1-yl | 621.3 (M + 1)⁺. |
| 192 | 2-pyridin-3-yl-pyrrolidin- | R_{f} = 0.44 (2:1 hexanes/EtOAc); MS(ES) |
| | 1-yl | 546.1 (M + 1)⁺. |
| 193 | 2-methyl-pyrrolidin-1-yl | R_{f} = 0.21 (2:1 hexanes/EtOAc); MS(ES) |
| | | 483.2 (M + 1)⁺. |
| 194 | (R)-2-methoxymethyl- | R_{f} = 0.12 (2:1 hexanes/EtOAc); MS(ES) |
| | pyrrolidin-1-yl | 513.2 (M + 1)⁺. |
| 195 | (S)-2-pyrrolidin-1- | R_{f} = 0.18 (2:1 hexanes/EtOAc); MS(ES) |
| | ylmethyl-pyrrolidin-1-yl | 552.2 (M + 1)⁺. |
| 196 | 2-(2-chloro-phenyl)- | R_{f} = 0.18 (2:1 hexanes/EtOAc); MS(ES) |
| | thiazolidin-3-yl | 597.2 (M + 1)⁺. |
| 197 | 2-(2-chloro-phenyl)- | R_{f} = 0.18 (2:1 hexanes/EtOAc); MS(ES) |
| | pyrrolidin-1-yl | 579.1 (M + 1)⁺. |
| 198 | (S)-2-methoxymethyl- | R_{f} = 0.15 (2:1 hexanes/EtOAc); MS(ES) |
| | pyrrolidin-1-yl | 513.2 (M + 1)⁺. |
| 199 | 9-methyl-1,3,4,5- | R_{f} = 0.26 (2:1 hexanes/EtOAc); MS(ES) |
| | tetrahydro- | 559.2 (M + 1)⁺. |
| | benzo[c]azepin-2-yl | |
| 200 | 1,3,4,5-tetrahydro- | R_{f} = 0.20 (2:1 hexanes/EtOAc); MS(ES) |
| | benzo[d]azepin-2-yl | 545.2 (M + 1)⁺. |
| 201 | 4-benzyl-piperidino | R_{f} = 0.26 (2:1 hexanes/EtOAc); MS(ES) |
| | | 573.2 (M + 1)⁺. |
| 202 | 2-methyl-3,4-dihydro- | R_{f} = 0.25 (2:1 hexanes/EtOAc); MS(ES) |
| | 2H-quinolin-1-yl | 545.1 (M + 1)⁺. |
| 203 | 3,4-dihydro-2H-quinolin- | R_{f} = 0.20 (2:1 hexanes/EtOAc); MS(ES) |
| | 1-yl | 531.1 (M + 1)⁺. |
| 204 | 4-cyclohexyl-piperazin- | R_{f} = 0.25 (2:1 hexanes/EtOAc); MS(ES) |
| | 1-yl | 566.2 (M + 1)⁺. |
| 205 | 4-(4-fluoro-benzyl)- | R_{f} = 0.34 (2:1 hexanes/EtOAc); MS(ES) |
| | piperazin-1-yl | 592.2 (M + 1)⁺. |
| 206 | 2,3-dihydro-indol-1-yl | R_{f} = 0.50 (2:1 hexanes/EtOAc); MS(ES) |
| | | 517.2 (M + 1)⁺. |
| 207 | 4-(4-fluoro-phenyl)- | R_{f} = 0.16 (2:1 hexanes/EtOAc); MS(ES) |
| | piperazin-1-yl | 578.3 (M + 1)⁺. |
| 208 | 3,4-dihydro-1H- | R_{f} = 0.28 (2:1 hexanes/EtOAc); MS(ES) |
| | isoquinolin-2-yl | 531.0 (M + 1)⁺. |

By a method similar to Example 100, using the appropriate starting materials, the following compounds may be prepared and isolated.

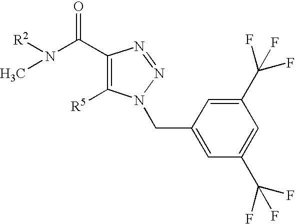

| Ex. # | R² | R³ | Data |
|---|---|---|---|
| 209 | 2,3-dichloro- | 4-fluoro-phenyl | R_{f} = 0.25 (2:1 Hex/EtOAc); |
| | benzyl | | MS(ES) 605.1 (M + 1)⁺. |
| 210 | 2-bromo-benzyl | 4-fluoro-phenyl) | R_{f} = 0.28 (2:1 Hex/EtOAc); |
| | | | MS(ES) 615.1 (M+), |
| | | | 617.1 (M + 2)⁺. |
| 211 | 2-chloro-4-fluoro- | 4-fluoro-phenyl | R_{f} = 0.26 (2:1 Hex/EtOAc); |
| | benzyl | | MS(ES) 589.2 (M + 1)⁺. |
| 212 | 2-chloro-6-fluoro- | 4-fluoro-phenyl | R_{f} = 0.36 (2:1 Hex/EtOAc); |
| | benzyl | | MS(ES) 589.1 (M + 1)⁺. |
| 213 | 2-chloro-benzyl | 2-fluoro-phenyl | R_{f} = 0.29 (2:1 Hex/EtOAc); |
| | | | MS(ES) 571.16 (M + 1)⁺. |
| 214 | 2-chloro-benzyl | 4-methyl-phenyl | R_{f} = 0.29 (2:1 Hex/EtOAc); |
| | | | MS(ES) 567.18 (M + 1)⁺. |
| 215 | 2-chloro-benzyl | 4-methoxy-phenyl | R_{f} = 0.26 (2:1 Hex/EtOAc); |
| | | | MS(ES) 583.2 (M + 1)⁺. |
| 216 | 2-chloro-benzyl | 2-chloro-phenyl | R_{f} = 0.27 (2:1 Hex/EtOAc); |
| | | | MS(ES) 587.13 (M + 1)⁺. |
| 217 | 2-chloro-benzyl | 4-chloro-phenyl | MS(ES): 587.13 (M + 1)⁺ |
| 218 | 2-chloro-benzyl | 3-methyl-phenyl | R_{f} = 0.34 (2:1 Hex/EtOAc); |
| | | | MS(ES) 567.2 (M + 1)⁺. |
| 219 | 2-chloro-benzyl | 4-fluoro-phenyl | R_{f} = 0.27 (2:1 Hex/EtOAc); |
| | | | MS(ES) 571.16 (M + 1)⁺. |
| 220 | phenyl | 4-fluoro-phenyl | R_{f} = 0.16 (2:1 Hex/EtOAc); |
| | | | MS(ES) 523.17 (M + 1)⁺. |
| 221 | phenyl | 2-chloro-phenyl | R_{f} = 0.17 (2:1 Hex/EtOAc); |
| | | | MS(ES) 539.15 (M + 1)⁺. |
| 222 | phenyl | 3-methoxy-phenyl | R_{f} = 0.14 (2:1 Hex/EtOAc); |
| | | | MS(ES) 535.19 (M + 1)⁺. |
| 223 | 2-chloro-benzyl | 3-methoxy-phenyl | R_{f} = 0.25 (2:1 Hex/EtOAc); |
| | | | MS(ES) 583.18 (M + 1)⁺. |
| 224 | phenyl | 4-methyl-phenyl | R_{f} = 0.17 (2:1 Hex/EtOAc); |
| | | | MS(ES) 519.2 (M + 1)⁺. |
| 225 | phenyl | 4-methoxy-phenyl | R_{f} = 0.11 (2:1 Hex/EtOAc); |
| | | | MS(ES) 535.2 (M + 1)⁺. |
| 226 | phenyl | 4-chloro-phenyl | R_{f} = 0.21 (2:1 Hex/EtOAc); |
| | | | MS(ES) 539.15 (M + 1)⁺. |
| 227 | 2-chloro-benzyl | 3-trifluoromethyl- | R_{f} = 0.33 (2:1 Hex/EtOAc); |
| | | phenyl | MS(ES) 621.17 (M + 1)⁺. |
| 228 | phenyl | 3-trifluoromethyl- | R_{f} = 0.19 (2:1 Hex/EtOAc); |
| | | phenyl | MS(ES) 573.18 (M + )⁺. |
| 229 | phenyl | 3-methyl-phenyl | R_{f} = 0.19 (2:1 Hex/EtOAc); |
| | | | MS(ES) 519.2 (M + 1)⁺. |
| 230 | phenyl | 2-fluoro-phenyl | R_{f} = 0.12 (2:1 Hex/EtOAc); |
| | | | MS(ES) 523.17 (M + 1)⁺. |

By a method analogous to Example 100, with the appropriate starting materials, the following compounds may be prepared and isolated.

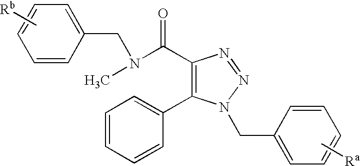

| Ex. # | R^{a} | R^{b} | Data |
|---|---|---|---|
| 231 | 3,5-dimethoxy | hydrogen | R_{f} = 0.25 (2:1 Hex/EtOAc); |
| | | | MS(ES) 443.2 (M + 1)⁺. |
| 232 | 3,5-dimethoxy | 2-chloro | R_{f} = 0.32 (2:1 Hex/EtOAc); |
| | | | MS(ES) 477.1 (M + 1)⁺. |

### EXAMPLE 233

### 1-(2-Chloro-benzyl)-1H-[1,2,3 ]triazole-4-carboxylic acid benzyl-methyl-amide

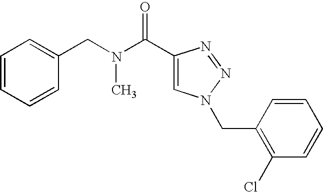

In a screw cap test tube, dissolve 1-(2-chloro-benzyl)-1H-[1,2,3]triazole-4-carboxylic acid ethyl ester (133 mg, 0.5 mmol) in EtOH (0.5 mL), add N-benzyl-N-methylamine (182 mg, 1.5 mmol) and NaCN (5 mg, 0.1 mmol). Seal the test tube and heat at 100° C. in a block heater placed on an orbital shaker for agitation. After 12 hr, cool to room temp. and add H₂O (5 mL) and extract with EtOAc. Dry the organic layer (MgSO₄), filter, and concentrate. Purify the residue by chromatography on silica gel using a hexane/EtOAc gradient to provide the title compound (101 mg, 59%) as an oil. R_{f}=0.33 (1:1 hex/EtOAc); MS(ES) 341.1 (M+l)⁺.

### EXAMPLE 234

### 1-(3,5-Bis-trifluoromethyl-benzyl)-1H-[1,2,3]triazole-4-carboxylic acid benzyl-methyl-amide

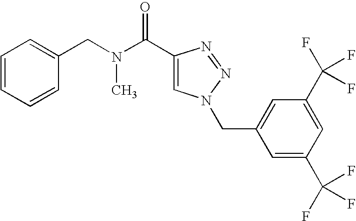

Using a procedure analogous to that for Example 233 and using the appropriate starting materials, the title compound was prepared and isolated. R_{f}=0.21 (2:1 hex/EtOAc); MS(ES) 443.2 (M+1)⁺.

### EXAMPLE 235

### 1-Phenethyl-5-phenyl-1H-imidazole-4-carboxylic acid (2-chloro-benzyl)-methyl-amide

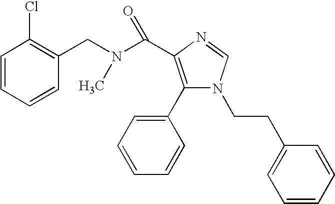

Suspend 1-phenethyl-5-phenyl-1H-imidazole-4-carboxylic acid (1.36 g, 0.328 mmol) and 1-hydroxybenzotriazole-H₂O (0.89 g, 0.656 mmol) in 3 mL of CH₂Cl₂ at RT. Add 2-chloro-N-methylbenzyl amine (0.131 g, 0.656 mmol) and triethylamine (0.23 mL, 1.64 mmol), then EDCl(0.126 g,0.656 mmol) and stir the resulting orange mixture at RT for 16 h. Dilute with CH₂Cl₂ and wash with saturated aqueous NaHCO₃. Dry over MgSO₄, filter, and concentrate. Purify by chromatography (SiO₂, hexanes/EtOAc gradient to yield 0.044 g (60%) of the title compound. ¹H-NMR is consistent with structure; MS(ES) 430.1 (M+1)⁺; Anal. Calc'd for C₁₈H₂₆N₂O₄: C, 64.65; H, 7.83; N, 8.34. Found: C, 64.45; H, 7.90; N, 8.38.

By a method analogous to Example 235, using the appropriate starting materials, the following compounds may be prepared and isolated.

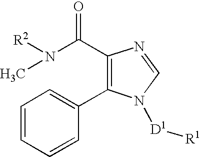

| Ex. # | D¹—R¹ | R² | Data |
|---|---|---|---|
| 236 | phenethyl | 2-bromo- | R_{f} = 0.13 (10:1 CHCl₃/MeOH); |
| | | benzyl | MS(ES) 474.1 (M+), |
| | | | 476.1 (M + 2)⁺. |
| 237 | phenethyl | 2-methoxy- | R_{f} = 0.16 (10:1 CHCl₃/MeOH); |
| | | benzyl | MS(ES) 426.2 (M + 1)⁺ |
| 238 | phenethyl | 3,5-bis- | R_{f} = 0.22 (10:1 CHCl₃/MeOH); |
| | | trifluoro- | MS(ES) 532.2 (M + 1)⁺. |
| | | methyl- | |
| | | benzyl | |
| 239 | 3,5-bis-trifluoro- | 4-chloro- | R_{f} = 0.17 (10:1 CHCl₃/MeOH); |
| | methyl-benzyl | benzyl | MS(ES) 552.1 (M + 1)⁺. |
| 240 | 3,5-bis-trifluoro- | 2-trifluoro- | R_{f} = 0.23 (10:1 CHCl₃/MeOH); |
| | methyl-benzyl | methoxy- | MS(ES) 602.2 (M + )⁺. |
| | | benzyl | |
| 241 | 3,5-bis-trifluoro- | 4-methoxy- | R_{f} = 0.17 (10:1 CHCl₃/MeOH); |
| | methyl-benzyl | benzyl | MS(ES) 548.2 (M + 1)⁺. |
| 242 | 3,5-bis-trifluoro- | phenyl | R_{f} = 0.20 (10:1 CHCl₃/MeOH); |
| | methyl-benzyl | | MS(ES) 504.2 (M + 1)⁺. |
| 243 | 3,5-bis-trifluoro- | phenethyl | R_{f} = 0.13 (10:1 CHCl₃/MeOH); |
| | methyl-benzyl | | MS(ES) 532.2 (M + 1)⁺. |
| 244 | 3,5-bis-trifluoro- | 4-methyl- | R_{f} = 0.20 (10:1 CHCl₃/MeOH); |
| | methyl-benzyl | phenyl | MS(ES)) 518.2 (M + 1)⁺. |
| 245 | 3,5-bis-trifluoro- | 4-methyl- | R_{f} = 0.13 (10:1 CHCl₃/MeOH); |
| | methyl-benzyl | benzyl | MS(ES) 532.2 (M + 1)⁺. |
| 246 | 3,5-bis-trifluoro- | 3-methyl- | R_{f} = 0.20 (10:1 CHCl₃/MeOH); |
| | methyl-benzyl | benzyl | MS(ES) 532.2 (M + 1)⁺. |
| 247 | 3,5-bis-trifluoro- | 2-methyl- | R_{f} = 0.17 (10:1 CHCl₃/MeOH); |
| | methyl-benzyl | benzyl | MS(ES) 532.3 (M + 1)⁺. |
| 248 | 3,5-bis-trifluoro- | 3-methoxy- | R_{f} = 0.23 (10:1 CHCl₃/MeOH); |
| | methyl-benzyl | benzyl | MS(ES) 548.3 (M + 1)⁺. |
| 249 | 3,5-bis-trifluoro- | 2-bromo- | R_{f} = 0.08 (10:1 CHCl₃/MeOH); |
| | methyl-benzyl | benzyl | MS(ES) 596.2 (M+), |
| | | | 598.2 (M + 2)⁺. |
| 250 | 3,5-bis-trifluoro- | 2,3-dichloro- | MS 586.4; MS(ES) |
| | methyl-benzyl | benzyl | 586.2 (M + 1)⁺. |
| 251 | 3,5-bis-trifluoro- | 2-methoxy- | R_{f} = 0.15 (10:1 CHCl₃/MeOH); |
| | methyl-benzyl | benzyl | MS(ES) 548.3 (M + 1)⁺. |
| 252 | 3,5-bis-trifluoro- | 3-chloro- | R_{f} = 0.20 (10:1 CHCl₃/MeOH); |
| | methyl-benzyl | benzyl | MS(ES) 552.2 (M + 1)⁺. |
| 253 | 3,5-bis-trifluoro- | 4-fluoro- | R_{f} = 0.13 (10:1 CHCl₃/MeOH); |
| | methyl-benzyl | benzyl | MS(ES) 536.2 (M + 1)⁺. |
| 254 | 3,5-bis-trifluoro- | 2-chloro-4- | R_{f} = 0.20 (10:1 CHCl₃/MeOH); |
| | methyl-benzyl | fluoro- | MS(ES) 570.2 (M + 1 )⁺. |
| | | benzyl | |
| 255 | 3,5-bis-trifluoro- | benzyl | R_{f} = 0.20 (10:1 CHCl₃/MeOH); |
| | methyl-benzyl | | MS(ES) 518.3 (M + 1)⁺. |

### EXAMPLE 256

### [1-(3,5-Bis-trifluoromethyl-benzyl)-5-phenyl-1H-imidazol-4-yl]-[2-(2-chloro-phenyl)-pyrrolidin-1-yl]-methanone

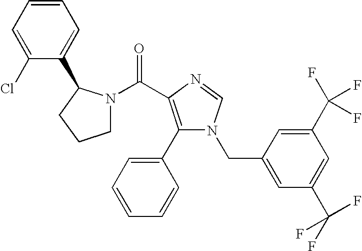

Using a method analogous to Example 235, the title compound may be prepared and isolated. R_{f}=0.10 (10:1 CHCl₃/MeOH); MS(ES) 578.2 (M+1).

### EXAMPLE 257

### 1-(3,5-Bis-trifluoromethyl-benzyl)-5-chloro-1H-[1,2,3]triazole-4-carboxylic acid (2-chloro-benzyl)-methyl-amide

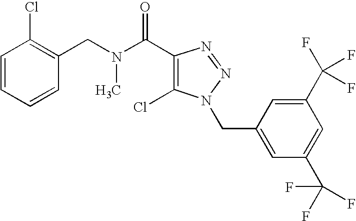

Combine a solution of 1-(3,5-bis-trifluoromethyl-benzyl)-5-chloro-1H-[1,2,3]triazole-4-carboxylic acid (2.75g, 7.36 mmol) in CH₂Cl₂ (60 mL) with (2-chloro-benzyl)-methyl-amine (1.39 g, 8.93 mmol), DMAP (1.18 g, 9.66 mmol), and EDCI (1.62 g, 8.45 mmol). Stir at RT for 16 h then heat to reflux for an additional 3 h. Cool back to RT and dilute the solution with CH₂Cl₂ (40 mL). Wash with saturated NaHCO₃ (50 mL), H₂O (50 mL), and brine (50 mL), then dry, filter, and concentrate. Purify the crude material by flash chromatography, using a linear gradient of 15% to 40% EtOAc/hexanes, to afford the title compound (3.15 g, 84%) as a clear viscous oil. MS(ES) 511.0 (M+1)⁺. ¹H NMR (400 MHz, CHCl₃, mixture of amide rotamers) δ 7.88 (s, 0.5 H), 7.87 (s, 0.5 H), 7.82 (s, 1 H), 7.76 (s, 1 H), 7.20-7.38 (m, 4 H), 5.65 (s, 1 H), 5.61 (s, 1 H), 5.10 (s, 1 H), 4.88 (s, 1 H), 3.32 (s, 1.5 H), 3.03 (s, 1.5 H).

### EXAMPLE 258

### 1-(3,5-bis-trifluoromethyl-benzyl)-5-chloro-4-yl-1H-[1,2,3]triazole-4-carboxylic acid (2-5 chloro-benzyl)-(2-methoxy-ethyl)-amide

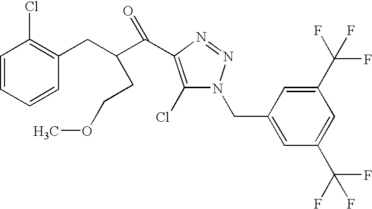

Combine 1-(3,5-Bis-trifluoromethyl-benzyl)-5-chloro-1H-[1,2,3]triazole-4-carboxylic acid (180 mg, 1 eq), N-(2-chloro-benzyl)-N-(2-methoxy-ethyl)-amine (105 mg, 1.5 eq), EDCI (100 mg, 1.1 eq.), HOAt (70 mg, 1.1 eq.), TEA (0.1 mL, 1.1 eq.) and DMAP (5 mg) in DMF (5 mL) and stir overnight at RT. Concentrate to dryness then dissolve in 20% iPrOH/CHCl₃ and wash with saturated aqueous NaHCO₃ and brine. Dry (Na₂SO₄), filter, and concentrate to dryness. Purify the residue by chromatography on silica gel to provide the title compound (47% yield). MS(ES) 554.9 (M+1)⁺; R_{f}=0.60 (1:1 EtOAc/hexanes).

### EXAMPLE 259

### 1-(3,5-Bis-trifluoromethyl-benzyl)-5-phenoxy-1H-[1,2,3]tri-azole-4-carboxylic acid (2-chloro-benzyl)-methyl-amide

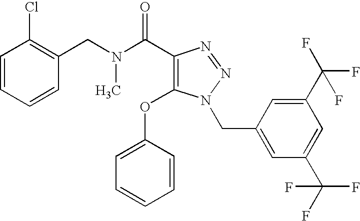

Combine a solution of 1-(3,5-bis-trifluoromethyl-benzyl)-5-chloro-1H-[1,2,3]triazole-4-carboxylic acid (2-chloro-benzyl)-methyl-amide (80 mg, 0.16 mmol) in DMF (1.0 mL) with phenol (56 mg, 0.60 mmol) and Cs₂CO₃ (188 mg, 0.58 mmol) and heat to 70° C. for 18 h. Dilute mixture with H₂O and extract with EtOAc (25 mL). Wash the organic phase with 2N Na₂CO₃ (10 mL) and brine (10 mL), then dry, filter, and concentrate. Purify the crude material by flash chromatography, using a linear gradient of 15% to 40% EtOAc/hexanes, to give the title compound (53 mg, 60%) as a yellow viscpous oil. MS(ES) 569.2 (M+1)⁺; ¹H NMR (400 MHz, CHCl₃, 1:1 mixture of amide rotamers) δ 7.79 (s, 0.5H), 7.76 (s, 0.5H), 7.71 (s, 1H), 7.63 (s, 1H), 6.92-7.35 (m, 7H), 6.83 (d, 1H, J=7.4 Hz), 6.78 (d, 1H, J=7.8 Hz), 5.50 (s, 1H), 5.42 (s, 1H), 5.17 (s, 1H), 4.70 (s, 1H), 3.27 (s, 1.5H), 2.89 (s, 1.5H).

Using a method similar to Example 259, with the appropriate starting materials, the following compounds may be prepared and isolated.

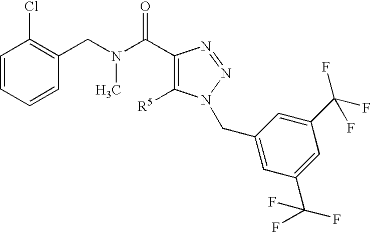

| Ex. # | R⁵ | Data |
|---|---|---|
| 260 | 4-chloro-phenoxy | MS(ES) 603.1 (M + 1)⁺. |
| 261 | 4-methyl-phenoxy | MS(ES) 583.2 (M + 1)⁺. |
| 262 | 3-chloro-phenoxy | MS(ES) 603.1 (M + 1)⁺. |
| 263 | 4-methoxy-phenoxy | MS(ES) 599.2 (M + 1)⁺. |
| 264 | 3-pyridyloxy | MS(ES) 570.1 (M + 1)⁺. |
| 265 | 2-pyridyloxy | MS(ES) 570.0 (M + 1)⁺. |

### EXAMPLE 266

### 1-(3,5-Bis-trifluoromethyl-benzyl)-5-phenylsulfanyl-1H-[1,2,3]triazole-4-carboxylic acid (2-chloro-benzyl)-methyl-amide

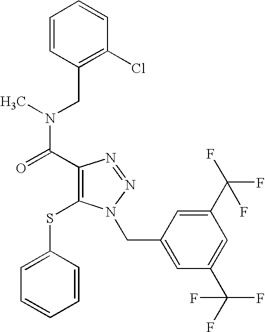

Combine a solution of 1-(3,5-bis-trifluoromethyl-benzyl)-5-chloro-1H-[1,2,3]triazole-4-carboxylic acid (2-chloro-benzyl)-methyl-amide (69 mg, 0.14 mmol) and benzenethiol (20 μL, 0.19 mmol) in DMF (1.3 mL) and stir at RT. After 60 h., dilute the mixture with H₂O (10 mL) and extract with EtOAc (25 mL). Wash the organic layer with 2N Na₂CO₃ (10 mL) and brine (10 mL), then dry, filter, and concentrate. Purify crude material by flash chromatography using a linear gradient of 15% to 40% EtOAc/hexanes to afford the title compound (40 mg, 50%) as a yellow, viscous oil. MS(ES) 585.2 (M+1)⁺; ¹H NMR (400 MHz, CHCl₃1:1 mixture of amide rotamers) δ 7.70 (s, 0.5H), 7.67 (s, 0.5H), 7.53 (s, 1H), 7.45 (s, 1H), 7.02-7.36 (m, 9H), 5.65 (s, 1H), 5.57 (s, 1H), 4.92 (s, 1H), 4.87 (s, 1H), 3.13 (s, 1.5H), 3.04 (s, 1.5H).

Using a method similar to Example 266, with the appropriate starting materials, wing compounds may be prepared and isolated.

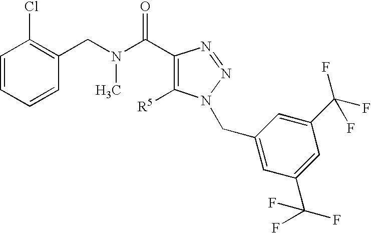

| Ex. # | R⁵ | Data |
|---|---|---|
| 267 | 4-chloro-phenyl-sulfanyl | MS(ES) 619.1 (M + 1)⁺. |
| 268 | 3-chloro-phenyl-sulfanyl | MS(ES) 619.1 (M + 1)⁺. |
| 269 | 4-methoxy-phenyl-sulfanyl | MS(ES) 599.2 (M + 1)⁺. |
| 270 | 3-methyl-phenyl-sulfanyl | MS(ES) 615.0 (M + 1)⁺. |

### EXAMPLE 271

### 1-(3,5-Bis-trifluoromethyl-benzyl)-5-phenylamino-1H-[1,2,3]triazole-4-carboxylic acid (2-chloro-benzyl)-methyl-amide

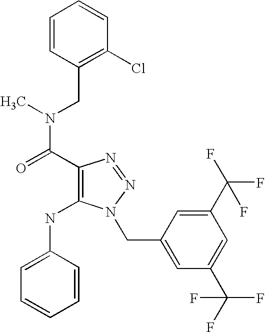

Combine a solution of aniline (45 μL, 0.49 mmol) in THF (0.5 mL) with methyllithium (0.22 mL of a 1.4M soln in ether, 0.31 mmol). Add 1-(3,5-bis-trifluoromethyl-benzyl)-5-chloro-1H-[1,2,3]triazole-4-carboxylic acid (2-chloro-benzyl)-methyl-amide (64 mg, 0.12 mmol) as a solution in THF (1.0 mL) and stir at RT. After 20 min., dilute with ether (10 mL) and wash the organic solution with saturated aqueous NH₄Cl (2×5 mL) then dry, filter, and concentrate. Purify the crude material by flash chromatography using a linear gradient of 10% to 40% EtOAc/hexanes to afford the title compound (54 mg, 76%) as a red viscous oil. MS(ES) 568.2 (M+1)⁺; ¹H NMR (400 MHz, CHCl₃, 1:1 mixture of amide rotamers) δ 8.39 (s, 0.5H), 8.32 (s, 0.5H), 7.75 (s, 1H), 7.12-7.38 (m, 9H), 6.80 (m, 2H), 5.54 (s, 1H), 5.30 (s, 1H), 5.25 (s, 1H), 4.83 (s, 1H),3.67 (s, 1.5H),3.01 (s, 1.5H).

### EXAMPLE 272

### 1-(3,5-Bis-trifluoromethyl-benzyl)-1H-[1,2,3]triazole-4-carboxylic acid (2-chloro-benzyl)-methyl-amide

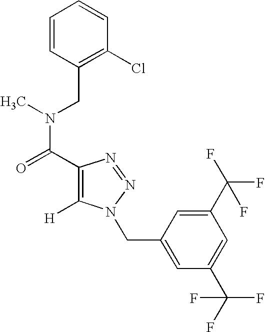

Add EDCI (86 mg, 0.45 mmol) to a solution of (2-chloro-benzyl)-methyl-amine (91 mg, 0.58 mmol), 1-(3,5-bis-trifluoromethyl-benzyl)-1H-[1,2,3]triazole-4-carboxylic acid (99 mg, 0.29 mmol), and DMAP (89 mg, 0.73 mmol) in CH₂Cl₂ (3.0 mL) and stir at RT. After 24 h., dilute the solution with CH₂Cl₂ (10 mL) and wash with saturated aqueous NH₄Cl (10 mL) and saturated aqueous NaHCO₃ (10 mL) then dry, filter and concentrate. Purify the crude material by flash chromatography using a linear gradient of 10% to 40% EtOAcihexanes to afford the title compound (108 mg, 77%) as a white solid. MS(ES) 477.0 (M+1)⁺, ¹H NMR (400 MHz, CHCl₃, 1:1 mixture of amide rotamers) δ 8.21 (s, 0.5H), 8.16 (s, 0.5H), 7.88 (s, 0.5H), 7.87 (s, 0.5H), 7.81 (s, 1H), 7.73 (s, 1H), 7.19-7.37 (m, 4H), 5.66 (s, 1H), 5.63 (s, 1H), 5.39 (s, 1H), 4.86 (s, 1H), 3.53 (s, 1.5H), 3.03 (s, 1.5H).

Using a method analogous to Example 272, with the appropriate starting materials, the following compounds may be prepared and isolated.

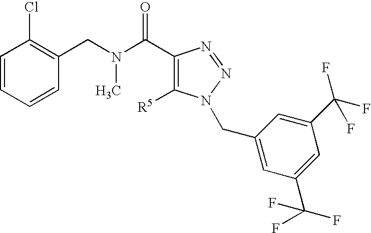

| Ex. # | R⁵ | Data |
|---|---|---|
| 273 | methyl | MS(ES) 491.1 (M + 1)⁺. |
| 274 | Ethyl | MS(ES) 505.2 (M + 1)⁺. |
| 275 | n-propyl | MS(ES) 519.1 (M + 1)⁺. |
| 276 | n-butyl | MS(ES) 533.1 (M + 1)⁺. |
| 277 | trifluoromethyl | MS(ES) 545.2 (M + 1)⁺. |

### EXAMPLE 278

### 1-(3,5-Bis-trifluoromethyl-benzyl)-5-butoxy-1H-[1,2,3]triazole-4-carboxylic acid (2-chloro-benzyl)-methyl-amide

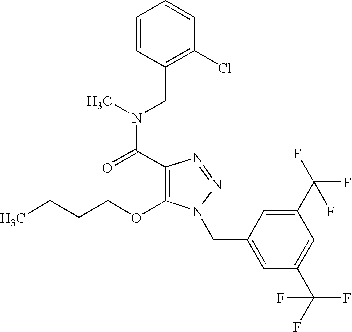

Combine a solution of 1-(3,5-bis-trifluoromethyl-benzyl)-5-butoxy-1H-[1,2,3)triazole-4-carboxylic acid (42 mg, 0.10 mmol), (2-Chloro-benzyl)-methyl-amine (67 mg, 0.43 mmol), and DMAP (69 mg, 0.56 mmol) in CH₂Cl₂ (1.0 mL) with EDCI (54 mg, 0.28 mmol) and stir at RT. After 60 h., dilute solution with CH₂Cl₂ (20 mL) and wash with aqueous 0.5N HCl (10 mL), H₂O (10 mL), and saturated NaHCO₃ (10 mL). Dry, filter, and concentrate the organic solution. Purify the crude material by flash chromatography using a linear gradient of 0% to 40% EtOAc/hexanes to afford the title compound (48 mg, 86%) as a clear, colorless oil. MS(ES) 549.2 (M+1)⁺; ¹H NMR (400 MHz, CHCl₃, 1:1 mixture of amide rotamers) δ 7.85 (s, 1H), 7.80 (s, 1H), 7.76 (s, 1H), 7.20-7.36 (m, 4H), 5.42 (s, 1H), 5.38 (s, 1H), 5.05 (s, 1H), 4.86 (s, 1H), 4.38 (q, 2H, J=4.9 Hz), 3.26 (s, 1.5H), 3.00 (s, 1.5H), 1.64 (m, 2H), 1.35 (m, 2H), 0.89 (t, 3H, J=7.3 Hz).

### EXAMPLE 279

### 5-Benzyloxy-1-(3,5-bis-trifluoromethyl-benzyl)-1H-[1,2,3]triazole-4-carboxylic acid (2-chloro-benzyl)-methyl-amide

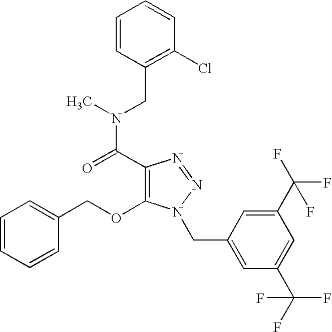

Using a similar method to Example 278, except using 5-benzyloxy-1-(3,5-bis-trifluoromethyl-benzyl)-1H-[1,2,3]triazole-4-carboxylic acid (61 mg, 0.14 mmol), affords the title compound (30 mg, 37%) as a clear, colorless oil. MS(ES) 583.2 (M+1)⁺. ¹H NMR (400 MHz, CHCl₃, 1:1 mixture of amide rotamers) δ 7.85 (s, 0.5H), 7.83 (s, 0.5H), 7.69 (s, 1H), 7.64 (s, 1H), 7.18-7.40 (m, 9H), 5.48 (s, 1H), 5.47 (s, 1H), 5.32 (s, 1H), 5.26 (s, 1H), 4.95 (s, 1H), 4.8.9 (s, 1H), 3.19 (s, 1.5H), 3.03 (s, 1.5H).

### EXAMPLE 280

### 1-(3,5-Bis-trifluoromethyl-benzyl)-5-piperazin-1-yl-1H-[1,2,3]triazole-4-carboxylic acid (2-chloro-benzyl)-methyl-amide

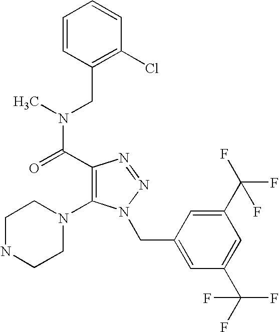

Combine piperazine (210 mg, 2.44 mmol) with a solution of 1-(3,5-bis-trifluoromethyl-benzyl)-5-chloro-1H-[1,2,3]triazole-4-carboxylic acid (2-chloro-benzyl)-methyl-amide (60 mg, 0.12 mmol) in THF (0.50 mL) and heat to 80° C. in a sealed tube. After 16 h, cool the solution to RT and dilute with Et₂O (30 mL). Wash with H₂O (3×10 mL), saturated aqueous NH₄Cl (10 mL), and saturated aqueous NaHCO₃ (10 mL), then dry, filter, and concentrate. Purify crude material by dissolving in methanol (0.5 mL) and applying to a Varian SCX column. Elute first with methanol (30 mL) to remove unreacted 1-(3,5-bis-trifluoromethyl-benzyl)-5-chloro-1H-[1,2,3]tri-azole-4-carboxylic acid (2-chloro-benzyl)-methyl-amide, then elute with 2M NH₃/MeOH (30 mL) to afford the title compound (50 mg, 76%) as a clear, colorless oil. MS(ES) 561.1 (M+1)⁺, ¹H NMR (400 MHz, CHCl₃, 1:1 mixture of amide rotamers) δ 7.83 (m, 2H), 7.79 (s, 1H), 7.18-7.37 (m, 4H), 5.53 (s, 1H), 5.48 (s, 1H), 5.08 (s, 1H), 4.86 (s, 1H), 3.25 (s, 1.5H), 3.02 (s, 1.5H), 2.96 (m, 8H), 2.35 (br s, 1H).

Using a method similar to Example 280, with the appropriate starting materials, the following compounds may be prepared and isolated.

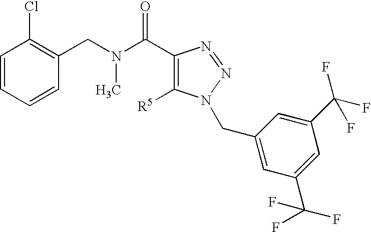

| Ex. # | R⁵ | Data |
|---|---|---|
| 281 | 4-methyl-piperazin-1-yl | MS(ES) 575.0 (M + 1)⁺. |
| 282 | 2-dimethlamino-ethylamino | MS(ES) 563.2 (M + 1)⁺. |

### EXAMPLE 283

### 1-(3,5-Bis-trifluoromethyl-benzyl)-5-morpholin-4-yl-1H-[1,2,3 ]triazole-4-carboxylic acid (2-chloro-benzyl)-methyl-amide

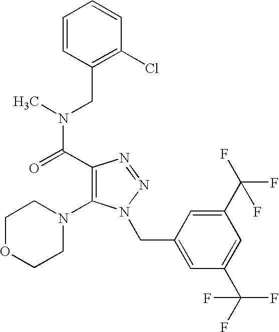

Dissolve 1-(3,5-bis-trifluoromethyl-benzyl)-5-chloro-1H-[1,2,3]triazole-4-carboxylic acid (2-chloro-benzyl)-methyl-amide (64 mg, 0.12 mmol) in morpholine (0.8 mL) and heat to 80° C. After 16 h, cool to RT and dilute the solution with EtOAc (25 ml). Wash with saturated aqueous NH₄Cl (2×15 mL), H₂O (15 mL), and saturated aqueous NaHCO₃ (15 mL). Dry, filter, and concentrate, then purify by flash chromatography using a linear gradient of 10% to 40% EtOAc/hexanes to afford the title compound (61 mg, 87%) as a clear, colorless oil. MS(ES) 562.1 (M+1 )⁺; ¹H NMR (400 MHz, CHCl₃, 1:1 mixture of amide rotamers) δ 7.85 (s, 0.5H), 7.84 (s, 0.5H), 7.82 (s, 1H), 7.77 (s, 1H), 7.18-7.38 (m, 4H), 5.54 (s, 1H), 5.50 (s, 1H), 5.08 (s, 1H), 4.88 (s, 1H), 3.72 (m, 4H), 3.25 (s, 1.5H), 3.03 (s, 1.5H), 2.99 (m, 4H).

### EXAMPLE 284

### 1-(3,5-Bis-trifluoromethyl-benzyl)-5-pyrrolidin-1-yl-1H-[1,2,3]triazole4-carboxylic acid (2-chloro-benzyl)-methyl-amide

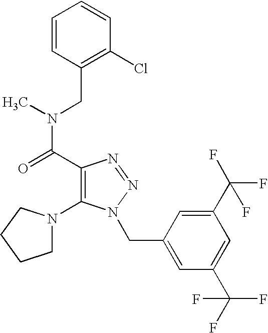

Add pyrrolidine (17 μL) to a solution of 1-(3,5-bis-trifluoromethyl-benzyl)-5-chloro-1H-[1,2,3)triazole-4-carboxylic acid (2-chloro-benzyl)-methyl-amide (46 mg, 0.09 mmol) in THF (1.0 mL) and stir at RT in a sealed tube. After 16 h, heat the solution to 80° C. for 24 h, then add additional pyrrolidine (34 μL, 0.18 mmol) and heat to 90° C. for and additional 16 h. Cool the solution to RT and dilute with EtOAc (20 mL), then wash with 0.2N HCl (10 mL) and saturated aqueous NaHCO₃ (10 mL). Dry, filter, and concentrate the organic solution, then purify crude material by flash chromatography using a linear gradient of 15% to 45% EtOAc/hexanes to afford the title compound (31 mg, 63%) as a clear, colorless oil. MS (ES) 546.1 (M+1)⁺; ¹H NMR (400 MHz, CHCl₃, 1:1 mixture of amide rotamers) δ 7.83 (s, 0.5H), 7.82 (s, 0.5H), 7.72 (s, 1H), 7.68 (s, 1H), 7.18-7.37 (m, 4H), 5.55 (s, 1H), 5.50 (s, 1H), 5.06 (s, 1H), 4.86 (s, 1H), 3.24 (s, 1.5H), 3.16 (m, 4H), 3.00 (s, 1.5H), 1.92 (m, 4H).

### EXAMPLE 285

### 1-(3,5-Bis-trifluoromethyl-benzyl)-5-piperidin-1-yl-1H-[1,2,3]triazole-4-carboxylic acid (2-chloro-benzyl)-methyl-amide

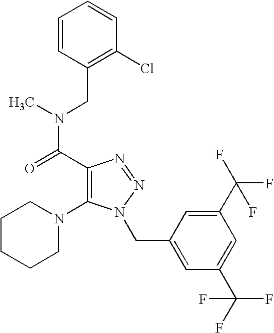

Dissolve 1-(3,5-bis-trifluoromethyl-benzyl)-5-chloro-1H-[1,2,3]triazole-4-carboxylic acid (2-chloro-benzyl)-methyl-amide (52 mg, 0.10 mmol) in piperidine (1.0 mL) and heat to 80° C. for 16 h in a sealed tube. Cool to RT and dilute with EtOAc (50 mL). Wash organic solution with 1N HCl (10 mL), H₂O (10 mL), and saturated aqueous NaHCO₃ (10 mL) then dry, filter, and concentrate. Purify crude material by flash chromatography using a linear gradient of 10% to 40% EtOAc to afford the title compound (57 mg, 100%) as a clear, colorless oil. MS(ES) 560.1 (M+1)⁺, ¹H NMR (400 MHz, CHCl₃, 1:1 mixture of amide rotamers) δ 7.84 (m, 2H), 7.79 (s, 1H), 7.17-7.37 (m, 4H), 5.49 (s, 1H), 5.45 (s, 1H), 5.06 (s, 1H), 4.87 (s, 1H), 3.23 (s, 1.5H), 3.02 (s, 1.5H), 2.92 (m, 4H), 1.92 (m, 6H).

### EXAMPLE 286

### 1-(3,5-Bis-trifluoromethyl-benzyl)-5-dimethylamino-1H-[1,2,3)triazole-4-carboxylic acid (2-chloro-benzyl)-methyl-amide

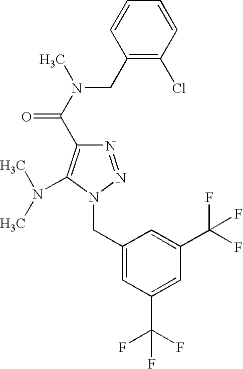

Add dimethylamine (4.0 mL, 2M in MeOH) to 1-(3,5-bis-trifluoromethyl-benzyl)-5-chloro-1H-[1,2,3]triazole-4-carboxylic acid (2-chloro-benzyl)-methyl-amide (80.0 mg, 0.16 mmol) and heat at 100° C. for 16 h in a sealed tube. Concentrate the reaction mixture and purify by flash chromatography using a linear gradient of 10 to 40% EtOAc in hexanes to afford the title compound (50 mg, 62%) as a clear colorless oil. MS(ES) 520.27 (M+1)⁺. ¹H NMR (400 MHz, CDCl₃, 1:1 mixture of amide rotamers) δ 7.85 (m, 1H), 7.83 (s, 1H), 7.80 (s, 1H), 7.20-7.40 (m, 4H), 5.53 (s, 1H), 5.49 (s, 1H), 5.13 (s,1H), 4.89 (s, 1H), 3.30 (s, 1.5H), 3.05 (s, 1.5H), 2.74 (s, 3H), 2.72 (s, 3H).

Using a method analogous to the above example, with the appropriate starting materials, the following compounds may be prepared and isolated.

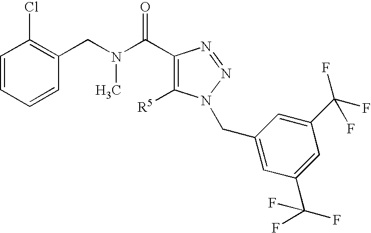

| Ex. # | R⁵ | Data |
|---|---|---|
| 287 | diethylamino | MS(ES) 548.1 (M + 1)⁺ |
| 288 | ethylamino | MS(ES) 520.1 (M + 1)⁺ |

### EXAMPLE 289

### 1-(3,5)-Bis-trifluoromethyl-benzyl)-5-isopropylamino-1H-[1,2,3]triazole-4-carboxylic acid (2-chloro-benzyl)-methyl-amide

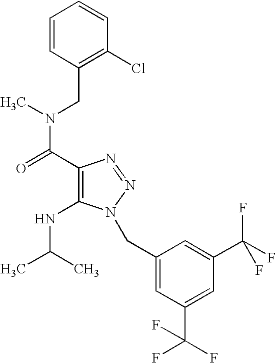

Add 2M solution of isopropylamine in MeOH (10.0 mL, 20.0 mmol) to 1-(3,5-bis-trifluoromethyl-benzyl)-5-chloro-1H-[1,2,3]triazole-4-carboxylic acid (2-chloro-benzyl)-methyl-amide (0.05 g, 0.10 mmoL) and heat at 100° C. for 16 h in a sealed tube. Concentrate the reaction mixture and purify by flash chromatography using a linear gradient of 10 to 40% EtOAc in hexane to give the title compound (0.04 g, 86%). MS(ES) 534.1 (M+1)⁺. ¹H NMR (400 MHz, CDCl₃, 1:1 mixture of amide rotamers) δ 7.87 (s, 0.5H), 7.86 (s, 0.5H), 7.71 (s, 1H), 7.65 (s, 1H), 7.37 (m, 1H), 7.23 (m, 3H), 6.50 (brs, 1H), 5.56 (m, 3H), 4.86 (s, 1H), 3.65 (s, 1.5H), 3.39 (m, 1H), 3.03 (s, 1.5H), 1.13 (m, 6H).

Using a method analogous to the above example, with the appropriate starting materials, the following compounds may be prepared and isolated.

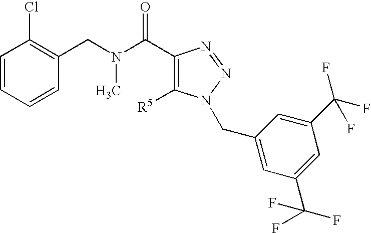

| Ex. # | R⁵ | Data |
|---|---|---|
| 290 | 2-methoxy-ethylamino | MS(ES) 550.0 (M + 1)⁺ |
| 291 | methylamino | MS(ES) 506.0 (M + 1)⁺ |
| 292 | thiomorpholin-4-yl | MS(ES) 578.0 (M + 1)⁺ |
| 293 | propylamino | MS(ES) 534.1 (M + 1)⁺ |
| 294 | azepan-1-yl | MS(ES) 574.4 (M + 1)⁺ |
| 295 | azetidin-1yl | MS(ES) 532.3 (M + 1)⁺ |
| 296 | cyclopropylamino | MS(ES) 532.1 (M + 1)⁺ |
| 297 | 4-hydroxy-piperidino | MS(ES) 576.5 (M + 1)⁺ |

### EXAMPLE 298

### 5-(4-Acetyl-piperazin-1-yl)-1-(3,5-bis-trifluoromethyl-benzyl)-1H-[1,2,3]triazole-4-carboxylic acid (2-chloro-benzyl)-methyl-amide

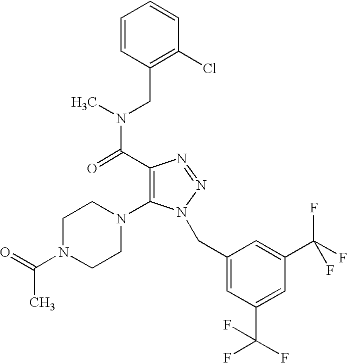

Add acetyl chloride (0.1 mL, 1.3 mmol) to a solution of 1-(3,5-bis-trifluoromethyl-benzyl)-5-piperazin-1-yl-1H-[1,2,3]triazole-4-carboxylic acid (2-chloro-benzyl)-methyl-amide (0.05 g, 0.10 mmol) and triethylamine (2.0 mL, 1.4 mmol) in dichloromethane (4.0 mL). Stir at RT for 4 h, dilute with water and extract with dichloromethane. Wash organic extract with 1N HCl, water, and brine, then dry and concentrate. Purify by flash chromatography using a linear gradient of 1 to 2% MeOH in dichloromethane to give the title compound (0.05 g, 94%). MS(ES) 603.1 (M+1)⁺. ¹H NMR (400 MHz, CDCl₃. 1:1 mixture of amide rotamers) δ 7.88 (s, 0.5H), 7.87 (s, 0.5H), 7.83 (s, 1H), 7.78 (s, 1H), 7.39 (m, 0.5H), 7.33 (m, 0.5H), 7.28 (m, 1H), 7.23 (m, 2H), 5.57 (s, 1H), 5.53 (s, 1H), 5.13 (s, 1H), 4.87 (s, 1H), 3.66 (m, 2H), 3.48 (m, 2H), 3.30 (s, 1.5H), 2.95-3.05 (s, 5.5H), 2.10 (s, 1.5H), 2.08 (s, 1.5H).

### EXAMPLE 299

### 1-(3,5-Bis-trifluoromethyl-benzyl)-5-(1-oxo-1λ⁴-thiomorpholin-4-yl)-1H-[1,2,3]triazole-4-carboxylic acid (2-chloro-benzyl)-methyl-amide

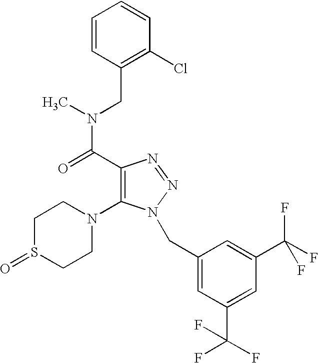

Add 30% aqueous hydrogen peroxide (10.0 uL, 0.1 mmol) to a solution of 1-(3,5-bis-trifluoromethyl-benzyl)-5-thiomorpholin4-yl-1H-[1,2,3]triazole-4-carboxylic acid (2-chloro-benzyl)-methyl-amide (0.05 g, 0.1 mmol) in MeOH (2.0 mL) and stir at RT for 24 h. Add water and extract with EtOAc, then dry, filter, and concentrate. Purify by flash chromatography using a linear gradient of 3 to 5% MeOH in dichloromethane to give the title compound (0.05 g, 95%). MS(ES) 594.2 (M+1)⁺; ¹H NMR (400 MHz, CDCl₃, 1:1 mixture of amide rotamers) δ 7.89 (s, 0.5H), 7.88 (s, 0.5H), 7.82 (s, 1H), 7.77 (s, 1H), 7.39 (m, 0.5H), 7.28-7.35 (m, 1.5H), 7.23 (m, 2H), 5.57 (s, 1H), 5.53 (s, 1H), 5.15 (s, 1H), 4.89 (s, 1H), 3.63 (m, 2H), 3.32 (s, 1.5H), 3.18 (m, 2H), 3.04 (m, 3.5H), 2.87 (m, 2H).

### EXAMPLE 300

### 1-(3,5-Bis-trifluoromethyl-benzyl)-5-propoxy-1H-[1,2,3]triazole-4-carboxylic acid (2-chloro-benzyl)-methyl-amide

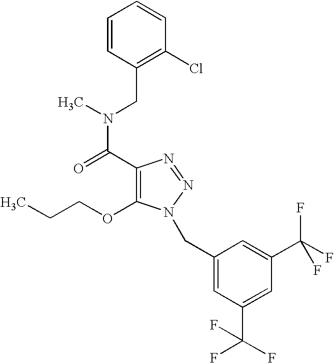

Combine EDC.HCl (0.18 g, 0.94 mmol) with a solution of 1-(3,5-bis-trifluoromethyl-benzyl)-5-propoxy-1H-[1,2,3]triazole-4-carboxylic acid (0.25 g, 0.63 mmol), (2-chloro-benzyl)-methyl-amine (0.18 g, 1.16 mmol), and DMAP (0.12 g, 0.94 mmol) in dichloromethane (10.0 mL) and stir mixture for 48 h. Add saturated NaHCO₃ and extract mixture with dichloromethane. Wash the organic layer with water and brine, then dry, concentrate, and purify by flash chromatography using a linear gradient of 10 to 40% EtOAc in hexane to give the title compound (0.30 g, 90%). MS(ES) 535.0 (M+1)⁺; ¹H NMR (400 MHz, CDCl₃, 1:1 mixture of amide rotamers) δ 7.89 (s, 0.5H), 7.88 (s, 0.5H), 7.82 (s, 1H), 7.77 (s, 1H), 7.39 (m, 0.5H), 7.28-7.35 (m, 1.5H), 7.23 (m, 2H), 5.44 (s, 1H), 5.40 (s, 1H), 5.06 (s, 1H), 4.87 (s, 1H), 4.34 (q, 2H, J=6.8), 3.27 (s, 1.5H), 3.01 (s, 1.5H), 1.72 (m, 2H), 0.94 (t, 3H, J=6.8).

Using a method similar to the above example, with the appropriate starting materials, the following compounds may be prepared and isolated.

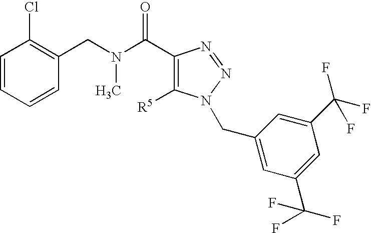

| Ex. # | R⁵ | Data |
|---|---|---|
| 301 | Ethoxy | MS(ES) 521.2 (M + 1)⁺ |
| 302 | Methoxy | MS(ES) 507.3 (M + 1)⁺ |

### EXAMPLE 303

### 1-(3,5-Bis-trifluoromethyl-benzyl)-5-( 1,1-dioxo-1λ⁶-thiomorpholin-4-yl)-1H-[1,2,3]triazole-4-carboxylic acid (2-chloro-benzyl)-methyl-amide

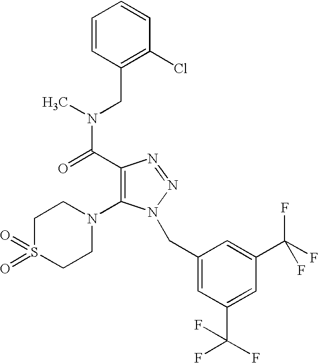

Add 30% aqueous hydrogen peroxide (20.0 μL, 0.2 mmol) to a solution of 1-(3,5-bis-trifluoromethyl-benzyl)-5-thiomorpholin-4-yl-1H-[1,2,3]triazole-4-carboxylic acid (2-chloro-benzyl)-methyl-amide (0.05 g, 0.1 mmol) in MeOH (3.0 mL) and stir at reflux for 24 h. Add water and extract with EtOAc, then dry, filter, and concentrate. Purify by flash chromatography using a linear gradient of 60 to 80% EtOAc in hexane to give the title compound (0.03 g, 60%). MS(ES) 609.9 (M+1)⁺. ¹H NMR (400 MHz, CDCl₃, 1:1 mixture of amide rotamers) δ 7.91 (s, 0.5H), 7.90 (s, 0.5H), 7.79 (s, 1H), 7.74 (s, 1H), 7.35 (m, 1H), 7.30 (m, 0.5H), 7.23 (m, 2.5H), 5.57 (s, 1H), 5.53 (s, 1H), 5.18 (s, 1H), 4.91 (s, 1H), 3.52 (m, 4H), 3.35 (s, 1.5H), 3.13 (m, 4H), 3.06 (m, 1.5H).

### EXAMPLE 304

### 5-Chloro-1-(3,5-dichloro-benzyl)-1H-[1,2,3]triazole-4-carboxylic acid (2-chloro-benzyl)-isopropyl-amide

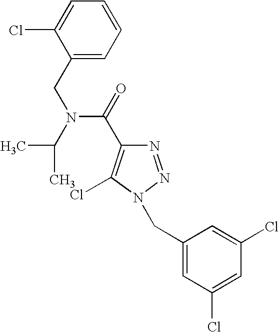

Combine (2-chloro-benzyl)-isopropyl-amine (240 mg, 1.31 mmol) with 5-chloro-1-(3,5-dichloro-benzyl)-1H-[1,2,3]triazole-4-carboxylic acid (400 mg, 1.31 mmol), EDCI (250 mg, 1.30 mmol), HOAt (178 mg, 1.31 mmol), and DIEA (0.20 mL, 1.15 mmol), in DMF (8 mL) and stir the mixture at RT. After 72 h, concentrate the mixture in vacuo and partition the residue between water and EtOAc. Dry the combined extracts over sodium sulfate and concentrate in vacuo. Purify the residue by chromatography over silica gel using a MeOH/CH₂Cl₂ gradient to isolate pure product (103 mg, 17%) as a white solid. R_{f}=0.19 (CH₂Cl₂); MS(ES) 571.0 (M+1)⁺.

### EXAMPLE 305

### 1-(3,5-dichloro-benzyl)-5-morpholin-4yl-1H-[1,2,3]triazole-4-carboxylic acid (2-chloro-benzyl)-isopropyl-amide

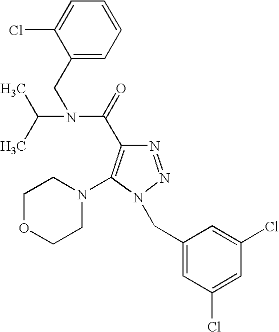

Combine 5-chloro-1-(3,5-dichloro-benzyl)-1H-[1,2,3]triazole-4-carboxylic acid (2-chloro-benzyl)-isopropyl-amide (75 mg, 0. 16 mmol) with morpholine (2 mL) and heat the mixture at 100° C. overnight under N₂. Concentrate the mixture in vacuo, then dissolve in EtOAc and wash with water. Dry over sodium sulfate and concentrate in vacuo. Purify the residue by chromatography over silica gel using a MeOH/CH₂Cl₂ gradient to isolate pure product (38 mg, 46%). MS(ES) 522.1 (M+1); R_{f}=0.03 (CH₂Cl₂).

### EXAMPLE 306

### 1-(3,5-Bis-trifluoromethyl-benzyl)-5-pyridin-4-yl-1H-[1,2,3]triazole-4-carboxylic acid isopropyl-(2-methoxy-5-trifluoromethoxy-benzyl)-amide

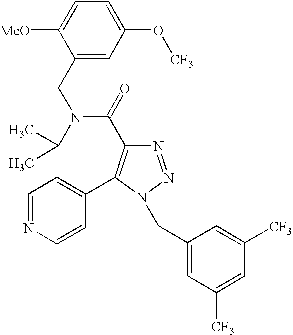

Combine isopropyl-(2-methoxy-5-trifluoromethoxy-benzyl)-amine (126 mg, 0.48 mmol) with 1-(3,5-Bis-trifluoromethyl-benzyl)-5-pyridin-4-yl-1H-[1,2,3]triazole-4-carboxylic acid (200 mg, 0.48 mmol), EDCI (92 mg, 0.48 mmol), HOAt (65 mg, 0.48 mmol), and DIEA (0.10 mL, 0.57 mmol), in DMF (5 mL) and stir the mixture at RT. After 72 h, concentrate the mixture in vacuo, dissolve the residue in EtOAc and wash with water. Dry over sodium sulfate and concentrate in vacuo. Purify the residue by chromatography over silica gel using a MeOH/CH₂Cl₂ gradient to isolate the title compound (300 mg, 94%) as a thick oil. MS(ES) 662.18 (M+1)⁺.

### EXAMPLE 307

### 1-(2-methoxy-5-trifluoromethoxy-benzyl)-pyridin-4-yl-1H-[1,2,3]triazole-4-carboxylic acid (2-chloro-benzyl)-isopropyl-amide

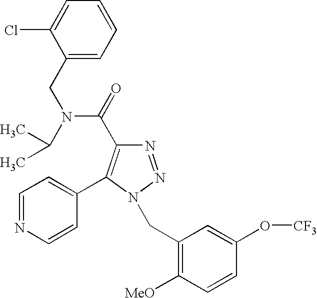

Combine (2-chloro-benzyl)-isopropyl-amine (138 mg, 0.75 mmol) 1-(2-methoxy-5-trifluoromethoxy-benzyl)-5-pyridin-4-yl-1H-[1,2,3]triazole-4-carboxylic acid (295 mg, 0.75 mmol), EDCI (144 mg, 0.75 mmol), HOAt (102 mg, 0.75 mmol), and DIEA (0.10 mL, 0.57 mmol), in DMF (5 mL) and stir the mixture overnight at RT. Concentrate the mixture in vacuo and partition the residue between water and EtOAc. Dry the combined extracts over sodium sulfate and concentrate in vacuo. Chromatograph the residue over silica gel using MeOH/CH₂Cl₂ to isolate product (294 mg, 70%) as a thick oil which solidifies upon standing. ES(MS) 560.2 (M+1)⁺.

### EXAMPLE 308

### 1-(3,5-Bis-trifluoromethyl-benzyl)-5-pyrazol-1-yl-1H[1,2,3]triazole-4-carboxylic acid (2-chloro-benzyl)-methyl-amide

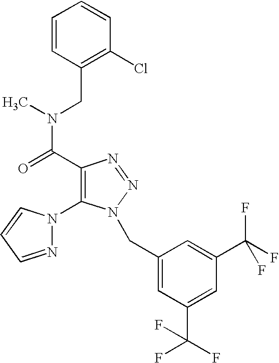

Add sodium hydride (17 mg, 0.43 mmol) to pyrazole (30 mg, 0.44 mmol), in THF (4.0 mL) at RT and stir under nitrogen. After 30 min., add 1-(3,5-bis-trifluoromethyl-benzyl)-5-chloro-1H-[1,2,3]triazole-4-carboxylic acid (2-chloro-benzyl)-methyl-amide (230 mg, 0.45 mmol) and stir for another 6-24h. Treat the reaction mixture with water and extract two times with ethyl acetate. Combine the organic layers and wash with water and brine; then dry (Na₂SO₄), filter, and concentrate under reduced pressure. Purification by flash chromatography, eluting with a linear gradient of 15% to 40% ethyl acetate in hexanes gives the title compound (140 mg, 60%). MS(ES) 543.3 (M+1)⁺; ¹H NMR (400 MHz, CHCl₃, 1:1 mixture of amide rotarmers) δ 8.17 (dd, 1H, J=7.7, 3.0), 7.87 (dd, 1H, J=5.1, 1.7), 7.80 (d, 1H, J=5.1), 7.65 (s, 1H), 7.61 (s, 1H), 7.20-7.38 (m, 4H), 6.46 (m, 1H), 5.88 (s, 1H), 5.85 (s, 1H), 4.98 (s, 1H), 4.84 (s, 1H), 3.23 (s, 1.5H), 2.98 (s, 1.5H).

Using a method analogous to Example 308, with the appropriate starting materials, the following compounds may be prepared and isolated.

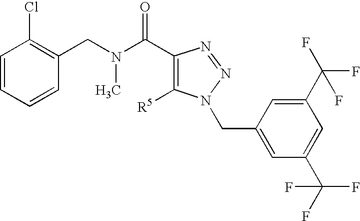

| Ex. # | R⁵ | Data |
|---|---|---|
| 309 | pyrrol-1-yl | MS(ES) 542.3 (M+1)⁺ |
| 310 | imidazol-1-yl | MS(ES) 543.5 (M+1)⁺ |

Using a method analogous to Example 308, with the appropriate starting materials, the following compounds may be prepared and isolated.

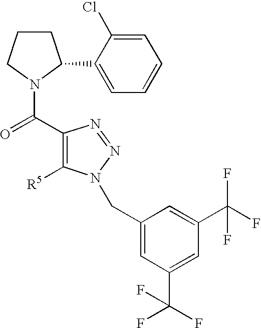

| Ex. # | R⁵ | Data |
|---|---|---|
| 311 | pyrazol-1-yl | MS(ES) 569.3 (M+1)⁺ |
| 312 | imidazol-1-yl | MS(ES) 569.3 (M+1)⁺ |

### EXAMPLE 313

### [1-(3,5-Bis-trifluoromethyl-benzyl)-5-pyrrol-1-yl-1H-[1,2,3]triazol-4-yl]-[2-(2-chloro-phenyl)-pyrrolidin-1-yl]-methanone

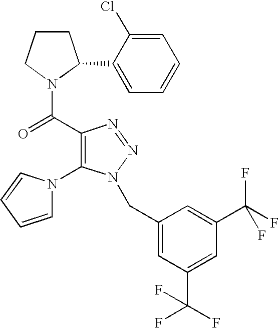

Combine EDCI (132 mg, 0.69 mmol) with a solution of 2-(2-chloro-phenyl)-pyrrolidine (125 mg, 0.69 mmol), 1-(3,5-bis-trifluoromethyl-benzyl)-5-pyrrol-1-yl-1H-[1,2,3]triazole-4-carboxylic acid (200 mg, 0.50 mmol), and DMAP (85 mg, 0.69 mmol) in CH₂Cl₂ (10.0 mL) and stir at RT. After 24 h, dilute the solution with CH₂Cl₂, wash with saturated aqueous NH₄Cl, saturated aqueous NaHCO₃, and water, then dry, filter, and concentrate the organic phase. Purification by flash chromatography eluting with a linear gradient of 15% to 30% EtOAc in hexanes gives the title compound in quantitative yield. MS(ES) 568.3.0 (M+1)⁺; ¹H NMR (400 MHz, CHCl₃, 1:1 mixture of amide rotamers) δ 7.82 (s, 0.5H), 7.79 (s, 0.5H), 7.48 (s, 1H), 7.35 (s, 1H), 7.30 (m, 0.5H), 7.21 (m, 0.5H), 7.13 (m, 1H), 7.03 (m, 1H), 6.94 (m, 0.5H), 6.69 (t, 1H, J=2.2), 6.43 (t, 1H, J=2.2), 6.37 (t, 1H, J=2.2), 6.34 (t, 1H, J=2.2), 6.19 (dd, 0.5H, J=7.9, 2.9), 5.6 (dd, 0.5H, J=7.9, 4.0), 5.48 (m, 1H), 5.28 (m, 1H), 4.41 (m, 0.5H), 3.95 (m, 1H), 3.83 (m, 1H), 2.32-2.52 (m 1H), 1.82-2.01 (m, 3H).

Using a method similar to the above method, with the appropriate starting materials, the following compounds may be prepared and isolated. DMF may be used as a solvent instead of CH₂Cl₂.

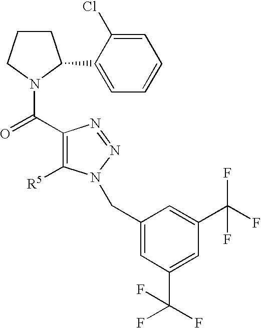

| Ex. # | R⁵ | Data |
|---|---|---|
| 314 | 1-methyl-1H-pyrrol-2-yl | MS(ES) 582.3 (M+1)⁺ |
| 315 | pyrazin-2-yl | MS(ES) 581.1 (M+1)⁺ |
| 316 | pyrimidin-5-yl | MS(ES) 581.2 (M+1)⁺ |
| 317 | 4-methylsulfanyl-phenyl | MS(EI) 625.1 (M+1)⁺ |

### EXAMPLE 318

### [1-(3,5-Bis-trifluoromethyl-benzyl)-5-(4-methanesulfinyl-phenyl)-1H-[1,2,3]triazol-4-yl]-[2-(2-chloro-phenyl)-pyrrolidin-1-yl]-methanone

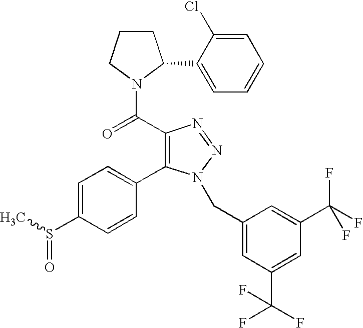

Add [1-(3,5-bis-trifluoromethyl-benzyl)-5-(4-methylsulfanyl-phenyl)-1H-[1,2,3]triazol-4-yl]-[2-(2-chloro-phenyl)-pyrrolidin-1-yl]-methanone (160 mg, 0.26 mmol) to hydrogen peroxide (0.05 mL of 30% aqueous solution, 0.52 mmol) in MeOH (1.0 mL) and stir at RT. After 18 h, quench with a saturated aqueous solution of NaHSO₃, and concentrate under reduced pressure. Purify the residue by flash chromatography, eluting with a linear gradient of 60% to 80% EtOAc in hexanes gives the title compound in quantitative yield. MS(EI) 64 1.0 (M⁺); ¹H NMR (400 MHz, CDCl₃, 1:1 mixture of amide rotamers.) δ, 7.80 (s, 0.5H), 7.76 (s, 0.5H), 7.67 (m, 2H), 7.44 (s, 1H), 7.41 (s, 1H), 7.27 (m, 1H), 7.18 (m, 2H), 7.12 (m, 1H), 7.01 (m, 1H), 6.91 (m, 0.5H), 6.26 (m, 0.5H), 5.56 (m, 1H), 5.37 (m, 1H), 4.52 (m, 0.5H), 4.09 (m, 0.5H), 3.78-3.89 (m, 1H), 2.75 (s, 1.5H), 2.72 (s, 1.5H), 2.45 (m, 1H), 1.85-1.98 (m, 3H).

### EXAMPLE 319

### [1-(3,5-Bis-trifluoromethyl-benzyl)-5-(4-methanesulfonyl-phenyl)-1H-[1,2,3)triazol-4-yl]-[2-(2-chloro-phenyl)-pyrrolidin-1-yl]-methanone

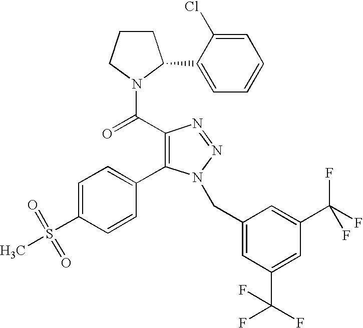

Add 3-chloroperoxybenzoic acid (I0.r mg, 0.45 mmol) to a solution of [1-(3,5-bis-trifluoromethyl-benzyl)-5-(4-methylsulfanyl-phenyl)-1H-[1,2,3 ]triazol4-yl]-[2-(2-chloro-phenyl)-pyrrolidin-1-yl]-methanone (134 mg, 0.21 mmol) in CH₂Cl₂ (3 mL) and stir at RT for 1-3 h. Treat the reaction mixture with 1N HCl and extract with CH₂Cl₂. Combine the organic layers and wash with water, brine, dry (Na₂SO₄), filter, and concentrate under reduced pressure. Add hexane to the residue, collect the precipitate, and dry under vacuum to give the title compound as a white powder in quantitative yield. MS(ES)657.4 (M⁺). ¹H NMR (400 MHz, CDCl₃) δ, 7.96 (s, 1H), 7.94 (s, 1H), 7.82 (s, 0.5H), 7.78 (s, 0.5H), 7.48 (s, 1H), 7.45 (m, 1H), 7.32 (s, 1H), 7.25 (m, 2H), 7.16 (m, 1H), 7.11 (m, 0.5H), 7.01 (m, 1H), 6.91 (m, 0.5H), 6.28 (dd, 0.5H, J=7.9, 2.6), 5.56 (m, 1.5H), 5.36 (m, 1H), 4.53 (m, 0.5H), 4.13 (m, 0.5H), 3.78-3.19 (m, 1H), 3.07 (s, 1.5H), 3.03 (s, 1.5H), 2.45 (m, 1H), 1.85-1.98 (m, 3H).

### EXAMPLE 320

### 1-(3,5-bis-trifluoromethyl-benzyl)-5-pyridin-2-yl-1H-[1,2,3]triazole-4-carboxylic acid (2-chloro-benzyl)-methyl-amide

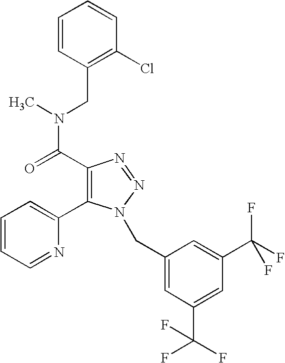

Add (2-chloro-benzyl)-methyl-amine (104 mg, 0.67 mmol), DMAP (62 mg, 0.51 mmol), and EDCI (81 mg, 0.42 mmol) to a solution of 1-(3,5-bis-trifluoromethyl-benzyl)-5-pyridin-2-yl-1H-[1,2,3]triazole-4-carboxylic acid (104 mg, 0.25 mmol) in CH₂Cl₂ (2.5 mL) and stir the solution at RT for 60 h. Dilute the solution with CH₂Cl₂ (25 mL) and wash with saturated aqueous NH₄Cl (10 mL), H₂O (10 mL), and saturated aqueous NaHCO₃ (10 mL). Dry, filter, and concentrate the organic phase, then purify by flash chromatography using a linear gradient of 20% to 40% EtOAc/hexanes to give the title compound (125 mg, 90%) as a clear, colorless oil. MS(ES) 554.2 (M+1)⁺. ¹H NMR (400 MHz, CDCl₃, 1:1 mixture of amide rotamers) δ 8.71 (m, 1H), 7.89 (m, 1H), 7.76 (m, 2H), 7.74 (s, 1H) 7.69 (s, 1H), 7.34 (m, 2H), 7.26 (m, 1H), 7.21 (m, 1H), 7.14 (m, 1H), 6.05 (s, 1H), 6.00 (s, 1H), 4.89 (s, 1H), 4.87 (s, 1H), 3.10 (s, 1.5H), 3.03 (s, 1.5H).

Using a method similar to the above method, with the appropriate starting carboxylic acid, the following compounds may be prepared and isolated.

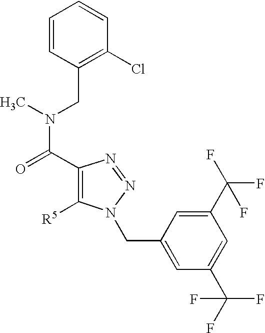

| Ex. # | R⁵ | Data |
|---|---|---|
| 321 | pyridin-3-yl | MS(ES) 554.2 (M+1)⁺; ¹H NMR (400 MHz, CDCl₃, |
| | | 1:1 mixture of amide rotamers) δ 8.74 (m, 1H), |
| | | 8.55 (s, 0.5H), 8.46 (s, 0.5H), 7.82 (s, 0.5H), 7.81 |
| | | (s, 0.5H), 7.67 (m, 0.5H), 7.64 (m, 0.5H), 7.47 (s, |
| | | 1H), 7.42 (s, 1H), 7.39 (s, 0.5H), 7.35 (m, 1.5H), |
| | | 7.22 (m, 3H), 5.60 (s, 1H), 5.54 (s, 1H), 5.14 (s, |
| | | 1H), 4.81 (s, 1H), 3.33 (s, 1,5H), 2.97 (s, 1.5H). |
| 322 | pyridin-4-yl | MS(ES) 554.2 (M+1)⁺; ¹H NMR (400 MHz, CDCl₃, |
| | | 1:1 mixture of amide rotamers.): δ 8.74 (m, 2H), |
| | | 7.84 (m, 1H), 7.52 (s, 1H), 7.47 (s, 1H), 7.34 (m, |
| | | 1H), 7.22 (m, 5H), 5.58 (s, 1H), 5.52 (s, 1H), 5.11 |
| | | (s, 1H), 4.81 (s, 1H), 3.30 (s, 1,5H), 2.98 (s, 1.5H). |
| 323 | furan-2-yl | MS(ES) 543.3 (M+1)⁺; ¹H NMR (400 MHz, CDCl₃, |
| | | 1:1 mixture of amide rotamers.) δ 7.81 (s, 0.5H), |
| | | 7.79 (s, 0.5H), 7.71 (s, 1H) 7.66 (s, 1H), 7.57 (m, |
| | | 1H), 7.30 (m, 2H), 7.22 (m, 1H), 7.17 (m, 2H), |
| | | 6.54 (m, 1H), 5.91 (s, 1H), 5.86 (s, 1H), 4.88 (s, |
| | | 2H), 3.15 (s, 1,5H), 3.02 (s, 1.5H). |
| 324 | furan-3-yl | MS(ES) 543.3 (M+1)⁺; ¹H NMR (400 MHz, |
| | | CDCl₃): δ 7.83 (s, 0.5H), 7.81 (s, 0.5H), 7.75 (s, |
| | | 0.5H) 7.71 (s, 0.5H), 7.60 (m, 1H), 7.54 (m, 2H), |
| | | 7.16-7.36 (m, 4H), 6.43 (m, 1H), 5.66 (s, 1H), 5.60 |
| | | (s, 1H), 4.96 (s, 1H), 4.84 (s, 1H), 3.19 (s, 1,5H), |
| | | 2.99 (s, 1.5H). |
| 325 | thiophen-2- | MS(ES) 559.2 (M+1)⁺; ¹H NMR (400 MHz, |
| | yl | CDCl₃): δ 7.81 (s, 0.5H), 7.79 (s, 0.5H), 7.56 (m, |
| | | 1H) 7.55 (s, 1H), 7.49 (s, 1H), 7.32 (m, 1H), 7.17 |
| | | (m, 5H), 5.67 (s, 1H), 5.62 (s, 1H), 4.95 (s, 1H), |
| | | 4.83 (s, 1H), 3.15 (s, 1,5H), 2.98 (s, 1.5H). |
| 326 | 5-methyl- | MS(ES) 573.3 (M+1)⁺; ¹H NMR (400 MHz, |
| | thiophen-2- | CDCl₃): δ 7.81 (s, 0.5H), 7.79 (s, 0.5H), 7.55 (s, |
| | yl | 1H) 7.50 (s, 1H), 7.32 (m, 1H), 7.24 (m, 1H), 7.19 |
| | | (m, 2H), 6.94 (dd, 1H, J = 3.4, 14.7), 6.78 (m, 1H), |
| | | 5.67 (s, 1H), 5.60 (s, 1H), 4.95 (s, 1H), 4.84 (s, |
| | | 1H), 3.15 (s, 1,5H), 2.98 (s, 1.5H), 2.50 (s, 3H). |

### EXAMPLE 327

### (±)-[1-(3,5-Bis-trifluoromethyl-benzyl)-5-pyridin-2-yl-1H-[1,2,3]triazol-4-yl)-[-]2-(2-chloro-phenyl)-pyrrolidin-1-yl]-methanone

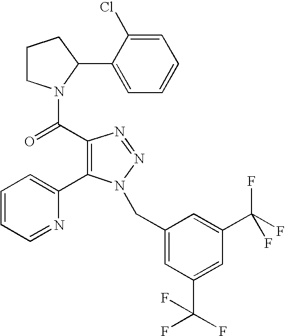

Dissolve 1-(3,5-bis-trifluoromethyl-benzyl)-5-pyridin-2-yl-1H-[1,2,3]triazole4-carboxylic acid (413 mg, 0.99 mmol), (±)-2-(2-chloro-phenyl)-pyrrolidine (196 mg, 1.08 mmol), and DMAP (250 mg, 2.05 mmol) in CH₂Cl₂ (4.0 mL) and treat with EDCI (248 mg, 1.29 mmol). Stir the solution at RT for 60 h, then dilute with additional CH₂Cl2 (20 mL) and wash with saturated NH₄Cl (10 mL), H₂O (10 mL), and saturated NaHCO₃ (10 mL). Dry, filter, and concentrate the organic phase. Purify the crude material by flash chromatography using a linear gradient of 15% to 40% EtOAc/hexanes to give the title compound (463 mg, 81%) as a white foam. MS(ES) 580.2 (M+1)⁺. ¹H NMR (400MHz, CDCl₃): δ 8.68 (d, 0.5H, J=4.9), 8.57 (d, 0.5H, J=4.9), 7.90 (d, 0.5H, J=7.8), 7.80 (d, 0.5H, J=8.3), 7.66-7.74 (m, 5H), 7.11-7.34 (m, 3H), 6.67-6.95 (m, 2H), 5.97 (m, 1H), 5.88 (m, 0.5H), 5.78 (m, 1H), 5.59 (m, 0.5H), 4.29 (m, 0.5H), 3.92 (m, 1.5H), 2.43 (m, 1H), 1.92 (m, 3H).

Using a method similar to the above method, with the appropriate starting carboxylic acid and (+)-(2R)-2-(2-chloro-phenyl)-pyrrolidine, the following compounds may be prepared and isolated.

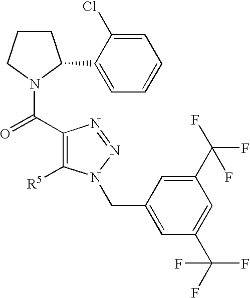

| Ex. # | R⁵ | Data |
|---|---|---|
| 328 | pyridin-3-yl | MS(ES) 580.3 (M+1)⁺; ¹H NMR (400 MHz, CDCl₃, |
| | | 1:1 mixture of amide rotamers): δ 8.67 (m, 1H), |
| | | 8.51 (d, 0.5H, J = 2.0), 8.17 (d, 0.5H, J = 2.0), 7.80 |
| | | (s, 0.5H), 7.77 (s, 0.5H), 7.63 (m, 0.5H), 7.51 (m, |
| | | 0.5H), 7.44 (s, 1H), 6.86-7.37 (m, 6H), 6.28 (m, |
| | | 0.5H), 5.58 (d, 1H, J = 9.3), 5.55(m, 0.5H), 5.38 (s, |
| | | 1H), 4.53 (m, 0.5H), 4.10 (m, 0.5H), 3.88 (m, |
| | | 0.5H), 3.81 (m, 0.5H), 2.49 (m, 0.5H), 2.39 (m, |
| | | 0.5H), 1.83-2.00 (m, 3H). |
| 329 | pyridin-4-yl | MS(ES) 580.2 (M+1)⁺; ¹H NMR (400 MHz, CDCl₃, |
| | | mixture of amide rotamers): δ 8.67 (m, 2H), 7.83 (s, |
| | | 0.5H), 7.80 (s, 0.5H), 7.50 (s, 1H), 7.36 (s, 1H), |
| | | 6.88-7.36 (m, 6H), 6.24 (m, 0.5H), 5.53 (m, 1.5H), |
| | | 5.35(m, 1H), 4.51 (m, 0.5H), 4.09 (m, 0.5H), 3.85 |
| | | (m, 1H), 2.38-2.49 (m, 1H), 1.89-2.05 (m, 3H). |
| 330 | pyridazin-4- | MS(ES) 581.3 (M+1)⁺; ¹H NMR (400 MHz, CDCl₃, |
| | yl | 1:1 mixture of amide rotamers) δ 9.22 (dd, 0.5H, |
| | | J = 1.4, 5.4), 9.18 (dd, 0.5H, J = 1.0, 5.4), 9.02(m, |
| | | 0.5H), 8.73 (m, 0.5H), 7.84 (s, 0.5H), 7.81 (s, |
| | | 0.5H), 7.52 (s, 1H), 7.43 (dd, 0.5H, J = 2.4, 5.4), |
| | | 7.38 (s, 1H), 7.34 (dd, 0.5H, J = 2.4, 5.4), 7.28 (m, |
| | | 0.5H), 7.22 (m, 0.5H), 7.13 (m, 1.5H), 7.04 (dt, |
| | | 0.5H, J = 1.4, 6.0), 6.97 (dt, 0.5H, J = 1.4, 6.0), |
| | | 6.85 (dd, 0.5H, J = 1.7, 7.8), 6.25 (dd, 0.5H, J = |
| | | 3.1, 8.3), 5.59 (m, 1H), 5.53 (dd, 0.5H, J = 4.0, |
| | | 8.1), 5.41 (m, 1H), 4.54 (m, 0.5H), 4.13 (m, 0.5H), |
| | | 3.84 (m, 1H), 2.42 (m, 1H), 1.99 (m, 2.5H), 1.87 |
| | | (m, 0.5H). |
| 331 | furan-2-yl | MS(ES) 569.3 (M+1)⁺; ¹H NMR (400 MHz, CDCl₃, |
| | | mixture of amide rotamers) δ 7.79 (m, 1H), 7.69 (s, |
| | | 1H), 7.60 (s, 1H), 7.54 (s, 0.5H), 7.49 (m, 0.5H), |
| | | 7.33 (m, 0.5H), 7.26 (s, 1.5H), 7.15 (m, 1.5H), 6.99 |
| | | (m, 0.5H), 6.84 (m, 1H), 6.49 (m, 1H), 5.96 (m, |
| | | 0.5H), 5.90 (s, 1H), 5.63 (m, 1.5H), 4.32 (m, 0.5H), |
| | | 3.92 (m, 1.5H), 2.45 (m, 1H), 1.94 (m, 3H). |
| 332 | thiophen-2- | MS(ES) 585.2 (M+1)⁺. ¹H NMR (400 MHz, |
| | yl | CDCl₃): δ 7.80 (s, 0.5H), 7.77 (s, 0.5H), 7.52 (s, |
| | | 1H), 7.51 (m, 1H), 7.39 (s, 1H), 7.29 (m, 0.5H), |
| | | 7.16 (s, 2H), 7.09 (m, 1.5H), 6.95 (m, 2H), 6.11 (m, |
| | | 0.5H), 5.64 (s, 1H), 5.59 (m, 0.5H), 5.43 (m, 1H), |
| | | 4.37 (m, 0.5H), 3.89 (m, 1.5H), 2.43 (m, 1H), 1.92 |
| | | (m, 3H). |
| 333 | 5-methyl- | MS(ES) 599.3 (M+1)⁺. ¹NMR (400 MHz, CDCl₃): |
| | thiophen-2- | δ 7.80 (s, 0.5H), 7.77 (s, 0.5H), 7.53 (s, 1H), 7.40 |
| | yl | (m, 1H), 7.29 (m, 0.5H), 7.14 (m, 2H), 6.95 (m, |
| | | 2H), 6.73 (m, 1.5H), 6.13 (dd, 0.5H, J = 3.4, 7.8), |
| | | 5.64 (s, 1H), 5.60 (dd, 0.5H, J = 3.4, 7.8), 5.42 (m, |
| | | 1H), 4.36 (m, 0.5H), 3.91 (m, 1.5H), 2.46 (d, 3H, |
| | | J = 5.4), 2.43 (m, 1H), 1.93 (m, 3H). |
| 334 | chloro | MS(ES) 537.0 (M+1)⁺; ¹H NMR (400 MHz, |
| | | CDCl₃): δ 7.88 (s, 0.5H), 7.84 (s, 0.5H), 7.80 (s, |
| | | 1H), 7.64 (s, 1H), 7.33 (m, 0.5H), 7.16 (m, 2H), |
| | | 7.00 (m, 1.5H), 6.23 (m, 0.5H), 5.64 (m, 1.5H), |
| | | 5.46 (s, 1H), 4.44 (m, 0.5H), 4.12 (m, 0.5H), 4.01 |
| | | (m, 0.5H), 3.87 (m, 0.5H), 2.43 (m, 1H), 2.00 (m, |
| | | 2H), 1.88 (m, 1H). |
| 335 | isopropyl | ¹H NMR (400 MHz, CDCl₃) δ 7.85 (s, 0.5H), 7.80 |
| | | (s, 0.5H), 7.61 (s, 1H), 7.44 (s, 1H), 7.33 (m, 0.5H), |
| | | 7.24 (m, 0.5H), 7.10-7.20 (m, 1.5H), 6.98-7.04 |
| | | (m, 1.5H), 6.34 (m, 0.5H), 5.66 (s, 1H), 5.64 (m, |
| | | 0.5H), 5.48 (m, 1H), 4.28 (m, 0.5H), 3.85-4.03 (m, |
| | | 1.5H), 3.33 (m, 0.5H), 3.09 (m, 0.5H), 2.40-2.56 |
| | | (m, 1H), 1.96 (m, 3H), 1.08-1.22 (m, 6H). |

### EXAMPLE 336

### (±)-[1-(3,5-Bis-trifluoromethyl-benzyl)-5-furan-3-yl-1H-[1,2,3]triazol-4-yl]-[2-chloro-phenyl)-pyrrolidin-1-yl]-methanone

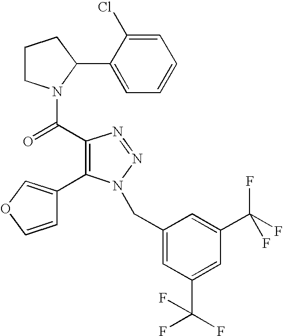

Using a method similar to Example 327, with the appropriate starting carboxylic acid, the title compound may be prepared and isolated. MS(ES) 569.3 (M+1)⁺. ¹H NMR (400 MHz, CDCl₃): δ 7.83 (s, 0.5H), 7.80 (s, 0.5H), 7.73 (m, 0.5H), 7.59 (s, 1H), 7.50 (m, 1.5H), 7.45 (m, 1H), 7.32 (m, 0.5H), 7.22 (s, 0.5H), 7.15 (m, 1.5H), 6.95 (m, 1.5H), 6.42 (m, 0.5H), 6.20 (m, 0.5H), 6.13 (m, 0.5H), 5.64 (s, 1H), 5.61 (m, 0.5H), 5.41 (m, 1H), 4.42 (m, 0.5H), 3.93 (m, 1.5H), 2.44 (m, 1H), 1.94 (m, 3H).

### EXAMPLE 337

### (+)-[1-(3,5-Bis-trifluoromethyl-benzyl)-5-morpholin-4-yl-1H-[1,2,3]triazol-4-yl]-[2-(2-chloro-phenyl)-pyrrolidin-1-yl]-methanone

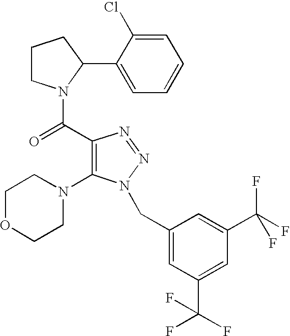

Heat a solution of (+)-[1-(3,5-bis-trifluoromethyl-benzyl)-5-chloro-1H-[1,2,3]triazol-4-yl]-[2-(2-chloro-phenyl)-pyrrolidin-1-yl]-methanone (1.10 g, 2.05 mmol) in morpholine (20 mL) to 110° C. for 18 h. Cool to RT and dilute with EtOAc (60 mL) then wash with 2.5N HCl (2×50 mL), H₂O (50 mL), and saturated NaHCO₃ (50 mL). Dry, filter, and concentrate the organic phase. Purify the crude material by flash chromatography using a linear gradient of 10% to 40% EtOAc/hexanes to give (+)-[1-(3,5-bis-trifluoromethyl-benzyl)-5-morpholin-4-yl-1H-[1,2,3]triazol-4-yl]-[2-(2-chloro-phenyl)-pyrrolidin-1-yl]-methanone (1.20 g, 99%) as a white foam. [α]_{D}=+43.1 (c=1.02, MeOH). ¹H NMR (400 MHz, CDCl₃, mixture of amide rotamers) δ 7.85 (s, 0.5H), 7.83 (s, 1H), 7.81 (s, 0.5H), 7.65 (s, 1H), 7.34 (m, 0.5H), 7.16 (m, 2H), 7.96 (m, 1.5H), 6.31 (m, 0.5H), 5.64 (m, 0.5H), 5.54 (s, 1H), 5.36 (d, 1H, J=3.4), 4.37 (m, 0.5H), 3.99 (m, 1H), 3.90 (m, 0.5H), 3.59-3.73 (m, 4H), 2.87-2.98 (m, 3H), 2.74 (m, 1H), 2.46 (m, 1H), 1.96 (m, 3H). Analytical (C₂₆H₂₄ClF₆N₅O₂): Calculated C, 53.11;H, 4.11; N, 11.91. Found C, 53.41;H, 4.26; N, 11.77.

### EXAMPLE 338

### [1-(3,5-Bis-trifluoromethyl-benzyl)-5-(4-methyl-piperazin-1-yl)-1H-[1,2,3]triazol-4-yl]-[2-(2-chloro-phenyl)-pyrrolidin-1-yl]-methanone

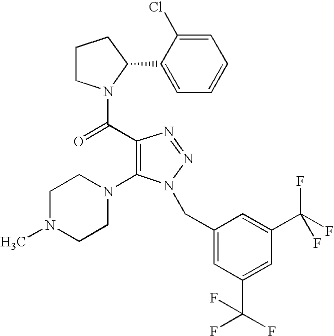

Heat a solution of (+)-[1-(3,5-bis-trifluoromethyl-benzyl)-5-chloro-1H-[1,2,3]triazol-4-yl]-[2-(2-chloro-phenyl)-pyrrolidin-1-yl]-methanone (162 mg, 0.30 mmol) in 4-methylpiperazine (2.0 mL) to 100° C. After 18 h., cool to RT and dilute with EtOAc (60 mL), then wash with IN HCl (2×10 mL), H₂O (10 mL), and saturated NaHCO₃ (10 mL). Dry, filter, and concentrate the organic phase, and purify the crude material by dissolving in MeOH (2.0 mL) and applying to a Varian SCX column. Elute first with MeOH (30 mL) to remove unreacted (+)-[1-(3,5-bis-trifluoromethyl-benzyl)-5-chloro-1H-[1,2,3]triazol-4-yl]-[2-(2-chloro-phenyl)-pyrrolidin-1-yl]-methanone and then elute with 2N NH₃/MeOH to give the title compound (173 mg, 96%) as a white foam upon concentration of solvent. MS(ES) 601.4 (M+1)⁺; ¹H NMR (400 MHz, CDCl₃, mixture of amide rotamers) δ 7.84 (s, 0.5H), 7.83 (s, 1H), 7.80 (s, 0.5H), 7.65 (s, 1H), 7.32 (m, 0.5H), 7.12 (m, 2H), 7.96 (m, 1.5H), 6.25 (m, 0.5H), 5.62 (m, 0.5H), 5.50 (s, 1H), 5.32 (m, 1H), 4.31 (m, 0.5H), 3.97 (m, 1H), 3.86 (m, 0.5H), 2.97 (m, 3H), 2.75 (m, 1H), 2.41 (m, 5H), 2.27 (s, 1.5H), 2.25 (s, 1.5H), 1.94 (m, 3H).

### EXAMPLE 339

### 1-[1-(3,5-bis-trifluoromethyl-benzyl)-5-chloro-1H-[1,2,3]triazole-4-carbonyl]-5,5-dimethyl-2-phenyl-pyrazolidin-3-one

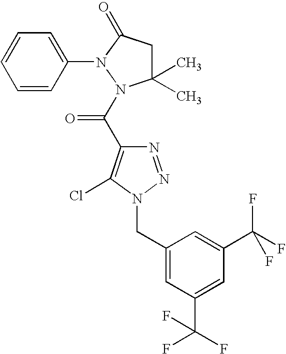

Dissolve 1-(3,5-bis-trifluoromethyl-benzyl)-5-chloro-1H[1,2,3]triazole-4-carboxylic acid (250 mg, 0.67 mmol) in CH₂Cl₂ (5 mL) and DMF (1 drop) and add oxalyl chloride (0.12 mL, 1.34 mmol). Stir 1.5 h at RT, then concentrate to dryness. Slurry in 1,2-dichloroethane and concentrate to dryness 2×. Dissolve the residue in pyridine (3 mL) in a sealed tube. Add a catalytic amount of DMAP (5 mg) and 5,5-dimethyl-2-phenyl-3-pyrazolidinone (128 mg, 0.67 mmol). Heat for 2 h at 100° C., then concentrate to dryness. Dissolve in 20% iPrOH/CHCl₃. Wash with saturated aqueous NaHCO₃, and brine, dry over Na₂SO₄, filter and concentrate. Purify the residue via radial chromatography using a MeOH/CHCl₃ gradient to afford 147 mg (40%) of the title compound as a white foam. ES(MS) 546.3 (M+1)⁺; R_{f}=0.58 (5% MeOH/CHCl₃).

Using a method similar to Example 339, with the appropriate starting materials, the following compounds may be prepared and isolated.

| Ex. # | Product | Data |
|---|---|---|
| 340 | 1-[1-(3,5-bis-trifluoromethyl-benzyl)-5-phenyl- | MS(ES) 588.2(M+1)⁺; |
| | 1H-[1,2,3]triazole-4-carbonyl]-2-phenyl- | |
| | pyrazolidin-3-one | |
| 341 | 1-[1-(3,5-bis-trifluoromethyl-benzyl)-5-pyridin-4- | MS(ES) 589.1(M+1)⁺; |
| | yl-1H-[1,2,3]triazole-4-carbonyl]-5,5-dimethyl-2- | R_{f}=0.44(10% MeOH/CHCl₃) |
| | phenyl-pyrazolidin-3-one | |
| 342 | [1-(3,5-bis-trifluoromethyl-benzyl)-5-chloro-1H- | MS(ES) 538.2(M+1)⁺; |
| | [1,2,3]triazol-4-yl]-[2-(2-chloro-phenyl)- | R_{f}=0.55(5% MeOH/CHCl₃) |
| | pyrazolidin-1-yl]-methanone | |
| 343 | [1-(3,5-bis-trifluoromethyl-benzyl)-5-phenyl-1H- | MS(ES) 580.4(M+1)⁺; |
| | [1,2,3]triazol-4-yl]-[2-(2-chloro-phenyl)- | |
| | pyrazolidin-1-yl]-methanone | |
| 344 | (R,S)-[1-(3,5-bis-trifluoromethyl-benzyl)-5-(4- | MS(ES) 598.0(M+1)⁺; |
| | fluoro-phenyl)-1H-[1,2,3]triazol-4-yl]-[2-(2- | R_{f}=0.38(5% MeOH/CHCl₃) |
| | chloro-phenyl)-pyrazolidin-1-yl]-methanone | |
| 345 | (R)-[1-(3,5-bis-trifluoromethyl-benzyl)-5-(4- | MS(ES) 597.0(M+1)⁺; |
| | fluoro-phenyl)-1H-[1,2,3]triazol-4-yl]-[2-(2- | R_{f}=0.28(1:1 EtOAc/hexanes) |
| | chloro-phenyl)-pyrazolidin-1-yl]-methanone | |
| 346 | [1-(3,5-bis-trifluoromethyl-benzyl)-5-pyridin-4- | MS(ES) 581.0(M+1)⁺; |
| | yl-1H-[1,2,3]triazol-4-yl]-[2-(2-chloro-phenyl)- | R_{f}=0.23(10% MeOH/CHCl₃) |
| | pyrazolidin-1-yl]-methanone | |
| 347 | [1-(3,5-bis-trifluoromethyl-benzyl)-5-pyridin-4- | MS(ES) 581.0(M+1)⁺; |
| | yl-1H-[1,2,3]triazol-4-yl]-[2-(2-chloro-phenyl)- | R_{f}=0.61(10% MeOH/CHCl₃) |
| | pyrazolidin-1-yl]-methanone | |
| 348 | [1-(3,5-bis-trifluoromethyl-benzyl)-5-pyrazin-2- | MS(ES) 582.0(M+1)⁺; |
| | yl-1H-[1,2,3]triazol-4-yl]-[2-(2-chloro-phenyl)- | R_{f}=0.50(10% MeOH/CHCl₃) |
| | pyrazolidin-1-yl]-methanone | |
| 349 | [1-(3,5-bis-trifluoromethyl-benzyl)-5-pyridin-4- | MS(ES) 649.1(M+1)⁺; |
| | yl-1H-[1,2,3]triazol-4-yl]-[2-(2-chloro-4- | R_{f}=0.40(10% MeOH/CHCl₃) |
| | trifluoromethyl-phenyl)-pyrazolidin-1-yl]- | |
| | methanone | |
| 350 | [1-(3,5-bis-trifluoromethyl-benzyl)-5-pyridin-3- | MS(ES) 649.1(M+1)⁺; |
| | yl-1H-[1,2,3]triazol-4-yl]-[2-(2-chloro-4- | R_{f}=0.60(10% MeOH/CHCl₃) |
| | trifluoromethyl-phenyl)-pyrazolidin-1-yl]- | |
| | methanone | |
| 351 | [1-(3,5-bis-trifluoromethyl-benzyl)-5-pyridin-3- | MS(ES) 583.1(M+1)⁺; |
| | yl-1H-[1,2,3]triazol-4-yl]-[2-(2,4-difluoro- | R_{f}=0.38(10% MeOH/CHCl₃) |
| | phenyl)-pyrazolidin-1-yl]-methanone | |
| 352 | [1-(3,5-bis-trifluoromethyl-benzyl)-5-pyridin-4- | MS(ES) 583.1(M+1)⁺; |
| | yl-1H-[1,2,3]triazol-4-yl]-[2-(2,4-difluoro- | R_{f}=0.33(10% MeOH/CHCl₃) |
| | phenyl)-pyrazolidin-1-yl]-methanone | |
| 353 | [1-(3,5-bis-trifluoromethyl-benzyl)-5-pyridin-3- | MS(ES) 595.1(M+1)⁺; |
| | yl-1H-[1,2,3]triazol-4-yl]-[2-(2-chloro-phenyl)- | R_{f}=0.43(10% MeOH/CHCl₃) |
| | tetrahydro-pyridazin-1-yl]-methanone | |
| 354 | [1-(3,5-bis-trifluoromethyl-benzyl)-5-pyridin-4- | MS(ES) 595.1(M+1)⁺; |
| | yl-1H-[1,2,3]triazol-4-yl]-[2-(2-chloro-phenyl)- | R_{f}=0.43(10% MeOH/CHCl₃) |
| | tetrahydro-pyridazin-1-yl]-methanone | |
| 355 | [1-(3,5-bis-trifluoromethyl-benzyl)-5-pyridin-4- | MS(ES) 565.9(M+1)⁺; |
| | yl-1H-[1,2,3]triazol-4-yl]-(8-chloro-3,4-dihydro- | R_{f}=0.43(10% MeOH/CHCl₃) |
| | 2H-quinolin-1-yl)-methanone | |
| 356 | [1-(3,5-bis-trifluoromethyl-benzyl)-5-chloro-1H- | MS(ES) 522.9(M+1)⁺; |
| | [1,2,3]triazol-4-yl]-(8-chloro-3,4-dihydro-2H- | R_{f}=0.60(1:1 EtOAc/hexanes) |
| | quinolin-1-yl)-methanone | |
| 357 | cis-(R/S)-[1-(3,5-bis-trifluoromethyl-benzyl)-5- | MS(ES) 622(M+1)⁺; |
| | pyridin-4-yl-1H-[1,2,3]triazol-4-yl]-(2,4- | R_{f}=0.48(1:1 EtOAc/hexanes) |
| | diphenyl-pyrrolidin-1-yl)-methanone | |

### EXAMPLE 358

### 1-[1-(3,5-bis-trifluoromethyl-benzyl)-5-morpholin-4-yl-1H-[1,2,3]triazole-4-carbonyl]-5,5-dimethyl-2-phenyl-pyrazolidin-3-one

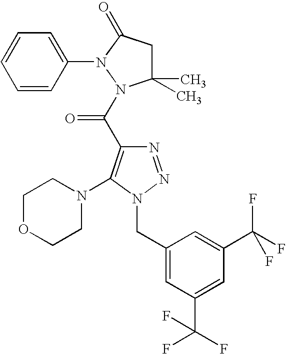

Dissolve 1-[1-(3,5-bis-trifluoromethyl-benzyl)-5-chloro-1H-[1,2,3]triazole-4-carbonyl]-5,5-dimethyl-2-phenyl-pyrazolidin-3-one (120 mg, 0.22 mmol) in morpholine (3 mL). Heat overnight at 100° C. in a sealed tube, then concentrate to dryness. Dissolve the residue in 20% iPrOH/CHCl₃. Wash with saturated aqueous NaHCO₃, and brine, dry over sodium sulfate, filter, and concentrate to dryness. Purify the residue via radial chromatography using a MeOH/CHCl₃ gradient to afford 16.4 mg (12.5%) of the title compound MS(ES) 597.4 (M+1)⁺; R_{f}=0.76 (10% MeOH/CHCl₃).

Using a method similar to the above example, with the appropriate starting materials, the following compounds may be prepared and isolated.

| Ex. # | Product | Data |
|---|---|---|
| 359 | [1-(3,5-bis-trifluoromethyl-benzyl)- | MS(ES) 589.3(M+1)⁺; |
| | 5-morpholin-4-yl-1H-[1,2,3]triazol- | R_{f}=0.5(10% MeOH/CHCl₃) |
| | 4-yl]-[2-(2-chloro-phenyl)- | |
| | pyrazolidin-1-yl]-methanone | |
| 360 | [1-(3,5-bis-trifluoromethyl-benzyl)- | MS(IS) 522.9(M+); TLC |
| | 5-morpholin-4-yl-1H-[1,2,3]triazol- | R_{f}=0.5(1:1 EtOAc/hexanes) |
| | 4-yl]-(8-chloro-3,4-dihydro- | |
| | 2H-quinolin-1-yl)-methanone | |

### EXAMPLE 361

### [1-(3,5-bis-trifluoromethyl-benzyl)-5-(4-fluoro-phenyl)-1H-[1,2,3]triazol-4-yl]-[2-(2-chloro-phenyl)-pyrrolidin-1-yl]-methanone

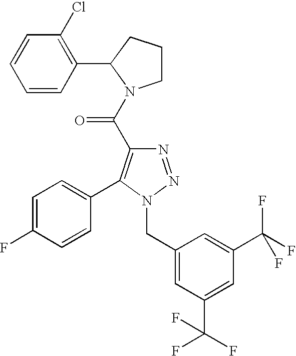

Dissolve 1-(3,5-bis-trifluoromethyl-benzyl)-5-(4-fluoro-phenyl)-1H-[1,2,3]triazole-4-carboxylic acid (100 mg,-0.23 mmol) in DMF (5 mL). Add 2-(2-chlorophenyl)-pyrrolidine (46 mg, 0.25 mmol), hydroxy-azabenzotriazole (HOAt)(50 mg, 0.25 mmol), EDCI (35 mg, 0.25 mmol), DMAP (5 mg) and TEA (0.1 mL, 0.69 mmol). Stir overnight at RT, then concentrate to dryness. Purify by radial chromatography using a MeOH/CHCl₃ gradient. Slurry the residue in ether/hexanes and concentrate to dryness to afford 87 mg (63%) of the title compound as a white foam. MS(ES) 597.0 (M+1)⁺; R_{f}=0.67 (5% MeOH/CHCl₃).

### EXAMPLE 362

### 1(3,5-bis-trifluoromethyl-benzyl)-5-chloro-1H-[1,2,3]triazole-4-carboxylic acid (2-chloro-phenyl)-methyl-amide

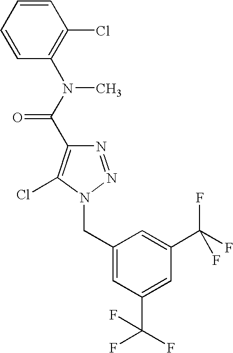

Dissolve 1-(3,5-bis-trifluoromethyl-benzyl)-5-chloro-1H[1,2,3]triazole-4-carboxylic acid (300 mg, 0.8 mmol) in CH₂Cl₂ (5 mL) and DMF (2 drops) and add oxalyl chloride (0.14 mL, 1.6 mmol). Stir for 1 h at RT, then concentrate the mixture to dryness. Slurry the residue in 1,2-dichloroethane and concentrate to dryness twice. Dissolve the residue in pyridine (3 mL) in a sealed tube. Add DMAP (5 mg, catalytic) and N-methyl-2-chloroaniline (120 mg, 0.8 mmol). Heat for 1 h at 80° C., then concentrate to dryness. Dissolve in 20% iPrOH/CHCl₃. Wash with saturated aqueous NaHCO₃, and brine, then dry over Na₂SO₄, filter, and concentrate. Purify the residue via radial chromatography using an ethyl acetate/hexanes gradient to afford 200 mg (50%) of the title compound as a colorless oil. MS(ES) 497.2 (M+1)⁺; R_{f}=0.625 (50% EtOAc/hexanes).

Using a similar method to that described above and the appropriate starting materials, the following compounds may be prepared and isolated.

| Ex. # | Product | Data |
|---|---|---|
| 363 | 1-(3,5-bis-trifluoromethyl-benzyl)-5-(4-fluoro- | MS(ES) 557.0(M+1)⁺; |
| | phenyl)-1H-[1,2,3]triazole-4-carboxylic acid (2- | R_{f}=0.52(5% MeOH/CHCl₃) |
| | chloro-phenyl)-methyl-amide | |
| 364 | 1-(3,5-bis-trifluoromethyl-benzyl)-5-(pyridin-4- | MS(ES) 540.0(M+1)⁺; |
| | yl)-1H-[1,2,3]triazole-4-carboxylic acid (2- | R_{f}=0.58(5% MeOH/CHCl₃) |
| | chloro-phenyl)-methyl-amide | |
| 365 | 1-(3,5-bis-trifluoromethyl-benzyl)-5-pyridin-3-yl- | MS(ES) 540.0(M+1)⁺; |
| | 1H-[1,2,3]triazole-4-carboxylic acid (2-chloro- | R_{f}=0.50(10% MeOH/CHCl₃) |
| | phenyl)-methyl-amide | |
| 366 | 1-(3,5-bis-trifluoromethyl-benzyl)-5-chloro-1H- | MS(ES) 530.9(M+1)⁺; |
| | [1,2,3]triazole-4-carboxylic acid (2,4-dichloro- | R_{f}=0.75(5% MeOH/CHCl₃) |
| | phenyl)-methyl-amide | |
| 367 | 1-(3,5-bis-trifluoromethyl-benzyl)-5-pyridin-3-yl- | MS(ES) 554.0(M+1)⁺; |
| | 1H-[1,2,3]triazole-4-carboxylic acid (2-chloro-4- | R_{f}=0.43(10% MeOH/CHCl₃) |
| | methyl-phenyl)-methyl-amide | |
| 368 | 1-(3,5-bis-trifluoromethyl-benzyl)-5-pyridin-4-yl- | MS(ES) 573.0(M+1)⁺; |
| | 1H-[1,2,3]triazole-4-carboxylic acid (2,4- | R_{f}=0.70(5% MeOH/CHCl₃) |
| | dichloro-phenyl)-methyl-amide | |
| 369 | 1-(3,5-bis-trifluoromethyl-benzyl)-5-chloro-1H- | MS(ES) 515.0(M+1)⁺; |
| | [1,2,3]triazole-4-carboxylic acid (2-chloro-4- | R_{f}=0.61(5% MeOH/CHCl₃) |
| | fluoro-phenyl)-methyl-amide | |
| 370 | 1-(3,5-bis-trifluoromethyl-benzyl)-5-pyridin-3-yl- | MS(ES) 558.0(M+1)⁺; |
| | 1H-[1,2,3]triazole-4-carboxylic acid (2-chloro-4- | R_{f}=0.44(10% MeOH/CHCl₃) |
| | fluoro-phenyl)-methyl-amide | |
| 371 | 1-(3,5-bis-trifluoromethyl-benzyl)-5-pyridin-3-yl- | MS(IS) 574.0(M+1)⁺; TLC |
| | 1H-[1,2,3]triazole-4-carboxylic acid (2,4- | R_{f}=0.50(10% MeOH/CHCl₃) |
| | dichlorophenyl)-methyl-amide | |
| 372 | 1-(3,5-bis-trifluoromethyl-benzyl)-5-pyridin-4-yl- | MS(ES) 558.0(M+1)⁺; |
| | 1H-[1,2,3]triazole-4-carboxylic acid (2-chloro-4- | R_{f}=0.38(10% MeOH/CHCl₃) |
| | fluoro-phenyl)-methyl-amide | |
| 373 | 1-(3,5-bis-trifluoromethyl-benzyl)-5-chloro-1H- | MS(ES) 511.0(M+1)⁺; |
| | [1,2,3]triazole-4-carboxylic acid (2-chloro,4- | R_{f}=0.57(5% MeOH/CHCl₃) |
| | methyl-phenyl)-methyl-amide | |
| 374 | 1-(3,5-bis-trifluoromethyl-benzyl)-5-pyridin-4-yl- | MS(ES) 554.0(M+1)⁺; |
| | 1H-[1,2,3]triazole-4-carboxylic acid (2-chloro-4- | R_{f}=0.48(1:1 EtOAc/hexanes) |
| | methyl-phenyl)-methyl-amide | |
| 375 | 1-(3,5-bis-trifluoromethyl-benzyl)-5-pyridin-3-yl- | MS(ES) 574.0(M+1)⁺; |
| | 1H-[1,2,3]triazole-4-carboxylic acid (2,4- | R_{f}=0.36(10% MeOH/CHCl₃) |
| | dichloro-phenyl)-methyl-amide | |
| 376 | 1-(3,5-bis-trifluoromethyl-benzyl)-5-pyridin-4-yl- | MS(ES) 574.0(M+1)⁺; |
| | 1H-[1,2,3]triazole-4-carboxylic acid (3,4- | R_{f}=0.40(10% MeOH/CHCl₃) |
| | dichloro-phenyl)-methyl-amide | |
| 377 | 1-(3,5-bis-trifluoromethyl-benzyl)-5-pyridin-3-yl- | MS(ES) 542.1(M+1)⁺; |
| | 1H-[1,2,3]triazole-4-carboxylic acid (3,4- | R_{f}=0.50(10% MeOH/CHCl₃) |
| | difluoro-phenyl)-methyl-amide | |
| 378 | 1-(3,5-bis-trifluoromethyl-benzyl)-5-pyridin-3-yl- | MS(ES) 602.0(M+1)⁺; |
| | 1H-[1,2,3]triazole-4-carboxylic acid (2,4- | R_{f}=0.62(10% MeOH/CHCl₃) |
| | dichloro-phenyl)-isopropyl-amide | |
| 379 | 1-(3,5-bis-trifluoromethyl-benzyl)-5-pyridin-4-yl- | MS(ES) 602.0(M+1)⁺; |
| | 1H-[1,2,3]triazole-4-carboxylic acid (2,4- | R_{f}=0.40(10% MeOH/CHCl₃) |
| | dichloro-phenyl)-isopropyl-amide | |
| 380 | 1-(3,5-bis-trifluoromethyl-benzyl)-5-pyridin-3-yl- | MS(ES) 586.1(M+1)⁺; |
| | 1H-[1,2,3]triazole-4-carboxylic acid (2-chloro-4- | R_{f}=0.50(10% MeOH/CHCl₃) |
| | fluoro-phenyl)-isopropyl-amide | |
| 381 | 1-(3,5-bis-trifluoromethyl-benzyl)-5-pyridin-4-yl- | MS(ES) 586.1(M+1)⁺; |
| | 1H-[1,2,3]triazole-4-carboxylic acid (2-chloro-4- | R_{f}=0.57(10% MeOH/CHCl₃) |
| | fluoro-phenyl)-isopropyl-amide | |
| 382 | 1-(3,5-bis-trifluoromethyl-benzyl)-5-pyridin-3-yl- | MS(ES) 636.1(M+1)⁺; |
| | 1H-[1,2,3]triazole-4-carboxylic acid (2-chloro-4- | R_{f}=0.31(10% MeOH/CHCl₃) |
| | trifluoromethyl-phenyl)-isopropyl-amide | |
| 383 | 1-(3,5-bis-trifluoromethyl-benzyl)-5-pyridin-4-yl- | MS(ES) 636.1(M+1)⁺; |
| | 1H-[1,2,3]triazole-4-carboxylic acid (2-chloro-4- | R_{f}=0.68(10% MeOH/CHCl₃) |
| | trifluoromethyl-phenyl)-isopropyl-amide | |
| 384 | 1-(3,5-bis-trifluoromethyl-benzyl)-5-pyridin-3-yl- | MS(ES) 570.1(M+1)⁺; |
| | 1H-[1,2,3]triazole-4-carboxylic acid (3,4- | R_{f}=0.50(10% MeOH/CHCl₃) |
| | difluoro-phenyl)-isopropyl-amide | |
| 385 | 1-(3,5-bis-trifluoromethyl-benzyl)-5-pyridin-4-yl- | MS(ES) 570.1(M+1)⁺; |
| | 1H-[1,2,3]triazole-4-carboxylic acid (3,4- | R_{f}=0.50(10% MeOH/CHCl₃) |
| | difluoro-phenyl)-isopropyl-amide | |
| 386 | 1-(3,5-bis-trifluoromethyl-benzyl)-5-pyridin-3-yl- | MS(ES) 616.0(M+1)⁺; |
| | 1H-[1,2,3]triazole-4-carboxylic acid (2,4- | R_{f}=0.50(10% MeOH/CHCl₃) |
| | dichloro-benzyl)-isopropyl-amide | |
| 387 | 1-(3,5-bis-trifluoromethyl-benzyl)-5-pyridin-4-yl- | MS(ES) 616.1(M+1)⁺; |
| | 1H-[1,2,3]triazole-4-carboxylic acid (2,4- | R_{f}=0.58(10% MeOH/CHCl₃) |
| | dichloro-benzyl)-isopropyl-amide | |
| 388 | 1-(3,5-bis-trifluoromethyl-benzyl)-5-pyridin-3-yl- | MS(ES) 584.1(M+1)⁺; |
| | 1H-[1,2,3]triazole-4-carboxylic acid (3,4- | R_{f}=0.50(10% MeOH/CHCl₃) |
| | difluoro-benzyl)-isopropyl-amide | |
| 389 | 1-(3,5-bis-trifluoromethyl-benzyl)-5-pyridin-4-yl- | MS(ES) 584.1(M+1)⁺; |
| | 1H-[1,2,3]triazole-4-carboxylic acid (3,4- | R_{f}=0.37(10% MeOH/CHCl₃) |
| | difluoro-benzyl)-isopropyl-amide | |
| 390 | 1-(3,5-bis-trifluoromethyl-benzyl)-5-pyridin-3-yl- | MS(ES) 582.1(M+1)⁺; |
| | 1H-[1,2,3]triazole-4-carboxylic acid (2-chloro- | R_{f}=0.57(10% MeOH/CHCl₃) |
| | benzyl)-isopropyl-amide | |
| 391 | 1-(3,5-bis-trifluoromethyl-benzyl)-5-pyridin-4-yl- | MS(ES) 582.1(M+1)⁺; |
| | 1H-[1,2,3]triazole-4-carboxylic acid (2-chloro- | R_{f}=0.33(10% MeOH/CHCl₃) |
| | benzyl)-isopropyl-amide | |
| 392 | 1-(3,5-bis-trifluoromethyl-benzyl)-5-pyridin-4-yl- | MS(ES) 600.0(M+1)⁺; |
| | 1H-[1,2,3]triazole-4-carboxylic acid (2-chloro-4- | R_{f}=0.57(10% MeOH/CHCl₃) |
| | fluoro-benzyl)-isopropyl-amide | |
| 393 | 1-(3,5-bis-trifluoromethyl-benzyl)-5-chloro-1H- | MS(ES) 557.0(M+1)⁺; |
| | [1,2,3]triazole-4-carboxylic acid (2-chloro-4- | R_{f}=0.67(1:1 EtOAc/hexanes) |
| | fluoro-benzyl)-isopropyl-amide | |
| 394 | 1-(3,5-bis-trifluoromethyl-benzyl)-5-pyridin-4-yl- | MS(ES) 705.9(M+1)⁺ |
| | 1H-[1,2,3]triazole-4-carboxylic acid bis-(2,5- | |
| | dichloro-phenyl)-amide trifluoroacetate | |
| 395 | 1-(3,5-bis-trifluoromethyl-benzyl)-5-pyridin-3-yl- | MS(ES) 623.2(M+1)⁺; |
| | 1H-[1,2,3]triazole-4-carboxylic acid (2-chloro- | R_{f}=0.21(10% MeOH/CHCl₃) |
| | phenyl)-(2-pyrrolidin-1-yl-ethyl)-amide | |
| 396 | 1-(3,5-bis-trifluoromethyl-benzyl)-5-pyridin-4-yl- | MS(ES) 623.2(M+1)⁺; |
| | 1H-[1,2,3]triazole-4-carboxylic acid (2-chloro- | R_{f}=0.23(10% MeOH/CHCl₃) |
| | phenyl)-(2-pyrrolidin-1-yl-ethyl)-amide | |
| 397 | 1-(3,5-bis-trifluoromethyl-benzyl)-5-chloro-1H- | MS(ES) 580.0(M+1)⁺; |
| | [1,2,3]triazole-4-carboxylic acid (2-chloro- | R_{f}=0.24(10% MeOH/CHCl₃) |
| | phenyl)-(2-pyrrolidin-1-yl-ethyl)-amide | |
| 398 | 1-(3,5-bis-trifluoromethyl-benzyl)-5-pyridin-3-yl- | MS(ES) 597.2(M+1)⁺; |
| | 1H-[1,2,3]triazole-4-carboxylic acid (2-chloro- | R_{f}=0.24(10% MeOH/CHCl₃) |
| | phenyl)-(2-dimethylamino-ethyl)-amide | |
| 399 | 1-(3,5-bis-trifluoromethyl-benzyl)-5-pyridin-4-yl- | MS(ES) 597.2(M+1)⁺; |
| | 1H-[1,2,3]triazole-4-carboxylic acid (2-chloro- | R_{f}=0.20(10% MeOH/CHCl₃) |
| | phenyl)-(2-dimethylamino-ethyl)-amide | |
| 400 | 1-(3,5-bis-trifluoromethyl-benzyl)-5-pyridin-3-yl- | MS(ES) 637.1(M+1)⁺ |
| | 1H-[1,2,3]triazole-4-carboxylic acid (2-chloro- | R_{f}=0.25(10% MeOH/CHCl₃) |
| | phenyl)-(2-piperidin-1-yl-ethyl)-amide | |
| 401 | 1-(3,5-bis-trifluoromethyl-benzyl)-5-pyridin-4-yl- | MS(ES) 637.2(M+1)⁺; |
| | 1H-[1,2,3]triazole-4-carboxylic acid (2-chloro- | R_{f}=0.27(10% MeOH/CHCl₃) |
| | phenyl)-(2-piperidin-1-yl-ethyl)-amide | |
| 402 | 1-(3,5-bis-trifluoromethyl-benzyl)-5-pyridin-3-yl- | MS(ES) 639.2(M+1)⁺; |
| | 1H-[1,2,3]triazole-4-carboxylic acid (2-chloro- | R_{f}=0.33(10% MeOH/CHCl₃) |
| | phenyl)-(2-morpholin-4-yl-ethyl)-amide | |
| 403 | 1-(3,5-bis-trifluoromethyl-benzyl)-5-pyridin-4-yl- | MS(ES) 639.2(M+1)⁺; |
| | 1H-[1,2,3]triazole-4-carboxylic acid (2-chloro- | R_{f}=0.25(10% MeOH/CHCl₃) |
| | phenyl)-(2-morpholin-4-yl-ethyl)-amide | |
| 404 | 1-(3,5-bis-trifluoromethyl-benzyl)-5-pyridin-3-yl- | MS(ES) 641.2(M+1)⁺; |
| | 1H-[1,2,3]triazole-4-carboxylic acid (2-chloro-4- | R_{f}=0.33(10% MeOH/CHCl₃) |
| | fluoro-phenyl)-(2-pyrrolidin-1-yl-ethyl)-amide | |
| 405 | 1-(3,5-bis-trifluoromethyl-benzyl)-5-pyridin-4-yl- | MS(ES) 641.2(M+1)⁺; |
| | 1H-[1,2,3]triazole-4-carboxylic acid (2-chloro-4- | R_{f}=0.34(10% MeOH/CHCl₃) |
| | fluoro-phenyl)-(2-pyrrolidin-1-yl-ethyl)-amide | |
| 406 | 1-(3,5-bis-trifluoromethyl-benzyl)-5-pyridin-3-yl- | MS(ES) 615.1(M+1)⁺; |
| | 1H-[1,2,3]triazole-4-carboxylic acid (2-chloro-4- | R_{f}=0.33(10% MeOH/CHCl₃) |
| | fluoro-phenyl)-(2-dimethylamino-ethyl)-amide | |
| 407 | 1-(3,5-bis-trifluoromethyl-benzyl)-5-pyridin-4-yl- | MS(ES) 615.1(M+1)⁺; |
| | 1H-[1,2,3]triazole-4-carboxylic acid (2-chloro-4- | R_{f}=0.20(10% MeOH/CHCl₃) |
| | fluoro-phenyl)-(2-dimethylamino-ethyl)-amide | |
| 408 | 1-(3,5-bis-trifluoromethyl-benzyl)-5-pyridin-3-yl- | MS(ES) 657.0(M+1)⁺; |
| | 1H-[1,2,3]triazole-4-carboxylic acid (2-chloro-4- | R_{f}=0.28(10% MeOH/CHCl₃) |
| | fluoro-phenyl)-(2-morpholin-4-yl-ethyl)-amide | |
| 409 | 1-(3,5-bis-trifluoromethyl-benzyl)-5-pyridin-4-yl- | MS(ES) 656.9(M+1)⁺; |
| | 1H-[1,2,3]triazole-4-carboxylic acid (2-chloro-4- | R_{f}=0.33(10% MeOH/CHCl₃) |
| | fluoro-phenyl)-(2-morpholin-4-yl-ethyl)-amide | |
| 410 | 1-(3,5-bis-trifluoromethyl-benzyl)-5-pyridin-3-yl- | MS(ES) 655.2(M+1); |
| | 1H-[1,2,3]triazole-4-carboxylic acid (2-chloro-4- | R_{f}=0.33(10% MeOH/CHCl₃) |
| | fluoro-phenyl)-(2-piperidin-1-yl-ethyl)-amide | |
| 411 | 1-(3,5-bis-trifluoromethyl-benzyl)-5-pyridin-4-yl- | MS(ES) 655.1(M+1)⁺; |
| | 1H-[1,2,3]triazole-4-carboxylic acid (2-chloro-4- | R_{f}=0.30(10% MeOH/CHCl₃) |
| | fluoro-phenyl)-(2-piperidin-1-yl-ethyl)-amide | |
| 412 | 1-(3,5-bis-trifluoromethyl-benzyl)-5-pyridin-3-yl- | MS(ES) 631.1(M+1)⁺; |
| | 1H-[1,2,3]triazole-4-carboxylic acid (2,4- | R_{f}=0.33(10% MeOH/CHCl₃) |
| | dichloro-phenyl)-(2-dimethylamino-ethyl)-amide | |
| 413 | 1-(3,5-bis-trifluoromethyl-benzyl)-5-pyridin-4-yl- | MS(ES) 631.1(M+1)⁺; |
| | 1H-[1,2,3]triazole-4-carboxylic acid (2,4- | R_{f}=0.57(20% MeOH/CHCl₃) |
| | dichloro-phenyl)-(2-dimethylamino-ethyl)-amide | |
| 414 | 1-(3,5-bis-trifluoromethyl-benzyl)-5-pyridin-4-yl- | MS(ES) 657.0(M+1)⁺; |
| | 1H-[1,2,3]triazole-4-carboxylic acid (2,4- | R_{f}=0.40(10% MeOH/CHCl₃) |
| | dichloro-phenyl)-(2-pyrrolidin-1-yl-ethyl)-amide | |
| 415 | 1-(3,5-bis-trifluoromethyl-benzyl)-5-chloro-1H- | MS(ES) 572.0(M+1)⁺; |
| | [1,2,3]triazole-4-carboxylic acid (2-chloro-4- | R_{f}=0.50(10% MeOH/CHCl₃) |
| | fluoro-phenyl)-(2-dimethylamino-ethyl)-amide | |
| 416 | 1-(3,5-bis-trifluoromethyl-benzyl)-5-chloro-1H- | MS(ES) 598.0(M+1)⁺; |
| | [1,2,3]triazole-4-carboxylic acid (2-chloro-4- | R_{f}=0.50(10% MeOH/CHCl₃) |
| | fluoro-phenyl)-(2-pyrrolidin-1-yl-ethyl)-amide | |
| 417 | 1-(3,5-bis-trifluoromethyl-benzyl)-5-chloro-1H- | MS(ES) 555.1(M+1)⁺ |
| | [1,2,3]triazole-4-carboxylic acid (2-chloro- | R_{f}=0.40(10% MeOH/CHCl₃) |
| | phenyl)-(2-dimethylamino-ethyl)-amide | |
| 418 | 1-(3,5-bis-trifluoromethyl-benzyl)-5-pyridin-4-yl- | MS(ES) 570.0(M+1)⁺. |
| | 1H-[1,2,3]triazole-4-carboxylic acid (2-chloro- | |
| | phenyl)-(2-hydroxy-ethyl)-amide | |
| 419 | (R)-1-(3,5-bis-trifluoromethyl-benzyl)-5-(2H- | MS(ES) 651.1(M+1)⁺; |
| | pyrazin-1-yl)-1H-[1,2,3]triazole-4-carboxylic acid | R_{f}=0.23(10% MeOH/CHCl₃) |
| | [1-(2-chloro-phenyl)-ethyl]-(2-pyrrolidin-1-yl- | |
| | ethyl)-amide | |
| 420 | 1-(3,5-bis-trifluoromethyl-benzyl)-5-pyridin-3-yl- | MS(ES) 596.2(M+1)⁺ |
| | 1H-[1,2,3]triazole-4-carboxylic acid [1-(2-chloro- | R_{f}=0.48(5% MeOH/CHCl₃) |
| | phenyl)-ethyl]-isopropyl-amide | |
| 421 | 1-(3,5-bis-trifluoromethyl-benzyl)-5-pyridin-4-yl- | MS(ES) 596.1(M+1)⁺ |
| | 1H-[1,2,3]triazole-4-carboxylic acid [1-(2-chloro- | R_{f}=0.50(5% MeOH/CHCl₃) |
| | phenyl)-ethyl]-isopropyl-amide | |

### EXAMPLE 422

### 1-(3,5-bis-trifluoromethyl-benzyl)-5-(1-oxo-1-λ⁴-thiomorpholin-4-yl)-1H-[1,2,3]triazole-4-carboxylic acid (2-chloro-phenyl)-methyl-amide

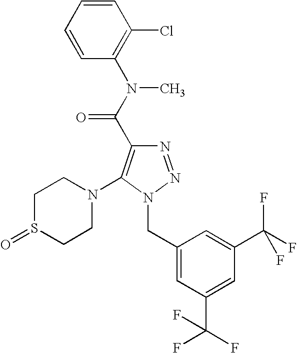

Add m-chloroperbenzoic acid (40 mg, 0.176 mmol) to a solution of 1-(3,5-bis-trifluoromethyl-benzyl)-5-thiomorpholin-4-yl-1H-[1,2,3]triazole-4-carboxylic acid (2-chloro-phenyl)-methyl-amide (90 mg, 0.16 mmol) in CH₂Cl₂ (5 mL) at −78° C. After 30 min, quench with saturated K₂CO₃. Wash the organic layer with saturated aqueous NaHCO₃, and brine, dry over sodium sulfate, filter, and concentrate. Purify by radial chromatography using a MeOH/CHCl₃ gradient to afford 75 mg (81%) of the title compound as a white foam. MS(ES) 580.0 (M+1); R_{f}=0.34 (10% MeOH/CHCl₃).

### EXAMPLE 423

### 1-(3,5-bis-trifluoromethyl-benzyl)-5-( 1,1-dioxo-1λ⁶-thiomorpholin-4-yl)-1H-[1,2,3]triazole4-carboxylic acid (2-chloro-phenyl)-methyl-amide

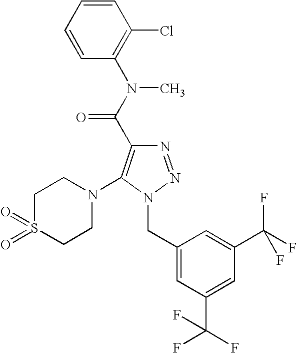

Add m-chloroperbenzoic acid (93 mg, 0.4 mmol) to a solution of 1-(3,5-bis-trifluoromethyl-benzyl)-5-thiomorpholin-4-yl-1H-[1,2,3]triazole-4-carboxylic acid (2-chloro-phenyl)-methyl-amide (90 mg, 0.16 mmol) in CH₂Cl₂ (5 mL) at 0° C. After 30 min, quench with saturated K₂CO₃. Wash the organic layer with saturated aqueous NaHCO₃, and brine. Dry over sodium sulfate, filter, and concentrate. Purify by radial chromatography using a MeOH/CHCl₃ gradient to afford 53.1 mg (56%) of the title compound as a white foam. MS(ES) 596.0 (M+1); R_{f}=0.54 (I 0% MeOH/CHCl₃).

### EXAMPLE 424

### 5-(4-acetyl-piperazin-1-yl)-1-(3,5-bis-trifluoromethyl-benzyl)-1H-[1,2,3]triazole-4-carboxylic acid (2-chloro-phenyl)-methyl-amide

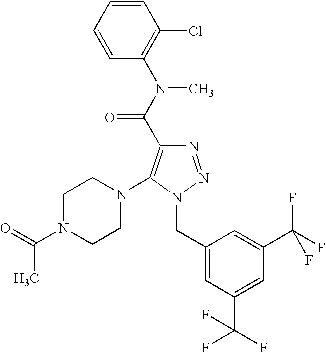

Dissolve 1-(3,5-bis-trifluoromethyl-benzyl)-5-piperazin-1-yl-1H-[1,2,3]triazole-4-carboxylic acid (2-chloro-phenyl)-methyl-amide (100 mg, 0.18 mmol) in CH₂Cl₂ (5 mL). Add TEA (0.1 mL, 0.54 mmol), acetic anhydride (0.019 mL, 0.2 mmol) and DMAP (5 mg). Stir overnight at RT, then add water. Wash with saturated aqueous NaHCO₃, and brine. Dry over sodium sulfate, filter, and concentrate. Purify by radial chromatography using a MeOH/CHCl3 gradient afford 97 mg (92%) of the title compound as a tan foam. MS(ES) 589.1 (M+1); R_{f}=0.58 (10% MeOH/CHCl₃).

### EXAMPLE 425

### 1-(3,5-bis-trifluoromethyl-benzyl)-5-morpholin-4-yl-1H-[1,2,3]triazole-4-carboxylic acid (2-chloro-phenyl)-methyl-amide

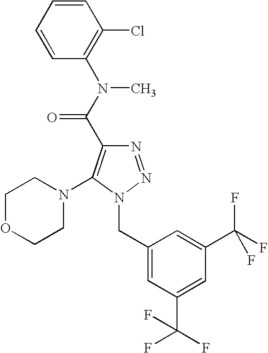

Dissolve 1-(3,5-bis-trifluoromethyl-benzyl)-5-chloro-1H-[1,2,3]triazole-4-carboxylic acid (2-chloro-phenyl)-methyl-amide (200 mg, 0.4 mmol) in warm morpholine (5 mL). Heat overnight at 100° C. in a sealed tube, then concentrate to dryness. Dissolve the residue in 20% iPrOH/CHCl₃. Wash with saturated aqueous NaHCO₃ and brine, dry over sodium sulfate, filter, and concentrate. Purify the residue via radial chromatography using an ethyl acetate/hexanes gradient to afford 155 mg (70%) of the title compound. MS(ES) 548.2 (M+1); R_{f}=0.41 (50% EtOAc/hexanes).

Using a similar method and the appropriate starting materials, the following compounds may be prepared and isolated.

| Ex. # | Product | Data |
|---|---|---|
| 426 | 1-(3,5-bis-trifluoromethyl-benzyl)-5-thiomorpholin-4-yl- | MS(ES) 564.4(M+1); |
| | 1H-[1,2,3]triazole-4-carboxylic acid (2-chloro-phenyl)- | R_{f}=0.63(1:1 |
| | methyl-amide | EtOAc/hex) |
| 427 | 1-(3,5-bis-trifluoromethyl-benzyl)-5-piperazin-1-yl-1H- | MS(ES) 547.1(M+1); |
| | [1,2,3]triazole-4-carboxylic acid (2-chloro-phenyl)- | R_{f}=0.38(20% |
| | methyl-amide | MeOH/CHCl₃) |
| 428 | 1-(3,5-bis-trifluoromethyl-benzyl)-5-dimethylamino-1H- | MS(ES) 506.1(M+1); |
| | [1,2,3]triazole-4-carboxylic acid (2-chloro-phenyl)- | R_{f}=0.57(1:1 |
| | methyl-amide | EtOAc/hexanes) |
| 429 | 1-(3,5-bis-trifluoromethyl-benzyl)-5-(4-methyl-piperazin- | MS(ES) 561.3(M+1); |
| | 1-yl)-1H-[1,2,3]triazole-4-carboxylic acid (2-chloro- | R_{f}=0.38(10% |
| | phenyl)-methyl-amide | MeOH/CHCl₃) |
| 430 | 1-(3,5-bis-trifluoromethyl-benzyl)-5-morpholin-4-yl-1H- | MS(ES) 582.0(M+1); |
| | [1,2,3]triazole-4-carboxylic acid (2,4-dichloro-phenyl)- | R_{f}=0.47(5% |
| | methyl-amide | MeOH/CHCl₃) |
| 431 | 1-(3,5-bis-trifluoromethyl-benzyl)-5-morpholin-4-yl-1H- | MS(ES) 566.0(M+1)⁺; |
| | [1,2,3]triazole-4-carboxylic acid (2-chloro-4-flouro- | R_{f}=0.61(5% |
| | phenyl)-methyl-amide | MeOH/CHCl₃) |
| 432 | 1-(3,5-bis-trifluoromethyl-benzyl)-5-morpholin-4-yl-1H- | MS(ES) 562.0(M+1); |
| | [1,2,3]triazole-4-carboxylic acid (2-chloro-4-methyl- | R_{f}=0.54(5% |
| | phenyl)-methyl-amide | MeOH/CHCl₃) |
| 433 | 1-(3,5-bis-trifluoromethyl-benzyl)-5-morpholin-4-yl-1H- | MS(ES) 608.1(M+1); |
| | [1,2,3]triazole-4-carboxylic acid (2-chloro-4-fluoro- | R_{f}=0.68(1:1 |
| | benzyl)-isopropyl-amide | EtOAc/hexanes) |
| 434 | 1-(3,5-bis-trifluoromethyl-benzyl)-5-morpholin-4-yl-1H- | MS(ES) 631.2(M+1); |
| | [1,2,3]triazole-4-carboxylic acid (2-chloro-phenyl)-(2- | R_{f}=0.76(20% |
| | pyrrolidin-1-yl-ethyl)-amide | MeOH/CHCl₃) |
| 435 | (R,S)-(2-{[1-(3,5-bis-trifluoromethyl-benzyl)-5- | MS(ES) 705.0(M+1); |
| | morpholin-4-yl-1H-[1,2,3]triazole-4-carbonyl]-[1-(2- | R_{f}=0.50(1:1 |
| | chloro-phenyl)-ethyl]-amino}-ethyl)-carbamic acid tert- | EtOAc/hexanes) |
| | butyl ester | |
| 436 | {2-[[1-(3,5-bis-trifluoromethyl-benzyl)-5-morpholin-4-yl- | MS(ES) 691.1(M+1); |
| | 1H-[1,2,3]triazole-4-carbonyl]-(2-chloro-benzyl)-amino]- | R_{f}=0.40(1:1 |
| | ethyl}-carbamic acid tert-butyl ester | EtOAc/hexanes) |
| 437 | {2-[[1-(3,5-bis-trifluoromethyl-benzyl)-5-methylamino- | MS(ES) 635.1(M+1); |
| | 1H-[1,2,3]triazole-4-carbonyl]-(2-chloro-benzyl)-amino]- | R_{f}=0.73(1:1 |
| | ethyl}-carbamic acid tert-butyl ester | EtOAc/hexanes) |
| 438 | {2-[[1-(3,5-bis-trifluoromethyl-benzyl)-5-dimethylamino- | MS(ES) 649.0(M+1); |
| | 1H-[1,2,3]triazole-4-carbonyl]-(2-chloro-benzyl)-amino]- | R_{f}=0.65(1:1 |
| | ethyl}-carbamic acid tert-butyl ester | EtOAc/hexanes) |
| 439 | {2-[[1-(3,5-bis-trifluoromethyl-benzyl)-5-morpholin-4-yl- | MS(ES) 709.1(M+1); |
| | 1H-[1,2,3]triazole-4-carbonyl]-(2-chloro-4-fluoro- | R_{f}=0.51(1:1 |
| | benzyl)-amino]-ethyl}-carbamic acid tert-butyl ester | EtOAc/hexanes) |
| 440 | 1-(3,5-bis-trifluoromethyl-benzyl)-5-morpholin-4-yl-1H- | MS(ES) 639.0(M+1); |
| | [1,2,3]triazole-4-carboxylic acid (2-chloro-benzyl)- | R_{f}=0.50(10% |
| | pyridin-4-ylmethyl-amide | MeOH/CHCl₃) |
| 441 | 1-(3,5-Bis-trifluoromethyl-benzyl)-5-morpholin-4-yl-1H- | MS(IS) 606.0(M+); |
| | [1,2,3]triazole-4-carboxylic acid (2-chloro- | TLC R_{f}=0.44(1:1 |
| | benzyl)-(2-methoxy-ethyl)-amide | EtOAc/hexanes) |

### EXAMPLE 442

### 1-(3,5-bis-trifluoromethyl-benzyl)-5-(4-methanesulfonyl-piperazin-1-yl)-1H-[1,2,3]triazole-4-carboxylic acid (2-chloro-phenyl)-methyl-amide

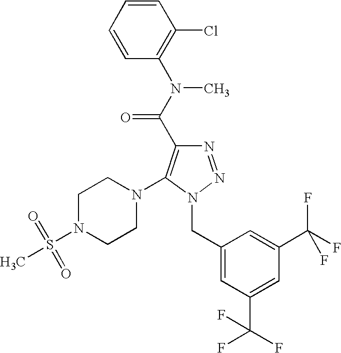

Dissolve 1-(3,5-bis-trifluoromethyl-benzyl)-5-piperazin-1-yl-1H-[1,2,3]triazole-4-carboxylic acid (2-chloro-phenyl)-methyl-amide (90 mg, 0.16 mmol) in CH₂Cl₂ (4 mL). Add TEA (0.1 mL, 0.48 mmol), methanesulfonyl chloride (0.014 mL, 0.176 mmol) and DMAP (5 mg). Stir overnight at RT, then add water. Extract with 20% iPrOH/CHCl₃. Wash with saturated aqueous NaHCO₃ and brine, dry over sodium sulfate, filter, and concentrate. Purify by radial chromatography using a MeOH/CHCl₃ gradient to afford 87 mg (87%) of the title compound as a tan foam. MS(ES) 625.0 (M+1)⁺; R_{f}=0.71 (10% MeOH/CHCl₃).

Using an analogous procedure and the appropriate starting materials, the following compounds may be prepared and isolated. Stereoisomers can be separated from the corresponding racemates via chiral chromatography.

| Ex. # | Product | Data |
|---|---|---|
| 443 | (R,S)-1-(3,5-bis-trifluoromethyl-benzyl)- | MS(ES) 674.9(M+1); |
| | 5-pyridin-4-yl-1H-[1,2,3]triazole- | R_{f}=0.30(10% |
| | 4-carboxylic acid [1-(2-chloro- | MeOH/CHCl₃) |
| | phenyl)-ethyl]-(2- | |
| | methanesulfonylamino-ethyl)-amide | |
| 444 | 1-(3,5-bis-trifluoromethyl-benzyl)- | MS(ES) 678.8(M+1). |
| | 5-(2H-pyrazin-1-yl)-1H-[1,2,3]triazole- | |
| | 4-carboxylic acid (2-chloro-4-fluoro- | |
| | benzyl)-(2-methanesulfonylamino- | |
| | ethyl)-amide | |
| 445 | 1-(3,5-bis-trifluoromethyl-benzyl)- | MS(ES) 688.9(M+1); |
| | 5-morpholin-4-yl-1H-[1,2,3]triazole- | R_{f}=0.50(10% |
| | 4-carboxylic acid (2-chloro-4-fluoro- | MeOH/CHCl₃) |
| | benzyl)-(2-methanesulfonylamino- | |
| | ethyl)-amide | |

### EXAMPLE 446

### (R,S)-(2-{[1-(3,5-bis-trifluoromethyl-benzyl)-5-pyridin-4-yl1H-[1,2,3]triazole-4-carbonyl]-[1-(2-chloro-phenyl)-ethyl]-amino-}-ethyl)-carbamic acid tert-butyl ester

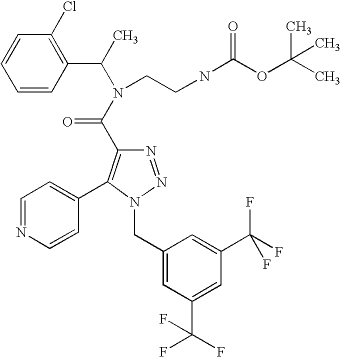

Dissolve 1-(3,5-bis-trifluoromethyl-benzyl)-5-pyrazin-2-yl-1H-[1,2,3]triazole-4-carboxylic acid (0.6 g, 1.4 mmol) in DMF (10 mL). Add (R,S)-{2-[1-(2-chloro-phenyl)-ethylamino]-ethyl}-carbamic acid tert-butyl ester (628 mg, 2.1 mmol), HOAt (208 mg, 1.5 mmol) EDCI (300 mg, 1.5 mmol), DMAP (5 mg) and TEA (0.22 mL, 1.5 mmol) in 10 mL of DMF and stir at RT. After 16 h, concentrate the mixture and dissolve the residue in 20% iPrOH/CHCl₃. Wash with saturated aqueous NaHCO₃ and brine, dry over sodium sulfate, filter, and concentrate. Purify the residue by column chromatography using a methanol/chloroform gradient to afford 718 mg (74%) of the title compound as a tan oil. MS(ES) 697.2 (M+1)⁺; R_{f}=0.40 (10% MeOH/CHCl₃).

Using a similar method and the appropriate starting materials, the following compounds may be prepared and isolated.

| Ex. # | Product | Data |
|---|---|---|
| 447 | (R,S)-(2-{[1-(3,5-bis-trifluoromethyl-benzyl)-5-chloro- | MS(ES) 654.0(M+1); |
| | 1H-[1,2,3]triazole-4-carbonyl]-[1-(2-chloro-phenyl)- | R_{f}=0.60(1:1 |
| | ethyl]-amino}-ethyl)-carbamic acid tert-butyl ester | EtOAc/hexanes) |
| 448 | {2-[[1-(3,5-bis-trifluoromethyl-benzyl)-5-pyridin-4-yl- | MS(ES) 683.06(M+1); |
| | 1H-[1,2,3]triazole-4-carbonyl]-(2-chloro-benzyl)- | Rf=0.29(10% |
| | amino]-ethyl}-carbamic acid tert-butyl ester | MeOH/CHCl₃) |
| 449 | {2[[1-(3,5-bis-trifluoromethyl-benzyl)-5-chloro-1H- | MS(ES) 640.0(M+1); |
| | [1,2,3]triazole-4-carbonyl]-(2-chloro-benzyl)-amino]- | Rf=0.60(1:1 |
| | ethyl}-carbamic acid tert-butyl ester | EtOAc/hexanes) |
| 450 | {2-[[1-(3,5-bis-trifluoromethyl-benzyl)-5-pyridin-4-yl- | MS(ES) 701.1(M+1); |
| | 1H-[1,2,3]triazole-4-carbonyl]-(2-chloro-4-fluoro- | Rf=0.37(10% |
| | benzyl)-amino]-ethyl}-carbamic acid tert-butyl ester | MeOH/CHCl₃) |
| 451 | {2-[[1-(3,5-bis-trifluoromethyl-benzyl)-5-chloro-1H- | MS(ES) 658.0(M+1); |
| | [1,2,3]triazole-4-carbonyl]-(2-chloro-4-fluoro-benzyl)- | Rf=0.61(1:1 |
| | amino]-ethyl}-carbamic acid tert-butyl ester | EtOAc/hexanes) |
| 452 | 1-(3,5-bis-trifluoromethyl-benzyl)-5-pyridin-4-yl-1H- | MS(ES) 630.9(M+1); |
| | [1,2,3]triazole-4-carboxylic acid (2-chloro-benzyl)- | Rf=0.75(20% |
| | pyridin-4-ylmethyl-amide | MeOH/CHCl₃) |
| 453 | 1-(3,5-bis-trifluoromethyl-benzyl)-5-chloro-1H- | MS(ES) 587.9(M+1); |
| | [1,2,3]triazole-4-carboxylic acid (2-chloro-benzyl)- | Rf=0.62(10% |
| | pyridin-4-ylmethyl-amide | MeOH/CHCl₃) |
| 454 | 1-(3,5-bis-trifluoromethyl-benzyl)-5-pyridin-4-yl-1H- | MS(ES) 597.9(M+1); |
| | [1,2,3]triazole-4-carboxylic acid (2-chloro-benzyl)-(2- | Rf=0.60(10% |
| | methoxy-ethyl)-amide | MeOH/CHCl₃) |

### EXAMPLE 455

### (R,S)-1-(3,5-bis-trifluoromethyl-benzyl)-5-pyridyl-4-yl-1H-[1,2,3]triazole-4-carboxylic acid (2-amino-ethyl)-[1-(2-chloro-phenyl)-ethyl]-amide dihydrochloride

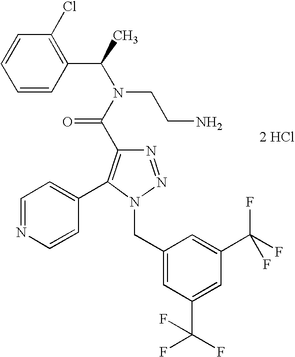

Dissolve (2-{[1-(3,5-bis-trifluoromethyl-benzyl)-5-pyridin-4-yl-1H-[1,2,3]triazole-4-carbonyl]-[1-(2-chloro-phenyl)-ethyl]-amino}-ethyl)-carbamic acid tert-butyl ester (1.07 g, 1.53 mmol) in HCl-saturated acetic acid (20 mL). Stir for 3 h at RT, then concentrate to dryness. Dissolve in CH₃CN and concentrate to dryness. Dry under vacuum to afford 1.02 g (100%) of the title compound as a white foam. MS(ES) 598.1 (M+1)⁺; Anal. Calc'd for C₂₇H₂₃ClF₆N₆O22HCl: C, 47.89;H, 3.75; N, 12.41. Found: C, 47.61;H, 3.81; N, 12.20.

Using a method analogous to the above method, with the appropriate starting materials, the following compounds may be prepared and isolated. Stereoisomers can be separated from the corresponding racemates via chiral chromatography.

| Ex. # | Product | Data |
|---|---|---|
| 456 | (R,S)-1-(3,5-bis-trifluoromethyl-benzyl)-5- | MS(ES) 605.2(M+1); Anal. |
| | morpholin-4-yl-1H-[1,2,3]triazole-4-carboxylic | Calc'd for |
| | acid (2-amino-ethyl)-[1-(2-chloro-phenyl)- | C₂₆H₂₇ClF₆N₆O₂.2.5HCl: C, 44.86; |
| | ethyl]-amide dihydrochloride | H, 4.27; N, 12.07. Found: C, |
| | | 44.82; H, 4.51; N, 11.60. |
| 457 | 1-(3,5-bis-trifluoromethyl-benzyl)-5-pyridin-4- | MS(ES) 583.1(M+1); Anal Calcd |
| | yl-1H-[1,2,3]triazole-4-carboxylic acid (2- | for C₂₆H₂₁ClF₆N₆0.2HCl: C, |
| | amino-ethyl)-(2-chloro-benzyl)-amide | 47.61; H, 3.53; N, 12.81. Found: |
| | dihydrochloride | C, 47.25; H, 3.42; N, 12.44. |
| 458 | 1-(3,5-bis-trifluoromethyl-benzyl)-5-pyridin-4- | MS(ES) 601.1(M+1); Anal Calcd |
| | yl-1H-[1,2,3]triazole-4-carboxylic acid (2- | for C₂₆H₂₀ClF₇N₆O.2HCl: C, |
| | amino-ethyl)-(2-chloro-4-fluoro-benzyl)-amide | 46.34; H, 3.29; N, 12.47. Found: |
| | dihydrochloride | C, 46.40; H, 3.65; N, 11.80. |
| 459 | 1-(3,5-bis-trifluoromethyl-benzyl)-5-morpholin- | MS(ES) 609.0(M+1); Anal. |
| | 4-yl-1H-[1,2,3]triazole-4-carboxylic acid (2- | Calc'd for C₂₅H₂₄ClF₇N₆O₂.HCl: |
| | amino-ethyl)-(2-chloro-4-fluoro-benzyl)-amide | C, 46.52; H, 3.90; N, 13.02. |
| | hydrochloride | Found: C, 46.50; H, 4.11; N, |
| | | 12.62 |
| 460 | 1-(3,5-bis-trifluoromethyl-benzyl)-5-morpholin- | MS(IS) 591.1(M+1); Anal. |
| | 4-yl-1H-[1,2,3]triazole-4-carboxylic acid (2- | Calc'd for C₂₅H₂₅ClF₆N₆O₂.HCl: |
| | amino-ethyl)-(2-chloro-benzyl-amide | C, 47.86; H, 4.18; N, 13.39. |
| | hydrochloride | Found: C, 47.71; H, 4.27; 13.06. |
| 461 | 1-(3,5-bis-trifluoromethyl-benzyl)-5- | MS(IS) 534.9(M+1); |
| | methylamino-1H-[1,2,3]triazole-4-carboxylic | |
| | acid (2-amino-ethyl)-(2-chloro-benzyl)-amide | |
| | hydrochloride | |
| 462 | 1-(3,5-bis-trifluoromethyl-benzyl)-5- | MS(IS) 548.9(M+1); Anal. |
| | dimethylamino-1H-[1,2,3]triazole-4-carboxylic | Calc'd for C₂₃H₂₃ClF₆N₆O.1.1HCl: |
| | acid (2-amino-ethyl)-(2-chloro-benzyl)-amide | C, 46.90; H, 4.12; N, 14.27. |
| | hydrochloride | Found: C, 46.76; H, 4.00; N, |
| | | 13.78. |

### EXAMPLE 463

### N-[1-(3,5-bis-trifluoromethyl-benzyl)-5-(2H-pyrazin-1-yl)-1H-[1,2,3]triazole-4-carbonyl]-2-chloro-N-methyl-benzenesulfonamide

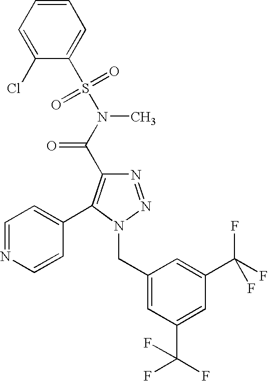

Dissolve 1-(3,5-Bis-trifluoromethyl-benzyl)-5-pyridin-3-yl-1H-[1,2,3]triazole4-carboxylic acid (300 mg.; 1.0 eq.) in CH₂Cl₂ (5 mL). Add 2-chloro-N-methylbenzenesulfonamide (178 mg., 1.0 eq.), DMAP (90 mg.; 1.0 eq.) and EDCI (280 mg, 1.0 eq.). Stir overnight at RT, then dilute with CH₂Cl₂ (10 mL) and wash with saturated aqueous NaHCO₃, and brine. Dry the organic layer over sodium sulfate, filter, and concentrate to dryness. Purify by chromatography. MS(ES) 603.9 (M+1)⁺; R_{f}=0.57 (10% MeOH/CHCl₃).

### EXAMPLE 464

### N-[1-(3,5-bis-trifluoromethyl-benzyl)-5-pyridin-4-yl-1H-[1,2,3]triazole-4-carbonyl)-2-chloro-N-methyl-benzamide

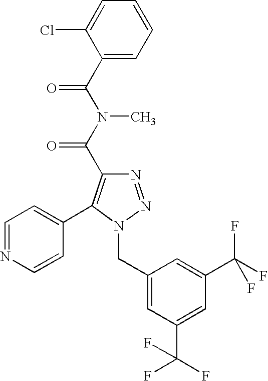

Dissolve 1-(3,5-bis-trifluoromethyl-benzyl)-5-pyridin-3-yl-1H-[1,2,3]triazole-4-carboxylic acid (600 mg., 1.0 eq.) in CH₂Cl₂ (10 mL) and DMF (I drop). Add oxalyl chloride (0.3 mL, 2.0 eq.) and stir for 2 hours at RT. Concentrate the mixture and slurry the residue in 1,2-dichloroethane and concentrate to dryness again. Dissolve in DMF and cool to 0° C. Separately, add 2-chloro-N-methyl-benzamide (250 mg., 1.0 eq.) to a slurry of NaH (70 mg, 1.2 eq.) in DMF at 0° C. Add the NaH mixture to the acid chloride solution. Stir 10 minutes, then remove the ice bath and stir overnight at RT. Concentrate the mixture in vacuo and dissolve the residue in 20% iPrOH/CHCl₃. Wash with saturated aqueous NaHCO₃, and brine, dry over Na₂SO₄, filter, and concentrate. Purify the residue by reverse phase chromatography. MS(ES) 567.9 (M+1); Rf=0.66 (10% MeOH/CHCl₃).

### EXAMPLE 465

### [1-(3,5-Bis-trifluoromethyl-benzyl)-5-morpholin-4-yl-1H-[1,2,3]triazol-4-yl]-[3-(2-chloro-phenyl)-piperidin-1-yl]-methanone

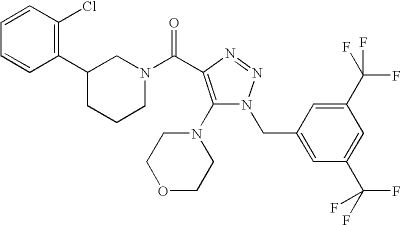

Dissolve [1-(3,5-Bis-trifluoromethyl-benzyl)-5-chloro-1H-[1,2,3 ]triazol-4-yl]-[3-(2-chloro-phenyl)-piperidin-1-yl]-methanone (60 mg, 0.11 mmol) in morpholine (1.2 mL) and heat solution at 100° C. in a sealed tube for 12 h. Concentrate the mixture and purify the residue by chromatography using a gradient of 10:1 to 1:5Hex/EtOAc to afford the title compound (46 mg, 70%). MS(ES) 602.5 (M+1).

### EXAMPLE 466

### [1-(3,5-Bis-trifluoromethyl-benzyl)-5-dimethylamino-1H-[1,2,3]triazol-4-yl]-[3-(2-chloro-phenyl)-piperidin-1-yl]-methanone

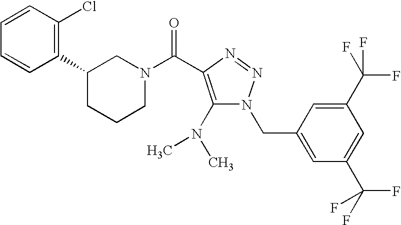

Add dimethyamine (1 ml, 2.0 M in THF) to [1-(3,5-Bis-trifluoromethyl-benzyl)-5-chloro-1H-[1,2,3]triazol-4-yl]-[3-(2-chloro-phenyl)-piperidin-1-yl]-methanone (60 mg, 0.11 mmol) and heat to 100° C. in a sealed tube for 12 h. Cool reaction to RT, add more dimethyamine (1 ml, 2.0 M in THF), and again heat to 100° C. After 12 h, add a third aliquot of dimethylamine (1 ml, 2.0 M in THF) and heat to 100° C. for another 12 h. Then 2 0 concentrate the mixture and purify the residue by chromatography using a gradient of 10:1 to 1:5Hex/EtOAc to afford title compound (23.6 mg, 38%). MS(ES) 560.1 (M+1); Rf=0.22 (2:1Hex/EtOAc).

### EXAMPLE 467

### [1-(3,5-Bis-trifluoromethyl-benzyl)-5-pyridin-4-yl-1H-[1,2,3]triazol-4-yl]-[3-(2-chloro-phenyl)-piperidin-1-yl]-methanone

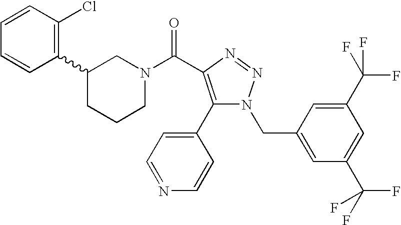

To a solution of 1-(3,5-Bis-trifluoromethyl-benzyl)-5-pyridin-4-yl-1H-[1,2,3]triazole-4-carboxylic acid (50 mg, 0.12 mmol) and HOBt (85 mg, 0.36 mmol) in CH₂Cl₂ (1 mL) add 3-(2-chloro-phenyl)-piperidine (33.4 mg, 0.17 mmol) and stir at RT. To this solution add TEA (83.5 μL, 0.60 mmol) and EDCI (69 mg, 0.36 mmol). Stir at RT for 24 h, then dilute the solution with CH₂Cl₂ (1 mL), and wash with 1N HCl (2×1.5 mL). Wash the organic layer with 1N NaOH (2×1.5 mL), saturated NaHCO₃ (1 mL) and brine (1 mL). Dry, filter and concentrate. Purify the residue by chromatography using a gradient of 10:1 to 1:5Hex/EtOAc to afford title compound (49.7 mg, 70%). MS (ES) 594.1 (M+1)⁺; Rf=0.41 (1:5Hex/EtOAc).

### EXAMPLE 468

### [1-(3,5-Bis-trifluoromethyl-benzyl)-5-pyridin-4-yl-1H-[1,2,3]triazol-4-yl]-[cis-2-(2-chloro-phenyl)-3-hydroxy-pyrrolidin-1-yl]-methanone

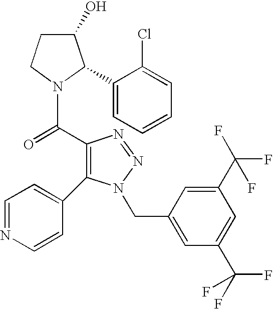

Treat acetic acid cis-2-(2-chloro-phenyl)-pyrrolidin-3-yl ester (615 mg, 2.57 mmol) and 1-(3,5-bis-trifluoromethyl-benzyl)-5-pyridin-4-yl-1H-[1,2,3]triazole-4-carboxylic acid hydrochloride (1.16 g, 2.57 mmol) in 20 mL of DMF with EDCI (591 mg, 3.08 mmol), HOBt (417 mg, 3.08 mmol) and a catalytic amount of DMAP. Stir at RT for 20 h, then dilute with saturated aqueous NaHCO₃ and extract with EtOAc (100 mL).

Wash the organic layer with brine, then dry over MgSO₄, filter, and concentrate. Purify by chromatography using 1% MeOH in dichloromethane to provide the acetate intermediate (acetic acid 1-[1-(3,5-bis-trifluoromethyl-benzyl)-5-pyridin-4-yl-1H-[1,2,3]triazole-4-carbonyl]-2-(2-chloro-phenyl)-pyrrolidin-3-yl ester. Dilute this material with a mix of dioxane and water (20 mL:5 mL) and add LiOH.H₂O (502 mg, 12 mmol). Stir at RT for 72 h, then concentrate in vacuo. Partition the residue between EtOAc and H₂O (75 mL each). Wash the organic layer with saturated aqueous NaHCO₃ and brine (75 mL each) and dry over Na₂SO₄, then filter and concentrate. Purify by chromatography using 1% MeOH in dichloromethane doped with a solution of 25% NH₄OH to give the title compound as an off-white solid (830 mg, 54% over 2 steps). ¹H NMR (CDCl₃, 400 MHz): δ 2.04-2.28 (m, 2H), 3.88-4.03 (m, 1H), 4.21-4.26 (m, 0.5H), 4.45-4.52 (m, 0.5H), 4.75-4.80 (m, 1H), 5.34 (AB q, J=16 Hz, Δv=48 Hz, 1H), 5.54 (AB q, J=16 Hz, Δv=23 Hz, 1H), 5.62 (d, J=5.2 Hz, 0.5H), 6.41 (d, J=5.6 Hz, 0.5H), 6.95-7.04 (m, 2.5H), 7.17-7.31 (m, 3H), 7.35-7.37 (m, 1.5H), 7.51 (s, 1H), 7.82 (s, 0.5H), 7.85 (s, 0.5H), 8.7 (s, 2H), MS(ES) 596.17 (M+1).

### EXAMPLE 469

### [1-(3,5-Bis-trifluoromethyl-benzyl)-5-pyridin-4-yl-1H-[1,2,3]triazol-4-yl]-[trans-2-(2-chloro-phenyl)-3-hydroxy-pyrrolidin-1-yl]-methanone

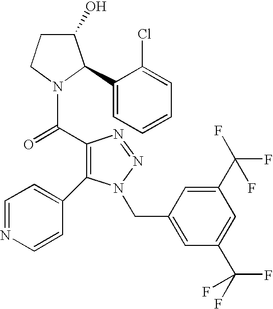

Treat [1-(3,5-bis-trifluoromethyl-benzyl)-5-pyridin-4-yl-1H-[1,2,3]triazol-4-yl]-[cis-2-(2-chloro-phenyl)-3-hydroxy-pyrrolidin-1-yl)-methanone (125 mg, 0.21 mmol) with 4-nitrobenzoic acid (141 mg, 0.84 mmol), DIAD (165 uL, 0.84 mmol) and triphenyl phosphine (221 mg, 0.84 mmol) in 3.1 mL of THF at 0° C. for 18 h. Dilute the mixture with EtOAc and wash two times with saturated aqueous NaHCO₃. Dry the organic layer over Na₂SO₄, filter and concentrate. Purify by chromatography using 2% MeOH in dichloromethane to provide the nitrobenzoate ester intermediate (4-nitro-benzoic acid 1-[1-(3,5-bis-trifluoromethyl-benzyl)-5-pyridin-4-yl-1H-[1,2,3]triazole4-carbonyl]-2-(2-chloro-phenyl)-pyrrolidin-3-yl ester). Dissolve this material in dioxane/water and add LiOH.H₂O (50 mg, 0.42 mmol). Stir at RT for 8 b, then concentrate and purify the residue by column chromatography using 30% EtOAc/hexanes to provide the title compound as an off-white foam (46 mg, 37% over 2 steps). ¹H NMR (CDCl₃, 400 MHz): δ 1.94-2.24 (m, 2H), 4.03 (dd, J=9.6, 5.6 Hz, 1H), 4.28 (ddd, J=11.6, 8, 8 Hz, 0.5H), 4.38 (s, 0.5H), 4.65 (s, 0.5H), 4.83 (t, J=9.2 Hz, 0.5H), 5.39 (s, 1H), 5.50-5.59 (m, 1.5H), 6.25 (s, 0.5H), 6.96 (d, J=7.6 Hz, 0.5H), 7.03 (d, J=5.6 Hz, 1H), 7.08-7.20 (m, 3.5H), 7.33-7.36 (m, 2H), 7.51 (s, 1H), 7.81 (s, 0.5H), 7.85 (s, 0.5H), 8.7 (s, 2H); MS(ES) 596.20 (M+1).

### EXAMPLE 470

### [1-(3,5-Bis-trifluoromethyl-benzyl)-5-pyridin-3-yl-1H-[1,2,3]triazol-4-yl]-[cis-2-(2-chloro-phenyl)-4-hydroxy-pyrrolidin-1-yl]-methanone

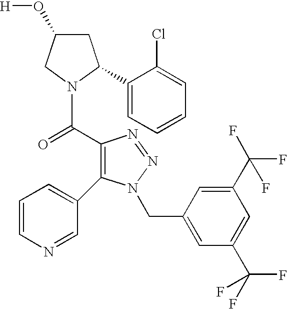

Dissolve cis-4-(tert-butyl-dimethyl-silanyloxy)-2-(2-chloro-phenyl)-pyrrolidine (150 mg, 0.48 mmol) and 1-(3,5-bis-trifluoromethyl-benzyl)-5-pyridin-4-yl-1H-[1,2,3]triazole-4-carboxylic acid hydrochloride (240 mg, 0.53 mmol) in 10 mL of dichloromethane and add EDCI (110 mg, 0.58 mmol), HOBt (78 mg, 0.58 mmol) and triethylamine (80 uL, 0.58 mmol). Stir the mixture at RT for 20 h, then dilute with saturated NaHCO₃ and extract with EtOAc(20 mL). Wash the organic layer with brine, dry, filter and concentrate. Dissolve the crude product, [1-(3,5-bis-trifluoromethyl-benzyl)-5-pyridin-3-yl-1H-[1,2,3]triazol-4-yl]-[4-(tert-butyl-dimethyl-silanyloxy)-2-(2-chloro-phenyl)-pyrrolidin-1-yl)-methanone (75 mg, 0.106 mmol), in THF (3 mL) and TBAF (120 uL of a 1M soln. in THF, 0.12 mmol). Stir the mixture for 1 h at RT, then dilute with EtOAc and wash with brine. Dry the organic layer over Na₂SO₄, filter and concentrate. Purify the residue by chromatography using 2% MeOH and 0.5% conc. NH₄OH in dichloromethane to give the title compound as a off-white foam (36 mg, 13% over 2 steps). ¹H NMR (CDCl₃, 400 MHz) δ 1.98 (ddd, J=12.8, 4.4, 4.4 Hz, 1H), 2.07-2.12 (m, 1H), 2.62 (ddd, J=14, 8.8, 5.6 Hz, 0.5H), 2.74 (ddd, J=14.4, 9.2, 6 Hz, 0.5H), 3.84 (d, J=12.4 Hz, 0.5H), 4.04 (dd, J=13.6, 5.6 Hz, 0.5H), 4.35 (dd, J=12.4, 5.2 Hz, 0.5H), 4.49 (d, J=12 Hz, 0.5H), 4.53-4.56 (m, 1H), 5.33 (s, 1H), 5.50-5.56 (m, 1.5H), 6.33 (dd, J=9.2, 3.6 Hz, 0.5H), 6.70-6.92 (m, 1H), 7.04-7.18 (m, 2H), 7.22-7.37 (m, 3H), 7.41 (s, 1H), 7.50 (d, J=7.6 Hz, 0.5H), 7.61 (d, J=8.5 Hz, 0.5H), 7.73 (s, 0.5H), 7.76 (s, 0.5H), 8.17 (s, 0.5H), 8.51 (s, 0.5H), 8.64 (s, 1H); R_{f}=0.46 (5% MeOH/CH₂Cl₂).

### EXAMPLE 471

### [1-(3,5-Bis-trifluoromethyl-benzyl)-5-pyridin-3-yl-1H-[1,2,3]triazol-4-yl]-[2-(2-chloro-phenyl)-4,4-difluoro-pyrrolidin-1-yl)-methanone

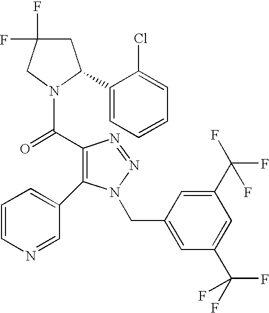

Dissolve [1-(3,5-bis-trifluoromethyl-benzyl)-5-pyridin-3-yl-1H-[1,2,3]triazol-4-yl]-[cis-2-(2-chloro-phenyl)-4-hydroxy-pyrrolidin-1-yl]-methanone (36 mg, 0.06 mmol) in dichloromethane (2.5 ml), chill to 0° C., and add Dess-Martin periodinane (31 mg, 0.073 mmol). Stir 12 h, allowing to warm to RT. Dilute with ethyl acetate (20 ml), wash with 5N aqueous sodium hydroxide (2×15 ml) and brine (20 ml). Dry organic phase over sodium sulfate, filter and concentrate. Chromatograph residue on silica gel (0.5% ammonium hydroxide/2% methanol/dichloromethane) [1-(3,5-bis-trifluoromethyl-benzyl)-5-pyridin-3-yl-1H-[1,2,3]triazol-4-yl]-[2-(2-chloro-phenyl)-4-oxo-pyrrolidin-1-yl]-methanone (30 mg, 80%). Dissolve this material in dichloromethane (2 ml) and add (diethylamino)sulfur trifluoride (50 μl, 0.38 mmol). Stir at RT for 12 h, then slowly add saturated aqueous sodium bicarbonate solution (5 ml). Extract with ethyl acetate (2×15 ml) and wash the organic phase with brine (10 ml). Dry over sodium sulfate, filter, and concentrate. Purify the residue by chromatography on silica gel (0.5% ammonium hydroxide/] % methanol/dichloromethane) to give the title compound as a light yellow solid (18 mg, 58%). MS(ES) 616.1 (M+1); ¹H NMR (CDCl₃, 400 MHz): δ 2.25-2.50 (m, 1H), 2.85-3.09 (m, 1H), 4.02-4.24 (m, 1H), 4.59 (dd, J=22.4, 12.4 Hz, 0.5H), 4.73 (dd, J=30, 14 Hz, 0.5H), 5.34 (s, 1H), 5.55 (AB q, J=15.6 Hz, Δv=16 Hz, 1H), 5.69 (dd, J=9.2, 6 Hz, 0.5H), 6.56 (dd, J=9.2, 4.4 Hz, 0.5H), 6.93-7.06 (m, 1.5H), 7.09-7.17 (m, 1.5H), 7.20-7.35 (m, 2.5H), 7.40-7.50 (m, 2H), 7.55 (dd, J=8 Hz, 1H), 7.30 (s, 0.5H), 7.76 (s, 1H), 8.17 (s, 0.5H), 8.51 (s, 0.5H), 8.65 (s, 1H).

### EXAMPLE 472

### [1-(3,5-Bis-trifluoromethyl-benzyl)-5-phenyl-1H-[1,2,3]triazol-4-yl]-[2-(2-chloro-phenyl)-pyrrol-1-yl]-methanone

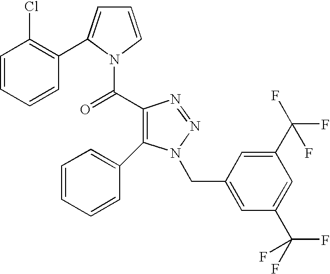

Suspend 1-(3,5-bis-trifluoromethyl-benzyl)-5-phenyl-1H-[1,2,3)triazole-4-carboxylic acid (1 g, 2.41 mmol) in dichloromethane (10 ml), add oxalyl chloride (2M in dichloromethane, 2.4 ml, 4.82 mmol) and two drops of dimethylformamide. Stir for 2 h, then remove solvent. Suspend the residue in dichloromethane (8 mL) and add the suspension to a solution of pyridine (1 ml, 12.4 mmol), 5-(2-chloro-phenyl)-3,4-dihydro-2H-pyrrole (865 mg, 4.82 mmol), and 4-dimethylaminopyridine (20 mg). Stir at RT. After 18 h, dilute with ethyl acetate (60 ml) and wash with 2N HCl (50 ml), brine (50 ml), and saturated aqueous NaHCO₃ (50 ml). Dry over sodium sulfate, filter, and concentrate. Dissolve residue in 1,4-dioxane and add 2,3-dichloro-5,6-dicyano-1,4-benzoquinone (600 mg, 2.64 mmol). Stir at RT for 18 h. Then remove the solvent and dissolve residue in ethyl acetate (60 ml). Wash with 1N NaOH (50 ml), and brine (50 ml). Dry over sodium sulfate, filter, and concentrate. Purify the residue by chromatography on silica gel (15% ethyl acetate/hexane) to give the title compound as a light purple solid (150 mg, 11% over 2 steps): ¹H NMR (CDCl₃, 400 MHz): δ 5.50 (s, 2H), 6.32 (dd, J=3.2, 1.6 Hz, 1H), 6.35 (t, J=3.6 Hz, 1H), 7.08-7.23 (m, 5H), 7.35 (dd, J=7.6, 1.6 Hz, 1H), 7.40-7.51 (m, 5H), 7.67 (dd, J=3.6, 1.6 Hz, 1H), 7.80 (s, 1H); MS(ES) 575.0 (M+1)⁺.

### EXAMPLE 473

### [1-(3,5-Bis-trifluoromethyl-benzyl)-5-pyridin-3-yl-1H-[1,2,3]triazol4-yl]-[2-(2-chloro-phenyl)-pyrrolidin-1-yl]-methanone

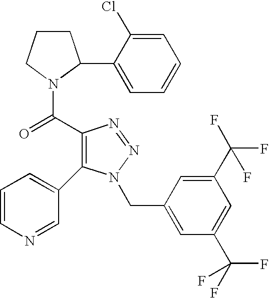

Treat a solution of 1-(3,5-bis-trifluoromethyl-benzyl)-5-pyridin-3-yl-1H-[1,2,3]triazole-4-carboxylic acid (0.20 g, 0.49 mmol) in CH₂Cl₂ (3.0 mL) with EDCI (0.20 g, 1.0 mmol), DMAP (0.13 g, 1.1 mmol) and (±)-2-(2-chloro-phenyl)-pyrrolidine (0.26 g, 0.95 mmol). Stir at RT overnight, then dilute with additional CH₂Cl₂ (20 mL) and wash with saturated NH₄Cl (10 mL), H₂O (10 mL), and saturated NaHCO₃ (10 mL). Dry, filter, and concentrate the organic solution, then purify by flash chromatography using a linear gradient of 70% EtOAc/hexanes to 100% EtOAc. Purify again by flash chromatography using a linear gradient of 100% CH₂Cl₂ to 10% MeOH/CH₂Cl₂ to give the title compound (0.17 g, 65%). MS (ES+) 580.3 (M+1)⁺; ¹H NMR (400 MHz, CDC13) δ 8.69 (m, 1H), 8.55 (m, 0.5H), 8.20 (m, 0.5H), 7.82 (s, 0.5H), 7.79 (s, 0.5H), 7.67 (m, 0.5H), 7.54 (m, 0.5H), 7.47 (m, 1H), 7.29-7.40 (m, 3H), 7.10-7.24 (m, 1.5H), 7.06 (m, 0.5H), 7.01 (m, 0.5H), 6.90 (m, 0.5H), 6.30 (m, 0.5H), 5.60 (m, 1.5H), 5.41 (m, 1H), 4.55 (m, 0.5H), 4.11 (m, 0.5H), 3.90 (m, 0.5H), 3.81 (m, 0.5H), 2.50 (m, 0.5H), 2.41 (m, 0.5H), 1.84-2.02 (m, 3.5H).

### EXAMPLE 474

### [1-(3,5-Bis-trifluoromethyl-benzyl)-5-pyridin-4-yl-1H-[1,2,3]triazol-4-yl]-[2-(2-chloro-phenyl)-pyrrolidin-1-yl]-methanone

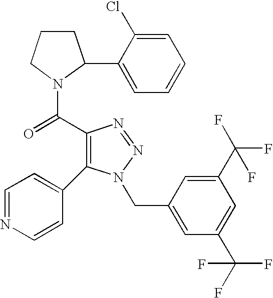

Using a method similar to that for [1-(3,5-Bis-trifluoromethyl-benzyl)-5-pyridin-3-yl-1H-[1,2,3]triazol-4-yl]-[2-(2-chloro-phenyl)-pyrrolidin-1-yl]-methanone, the title compound may be prepared. The racemate may be separated via chiral chromatography (Chiralcell OD 4.6mm×250mm, 20% isopropanol/heptane, 1 mL/min) to give (R)-[1-(3,5-Bis-trifluoromethyl-benzyl)-5-pyridin-4-yl-1H-[1,2,3 ]triazol-4-yl]-[2-(2-chloro-phenyl)-pyrrolidin-1-yl]-methanone. MS (ES+) 580.3 (M+1), MS (ES−) 578.5 (M−1). 1HNMR (400 MHz, CDCl3) δ 8.69 (s, 2H), 7.85 (s, 0.5H), 7.81 (s, 0.5H), 7.53 (s, 1H), 7.39 (s, 1H), 7.22-7.32 (m, 2H), 7.11-7.17 (m, 1.5H), 7.03 (m, 1.5H), 6.99 (m, 0.5H), 6.89 (m, 0.5H), 6.26 (m, 0.5H), 5.56-5.60 (m, 1.5H), 5.38 (m, 1H), 4.53 (m, 0.5H), 4.11 (m, 0.5H), 3.90 (m, 0.5H), 3.83 (m, 0.5H), 2.50 (m, 0.5H), 2.41 (m, 0.5H), 1.85-2.02 (m, 3.5H).

### EXAMPLE 475

### [1-(3,5-Bis-trifluoromethyl-benzyl)-5-(1-oxy-pyridin-4-yl)-1H-[1,2,3]triazol-4-yl]-[2-(2-chloro-phenyl)pyrrolidin-1-yl]-methanone

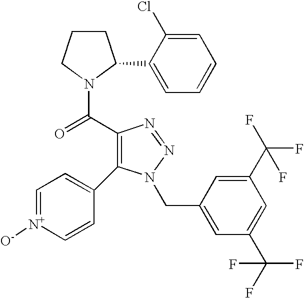

Treat a solution of [1-(3,5-bis-trifluoromethyl-benzyl)-5-pyridin-4-yl-1H-[1,2,3]triazol-4-yl]-[2-(2-chloro-phenyl)-pyrrolidin-1-yl]-methanone (81 mg, 0.14 mmol) in CH₂Cl₂ (1.5 mL) with mCPBA (52 mg, 0.30 mmol) and stir solution at RT overnight. Dilute solution with CH₂Cl₂ (20 mL) and wash with saturated aqueous NaHCO₃ (20 mL). Dry, filter, and concentrate the organic layer, and purify the crude material by flash chromatography by first eluting with 100% EtAc to remove unreacted starting material and then eluting with 10% MeOH/CH₂Cl₂ to give the title compound as a clear glass. Dissolve the solid in minimal amount of ether and precipitate with hexanes to give a white amorphous solid (66mg, 79%). MS(ES) 596.1 (M+1)⁺; ¹H NMR (400 MHz, CDCl₃, 1:1 mixture of amide rotamers) δ 8.16 (m, 2H), 7.85 (m, 1H), 7.59 (s, 1H), 7.45 (s, 1H), 7.32 (m, 0.5H), 7.20 (m, 1H), 7.17 (m, 2H), 7.00 (m, 1H), 6.96 (m, 1H), 6.87 (m, 0.5H), 6.22 (m, 0.5H), 5.57 (m, 0.5H), 5.56 (s, 1H), 5.37 (m, 1H), 4.52 (m, 0.5H), 4.08 (m, 0.5H), 3.87 (m, 1H), 2.44 (m, 1H), 1.98 (m, 2H), 1.89 (m, 1H).

### EXAMPLE 476

### [1-(3,5-Bis-trifluoromethyl-benzyl)-5-(1-oxy-pyridin-3-yl)-1H-[1,2,3]triazol-4-yl]-[2-(2-chloro-phenyl)-pyrrolidin-1-yl]-methanone

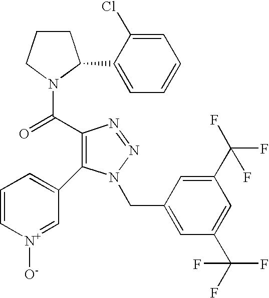

Treat a solution of [1-(3,5-bis-trifluoromethyl-benzyl)-5-pyridin-3-yl-1H-[1,2,3]triazol-3-yl]-[2-(2-chloro-phenyl)-pyrrolidin-1-yl]-methanone (77 mg, 0.13 mmol) in CH₂Cl₂ (1.5 mL) with mCPBA (90 mg, 0.52 mmol) and stir solution at RT for 60 h. Dilute the solution with CH₂Cl₂ (25 mL) and wash with saturated aqueous NaHCO₃ (15 mL). Dry, filter, and concentrate the organic layer. Dissolve the crude glassy material in a minimal amount of ether and precipitate with hexanes to give the title compound as a white amorphous solid. MS(ES) 596.1 (M+1)⁺; ¹H NMR (400 MHz, CDCl₃, 1:1 mixture of amide rotamers) δ 8.20 (m, 1H), 8.10 (s, 0.5H), 7.84 (s, 0.5H), 7.80 (m, 1H), 7.52 (s, 1H), 7.38 (s, 1H), 7.25 (m, 2H), 7.14 (m, 1H), 7.06 (m, 1H), 7.03 (m, 1H), 6.91 (m, 1H), 6.27 (m, 0.5H), 5.58 (m, 1H), 5.54 (m, 0.5H), 5.39 (s, 1H), 4.53 (m, 0.5H), 4.11 (m, 0.5H), 3.89 (m, 0.5H), 3.80 (m, 0.5H), 2.44 (m, 1H), 1.98 (m, 1H), 1.99 (m, 2H).

### EXAMPLE 477

### (±)-(1-(3,5-Bis-trifluoromethyl-benzyl)-5-chloro-1H-[1,2,3]triazol-4-yl]-[2-(2-chloro-phenyl)-pyrrolidin-1-yl]-methanone

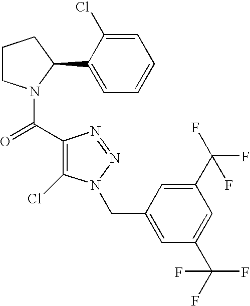

Dissolve 1-(3,5-bis-trifluoromethyl-benzyl)-5-chloro-1H-[1,2,3]triazole-4-carboxylic acid (1.8 g, 4.8 mmol), (±)-2-(2-chloro-phenyl)-pyrrolidine (1.1 g, 5.89 mmol) and DMAP (1.4 g, 11.4 mmol) in CH₂Cl₂ (45 mL) and add EDCI (1.4 g, 7.1 mmol). Stir the solution at RT for 24 h, then dilute with additional CH₂Cl₂ (50 mL) and wash with saturated NH₄Cl (50 mL) and saturated NaHCO₃ (50 mL). Dry, filter, and concentrate the organic phase. Purify crude material by flash chromatography using a linear gradient of 10% to 50% EtOAc/hexanes to give the title compound (2.1 g, 83%) as a white foam upon concentration of solvent. MS(ES) 537.0 (M+1)⁺; ¹H NMR (400 MHz, CDCl₃, mixture of amide rotamers) δ 7.88 (s, 0.5H), 7.84 (s, 0.5H), 7.80 (s, 1H), 7.64 (s, 1H), 7.33 (m, 0.5H), 7.16 (m, 2H), 7.00 (m, 1.5H), 6.23 (m, 0.5H), 5.64 (m, 1.5H), 5.46 (s, 1H), 4.44 (m, 0.5H), 4.12 (m, 0.5H), 4.01 (m, 0.5H), 3.87 (m, 0.5H), 2.43 (m, 1H), 2.00 (m, 2H), 1.88 (m, 1H).

### EXAMPLE 478

### (S)-[1-(3,5-Bis-trifluoromethyl-benzyl)-5-morpholin-4-yl -1H-[1,2,3]triazol-4-yl]-[2-(2-chloro-phenyl)-pyrrolidin-1-yl]-methanone

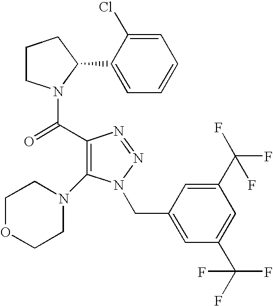

Heat a solution of (S)-[1-(3,5-bis-trifluoromethyl-benzyl)-5-chloro-1H-[1,2,3]triazol-4-yl]-[2-(2-chloro-phenyl)-pyrrolidin-1-yl]-methanone (63mg, 0.12mmol) in morpholine (1.0 mL) to 50-60° C. After 48 h, cool to RT and dilute with EtOAc (30 mL). Wash with 1N HCl (10 mL), H₂O (10 mL), and saturated NaHCO₃ (10 mL). Dry, filter, and concentrate the organic phase. Purify the crude material by flash chromatography using a linear gradient of 20% to 60% EtOAc/hexanes to give (−)-[1-(3,5-bis-trifluoromethyl-benzyl)-5-morpholin-4-yl-1H-[1,2,3]triazol-4-yl]-[2-(2-chloro-phenyl)-pyrrolidin-1-yl]-methanone (37 mg, 54%) as a white foam. MS(ES) 588.2 (M+])⁺; ¹H NMR (400 MHz, CDCl₃, mixture of amide rotamers) δ 7.85 (s, 0.5H), 7.83 (s, 1H), 7.81 (s, 0.5H), 7.65 (s, 1H), 7.34 (m, 0.5H), 7.16 (m, 2H), 7.96 (m, 1.5H), 6.31 (m, 0.5H), 5.64 (m, 0.5H), 5.54 (s, 1H), 5.36 (d, 1H, J=3.4 Hz), 4.37 (m, 0.5H), 3.99 (m, 1H), 3.90 (m, 0.5H), 3.59-3.73 (m, 4H), 2.87-2.98 (m, 3H), 2.74 (m, 1H), 2.46 (m, 1H), 1.96 (m, 3H).

Using a similar method to that above, with the appropriate starting materials, the following compound may be prepared.

| Ex. # | Product | Data |
|---|---|---|
| 479 | (S)-[1-(3,5-Bis- | MS(ES) 601.4(M+1)⁺; ¹H NMR(400MHz, CDCl₃, |
| | trifluoromethyl-benzyl)-5- | mixture of amide rotamers) δ 7.84(s, 0.5H), 7.83(s, |
| | (4-methyl-piperazin-1-yl)- | 1H), 7.80(s, 0.5H), 7.65(s, 1H), 7.32(m, 0.5H), |
| | 1H-[1,2,3]triazol-4-yl]-[2- | 7.12(m, 2H), 7.96(m, 1.5H), 6.25(m, 0.5H), 5.62(m, |
| | (2-chloro-phenyl)- | 0.5H), 5.50(s, 1H), 5.32(m, 1H), 4.31(m, 0.5H), |
| | pyrrolidin-1-yl]-methanone | 3.97(m, 1H), 3.86(m, 0.5H), 2.97(m, 3H), 2.75(m, 1H), |
| | | 2.41(m, 5H), 2.27(s, 1.5H), 2.25(s, 1.5H), 1.94(m, |
| | | 3H). |

### EXAMPLE 480

### (R)-[1-(3,5-Bis-trifluoromethyl-benzyl)-5-piperazin-1-yl-1H-[1,2,3]triazol4-yl]-[2-(2-chloro-phenyl)-pyrrolidin-1-yl]-methanone

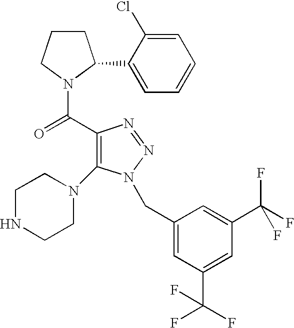

Add (R)-[1-(3,5-bis-trifluoromethyl-benzyl)-5-chloro-1H-[1,2,3]triazol-4-yl]-[(2-(2-chloro-phenyl)-pyrrolidin-1-yl]-methanone (0.25 g, 0.47 mmol) to piperazine (0.10 g, 1.16 mmol) and heat to 100° C. in a sealed tube for 16 h. Dilute the reaction mixture with ethyl acetate, wash with water and brine, then dry, and concentrate. Purify the residue by flash chromatography using a linear gradient of 5 to 9% MeOH in dichloromethane to give the title compound (0.25 g, 92%) as white solid. MS(ES) 587.3 (M+1)⁺; ¹H NMR (400 MHz, CDCl₃, mixture of amide rotamers) δ 7.86 (s, 1.5H), 7.82 (s, 0.5H), 7.68 (s, 1H), 7.36 (s, 0.5H), 7.14-7.19 (m, 2H), 6.97 (m, 1.5H), 6.32 (m, 0.5H), 5.65 (m, 0.5H), 5.54 (m, 1H), 5.36 (m, 1H), 4.36 (m, 0.5H), 3.96-4.08 (m, 1H), 3.90 (m, 0.5H), 2.85-2.91 (m, 8H), 2.70 (m, 1H), 2.46 (m, 1H), 1.91-2.03 (m, 3H).

Using an analogous procedure to(R)-[1-(3,5-Bis-trifluoromethyl-benzyl)-5-piperazin-1-yl-1H-[1,2,3]triazol-4-yl]-[2-(2-chloro-phenyl)-pyrrolidin-1-yl]-methanone described above, with the appropriate starting materials, the following compounds may be prepared.

| Ex. # | Product | Data |
|---|---|---|
| 481 | (R)-[1-(3,5-Bis- | MS(ES) 604.2(M+1)⁺; ¹H NMR(400MHz, CDCl₃, |
| | trifluoromethyl-benzyl)-5- | mixture of amide rotamers) δ 7.82(m, 2H), 7.64(s, |
| | thiomorpholin-4-yl-1H- | 1H), 7.34(m, 0.5H), 7.12-7.20(m, 2.5H), 6.98(m, |
| | [1,2,3]triazol-4-yl]-[2-(2- | 1H), 6.35(m, 0.5H), 5.65(m, 0.5H), 5.52(s, 1H), |
| | chloro-phenyl)-pyrrolidin-1- | 5.33(s, 1H), 4.34(m, 0.5H), 3.90-4.11(m, 1.5H), |
| | yl]-methanone | 2.66(s, 3H), 2.57(s, 3H), 2.45(m, 1H), |
| | | 1.87-2.02(m, 3H). |
| 482 | (R)-[1-(3,5-Bis- | MS(ES) 546.3(M+1)⁺. ¹H NMR(400MHz, CDCl₃, |
| | trifluoromethyl-benzyl)-5- | mixture of amide rotamers) δ 7.88(s, 0.5H), 7.83(s, |
| | dimethylamino-1H- | 1.5H), 7.64(s, 1H), 7.37(s, 0.5H), 7.17(m, 2H), |
| | [1,2,3]triazol-4-yl]-[2-(2- | 6.70(m, 1.5H), 6.36(m, 0.5H), 5.67(m, 0.5H), |
| | chloro-phenyl)-pyrrolidin-1- | 5.52(m, 1H), 5.35(m, 1H), 4.40(m, 0.5H), 4.02(m, 1H), |
| | yl]-methanone | 3.91(m, 0.5H), 3.12-3.22(m, 3H), 3.00(m, 0.5H), |
| | | 2.58-2.70(m, 3H), 2.48(m, 0.5H), 1.96(m, 3H). |
| 483 | (R)-[1-(3,5-Bis- | MS(ES) 602.2(M+1)⁺. ¹H NMR(400MHz, CDCl₃, |
| | trifluoromethyl-benzyl)-5-(4- | mixture of amide rotamers) δ 7.87(s, 0.5H), 7.85(s, |
| | hydroxy-piperidin-1-yl)-1H- | 1H), 7.82(s, 0.5H), 7.67(s, 1H), 7.35(m, 0.5H), |
| | [1,2,3]triazol-4-yl]-[2-(2- | 7.14-7.19(m, 2H), 6.96(m, 1.5H), 6.35(m, 0.5H), |
| | chloro-phenyl)-pyrrolidin-1- | 5.63(m, 0.5H), 5.55(m, 1H), 5.30(m, 1H), 4.37(m, |
| | yl]-methanone | 0.5H), 3.95-4.09(m, 1H), 3.79-3.92(m, 1.5H), |
| | | 3.01(m, 2H), 2.90(m, 1.5H), 2.48(m, 1.5H), |
| | | 1.86-2.03(m, 5H), 1.79(m, 0.5H), 1.45-1.60(m, 1.5H). |
| 484 | (R)-[1-(3,5-Bis- | MS(ES) 629.5(M+1)⁺. ¹H NMR(400MHz, CDCl₃, |
| | trifluoromethyl-benzyl)-5-(4- | mixture of amide rotamers): δ 7.85(s, 2H), 7.67(s, |
| | isopropyl-piperazin-1-yl)- | 1H), 7.35(m, 0.5H), 7.21(m, 0.5H), 7.12-7.18(m, |
| | 1H-[1,2,3]triazol-4-yl]-[2-(2- | 1.5H), 6.96(m, 1.5H), 6.28(d, 0.5H, J=7.4, 3.1), |
| | chloro-phenyl)-pyrrolidin-1- | 5.65(d, 0.5H, J=7.4, 3.1), 5.52(s, 1H), 5.30(m, |
| | yl]-methanone | 1H), 4.35(m, 0.5H), 3.85-4.00(m, 1.5H), 2.93(m, |
| | | 3H), 2.68(m, 2H), 2.50(m, 4.5H), 1.91-2.00(m, |
| | | 3.5H), 1.00(m, 6H). |
| 485 | [1-(3,5-Bis-trifluoromethyl- | MS(ES) 615.5(M+1)⁺. ¹H NMR(400MHz, CDCl₃, |
| | benzyl)-5-(3,5-dimethyl- | mixture of amide rotamers) δ 7.86(s, 1.5H), 7.81(s, |
| | piperazin-1-yl)-1H- | 0.5H), 7.68(s, 1H), 7.34(m, 0.5H), 7.12(m, 2.5H), |
| | [1,2,3]triazol-4-yl]-[2-(2- | 6.96(m, 1H), 6.26(d, 0.5H, J=7.0, 2.9), 5.62(d, |
| | chloro-phenyl)-pyrrolidin-1- | 0.5H, J=7.0, 2.9), 5.51(s, 1H), 5.34(s, 1H), |
| | yl]-methanone | 4.28(m, 0.5H), 4.08(m, 0.5H), 3.96(m, 0.5H), 3.88(m, |
| | | 0.5H), 2.65-2.93(m, 4.5H), 2.47(m, 2.5H), 1.97(m, |
| | | 3H), 0.92-1.00(m, 6H). |
| 486 | [1-(3,5-Bis-trifluoromethyl- | MS(ES) 616.5(M+1)⁺. ¹H NMR(400MHz, CDCl₃, |
| | benzyl)-5-(2,6-dimethyl- | mixture of amide rotamers) δ 7.86(s, 1.5H), 7.81(s, |
| | morpholin-4-yl)-1H- | 0.5H), 7.68(s, 1H), 7.34(m, 0.5H), 7.12(m, 2H), |
| | [1,2,3]triazol-4-yl]-[2-(2- | 6.96(m, 1.5H), 6.26(d, 0.5H, J=7.5, 2.9Hz), |
| | chloro-phenyl)-pyrrolidin-1- | 5.62(d, 0.5H, J=7.0, 2.9Hz), 5.51(s, 1H), 5.34(s, 1H), |
| | yl]-methanone | 4.31(m, 0.5H), 3.96-4.11(m, 1H), 3.88(m, 0.5H), |
| | | 3.47-3.70(m, 2H), 2.95-3.10(m, 2H), 2.34-2.50(m, |
| | | 2.5H), 1.88-2.01(m, 3.5H), 1.02-1.20(m, 6H). |

### EXAMPLE 487

### (R)-1-(4-{3-(3,5-Bis-trifluoromethyl-benzyl)-5-[2-(2-chloro-phenyl)-pyrrolidine-1-carbonyl]-3H-[1,2,3]triazol-4-yl}-piperazin-1-yl)-ethanone

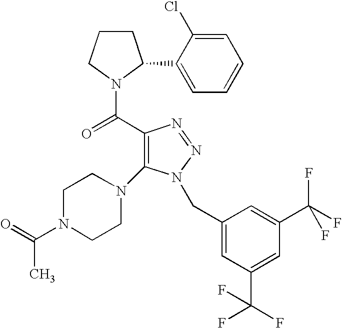

Add acetyl chloride (20.0 mg, 0.26 mmol) to a solution of (R)-[1-(3,5-bis-trifluoromethyl-benzyl)-5-piperazin-1-yl-1H-[1,2,3]triazol-4-yl]-[2-(2-chloro-phenyl)-pyrrolidin-1-yl]-methanone (0.10 g, 0.17 mmol) and triethylamine (50.0 μL, 0.35 mmol) in dichloromethane (3.0 mL). Stir at RT for 4 h, then dilute with water and extract with EtOAc. Wash the EtOAc extract with water and brine, then dry and concentrate. Purify the residue by flash chromatography using a linear gradient of 1 to 4% MeOH in dichloromethane to give the title compound (0.10 g, 95%). MS(ES) 629.4 (M+1)⁺. ¹H NMR (400 MHz, CDCl₃, 1:1 mixture of amide rotamers) δ 7.87 (s, 0.5H), 7.82 (s, 1.5H), 7.64 (s, 1H), 7.34 (s, 0.5H), 7.14-7.19 (m, 2H), 6.93-7.00 (m, 1.5H), 6.35 (m, 0.5H), 5.61 (m, 0.5H), 5.57 (m, 1H), 5.39 (m, 1H), 4.38 (m, 0.5H), 3.96-4.12 (m, 1H), 3.87 (m, 0.5H), 3.58-3.75 (m, 1.5H), 3.42 (m, 2H), 2.87-3.00 (m, 4H), 2.62 (m, 0.5H), 2.42-2.51 (m, 1H), 2.08 (s, 1.5H), 2.03 (s, 1.5H), 1.87-2.00 (m, 3H).

Using an analogous procedure to (R)-1-(4-{3-(3,5-Bis-trifluoromethyl-benzyl)-5-[2-(2-chloro-phenyl)-pyrrolidine-1-carbonyl]-3H-[1,2,3)triazol-4-yl}-piperazin-1-yl)-ethanone described above, with the appropriate starting materials, the following compounds may be prepared.

| Ex. # | Product | Data |
|---|---|---|
| 488 | (R)-[1-(3,5-Bis- | MS(ES) 665.4(M+1)⁺. ¹H NMR(400MHz, CDCl₃, |
| | trifluoromethyl-benzyl)-5-(4- | mixture of amide rotamers) δ 7.87(s, 0.5H), 7.83(s, |
| | methanesulfonyl-piperazin-1- | 0.5H), 7.81(s, 1H), 7.62(s, 1H), 7.35(m, 0.5H), |
| | yl)-1H[1,2,3]triazol-4-yl]-[2- | 7.16(m, 2H), 6.95-7.00(m, 1.5H), 6.33(m, 0.5H), |
| | (2-chloro-phenyl)-pyrrolidin- | 5.63(m, 0.5H), 5.55(m, 1H), 5.37(m, 1H), 4.40(m, |
| | 1-yl]-methanone | 0.5H), 3.96-4.10(m, 1H), 3.87(m, 0.5H), |
| | | 3.13-3.27(m, 4H), 2.98-3.06(m, 3H), 2.87(m, 1H), 2.81(s, |
| | | 1.5H), 2.77(s, 1.5H), 2.46(m, 1H), 1.88-2.03(m, |
| | | 3H). |
| 489 | (R)-N-{3-(3,5-Bis- | MS(ES) 674.4(M+1)⁺. ¹H NMR(400MHz, CDCl₃, |
| | trifluoromethyl-benzyl)-5-[2- | mixture of amide rotamers) δ 7.90(s, 0.5H), 7.84(s, |
| | (2-chloro-phenyl)- | 2H), 7.58(s, 0.5H), 7.34(m, 0.5H), 7.17(m, 2.5H), |
| | pyrrolidine-1-carbonyl]-3H- | 7.11(m, 0.5H), 7.02(m, 0.5H), 6.42(m, 0.5H), |
| | [1,2,3]triazol-4-yl}- | 5.72(m, 1H), 5.61(m, 1H), 4.10-4.27(m, 1H), 4.04(m, |
| | dimethanesulfonamide | 0.5H), 3.88(m, 0.5H), 3.48(s, 1.5H), 3.31(s, 1.5H), |
| | | 3.27(s, 1.5H), 3.24(s, 1.5H), 2.45(m, 1H), |
| | | 1.92-2.04(m, 3H). |

### EXAMPLE 490

### (R)-[1-(3,5-Bis-trifluoromethyl-benzyl)-5-(1-oxo-114-thiomorpholin-4-yl)-1H-[1,2,3]triazol-4-yl]-[2-(2-chloro-phenyl)-pyrrolidin-1-yl]-methanone

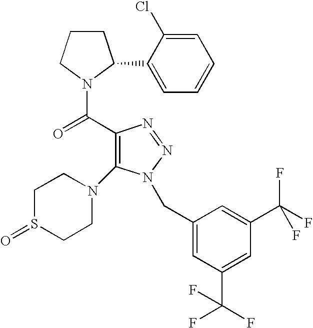

Add 30% aqueous hydrogen peroxide (2.0 mL, excess) to a solution of (R)-[1-(3,5-bis-trifluoromethyl-benzyl)-5-thiomorpholin-4-yl-1H-[1,2,3]triazol-4-yl]-[2-(2-chloro-phenyl)-pyrrolidin-1-yl]-methanone (0.08 g, 0.13 mmol) in MeOH (2.0 mL) and stir at RT. After 24 h, add water and extract with EtOAc, then dry (Na₂SO₄), filter, and concentrate. Purify the residue by flash chromatography using a linear gradient of 5 to 7% MeOH in dichloromethane to give the title compound (0.06 g, 75%). MS(ES) 620.3 (M+1)⁺; ¹H NMR (400 MHz, CDCl₃, mixture of amide rotamers) δ 7.88 (s, 0.5H), 7.84 (s, 0.5H), 7.82 (s, 1H), 7.63 (s, 1H), 7.34 (m, 0.5H), 7.12-7.20 (m, 2H), 6.98 (m, 1.5H), 6.35 (m, 0.5H), 5.63 (m, 0.5H), 5.56 (m, 1H), 5.38 (m, 1H), 4.43 (m, 0.5H), 3.96-4.08 (m, 1H), 3.87 (m, 0.5H), 3.44 (m, 2H), 3.28 (m, 1H), 2.92-3.11 (m, 3H), 2.81 (m, 2H), 2.40-2.51 (m, 1H), 1.87-2.02 (m, 3H).

### EXAMPLE 491

### (R)-[1-(3,5-Bis-trifluoromethyl-benzyl)-5-( 1,1-dioxo-1λ⁶-thiomorpholin-4-yl)-1H-[1,2,3]triazol-4-yl]-[2-(2-chloro-phenyl)-pyrrolidin-1-yl]-methanone

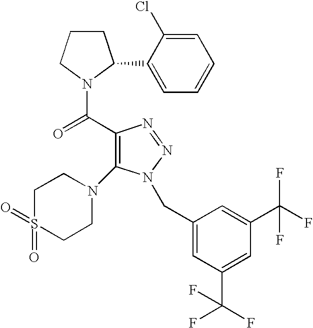

Add 30% aqueous hydrogen peroxide (5.0 mL, excess) to a solution of (R)-[1-(3,5-bis-trifluoromethyl-benzyl)-5-thiomorpholin-4-yl-1H-[1,2,3]triazol-4-yl]-[2-(2-chloro-phenyl)-pyrrolidin-1-yl]-methanone (0.06 g, 0.10 mmol) in MeOH (2.0 mL) and stir at 80° C. for 18 h. Add water and extract with EtOAc, then dry (Na₂SO₄), filter, and concentrate. Purify the residue by flash chromatography using a linear gradient of 3 to 4% MeOH in dichloromethane to give the title compound (0.06 g, 95%) as a white solid. MS(ES) 636.0 (M+1)⁺. ¹H NMR (400 MHz, CDCl₃, 1:1 mixture of amide rotamers) δ 7.91 (s, 0.5H), 7.86 (s, 0.5H), 7.79 (s, 1H), 7.60 (s, 1H), 7.34 (m, 0.5H), 7.16-7.23 (m, 2H), 6.97-7.04 (m, 1.5H), 6.37 (m, 0.5H), 5.66 (m, 0.5H), 5.56 (m, 1H), 5.40 (m, 1H), 4.47 (m, 0.5H), 4.06 (m, 1H), 3.90 (m, 0.5H), 3.48 (m, 2H), 3.30-3.42 (m, 2H), 3.04 (m, 4H), 2.41-2.54 (m, 1H), 1.88-2.03 (m, 3H).

### EXAMPLE 492

### (R)-1-{3-(3,5-Bis-trifluoromethyl-benzyl)-5-[2-(2-chloro-phenyl)-pyrrolidine-1-carbonyl]-3H-[1,2,3]triazol-4-yl}-piperidin-4-one

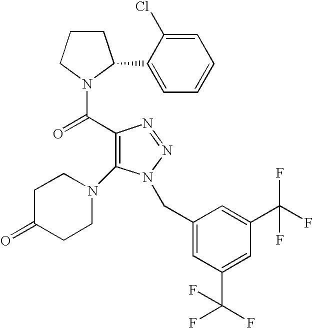

Add Dess-Martin periodinane (0.15 g, 0.35 mmol) to a solution of (R)-[1-(3,5-bis-trifluoromethyl-benzyl)-5-(4-hydroxy-piperidin-1-yl)-1H-[1,2,3]triazol-4-yl]-[2-(2-chloro-phenyl)-pyrrolidin-1-yl]-methanone (0.14 g, 0.23 mmol) in dichloromethane (3.0 mL) at 0° C. Stir the mixture at 0° C. for 30 min, then warm to RT for 3 h. Dilute with water and extract with EtOAc. Wash the organic layer with 1N NaOH, water, and brine, then dry (Na₂SO4), and concentrate. Purify the residue by flash chromatography using a linear gradient of 30 to 45% EtOAc in hexanes to give the title compound (0.13 g, 93%). MS(ES) 600.3 (M+1)⁺. ¹H NMR (400 MHz, CDCl₃, 1:1 mixture of amide rotamers) δ 7.88 (s, 0.5H), 7.84 (s, 1.5H), 7.66 (s, 1H), 7.34 (m, 0.5H), 7.19 (m, 0.5H), 7.15 (m, 1.5H), 6.94-7.01 (m, 1.5H), 6.38 (m, 0.5H), 5.62 (m, 1.5H), 5.45 (m, 1H), 4.41 (m, 0.5H), 4.07 (m, 0.5H), 3.97 (m, 0.5H), 3.87 (m, 0.5H), 3.27 (m, 3H), 3.09 (m, 1H), 2.46 (m, 5H), 1.98 (m, 3H).

### EXAMPLE 493

### (R)-[1-(3,5-Bis-trifluoromethyl-benzyl)-5-(3,6-dihydro-2H-pyridin-1-yl)-1H-[1,2,3]triazol-4-yl]-[2-(2-chloro-phenyl)-pyrrolidin-:1-yl]-methanone

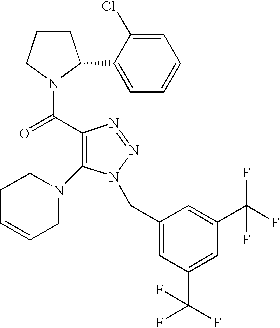

Add DAST (45.0 μL, 0.36 mmol) to a solution of (R)-[1-(3,5-bis-trifluoromethyl-benzyl)-5-(4-hydroxy-piperidin-1-yl)-1H-[1,2,3]triazol-4-yl]-[2-(2-chloro-phenyl)-pyrrolidin-1-yl]-methanone (0.11 g, 0.18 mmol) in dichloromethane (4.0 mL) at −78° C. Stir the mixture at −78° C. for 30 min, then warm to RT for 1 h. Dilute with dichloromethane and wash with water and brine, then dry, and concentrate. Purify the residue by flash chromatography using a linear gradient of 10 to 25% EtOAc in hexanes to give the title compound (0.03 g, 28%). MS(ES) 584.3 (M+1)⁺. ¹H NMR (400 MHz, CDCl₃, 1:1 mixture of amide rotamers) δ 7.85 (s, 1.5H), 7.80 (s, 0.5H), 7.67 (s, 1H), 7.13-7.19 (m, 2H), 6.98 (m, 1.5H), 6.35 (m, 0.5H), 5.78 (m, 1H), 5.51 (m, 1H), 5.33 (m, 1H), 4.39 (m, 0.5H), 4.08 (m, 0.5H), 3.97 (m, 0.5H), 3.88 (m, 0.5H), 3.42 (m, 1H), 3.30 (m, 1H), 3.00-3.11 (m, 1.5H), 2.82 (m, 0.5H), 2.47 (m, 1H), 2.11 (m, 2H), 1.88-2.04 (m, 3H).

### EXAMPLE 494

### (R)-[5-Amino-1-(3,5-bis-trifluoromethyl-benzyl)-1H-[1,2,3]triazol4-yl]-[2-(2-chloro-phenyl)-pyrrolidin-1-yl]-methanone

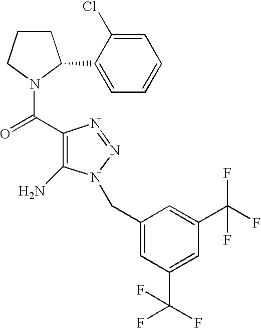

Combine EDCI (0.83 g, 0.44 mmol) with a solution of 5-amino-1-(3,5-bis-trifluoromethyl-benzyl)-1H-[1,2,3]triazole-4-carboxylic acid (0.11 g, 0.31 mmol), (R)-2-(2-chloro-phenyl)-pyrrolidine (0.08 g, 0.44 mmol), and DMAP (0.05 g, 0.44 mmol) in DMF (5.0 mL). After 48 h, treat the reaction mixture with saturated NaHCO₃ and extract with EtOAc. Wash the organic layer with 0.1N HCl, water, and brine, then dry and concentrate to give the title compound (0.12 g, 75%) as a 1:1 mixture of rotamers. MS(ES) 518 (M+1)⁺; ¹H NMR (400 MHz, DMSO-d₆ run at 100° C.) δ 7.95 (s, 1H), 7.90 (s, 2H), 7.38 (m, 1H), 7.22 (m, 1H), 7.19 (m, 1H), 7.14 (m, 1H), 6.40 (br s, 2H), 5.81 (br m, 1H), 5.58 (s, 2H), 4.20 (m, 1H), 4.14 (m, 1H), 2.41 (m, 1H), 2.02-1.86 (m, 2H), 1.81 (m, 1H).

### EXAMPLE 495

### 1-(3,5-Bis-trifluoromethyl-benzyl)-5-phenyl-1H-[1,2,3]triazole-4-carbothioic acid (2-fluoro-benzyl)-methyl-amide

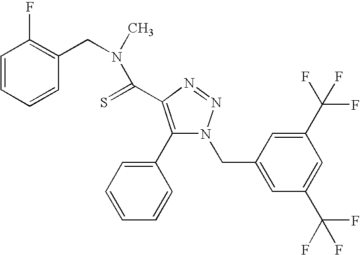

Combine 1-(3,5-Bis-trifluoromethyl-benzyl)-5-phenyl-1H-[1,2,3]triazole-4-carboxylic acid (2-fluoro-benzyl)-methyl-amide (1 eq., 0.071 g, 0.13 mmol) and Lawesson's reagent (0.55 eq., 0.029 g, 0.07 mmol) in toluene (3 mL, 0.025 M). Stir at 80° C. until complete by TLC. Add H₂O and extract with CH₂Cl₂, dry over Na₂SO₄, and concentrate in vacuo. Purify by chromatography (0 to 50% EtOAc/Hexane gradient) on silica gel. R_{f} 0.57 (50% EtOAc/ Hexane); MS(ES) 553.2 (M+1)⁺.

Using a similar procedure and the appropriate amide starting material, the following compounds may be prepared and isolated.

| Ex. # | Product | Data |
|---|---|---|
| 496 | 1-(3,5-Bis-trifluoromethyl-benzyl)- | R_{f}=0.55(50% EtOAc/ |
| | 5-phenyl-1H-[1,2,3]triazole-4- | Hexane); MS(ES) |
| | carbothioic acid (2-chloro- | 569.2(M+1)⁺. |
| | benzyl)-methyl-amide | |
| 497 | [1-(3,5-Bis-trifluoromethyl-benzyl)- | R_{f}=0.71(50% EtOAc/ |
| | 5-phenyl-1H-[1,2,3]triazol-4-yl]- | Hexane); MS(ES) |
| | [2-(2-chloro-phenyl)-pyrrolidin- | 595.3(M+1)⁺. |
| | 1-yl]-methanethione | |

### EXAMPLE 498

### 1-(3,5-Bis-trifluoromethyl-benzyl)-5-pyridin-4-yl-1H-[1,2,3]triazole-4-carboxylic acid isopropyl-(2-trifluoromethoxy-benzyl)-amide

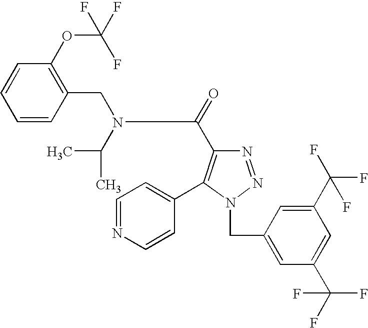

Combine 1-(3,5-Bis-trifluoromethyl-benzyl)-5-pyridin-4-yl-1H-[1,2,3]triazole-4-carboxylic acid (0.15 g, 0.36 mmol) with isopropyl-(2-trifluoromethoxy-benzyl)-amine(0.084 g, 0.36 mmol), EDCI (0.069 g, 0.36 mmol), HOAt (0.049 g, 0.36 mmol), and N,N-diisopropylethylamine (0.10 ml) in DMF (5 mL) and stir at RT until complete. Concentrate the mixture in vacuo, then dissolve the residue in EtOAc and wash with water and brine. Dry over Na₂SO₄, filter, and concentrate. Purify by chromatography on silica gel to provide the title compound. MS (ES) 632.2 (M+1)⁺. Rf=0.47 (6.7% MeOH/CH₂Cl₂).

Using a procedure similar to that used for 1-(3,5-Bis-trifluoromethyl-benzyl)-5-pyridin-4-yl-1H-[1,2,3]triazole-4-carboxylic acid isopropyl-(2-trifluoromethoxy-benzyl)-amide above, with the appropriate starting materials, the following compounds may be prepared and isolated.

| Ex. # | Product | Data |
|---|---|---|
| 499 | 1-(3,5-dichloro-benzyl)-5-pyridin-4yl-1H- | MS(ES) 514.1(M+1)⁺, |
| | [1,2,3]triazole-4-carboxylic acid (2-chloro- | 516.1(M+3)⁺. Rf=0.55(6.7% |
| | benzyl)-isopropyl-amide | MeOH/CH₂Cl₂). |
| 500 | [1-(3,5-Bis-trifluoromethyl-benzyl)-5- | MS(ES) 547.2(M+1)⁺, |
| | pyridin-4-yl-1H-[1,2,3]triazol-4-yl]- | 548.3(M+3)⁺. Anal. Calc'd C₂₆H₂₀F₆N₆O: |
| | (2-pyridin-4-yl-pyrrolidin-1-yl)-methanone | C, 57.15; H, 3.69; N, 15.38. Found: |
| | | C, 56.19; H, 3.88; N, 14.61. |
| 501 | [1-(3,5-Bis-trifluoromethyl-benzyl)-5- | MS(ES) 594.1(M+1)⁺. |
| | pyridin-4-yl-1H-[1,2,3]triazol-4-yl]- | Rf=0.26(6.7% MeOH/CH₂Cl₂). |
| | [2-(2-chloro-phenyl)-2-methyl-pyrrolidin-1- | |
| | yl]-methanone | |

### EXAMPLE 502

### 1-(3,5-Bis-trifluoromethyl-benzyl)-5-pyridin-4-yl -1H-[1,2,3]triazole-4-carboxylic acid (2-chloro-phenyl)-isopropyl-amide

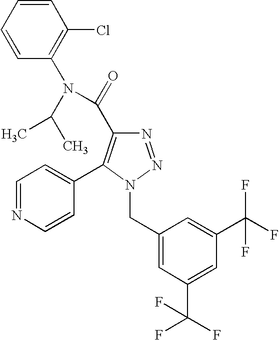

Combine 1-(3,5-Bis-trifluoromethyl-benzyl)-5-pyridin-4-yl-1H-[1,2,3]triazole-4-carboxylic acid (0.27 g, 0.65 mmol) with oxalyl chloride (0.17 mL, 1.95 mmol) and DMF (1 drop, catalytic) in CH₂Cl₂ (5 mL) and stir at RT until acid chloride formation is complete. Concentrate the mixture in vacuo, redissolve in Et₂O and concentrate again. Dissolve the residue in pyridine (5 mL) and add (2-chloro-phenyl)-isopropyl-amine(0.11 g, 0.65 mmol ) and DMAP (0.003 g, cat.) and heat until the reaction is complete. Then, quench with aqueous NaHCO₃ and extract with EtOAc twice. Dry the combined organic extracts over Na₂SO₄, filter, and concentrate. Purify the residue by chromatography on silica gel to provide the title compound. MS(ES) 568.1 (M+1)⁺.

Using a similar method and the appropriate starting materials, the following compounds may be prepared and isolated.

| Ex. # | Product | Data |
|---|---|---|
| 503 | 1-(3,5-Bis-trifluoromethyl-benzyl)-5-pyridin-3-yl- | MS(ES) 568.1(M+1)⁺. |
| | 1H-[1,2,3]triazole-4-carboxylic acid (2-chloro- | |
| | phenyl)-isopropyl-amide | |
| 504 | 1-(3,5-Bis-trifluoromethyl-benzyl)-5-pyridin-4-yl- | MS(ES) 622.1(M+1)⁺. |
| | 1H-[1,2,3]triazole-4-carboxylic acid (2-chloro- | Rf=0.57(6.7% MeOH/CH₂Cl₂). |
| | benzyl)-(2,2,2-trifluoro-ethyl)-amide | |
| 505 | 1-(3,5-Bis-trifluoromethyl-benzyl)-5-pyridin-3-yl- | MS(ES) 622.1058(M+1)⁺. |
| | 1H-[1,2,3]triazole-4-carboxylic acid (2-chloro- | Rf=0.73(6.7% MeOH/CH₂Cl₂). |
| | benzyl)-(2,2,2-trifluoro-ethyl)-amide | |
| 506 | 1-(3,5-Bis-trifluoromethyl-benzyl)-5-pyridin-4-yl- | MS(ES) 590.1(M+1)⁺; |
| | 1H-[1,2,3]triazole-4-carboxylic acid isopropyl-(2- | Rf=0.39(6.7% MeOH/CH₂Cl₂). |
| | trifluoromethoxy-benzyl)-amide | |
| 507 | [1-(3,5-Bis-trifluoromethyl-benzyl)-5-pyridin-3- | MS(ES) 594.1(M+1)⁺; |
| | yl-1H-[1,2,3]triazol-4-yl]- | Rf=0.29(6.7% MeOH/CH₂Cl₂). |
| | [2-(2-chloro-phenyl)-2-methyl-pyrrolidin-1-yl]- | |
| | methanone | |

### EXAMPLE 508

### 1-(3,5-Bis-trifluoromethyl-benzyl)-5-morpholin-4-yl-1H-[1,2,3]triazole-4-carboxylic acid (2-chloro-phenyl)-isopropyl-amide

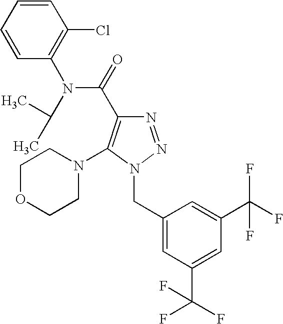

Combine 1-(3,5-Bis-trifluoromethyl-benzyl)-5-chloro-1H-[1,2,3]triazole-4-carboxylic acid (2-chloro-phenyl)-isopropyl-amide (0.11 g, 0.21 mmol) with an excess of morpholine and heat the mixture near 50° C. for 3-5 hours, and then allow to stir overnight at RT. Quench the mixture with aqueous NaHCO₃ and extract with EtOAc. Wash the combined organic extracts with water, dry over Na₂SO₄, filter and concentrate. Purify by chromatography on silica gel to provide the title compound. MS(ES) 576.1 (M+1)⁺; Rf=0.43 (6.25% MeOH/CH₂Cl₂).

### EXAMPLE 509

### 1-(3,5-Bis-trifluoromethyl-benzyl)-5-phenyl-1H-imidazole-4-carboxylic acid (2,6-dichloro-benzyl)-methyl-amide

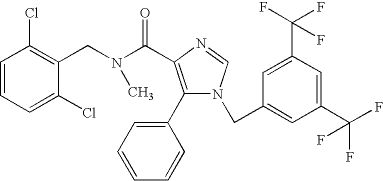

To a solution of 1-(3,5-bis-trifluoromethyl-benzyl)-5-phenyl-1H-imidazole-4-carboxylic acid (0.030 g, 0.072 mmol) in CH₂Cl₂ (0.7 mL) add HOBt-H₂O (0.020 g, 0.145 mmol), 2,6-dichloro-n-methyl benzyl amine (0.028 g, 0.145 mmol), NEt₃ (0.050 mL, 0.362 mmol) and EDCI (0.028 g, 0.145 mmol) and stir the resulting orange mixture at RT. After 16 h., pour the mixture into CH₂Cl₂, wash with saturated aqueous NaHCO₃ and extract the aqueous layer with CH₂Cl₂ twice. Dry the combined organics over MgSO₄, filter, concentrate. Purify the residue by chromatography over silica gel using a hexanes/EtOAc gradient to yield the title compound (0.030 g, 71%) as a yellow oil. ¹H NMR (400 MHz, CDCl₃) 7.79 (s, 1H), 7.15-7.45 (m, 11H), 5.19-5.30 (m, 2H), 5.05 (s,2H), 2.89 (s, 1.5H), 2.78 (s, 1.5H).

Using a method similar to the above Example, with the appropriate starting materials, the following compounds may be prepared and isolated.

| Ex. # | Product | Data |
|---|---|---|
| 510 | 1-(3,5-Bis-trifluoromethyl-benzyl)-5-phenyl- | ¹H NMR(400MHz, CDCl₃) |
| | 1H-imidazole-4-carboxylic acid (2-chloro- | 7.68(bd, J=12Hz, 1H), 7.59(s, 0.5H), |
| | benzyl)-methyl-amide | 7.27(s, 0.5H), 7.01-7.33(m, |
| | | 11H), 5.11(s, 1H), 5.01(s, 1H), |
| | | 4.92(s, 1H), 4.68(s, 1H), 2.97(s, |
| | | 1.5H), 2.81(s, 1.5H). |
| 511 | 1-(3,5-Bis-trifluoromethyl-benzyl)-5-phenyl- | R_{f}=0.13(100% EtOAc); |
| | 1H-imidazole-4-carboxylic acid cyclohexyl- | MS(ES) 510.2(M+1) |
| | methyl-amide | |
| 512 | 1-(3,5-Bis-trifluoromethyl-benzyl)-5-phenyl- | R_{f}=0.11(100% EtOAc) |
| | 1H-imidazole-4-carboxylic acid cyclopentyl- | MS(ES) 496.2(M+1) |
| | methyl-amide | |
| 513 | 1-(3,5-Bis-trifluoromethyl-benzyl)-5-phenyl- | R_{f}=0.27(100% EtOAc) |
| | 1H-imidazole-4-carboxylic acid (2-fluoro- | MS(ES) 526.2(M+1) |
| | benzyl)-methyl-amide | |
| 514 | 1-(3,5-Bis-trifluoromethyl-benzyl)-5-phenyl- | ¹H NMR(400MHz) δ |
| | 1H-imidazole-4-carboxylic acid (2- | 7.84-7.77(m, 2H), 7.70-7.55(m, 2H), |
| | trifluoromethyl-benzyl)-methyl-amide | 7.47-7.15(m, 9H), 5.24(s, 1H), |
| | | 5.14(s, 2H), 4.89(s, 2H), 3.07(s, 1.5H), |
| | | 2.94(s, 1.5H). |

### EXAMPLE 515

### [1-(3,5-Bis-trifluoromethyl-benzyl)-5-phenyl-1H-[1,2,3]triazol-4-yl]-[2-(2-chloro-phenyl)-pyrrolidin-1-yl]-methanone

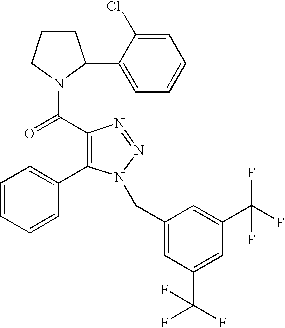

Dissolve 1-(3,5-bis-trifluoromethyl-benzyl)-5-phenyl-1H-[1,2,3]triazole-4-carboxylic acid (2.13 g, 18.2 mmol), (±)-2-(2-chloro-phenyl)-pyrrolidine (0.93 g, 5.12 mmol), and HOBt (0.86 g, 6.4 mmol) in a mixture of CH₂Cl₂ (50 mL) and triethylamine (2.14 mL, 15.4 mmol). Add EDCI (1.23 g, 6.4 mmol) and stir the solution at RT. After 24 h, dilute with CH₂Cl₂ (50 mL) and wash with 1 N HCl (100 mL), H₂O (100 mL), and saturated NaHCO₃ (100 mL). Dry the organic layer over MgSO₄, filter, and concentrate to give a pale yellow foam. Crystallize from EtOAc/hexanes (˜1:10) to provide 2.20 g (74%) of the title compound in two crops. The racemic mixture may be separated using using chiral chromatography (SS Whelk-01, 20% 3A alcohol/10% IPA/70% heptane) to give the (R)-enantiomer (earlier eluting) and the (S)-enantiomer (later eluting). MS(ES) 579.1 (M+1)⁺; R_{f}=0.18 (2:1 hexanes/EtOAc).

The compounds of the present invention can be administered alone or in the form of a pharmaceutical composition, that is, combined with pharmaceutically acceptable carriers, or excipients, the proportion and nature of which are determined by the solubility and chemical properties of the compound selected, the chosen route of administration, and standard pharmaceutical practice. The compounds of the present invention, while effective themselves, may be formulated and administered in the form of their pharmaceutically acceptable salts, for purposes of stability, convenience of crystallization, increased solubility, and the like.

Thus, the present invention provides pharmaceutical compositions comprising a compound of the Formula I and a pharmaceutically acceptable diluent.

The compounds of Formula I can be administered by a variety of routes. In effecting treatment of a patient afflicted with disorders described herein, a compound of Formula I can be administered in any form or mode that makes the compound bioavailable in an effective amount, including oral and parenteral routes. For example, compounds of Formula I can be administered orally, by inhalation, or by the subcutaneous, intramuscular, intravenous, transdermal, intranasal, rectal, occular, topical, sublingual, buccal, or other routes. Oral administration is generally preferred for treatment of the neurological and psychiatric disorders described herein.

One skilled in the art of preparing formulations can readily select the proper form and mode of administration depending upon the particular characteristics of the compound selected, the disorder or condition to be treated, the stage of the disorder or condition, and other relevant circumstances. (Remington's Pharmaceutical Sciences, 18th Edition, Mack Publishing Co. (1990)).

The pharmaceutical compositions are prepared in a manner well known in the pharmaceutical art. The carrier or excipient may be a solid, semi-solid, or liquid material that can serve as a vehicle or medium for the active ingredient. Suitable carriers or excipients are well known in the art. The pharmaceutical composition may be adapted for oral, inhalation, parenteral, or topical use and may be administered to the patient in the form of tablets, capsules, aerosols, inhalants, suppositories, solutions, suspensions, or the like.

The compounds of the present invention may be administered orally, for example, with an inert diluent or capsules or compressed into tablets. For the purpose of oral therapeutic administration, the compounds may be incorporated with excipients and used in the form of tablets, troches, capsules, elixirs, suspensions, syrups, wafers, chewing gums and the like. These preparations should contain at least 4% of the compound of the present invention, the active ingredient, but may be varied depending upon the particular form and may conveniently be between 4% to about 70% of the weight of the unit. The amount of the compound present in compositions is such that a suitable dosage will be obtained. Preferred compositions and preparations according to the present invention may be determined by a person skilled in the art.

The tablets, pills, capsules, troches, and the like may also contain one or more of the following adjuvants: binders such as povidone, hydroxypropyl cellulose, microcrystalline cellulose, gum tragacanth or gelatin; excipients such as dicalcium phosphate, starch, or lactose; disintegrating agents such as alginic acid, Primogel, corn starch and the like; lubricants such as talc, magnesium stearate or Sterotex; glidants such as colloidal silicon dioxide; and sweetening agents, such as sucrose, aspartame, or saccharin, or a flavoring agent, such as peppermint, methyl salicylate or orange flavoring, may be added. When the dosage unit form is a capsule, it may contain, in addition to materials of the above type, a liquid carrier such as polyethylene glycol or a fatty oil. Other dosage unit forms may contain other various materials that modify the physical form of the dosage unit, for example, coatings. Thus, tablets or pills may be coated with sugar, shellac, or other coating agents. A syrup may contain, in addition to the present compounds, sucrose as a sweetening agent and certain preservatives, dyes and colorings and flavors. Materials used in preparing these various compositions should be pharmaceutically pure and non-toxic in the amounts used.

For the purpose of parenteral therapeutic administration, the compounds of the present invention may be incorporated into a solution or suspension. These preparations typically contain at least 0.001% of a compound of the invention, but may be varied to be between 0.001 and about 90% of the weight thereof. The amount of the compound of Formula I present in such compositions is such that a suitable dosage will be obtained. The solutions or suspensions may also include one or more of the following adjuvants: sterile diluents, such as water for injection, saline solution, fixed oils, polyethylene glycols, glycerine, propylene glycol or other synthetic solvents; antibacterial agents, such as benzyl alcohol or methyl paraben; antioxidants, such as ascorbic acid or sodium bisulfite; chelating agents, such as ethylene diaminetetraacetic acid; buffers, such as acetates, citrates or phosphates; and agents for the adjustment of tonicity, such as sodium chloride or dextrose. The parenteral preparation can be enclosed in ampoules, disposable syringes or multiple dose vials made of glass or plastic. Preferred compositions and preparations are able to be determined by one skilled in the art.

The compounds of the present invention may also be administered topically, and when done so, the carrier may suitably comprise a solution, ointment, or gel base. The base, for example, may comprise one or more of the following: petrolatum, lanolin, polyethylene glycols, bees wax, mineral oil, diluents such as water and alcohol, and emulsifiers, and stabilizers. Topical formulations may contain a concentration of a compound of Formula I or its pharmaceutical salt from about 0.1 to about 10% w/v (weight per unit volume).

The compounds of Formula I are antagonists of NK-1 receptors. Furthermore, the compounds of Formula I selectively antagonize NK-1 receptors relative to other tachykinin receptors. The antagonist activity of NK-1 receptor antagonists may be determined by the methods below.

NK-1 Receptor Binding Assay

The IM-9 cell line is a well-characterized and readily available human cell line. See, e.g., Annals of the New York Academy of Science, 190: 221-234 (1972); Nature (London), 251:443-444 (1974); Proceedings of the National Academy of Sciences (USA), 71:84-88 (1974). These cells are routinely cultured in RPMI 1640 supplemented with 50 μg/ml gentamicin sulfate and 10% fetal calf serum.

The IM-9 cells are homogenized from cell pellets for crude membranes. The membranes are isolated by homogenizing tissue samples in 30 ml w/v with 50 mM Tris buffer (pH 7.4). After an initial spin at 900×g, the supernatant is transferred to a clean centrifuge tube and the membranes isolated by centrifugation at 38,000×g.

Approximately 25 μg of membranes are incubated with 0.2 nM [¹²⁵I]-substance P (NEN, Boston, Mass.) in a receptor binding assay. The assay buffer contains 50 mM Tris, 3 mM MnCl₂, 0.02% bovine serum albumin, 40 μg/ml bacitracin, 2 μg/ml chymostatin, 4 μg/ml leupeptin and 40 μg/ml thiorphan (pH 7.4). Binding studies are conducted in a final volume of 200 μl containing various concentrations of test compounds. Non-specific binding is determined by incubating some tubes in the presence of 1 μM substance P (Peninsula, Belmont, Calif.).

Binding is terminated 1 hour later by rapid filtration using a TOMTEC 96-well cell harvester (TOMTEC, Orange, Conn.) through GF/A filters that have been presoaked with 0.3% polyethyleneimine (Sigma, St Louis) for 1 hour. The filters are washed with 5 ml of ice-cold 50 mM Tris buffer (pH 7.4) and placed in a drying oven at 60° C. The dried filters are treated with MeltiLex A melt-on scintillator sheets (Wallac, Gaithersburg, Md.), and the radioactivity retained on the filters counted using the Wallac 1205 Betaplate scintillation counter. The results are analyzed using a Log-Logit plot from a Microsoft Excel™ workbook and converted to Ki values with the Cheng-Prusoff equation. Protein concentrations are measured using Coomassie® protein assay reagent (Pierce, Rockford, Ill.), with BSA for standards (Bradford, 1976).

Binding studies are carried out to evaluate the ability of compounds of the present invention to inhibit NK-1 receptor activation. Such studies provide in vitro data regarding the efficacy of the compounds of the present invention. Representative Examples of the compounds of Formula (I) were tested in the receptor binding assay described herein and were demonstrated to have binding affinities (Kᵢ values) of ≦100 nM.

Several preclinical laboratory animal models have been described for a number of the disorders associated with an excess of tachykinins. One such in vivo assay, described below, may be used to determine whether NK-1 receptor antagonists are CNS-penetrant.

Gerbil Foot-Tapping

The gerbil foot-tapping assay is well recognized in the art. For example, see Rupniak et al., Eur. J Pharmacol. (1997) 326: 201-209.

Male Gerbils (Mongolian), weighing between 20-40 gm (Harlan Labs, Indianapolis, Ind.) are used for the experiments. Animals are allowed to acclimate prior to any testing.

An NK-1 receptor agonist, such as GR73632 (δ-Aminovaleryl [Pro⁹, N—Me-Leu¹⁰]-Substance P(7-11)) (Peninsula Labs), is dissolved in acidified saline (1 ml acetic acid in 1 liter of 0.09% saline) to make a 1 mg/ml solution (corrected for peptide content). The stock solution is further diluted to 10 μg/ml in saline (0.9% normal saline), aliquoted and kept frozen until use. The stock solution is further diluted to 3 pmol/5 μl in saline for i.c.v. injections.

Test compounds are formulated in appropriate vehicle to a concentration of 1 ml/100 gm body weight. Compounds are dosed by oral gavage (p.o.) or subcutaneously (s.c.) or intraperitoneally (i.p.) at pre-determined times prior to intracerebroventricular (i.c.v.) challenge of agonist. For i.c.v. administration, test compound is co-injected with agonist.

Free hand i.c.v. injection is performed by direct vertical insertion of a cuffed 27-gauge needle with a Hamilton 50 μl syringe, to a depth of 4.5 mm below bregma. Light anesthesia with isoflurane may be needed prior to the injection, but is not used routinely.

Following i.c.v. injection of agonist, animals are placed in a plexiglas observation box, and hind foot tapping events are counted for 5 minutes. Data collection is computerized.

Data are analyzed by ANOVA followed by Dunnett's test using JMP statistical program (IBM platform). Data are expressed as number of events/5 minutes.

The results of NK-1 receptor binding studies demonstrate the ability of compounds of the present invention to act as antagonists of NK-1 receptors. It is recognized that the compounds of the present invention would be expected to inhibit the effects of NK-1 receptor activation. Thus, the compounds of the present invention are expected to be useful in the treatment of various disorders associated with excess tachykinins, as described to be treated herein, and other disorders that can be treated by such antagonists, as are appreciated by those skilled in the art.

In one embodiment, the present invention provides methods of treating disorders selected from the group consisting of anxiety, depression, psychosis, schizophrenia and other psychotic disorders, neurodegenerative disorders (including senile dementia of the Alzheimer's type, Alzheimer's disease, AIDS-associated dementia, and Down's syndrome), seizure disorders (including generalized and partial seizures), demyelinating diseases (including multiple sclerosis and amyotrophic lateral sclerosis), neuropathological disorders (including peripheral neuropathy, diabetic and chemotherapy-induced neuropathy, and post-herpetic and other neuralgias), acute and chronic obstructive airway diseases (including adult respiratory distress syndrome, bronchopneumonia, bronchospasm, chronic bronchitis, drivercough, and asthma), inflammatory diseases (including inflammatory bowel disease, psoriasis, fibrositis, osteoarthritis, and rheumatoid arthritis), disorders of the musculo-skeletal system (such as osteoporosis), allergies (including eczema and rhinitis), hypersensitivity disorders (such as poison ivy), ophthalmic diseases (such as conjunctivitis, vernal conjunctivitis, and the like), cutaneous diseases (including contact dermatitis), atopic dermatitis, urticaria, other eczematoid dermatites, addiction disorders (including alcoholism), stress-related somatic disorders, reflex sympathetic dystrophy (such as shoulder/hand syndrome), dysthymic disorders, adverse immunological reactions (such as rejection of transplanted tissues), disorders related to immune enhancement or suppression (such as systemic lupus erythematosis), gastrointestinal disorders, diseases associated with the neuronal control of viscera (such as ulcerative colitis, Crohn's disease and irritable bowel syndrome); disorders of bladder function (such as bladder detrusor hyper-reflexia and incontinence), atherosclerosis, fibrosis and collagen diseases (such as scleroderma and eosinophilic fascioliasis), irritative symptoms of benign prostatic hypertrophy, disorders associated with blood pressure (such as hypertension), disorders of blood flow caused by vasodilation or vasospastic diseases (such as angina, migraine, and Reynaud's disease), emesis (including chemotherapy-induced nausea and acute or delayed emesis), and pain or nociception (including that attributable to or associated with any of the foregoing conditions), comprising: administering to a patient in need thereof an effective amount of a compound of Formula I or a pharmaceutical composition thereof. That is, the present invention provides methods of treating disorders associated with an excess of tachykinins, comprising: administering to a patient in need thereof an effective amount of a compound of Formula I or a pharmaceutical composition thereof.

The present invention contemplates the various disorders described to be treated herein and others that can be treated by such antagonists, as appreciated by those skilled in the art.

The disorders associated with an excess of tachykinins are treated by administering an effective amount of a compound or pharmaceutical composition of Formula 1. An effective amount can be readily determined by the attending diagnostician, as one skilled in the art, by the use of conventional techniques and by observing results obtained under analogous circumstances. In determining an effective amount, the dose of a compound of Formula I, a number of factors are considered by the attending diagnostician, including, but not limited to: the compound of Formula I to be administered; the species of mammal—its size, age, and general health; the specific disorder involved; the degree of involvement or the severity of the disorder; the response of the individual patient; the mode of administration; the bioavailability characteristics of the preparation administered; the dose regimen selected; the use of other concomitant medication; and other relevant circumstances.

An effective amount of a compound of Formula I is expected to vary from about 0.001 milligram per kilogram of body weight per day (mg/kg/day) to about 100 mg/kg/day. Preferred amounts may be readily determined by one skilled in the art.

Of the disorders associated with an excess of tachykinins that are treated according to the present invention, the treatment of depression, anxiety, inflammatory bowel disease, irritable bowel syndrome, and emesis (chemotherapy-induced nausea and acute or delayed emesis) are particularly preferred.

Thus, in a preferred embodiment, the present invention provides a method for treating a depressive disorder, including major depressive disorder, comprising: administering to a patient in need thereof an effective amount of a compound of Formula I or a pharmaceutical composition thereof.

In another preferred embodiment, the present invention provides a method for treating anxiety, including generalized anxiety disorder, panic disorder, and obsessive-compulsive disorder, comprising: administering to a patient in need thereof an effective amount of a compound of Formula I or a pharmaceutical composition thereof.

Disorders of the central nervous system, including depressive and anxiety disorders, have been characterized in the Diagnostic and Statistical Manual of Mental Disorders (DSM-IV™) (1994, American Psychiatric Association, Washington, D.C.). The DSM-IV™ provides clear descriptions of diagnostic categories. The skilled artisan will recognize that there are alternative nomenclatures, nosologies, and classification systems for these disorders, and that these systems may evolve with medical scientific progress. For instance, the ICHPPC-2 (International Classification of Health Problems in Primary Care) (3^{rd} edition, 1983, Oxford University Press, Oxford) provides an alternative classification system. Thus, the terms “depression,” “depressive disorders,” “anxiety,” and “anxiety disorders” are intended to include like disorders that are described in other diagnostic sources.

According to the fourth edition of the DSM-IV™, major depressive disorders are characterized by one or more major depressive episodes, which consist of a period of at least two weeks of depressed mood or loss of pleasure, in addition to other symptoms. Thus, the skilled artisan will recognize that the present invention is useful for the treatment of either a single episode or recurrent episodes of major depressive disorder.

The skilled artisan will appreciate that other depressive disorders may also be treated by administering an effective amount of a compound of Formula (I). Such other depressive disorders include dysthymic disorder, and depressive disorders not otherwise specified (for example, premenstrual dysphoric disorder, minor depressive disorder, recurrent brief depressive disorder, or postpsychotic depressive disorder of schizophrenia). In addition, the treatment of depression by the compounds of Formula (I) may also include the treatment of mood disorders due to a general medical condition and substance-induced mood disorders.

The DSM-IV™ also provides a diagnostic tool for anxiety and related disorders. These disorders include: panic disorder with or without agoraphobia, agoraphobia without history of panic disorder, specific phobia, social phobia or social anxiety disorder, obsessive-compulsive disorder, post-traumatic stress disorder, acute stress disorder, generalized anxiety disorder, anxiety disorder due to a general medical condition, substance-induced anxiety disorder and anxiety disorder not otherwise specified. As used herein, the term “anxiety” includes treatment of those anxiety disorders and related disorders described in the DSM-IV.

1. A compound of Formula I: 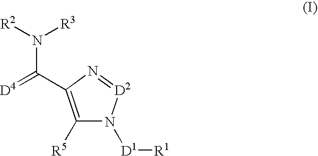
wherein:
D¹ is a C₁-C₃ alkane-diyl;
D² is CH or nitrogen;
D⁴ is oxygen or sulfur;
R¹ is phenyl,
which phenyl is optionally substituted with one to three substitutents independently selected from the group consisting of halo, C₁-C₄ alkyl, C₁-C₄ alkoxy, cyano, difluoromethyl, trifluoromethyl, and trifluoromethoxy;
R² is selected from the group consisting of hydroxy, C₁-C₄ alkyl, optionally substituted phenyl, naphthyl, C₃-C₁₀ cycloalkyl, pyridyl, optionally substituted pyrrolidinyl, optionally substituted piperidinyl,
which C₁-C₄ alkyl is optionally substituted with hydroxy, C₁-C₂ alkoxy, optionally substituted phenyl, pyridyl, —NR⁶R⁷, or naphthyl;
which pyridyl is further optionally substituted with one to two halo, C₁-C₃ alkyl;
R³ is C₁-C₄ alkyl, optionally substituted phenyl, —C(O)—R⁴, or —S(O)₂—R⁴,
which C₁-C₄ alkyl is further optionally substituted with R⁴;
R⁴ is optionally substituted phenyl;
or R² and R³, together with the nitrogen to which they are attached, form a 4-11 membered heterocyclic ring,
which heterocyclic ring is further optionally substituted with one to four substituents independently selected from the group consisting of optionally substituted phenyl, C₃-C₆ cycloalkyl, pyridyl, halo, hydroxy, oxo, and C₁-C₄ alkyl;
wherein the C₁-C₄ alkyl is further optionally substituted with one to two substituents selected from the group consisting of C₁-C₃ alkoxy, optionally substituted phenyl, oxo, phenoxy, pyridyl, and pyrrolidinyl;
R⁶ and R⁷ are each independently hydrogen, C₁-C₄ alkyl, —S(O)₂—CH₃, or C₁-C₄ alkoxycarbonyl, or R⁶ and R⁷, together with the nitrogen to which they are attached, form a 4-7 membered saturated heterocyclic ring;
R⁵ is hydrogen, halo, trifluoromethyl, C₁-C₄ alkyl, C₁-C₄ alkoxy, C₃-C₆ cycloalkyl, furyl, pyrazolyl, imidazolyl, —NR¹³R¹⁴, pyridyloxy, benzyloxy, phenyl, phenoxy, pyrrolyl, thienyl, phenylthio, or anilino,
which phenyl, phenoxy, pyrrolyl, thienyl, phenylthio, or anilino group may be optionally substituted on the ring with one to two substituents independently selected from the group consisting of halo, C₁-C₄ alkyl, C₁-C₄ alkoxy, trifluoromethyl, and —S(O)_{q}(C₁-C₄ alkyl),
or R⁵ is a radical selected from the group consisting of: 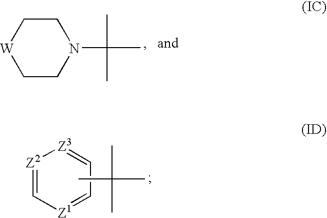
wherein
W is a bond, —CHR¹⁵—, —C(O)—, —O—, —NR¹⁵—, or —S(O)_{q}—;
q is 0, 1, or 2;
R¹⁵ is selected from the group consisting of hydrogen, hydroxy, C₁-C₄ alkyl, acetyl, carbamoyl, phenyl, benzyl, and —S(O)₂CH₃;
Z¹, Z², and Z³ are each independently CH or nitrogen;
R¹³ and R¹⁴ are each independently hydrogen, C₁-C₄ alkyl, —S(O)₂—CH₃ or C₃-C₆ cycloalkyl;
wherein the C₁-C₄ alkyl is optionally substituted with one C₁-C₂ alkoxy or di(C₁-C₂ alkyl)amino;
or R¹³ and R¹⁴, together with the nitrogen to which they are attached, form a 4-7 membered saturated heterocyclic ring;
which 4-7 membered saturated heterocyclic ring is further optionally substituted with one to two C₁-C₂ alkyl;
or a pharmaceutically acceptable salt thereof;
with the proviso that the following compounds are not claimed: [5-methyl-1-(3-pyrrolidin-1-ylpropyl)-1H-1,2,3-triazol-4-yl]piperazin-1-yl-methanone; {1-[2-(4-nitrophenyl)ethyl]-5-methyl-1H-1,2,3-triazol-4-yl}piperazin-1-yl-methanone; [1-(4-methoxybenzyl)-5-methyl-1H-1,2,3-triazol-4-yl]piperazin-1-yl-methanone; [5-methyl-1-(3-imidazol-1-ylpropyl)-1H-1,2,3-triazol-4-yl]piperazin-1-yl-methanone; (5-methyl-1-benzyl-1H-1,2,3-triazol-4-yl)piperazin-1-yl-methanone; (1-benzyl-5-methyl-1H-1,2,3-triazol-4-yl)-1,4-diazepan-1-yl-methanone; [1-(3,5-bis-trifluoromethyl-benzyl)-5-morpholin-4-yl-1H-[1,2,3]triazol-4-yl]-morpholin-4-yl-methanone; 1-(3,5-bis-trifluoromethyl-benzyl)-5-pyridin-4-yl-1H-[1,2,3]triazole-4-carboxylic acid (2-amino-ethyl)-(2-chloro-benzyl)-amide dihydrochloride; 1-(3,5-bis-trifluoromethyl-benzyl)-5-morpholin-4-yl-1H-[1,2,3]triazole-4-carboxylic acid (2-amino-ethyl)-(2-chloro-benzyl)-amide hydrochloride; 1-(3,5-bis-trifluoromethyl-benzyl)-5-morpholin-4-yl-1H-[1,2,3]triazole-4-carboxylic acid (2-amino-ethyl)-[1-(2-chloro-phenyl)-ethyl]-amide dihydrochloride; 1-(3,5-bis-trifluoromethyl-benzyl)-5-pyridyl-4-yl-1H-[1,2,3]triazole-4-carboxylic acid (2-amino-ethyl)-[1-(2-chloro-phenyl)-ethyl]-amide dihydrochloride; {2-[[1-(3,5-bis-trifluoromethyl-benzyl)-5-pyridin-4-yl-1H-[1,2,3]triazole-4-carbonyl-(2-chloro-benzyl)-amino]-ethyl}-carbamic acid tert-butyl ester; {2-[[1-(3,5-bis-trifluoromethyl-benzyl)-5-chloro-1H-[1,2,3]triazole-4-carbonyl]-(2-chloro-benzyl)-amino]-ethyl}-carbamic acid tert-butyl ester; (2-{[1-(3,5-bis-trifluoromethyl-benzyl)-5-chloro-1H-[1,2,3]triazole-4-carbonyl]-[1-(2-chloro-phenyl)-ethyl]-amino}-ethyl)-carbamic acid tert-butyl ester; (2-{[1-(3,5-bis-trifluoromethyl-benzyl)-5-pyridin-4-yl-1H-[1,2,3]triazole-4-carbonyl]-[1-(2-chloro-phenyl)-ethyl]-amino}-ethyl)-carbamic acid tert-butyl ester; {2-[[1-(3,5-bis-trifluoromethyl-benzyl)-5-morpholin-4-yl-1H-[1,2,3]triazole-4-carbonyl]-(2-chloro-benzyl)-amino]-ethyl}-carbamic acid tert-butyl ester; and (2-{[1-(3,5-bis-trifluoromethyl-benzyl)-5-morpholin-4-yl-1H-[1,2,3]triazole-4-carbonyl]-[1-(2-chloro-phenyl)-ethyl]-amino}-ethyl)-carbamic acid tert-butyl ester.

2. The compound of claim 1 wherein D⁴ is oxygen.

3. The compound of claim 2 wherein D² is nitrogen.

4. The compound of claim 3 wherein D¹ is methylene.

5. The compound of claim 4 wherein R¹ is 3,5-bis-trifluoromethyl-phenyl.

6. The compound of claim 5 wherein R⁵ is phenyl.

7. The compound of claim 6 wherein R² is C₁-C₄ alkyl, which is optionally substituted with optionally substituted phenyl.

8. The compound of claim 7 wherein R² is 2-chloro-benzyl.

9. The compound of claim 8 wherein R³ is C₁-C₄ alkyl, which C₁-C₄ alkyl is optionally substituted with R⁴.

10. The compound of claim 9 wherein R³ is methyl.

11. The compound of claim 6 wherein R² and R³, together with the nitrogen to which they are attached, form a 4-11 membered heterocyclic ring, which heterocyclic ring is further optionally substituted with one to four substituents independently selected from the group consisting of optionally substituted phenyl, C₃-C₆ cycloalkyl, pyridyl, halo, hydroxy, oxo, and C₁-C₄ alkyl,
wherein the C₁-C₄ alkyl is further optionally substituted with one to two substituents selected from the group consisting of C₁-C₃ alkoxy, optionally substituted phenyl, oxo, phenoxy, pyridyl, and pyrrolidinyl.

12. The compound of claim 11 wherein R² and R³, together with the nitrogen to which they are attached, form pyrrolidin-1-yl, which pyrrolidin-1-yl is further optionally substituted with one to four substituents independently selected from the group consisting of optionally substituted phenyl, C₃-C₆ cycloalkyl, pyridyl, halo, hydroxy, oxo, and C₁-C₄ alkyl,
wherein the C₁-C₄ alkyl is further optionally substituted with one to two substituents selected from the group consisting of C₁-C₃ alkoxy, optionally substituted phenyl, oxo, phenoxy, pyridyl, and pyrrolidinyl.

13. The compound of claim 12 wherein R² and R³, together with the nitrogen to which they are attached, form 2-(2-chloro-phenyl)-pyrrolidin-1-yl.

14. The compound of claim 1 wherein the compound is 1-(3,5-Bis-trifluoromethyl-benzyl)-5-phenyl-1H-[1,2,3]triazole-4-carboxylic acid (2-chloro-benzyl)-methyl-amide.

15. The compound of claim 1 wherein the compound is [1-(3,5-Bis-trifluoromethyl-benzyl)-5-phenyl-1H-[1,2,3]triazol-4-yl]-[2-(2-chloro-phenyl)-pyrrolidin-1-yl]-methanone.

16. A pharmaceutical composition comprising a compound of claim 1, or a pharmaceutically acceptable salt thereof, in combination with a pharmaceutically acceptable carrier, excipient, or diluent.

17. A method for treating a condition associated with an excess of tachykinins, comprising: administering to a patient in need thereof an effective amount of a compound of Formula (I): 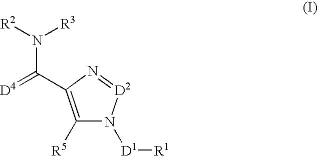
wherein:
D¹ is a C₁-C₃ alkane-diyl;
D² is CH or nitrogen;
D⁴ is oxygen or sulfur;
R¹ is phenyl,
which phenyl is optionally substituted with one to three substitutents independently selected from the group consisting of halo, C₁-C₄ alkyl, C₁-C₄ alkoxy, cyano, difluoromethyl, trifluoromethyl, and trifluoromethoxy;
R² is selected from the group consisting of hydroxy, C₁-C₄ alkyl, optionally substituted phenyl, naphthyl, C₃-C₁₀ cycloalkyl, pyridyl, optionally substituted pyrrolidinyl, optionally substituted piperidinyl,
which C₁-C₄ alkyl is optionally substituted with hydroxy, C₁-C₂ alkoxy, optionally substituted phenyl, pyridyl, —NR⁶R⁷, or naphthyl;
which pyridyl is further optionally substituted with one to two halo, C₁-C₃ alkyl;
R³ is C₁-C₄ alkyl, optionally substituted phenyl, —C(O)—R⁴; or —S(O)₂—R⁴,
which C₁-C₄ alkyl is further optionally substituted with R⁴;
R⁴is optionally substituted phenyl;
or R² and R³, together with the nitrogen to which they are attached, form a 4-11 membered heterocyclic ring,
which heterocyclic ring is further optionally substituted with one to four substituents independently selected from the group consisting of optionally substituted phenyl, C₃-C₆ cycloalkyl, pyridyl, halo, hydroxy, oxo, and C₁-C₄ alkyl;
wherein the C₁-C₄ alkyl is further optionally substituted with one to two substituents selected from the group consisting of C₁-C₃ alkoxy, optionally substituted phenyl, oxo, phenoxy, pyridyl, and pyrrolidinyl;
R⁶ and R⁷ are each independently hydrogen, C₁-C₄ alkyl, —S(O)₂—CH₃, or C₁-C₄ alkoxycarbonyl, or R⁶ and R⁷, together with the nitrogen to which they are attached, form a 4-7 membered saturated heterocyclic ring;
R⁵ is hydrogen, halo, trifluoromethyl, C₁-C₄ alkyl, C₁-C₄ alkoxy, C₃-C₆ cycloalkyl, furyl, pyrazolyl, imidazolyl, —NR¹³R¹⁴, pyridyloxy, benzyloxy, phenyl, phenoxy, pyrrolyl, thienyl, phenylthio, or anilino,
which phenyl, phenoxy, pyrrolyl, thienyl, phenylthio, or anilino group may be optionally substituted on the ring with one to two substituents independently selected from the group consisting of halo, C₁-C₄ alkyl, C₁-C₄ alkoxy, trifluoromethyl, and —S(O)_{q}(C₁-C₄ alkyl),
or R⁵ is a radical selected from the group consisting of: 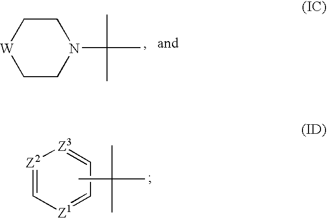
wherein
W is a bond, —CHR¹⁵—, —C(O)—, —O—, —NR¹⁵—, or —S(O)_{q}—;
q is 0, 1, or 2;
R¹⁵ is selected from the group consisting of hydrogen, hydroxy, C₁-C₄ alkyl, acetyl, carbamoyl, phenyl, benzyl, and —S(O)₂CH₃;
Z¹, Z², and Z³ are each independently CH or nitrogen;
R¹³ and R¹⁴ are each independently hydrogen, C₁-C₄ alkyl, —S(O)₂—CH₃ or C₃-C₆ cycloalkyl;
wherein the C₁-C₄ alkyl is optionally substituted with one C₁-C₂ alkoxy or di(C₁-C₂ alkyl)amino;
or R¹³ and R¹⁴, together with the nitrogen to which they are attached, form a 4-7 membered saturated heterocyclic ring;
which 4-7 membered saturated heterocyclic ring is further optionally substituted with one to two C₁-C₂ alkyl;
or a pharmaceutically acceptable salt thereof.

18. The method of claim 17 wherein the condition associated with an excess of tachykinins is selected from the group consisting of depression, anxiety, irritable bowel syndrome, and emesis.

19-20. (canceled)

21. A compound selected from the group consisting of: [1-(3,5-Bis-trifluoromethyl-benzyl)-5-(1-oxy-pyridin-4-yl)-1H-[1,2,3]triazol-4-yl]-[2-(2-chloro-phenyl)pyrrolidin-1-yl)-methanone, [1-(3,5-Bis-trifluoromethyl-benzyl)-5-(1-oxy-pyridin-3-yl)-1H-[1,2,3]triazol-4-yl]-[2-(2-chloro-phenyl)-pyrrolidin-1-yl]-methanone, and (R)-[1-(3,5-Bis-trifluoromethyl-benzyl)-5-(3,6-dihydro-2H-pyridin-1-yl)-1H-[1,2,3]triazol-4-yl]-[2-(2-chloro-phenyl)-pyrrolidin-1-yl]-methanone.
